(19) United States
(12) Patent Application Publication
(10) Pub. No.: US 2021/0395366 A1
(43) Pub. Date: Dec. 23, 2021

(54) TREATMENT WITH ANTI-TIGIT ANTIBODIES AND PD-1 AXIS BINDING ANTAGONISTS

(71) Applicants: Genentech, Inc., South San Francisco, CA (US); Hoffmann-La Roche Inc., Little Falls, NJ (US)

(72) Inventors: Shi LI, San Carlos, CA (US); Meghna Das Thakur HARRIS, South San Francisco, CA (US); Yifan WANG, Shanghai (CN); Edward Namserk CHA, San Carlos, CA (US); Feijiao GE, Shanghai (CN)

(21) Appl. No.: 17/159,063

(22) Filed: Jan. 26, 2021

(30) Foreign Application Priority Data
Jun. 18, 2020 (CN) PCT/CN2020/096746

(51) Int. Cl. C07K 16/28; A61P 35/00; A61K 33/243; A61N 5/00; A61K 31/337

(52) U.S. Cl. CPC C07K 16/2818; A61P 35/00; A61K 33/243; A61K 2039/545; C07K 16/2827; A61K 31/337; A61N 5/00

(57) ABSTRACT

The present invention relates to the treatment of esophageal cancer, e.g., esophageal squamous cell carcinoma (ESCC) (e.g., advanced ESCC (e.g., unresectable, locally advanced, recurrent, and/or metastatic ESCC)). More specifically, the invention pertains to the treatment of patients having esophageal cancer by administering a combination of an anti-T-cell immunoreceptor with Ig and ITIM domains (TIGIT) antagonist antibody and a programmed death-1 (PD-1) axis binding antagonist.

### CROSS-REFERENCES TO RELATED APPLICATIONS

This application claims priority to International Application No. PCT/CN2020/096746, filed on Jun. 18, 2020, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 26, 2021, is named 50474-236002_Sequence_Listing_1.26.21_5 T25 and is 30,046 bytes in size.

### FIELD OF THE INVENTION

The present invention relates to the treatment of esophageal cancer, e.g., esophageal squamous cell carcinoma (ESCC) (e.g., advanced ESCC (e.g., unresectable, locally advanced, recurrent, and/or metastatic ESCC)). More specifically, the invention pertains to the treatment of patients having esophageal cancer by administering a combination of an anti-T-cell immunoreceptor with Ig and ITIM domains (TIGIT) antagonist antibody and a programmed death-1 (PD-1) axis binding antagonist.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

Esophageal cancer is the seventh most commonly diagnosed cancer worldwide and the sixth most common cause of cancer-related death, with an incidence in 2018 of approximately 572,000 new cases and a mortality of 509,000.

Esophageal squamous cell carcinoma (ESCC) accounts for ˜78% of all esophageal cases worldwide. Most esophageal cancer patients are diagnosed with advanced disease, where the disease is frequently recurrent. Treatments can extend survival but are largely palliative, and median survival time is less than one year. The prognosis of esophageal squamous cell carcinoma remains poor, and 5-year survival rates are between 10% and 20% across the U.S., Europe, and Asia.

Thus, there is an unmet need in the field for the development of efficacious immunotherapies for the treatment of esophageal cancer, e.g., ESCC (e.g., advanced ESCC).

### SUMMARY OF THE INVENTION

The present invention involves methods of treating a subject having esophageal cancer (e.g., esophageal squamous cell carcinoma (ESCC), e.g., advanced ESCC) by administering a combination of an anti-TIGIT antagonist antibody (e.g., an anti-TIGIT antagonist antibody, e.g., tiragolumab) and a PD-1 axis binding antagonist (e.g., an anti-PD-L1 antagonist antibody (e.g., atezolizumab)). In some aspects, the invention involves methods of treating a subject or population of subjects that has previously received definitive chemoradiation treatment for esophageal cancer, e.g., ESCC. In some aspects, the invention involves methods of treating a subject or population of subjects that has an advanced esophageal cancer, e.g., advanced ESCC, wherein the subject or population of subjects has received no prior systemic treatment for the advanced esophageal cancer, e.g., advanced ESCC.

In one aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 30 mg to about 1200 mg every three weeks (e.g., about 30 mg to about 600 mg every three weeks, e.g., about 600 mg every three weeks)) and a PD-1 axis binding antagonist (e.g., at a fixed dose of about 80 mg to about 1600 mg every three weeks (e.g., about 800 mg to about 1400 mg every three weeks, e.g., about 1200 mg every three weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 30 mg to about 1200 mg every three weeks (e.g., about 30 mg to about 600 mg every three weeks, e.g., about 600 mg every three weeks)) and a PD-1 axis binding antagonist (e.g., at a fixed dose of about 80 mg to about 1600 mg every three weeks (e.g., about 800 mg to about 1400 mg every three weeks, e.g., about 1200 mg every three weeks)), wherein the subject or population of subjects previously received definitive chemoradiation treatment (e.g., definitive concurrent chemoradiation treatment) for ESCC. In some embodiments, the definitive chemoradiation treatment was completed no more than 89 days prior to administration with the anti-TIGIT antagonist antibody or the PD-1 axis binding antagonist. In some embodiments, the definitive chemoradiation treatment comprises at least two cycles of platinum-based chemotherapy and radiation therapy without evidence of radiographic disease progression. In some embodiments, no chemotherapy is administered to the subject or population of subjects during the one or more dosing cycles. In some embodiments, the anti-TIGIT antagonist antibody is administered at a fixed dose of about 600 mg every three weeks and the PD-1 axis binding antagonist is administered at a fixed dose of about 1200 mg every three weeks.

In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)) and a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)) and a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)), wherein the subject or population of subjects previously received definitive chemoradiation treatment (e.g., definitive concurrent chemoradiation treatment) for ESCC. In some embodiments, the anti-TIGIT antagonist antibody is administered at a fixed dose of about 420 mg every two weeks and the PD-1 axis binding antagonist is administered at a fixed dose of about 840 mg every two weeks.

In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks) and a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks) and a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)), wherein the subject or population of subjects previously received definitive chemoradiation treatment (e.g., definitive concurrent chemoradiation treatment) for ESCC. In some embodiments, the anti-TIGIT antagonist antibody is administered at a fixed dose of about 840 mg every four weeks and the PD-1 axis binding antagonist is administered at a fixed dose of about 1680 mg every four weeks.

In some embodiments, the anti-TIGIT antagonist antibody comprises the following hypervariable regions (HVRs): an HVR-H1 sequence comprising the amino acid sequence of SNSAAWN (SEQ ID NO: 1); an HVR-H2 sequence comprising the amino acid sequence of KTYYRFKWYSDYAVSVKG (SEQ ID NO: 2); an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6). In some embodiments, the anti-TIGIT antagonist antibody further comprises the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10). In some embodiments, the anti-TIGIT antagonist antibody further comprises the following heavy chain variable region FRs: an FR-H1 comprising the amino acid sequence of XiVQLQQSGPGLVKPSQTLSLTCAISGDSVS (SEQ ID NO: 11), wherein Xi is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In some embodiments, Xi is E. In other embodiments, Xi is Q. In some embodiments, the anti-TIGIT antagonist antibody comprises: (a) a heavy chain variable (VH) domain comprising an amino acid sequence having at least 95% sequence identity to the amino acid sequence of SEQ ID NO: 17 or 18; (b) a light chain variable (VL) domain comprising an amino acid sequence having at least 95% sequence identity to the amino acid sequence of SEQ ID NO: 19; or (c) a VH domain as in (a) and a VL domain as in (b). In some embodiments, the anti-TIGIT antagonist antibody comprises: (a) a VH domain comprising the amino acid sequence of SEQ ID NO: 17 or 18; and (b) a VL domain comprising the amino acid sequence of SEQ ID NO: 19.

In some embodiments, the anti-TIGIT antagonist antibody is a monoclonal antibody. In some embodiments, the anti-TIGIT antagonist antibody is a human antibody. In some embodiments, the anti-TIGIT antagonist antibody is a full-length antibody. In some embodiments, the anti-TIGIT antagonist antibody is tiragolumab.

In some embodiments, the anti-TIGIT antagonist antibody is an antibody fragment that binds TIGIT selected from the group consisting of Fab, Fab′, Fab′-SH, Fv, single chain variable fragment (scFv), and (Fab′)₂ fragments. In some embodiments, the anti-TIGIT antagonist antibody is an IgG class antibody (e.g., an IgG1 subclass antibody). In some embodiments, the PD-1 axis binding antagonist is a PD-L1 binding antagonist or a PD-1 binding antagonist. In some embodiments, the PD-1 binding antagonist is an anti-PD-1 antagonist antibody, e.g., nivolumab (MDX-1106), pembrolizumab (MK-3475), or AMP-224. In some embodiments, the PD-L1 binding antagonist is an anti-PD-L1 antagonist antibody (e.g., atezolizumab (MPDL3280A), MSB0010718C, MDX-1105, or MEDI4736). In some embodiments, the anti-PD-L1 antagonist antibody is atezolizumab. In some embodiments, the anti-PD-L1 antagonist antibody comprises the following HVRs: an HVR-H1 sequence comprising the amino acid sequence of GFTFSDSWIH (SEQ ID NO: 20); an HVR-H2 sequence comprising the amino acid sequence of AWISPYGGSTYYADSVKG (SEQ ID NO: 21); an HVR-H3 sequence comprising the amino acid sequence of RHWPGGFDY (SEQ ID NO: 22); an HVR-L1 sequence comprising the amino acid sequence of RASQDVSTAVA (SEQ ID NO: 23); an HVR-L2 sequence comprising the amino acid sequence of SASFLYS (SEQ ID NO: 24); and an HVR-L3 sequence comprising the amino acid sequence of QQYLYHPAT (SEQ ID NO: 25). In some embodiments, the anti-PD-L1 antagonist antibody comprises: (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b). In some embodiments, the anti-PD-L1 antagonist antibody comprises: a VH domain comprising the amino acid sequence of SEQ ID NO: 26; and a VL domain comprising the amino acid sequence of SEQ ID NO: 27. In some embodiments, the anti-PD-L1 antagonist antibody is a monoclonal antibody. In some embodiments, the anti-PD-L1 antagonist antibody is a humanized antibody. In some embodiments, the anti-PD-L1 antagonist antibody is a full-length antibody.

In some embodiments, the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments. In some embodiments, the anti-PD-L1 antagonist antibody is an IgG class antibody (e.g., an IgG1 subclass antibody).

In some embodiments, the length of each of the one or more dosing cycles is 21 days. In some embodiments, the method comprises administering to the subject or population of subjects the anti-TIGIT antagonist antibody and the PD-1 axis binding antagonist on about Day 1 of each of the one or more dosing cycles.

In some embodiments, the method comprises administering to the subject or population of subjects the PD-1 axis binding antagonist before the anti-TIGIT antagonist antibody. In some embodiments, the method comprises a first observation period following administration of the PD-1 axis binding antagonist and a second observation period following administration of the anti-TIGIT antagonist antibody. In some embodiment, the first observation period and the second observation period are each between about 30 minutes to about 60 minutes in length. In some embodiments, no infusion-related reaction (IRR) is observed in the first observation period and/or the second observation period.

In some embodiments, the method comprises administering to the subject or population of subjects the anti-TIGIT antagonist antibody before the PD-1 axis binding antagonist. In some embodiments, the method comprises a first observation period following administration of the anti-TIGIT antagonist antibody and a second observation period following administration of the PD-1 axis binding antagonist. In some embodiments, the first observation period and the second observation period are each between about 30 minutes to about 60 minutes in length. In some embodiments, no IRR is observed in the first observation period and/or the second observation period.

In some embodiments, the method comprises administering to the subject or population of subjects the anti-TIGIT antagonist antibody and the PD-1 axis binding antagonist simultaneously.

In some embodiments, the method comprises administering to the subject or population of subjects the anti-TIGIT antagonist antibody and the PD-1 axis binding antagonist intravenously. In some embodiments, the method comprises administering to the subject or population of subjects the anti-TIGIT antagonist antibody by intravenous infusion over 60±10 minutes. In some embodiments, the method comprises administering to the subject or population of subjects the PD-1 axis binding antagonist by intravenous infusion over 60±15 minutes. In some embodiments, the anti-TIGIT antagonist antibody is administered subcutaneously. In some embodiments, the PD-1 axis binding antagonist is administered subcutaneously. In some embodiments, the anti-TIGIT antagonist antibody and the PD-1 axis binding antagonist are administered subcutaneously.

In some embodiments of any of the preceding methods, an ESCC tumor sample obtained from the subject or population of subjects has been determined to have a detectable expression level of PD-L1 (e.g., a detectable protein expression level of PD-L1 or a detectable protein expression level of PD-L1 has been determined by an immunohistochemical (IHC) assay). In some embodiments, the IHC assay uses anti-PD-L1 antibody SP263, 22C3, SP142, or 28-8. In some embodiments, the IHC assay uses anti-PD-L1 antibody SP263. In some embodiments, the IHC assay is the Ventana SP263 IHC assay. In some embodiments, the ESCC tumor sample has been determined to have a tumor and tumor-associated immune cell (TIC) score of greater than, or equal to, 1%. In some embodiments, the TIC score is greater than, or equal to, 10%. In some embodiments, the ESCC tumor sample has been determined to have a TIC score of less than 10%. In some embodiments, the ESCC tumor sample has been determined to have a TIC score of greater than, or equal to, 10% and less than 50%. In some embodiments, the ESCC tumor sample has been determined to have a TIC score greater than, or equal to, 10%, as determined using the anti-PDL1 antibody SP263 as part of the Ventana SP263 IHC assay (companion CDx assay), and the PD-1 axis binding antagonist administered in combination with the anti-TIGIT antagonist antibody (e.g., tiragolumab) is atezolizumab.

In some embodiments, the IHC assay uses the anti-PD-L1 antibody 22C3 (e.g., for use in the pharmDx 22C3 IHC assay). In some embodiments, the ESCC tumor sample has been determined to have a combined positive score (CPS) of greater than, or equal to, 10. For example, in some embodiments, the ESCC tumor sample has been determined to have a CPS of greater than, or equal to, 10, as determined using the anti-PDL1 antibody 22C3 as part of the pharmDx22C3 IHC assay, and the PD-1 axis binding antagonist administered in combination with the anti-TIGIT antagonist antibody (e.g., tiragolumab) is pembrolizumab. In some embodiments, the ESCC tumor sample has been determined to have a CPS of greater than, or equal to, 10, as determined using the anti-PDL1 antibody 22C3 as part of the pharmDx22C3 IHC assay, and the PD-1 axis binding antagonist administered in combination with the anti-TIGIT antagonist antibody (e.g., tiragolumab) is atezolizumab. In some embodiments, the ESCC tumor sample has been determined to have a tumor proportion score (TPS) of greater than, or equal to, 1%. In some embodiments, the ESCC tumor sample has been determined to have a TPS of greater than, or equal to, 50%.

In some embodiments, the IHC assay uses the anti-PD-L1 antibody SP142 (e.g., for use in the Ventana SP142 IHC assay). In some embodiments, the IHC assay uses the anti-PD-L1 antibody 28-8 (e.g., for use in the pharmDx 28-8 IHC assay). In some embodiments, the detectable expression level of PD-L1 is a detectable nucleic acid expression level of PD-L1 (e.g., as determined by RNA-seq, RT-qPCR, qPCR, multiplex qPCR or RT-qPCR, microarray analysis, SAGE, MassARRAY technique, ISH, or a combination thereof).

In some embodiments, the ESCC is a locally advanced ESCC. In some embodiments, the ESCC is an unresectable ESCC. In some embodiments, the ESCC is a recurrent or metastatic ESCC. In some embodiments, the ESCC comprises a cervical esophageal tumor. In some embodiments, the ESCC is a Stage II ESCC, a Stage III ESCC, or a Stage IV ESCC. In some embodiments, the Stage IV ESCC is a Stage IVA ESCC or a Stage IVB ESCC with supraclavicular lymph node (SCLN) metastases only.

In some embodiments of any of the preceding methods, the treatment results in an increase in progression-free survival (PFS) of the subject or population of subjects as compared to treatment with the PD-1 axis binding antagonist without the anti-TIGIT antagonist antibody. In some embodiments, the treatment results in an increase in PFS of the subject or population of subjects as compared to treatment with the anti-TIGIT antagonist antibody without the PD-1 axis binding antagonist. In some embodiments, the treatment results in an increase in PFS of the subject or population of subjects as compared to treatment without the anti-TIGIT antagonist antibody and without the PD-1 axis binding antagonist. In some embodiments, the treatment extends the PFS of the subject or population of subjects by at least about 4 months or about 8 months. In some embodiments, the increase in PFS is about 8 months or more (e.g., about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more). In some embodiments, the increase in PFS is about 4 months, about 5 months, about 6 months, or about 7 months. In some embodiments, the treatment results in a median PFS of the population of subjects of about 15 months to about 23 months. In some embodiments, administration of the anti-TIGIT antagonist antibody (e.g., tiragolumab) and the PD-1 axis binding antagonist (e.g., atezolizumab) to a plurality of subjects results in a median PFS of at least about 15 months (e.g., about 15.5 months, about 16 months, about 16.5 months, about 17 months, about 17.5 months, about 18 months, or about 18.5 months) after the start of treatment with the anti-TIGIT antagonist antibody (e.g., tiragolumab) and the PD-1 axis binding antagonist (e.g., atezolizumab). In some embodiments, administration of the anti-TIGIT antagonist antibody (e.g., tiragolumab) and the PD-1 axis binding antagonist (e.g., atezolizumab) to a plurality of subjects results in a median PFS of at least about 19 months, (e.g., about 19.5 months, about 20 months, about 20.5 months, about 21 months, about 21.5 months, about 22 months, about 22.5 months, or about 23 months, or more) after the start of treatment with the anti-TIGIT antagonist antibody (e.g., tiragolumab) and the PD-1 axis binding antagonist (e.g., atezolizumab).

In some embodiments of any of the preceding methods, the treatment results in an increase in overall survival (OS) of the subject or population of subjects as compared to treatment with the PD-1 axis binding antagonist without the anti-TIGIT antagonist antibody. In some embodiments, the treatment results in an increase in OS of the subject or population of subjects as compared to treatment with the anti-TIGIT antagonist antibody and without treatment with the PD-1 axis binding antagonist. In some embodiments, the treatment results in an increase in OS of the subject or population of subjects as compared to treatment without the anti-TIGIT antagonist antibody and without the PD-1 axis binding antagonist. In some embodiments, the treatment extends the OS of the subject or population of subjects by at least about 7 months or about 12 months. In some embodiments, the increase in OS is about 7 months or more. In some embodiments, the increase in OS is about 12 months or more. In some embodiments, the increase in OS is about 4 months, about 5 months, about 6 months, about 7 months, about 8 months, about 9 months, about 10 months, about 11 months, about 12 months, about 12 months, about 13 months, about 14 months, about 15 months, about 16 months, about 17 months, about 18 months, about 19 months, about 20 months, about 21 months, about 22 months, about 23 months, about 24 months, or more. In some embodiments, the treatment results in a median OS of the population of subjects of about 24 months to about 36 months. In some embodiments, administration of the anti-TIGIT antagonist antibody (e.g., tiragolumab) and the PD-1 axis binding antagonist (e.g., atezolizumab) to a plurality of subjects results in a median OS of at least about 24 months or more (e.g., about 24.5 months, 25 months, 25.5 months, 26 months, 26.5 months, 27 months, 27.5 months, 28 months, 28.5 months, 29 months, 29.5 months, 30 months, 30.5 months, 31 months, 31.5 months, about 32 months, about 32.5 months, about 33 months, about 33.5 months, about 34 months, about 34.5 months, about 35 months, about 35.5 months, about 36 months, or more) after the start of treatment with the anti-TIGIT antagonist antibody (e.g., tiragolumab) and the PD-1 axis binding antagonist (e.g., atezolizumab).

In some embodiments, the treatment results in an increase in duration of objective response (DOR) in the subject or population of subjects as compared to treatment with the PD-1 axis binding antagonist without the anti-TIGIT antagonist antibody. In some embodiments, the treatment results in an increase in DOR in the subject or population of subjects as compared to treatment with the anti-TIGIT antagonist antibody without the PD-1 axis binding antagonist. In some embodiments, the treatment results in an increase in DOR in the subject or population of subjects as compared to treatment without the anti-TIGIT antagonist antibody and without the PD-1 axis binding antagonist. In some embodiments, the increase in DOR is about 4 months, about 5 months, about 6 months, about 7 months, about 8 months, about 9 months, about 10 months, about 11 months, about 12 months, about 12 months, about 13 months, about 14 months, about 15 months, about 16 months, about 17 months, about 18 months, about 19 months, about 20 months, about 21 months, about 22 months, about 23 months, about 24 months, or more. In some embodiments, administration of the anti-TIGIT antagonist antibody (e.g., tiragolumab) and the PD-1 axis binding antagonist (e.g., atezolizumab) to a plurality of subjects results in a median DOR of at least about 4 months or more (e.g., about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months or more) after the start of treatment with the anti-TIGIT antagonist antibody (e.g., tiragolumab) and the PD-1 axis binding antagonist (e.g., atezolizumab).

In some embodiments, the treatment results in a complete response or a partial response.

In some embodiments, the subject or population of subjects has not been treated previously with cancer immunotherapy.

In some embodiments, the subject or population of subjects has completed a previous cancer immunotherapy for ESCC.

In some embodiments, the method comprises administering to the subject or population of subjects at least five dosing cycles (e.g., at least six dosing cycles, at least seven dosing cycles, at least eight dosing cycles, at least nine dosing cycles, at least 10 dosing cycles, at least 11 dosing cycles, at least 12 dosing cycles, at least 13 dosing cycles, at least 14 dosing cycles, at least 15 dosing cycles, at least 16 dosing cycles, at least 17 dosing cycles, at least 18 dosing cycles, at least 19 dosing cycles, or at least 20 dosing cycles). In some embodiments, the method comprises administering to the subject or population of subjects 17 dosing cycles.

In another aspect, the invention features a method for treating a subject having an ESCC, the method comprising administering to the subject one or more dosing cycles of tiragolumab at a fixed dose of about 30 mg to about 1200 mg every three weeks and atezolizumab at a fixed dose of about 80 mg to about 1600 mg every three weeks, wherein the subject previously received definitive chemoradiation treatment (e.g., definitive concurrent chemoradiation treatment) for ESCC. In some embodiments, the tiragolumab is administered at a fixed dose of about 600 mg every three weeks and the atezolizumab is administered at a fixed dose of about 1200 mg every three weeks. In some embodiments, no chemotherapy is administered to the subject during the one or more dosing cycles. In some embodiments, an ESCC tumor sample obtained from the subject has been determined to have a TIC score of greater than, or equal to 10%, as determined by an IHC assay using anti-PD-L1 antibody SP263. In some embodiments, the method comprises administering to the subject at least five dosing cycles an ESCC tumor sample obtained from the subject has been determined to have a TIC score of less than 10%, as determined by an IHC assay using anti-PD-L1 antibody SP263. In some embodiments, the ESCC is a locally advanced ESCC, an unresectable ESCC, an unresectable locally advanced ESCC, a recurrent or metastatic ESCC, or an ESCC comprising a cervical esophageal tumor. In some embodiments, the ESCC is a Stage II ESCC, a Stage III ESCC, or a Stage IV ESCC (e.g., a Stage IVA ESCC or a Stage IVB ESCC with supraclavicular lymph node metastases only).

In another aspect, the invention provides a method for treating a subject having an ESCC, the method comprising administering to the subject one or more dosing cycles of tiragolumab at a fixed dose of about 300 mg to about 800 mg every two weeks and atezolizumab at a fixed dose of about 200 mg to about 1200 mg every two weeks, wherein the subject previously received definitive chemoradiation treatment (e.g., definitive concurrent chemoradiation treatment) for ESCC. In some embodiments, the tiragolumab is administered at a fixed dose of about 420 mg every two weeks and the atezolizumab is administered at a fixed dose of about 840 mg every two weeks. In some embodiments, an ESCC tumor sample obtained from the subject has been determined to have a TIC score of greater than, or equal to 10%, as determined by an IHC assay using anti-PD-L1 antibody SP263. In some embodiments, the method comprises administering to the subject at least five dosing cycles an ESCC tumor sample obtained from the subject has been determined to have a TIC score of less than 10%, as determined by an IHC assay using anti-PD-L1 antibody SP263. In some embodiments, the ESCC is a locally advanced ESCC, an unresectable ESCC, an unresectable locally advanced ESCC, a recurrent or metastatic ESCC, or an ESCC comprising a cervical esophageal tumor. In some embodiments, the ESCC is a Stage II ESCC, a Stage III ESCC, or a Stage IV ESCC (e.g., a Stage IVA ESCC or a Stage IVB ESCC with supraclavicular lymph node metastases only).

In another aspect, the invention provides a method for treating a subject having an ESCC, the method comprising administering to the subject one or more dosing cycles of tiragolumab at a fixed dose of about 700 mg to about 1000 mg every four weeks and atezolizumab at a fixed dose of about 400 mg to about 2000 mg every four weeks, wherein the subject previously received definitive chemoradiation treatment (e.g., definitive concurrent chemoradiation treatment) for ESCC. In some embodiments, the tiragolumab is administered at a fixed dose of about 840 mg every four weeks and the atezolizumab is administered at a fixed dose of about 1680 mg every four weeks. In some embodiments, an ESCC tumor sample obtained from the subject has been determined to have a TIC score of greater than, or equal to 10%, as determined by an IHC assay using anti-PD-L1 antibody SP263. In some embodiments, the method comprises administering to the subject at least five dosing cycles an ESCC tumor sample obtained from the subject has been determined to have a TIC score of less than 10%, as determined by an IHC assay using anti-PD-L1 antibody SP263. In some embodiments, the ESCC is a locally advanced ESCC, an unresectable ESCC, an unresectable locally advanced ESCC, a recurrent or metastatic ESCC, or an ESCC comprising a cervical esophageal tumor. In some embodiments, the ESCC is a Stage II ESCC, a Stage III ESCC, or a Stage IV ESCC (e.g., a Stage IVA ESCC or a Stage IVB ESCC with supraclavicular lymph node metastases only).

In some embodiments, the method comprises administering to the subject at least five dosing cycles (e.g., at least six dosing cycles, at least seven dosing cycles, at least eight dosing cycles, at least nine dosing cycles, at least 10 dosing cycles, at least 11 dosing cycles, at least 12 dosing cycles, at least 13 dosing cycles, at least 14 dosing cycles, at least 15 dosing cycles, at least 16 dosing cycles, at least 17 dosing cycles, at least 18 dosing cycles, at least 19 dosing cycles, or at least 20 dosing cycles). In some embodiments, the method comprises administering to the subject 17 dosing cycles.

In some embodiments of any of the preceding aspects, the subject is a human.

In another aspect, the invention provides a kit comprising an anti-TIGIT antagonist antibody for use in combination with a PD-1 axis binding antagonist for treating a subject having an ESCC according to the method of any one of the previous aspects. In some embodiments, the kit further comprises the PD-1 axis binding antagonist. In some embodiments, the anti-TIGIT antagonist antibody is tiragolumab and the PD-1 axis binding antagonist is atezolizumab.

In another aspect, the invention provides a kit comprising a PD-1 axis binding antagonist for use in combination with an anti-TIGIT antagonist antibody for treating a subject having an ESCC according to the method of any one of the previous aspects. In some embodiments, the kit further comprises the anti-TIGIT antagonist antibody. In some embodiments, the anti-TIGIT antagonist antibody is tiragolumab and the PD-1 axis binding antagonist is atezolizumab.

In another aspect, provided herein is an anti-TIGIT antagonist antibody and a PD-1 axis binding antagonist for use in a method of treating a subject having an ESCC, wherein the method is according to any one of the preceding aspects.

In another aspect, provided herein is a use of an anti-TIGIT antagonist antibody in the manufacture of a medicament for treating a subject having an ESCC in combination with a PD-1 axis binding antagonist, wherein the treatment is according to the method of any one of the preceding aspects. In some embodiments, the anti-TIGIT antagonist antibody and the PD-1 axis binding antagonist are formulated separately. In other embodiments, the anti-TIGIT antagonist antibody and the PD-1 axis binding antagonist are formulated together.

In another aspect, provided herein is a use of a PD-1 axis binding antagonist in the manufacture of a medicament for treating a subject having an ESCC in combination with an anti-TIGIT antagonist antibody, wherein the treatment is according to the method of any one of the preceding aspects. In some embodiments, the anti-TIGIT antagonist antibody and the PD-1 axis binding antagonist are formulated separately. In other embodiments, the anti-TIGIT antagonist antibody and the PD-1 axis binding antagonist are formulated together.

In another aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 30 mg to about 1200 mg every three weeks (e.g., at a fixed dose of about 30 mg to about 600 mg every three weeks, e.g., at a fixed dose of about 600 mg every three weeks)), a PD-1 axis binding antagonist (e.g., at a fixed dose of about 80 mg to about 1600 mg every three weeks (e.g., at a fixed dose of about 800 mg to about 1400 mg every three weeks, e.g., at a fixed dose of about 1200 mg every three weeks)), a taxane (e.g., at a dose of about 100-250 mg/m² every three weeks (e.g., at a dose of 150-200 mg/m² every three weeks, e.g., at a dose of about 175 mg/m² every three weeks)), and a platinum agent (e.g., at a dose of about 20-200 mg/m² every three weeks (e.g., at a dose of about 40-120 mg/m² every three weeks, e.g., at a dose of about 60-80 mg/m² every three weeks)). In some embodiments, the subject or population of subjects has received no prior systemic treatment for ESCC (e.g., advanced ESCC). In some embodiments, the subject or population of subjects has received no prior systemic treatment for non-advanced ESCC. In other embodiments, the subject or population of subjects has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., chemoradiotherapy or chemotherapy administered with curative intent or in an adjuvant or neoadjuvant setting). In some embodiments, the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks.

In another aspect, provided herein is a method for treating a subject or population of subjects having an advanced ESCC for whom surgery is unsuitable, the method comprising administering to the subject or population of subjects one or more dosing cycles of an anti-TIGIT antagonist antibody, a PD-1 axis binding antagonist, a taxane, and a platinum agent. In some embodiments, the subject or population of subjects has received no prior systemic treatment for advanced ESCC. In some embodiments, the subject or population of subjects has received no prior systemic treatment for non-advanced ESCC. In other embodiments, the subject or population of subjects has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., chemoradiotherapy or chemotherapy administered with curative intent or in an adjuvant or neoadjuvant setting). In some embodiments, the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks.

In another aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)), a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)), a taxane, and a platinum agent. In some embodiments, the anti-TIGIT antagonist antibody is administered at a fixed dose of about 420 mg every two weeks and the PD-1 axis binding antagonist is administered at a fixed dose of about 840 mg every two weeks. In some embodiments, the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

In another aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks), a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)), a taxane, and a platinum agent. In some embodiments, the anti-TIGIT antagonist antibody is administered at a fixed dose of about 840 mg every four weeks and the PD-1 axis binding antagonist is administered at a fixed dose of about 1680 mg every four weeks. In some embodiments, the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

In some embodiments, the taxane is administered once per week, once every two weeks, once every three weeks, twice every three weeks, once every four weeks, twice every four weeks, or three times every four weeks. In some embodiments, the platinum agent is administered once per week, once every two weeks, once every three weeks, twice every three weeks, once every four weeks, twice every four weeks, or three times every four weeks. In some embodiments, the taxane and the platinum agent are both administered once per week, once every two weeks, once every three weeks, twice every three weeks, once every four weeks, twice every four weeks, or three times every four weeks.

In some embodiments, the anti-TIGIT antagonist antibody comprises the following hypervariable regions (HVRs): an HVR-H1 sequence comprising the amino acid sequence of SNSAAWN (SEQ ID NO: 1); an HVR-H2 sequence comprising the amino acid sequence of KTYYRFKWYSDYAVSVKG (SEQ ID NO: 2); an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6). In some embodiments, the anti-TIGIT antagonist antibody further comprises the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10). In some embodiments, the anti-TIGIT antagonist antibody further comprises the following heavy chain variable region FRs: an FR-H1 comprising the amino acid sequence of XiVQLQQSGPGLVKPSQTLSLTCAISGDSVS (SEQ ID NO: 11), wherein Xi is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In some embodiments, Xi is E. In some embodiments, Xi is Q. In some embodiments, the anti-TIGIT antagonist antibody comprises: (a) a heavy chain variable (VH) domain comprising an amino acid sequence having at least 95% sequence identity to the amino acid sequence of SEQ ID NO: 17 or 18; (b) a light chain variable (VL) domain comprising an amino acid sequence having at least 95% sequence identity to the amino acid sequence of SEQ ID NO: 19; or (c) a VH domain as in (a) and a VL domain as in (b). In some embodiments, the anti-TIGIT antagonist antibody comprises: (a) a VH domain comprising the amino acid sequence of SEQ ID NO: 17 or 18; and (b) a VL domain comprising the amino acid sequence of SEQ ID NO: 19. In some embodiments, the anti-TIGIT antagonist antibody is a monoclonal antibody. In some embodiments, the anti-TIGIT antagonist antibody is a human antibody. In some embodiments, the anti-TIGIT antagonist antibody is a full-length antibody. In some embodiments, the anti-TIGIT antagonist antibody is tiragolumab.

In some embodiments, the anti-TIGIT antagonist antibody is an antibody fragment that binds TIGIT selected from the group consisting of Fab, Fab′, Fab′-SH, Fv, single chain variable fragment (scFv), and (Fab′)₂ fragments. In some embodiments, the anti-TIGIT antagonist antibody is an IgG class antibody (e.g., an IgG1 subclass antibody).

In some embodiments, the PD-1 axis binding antagonist is a PD-L1 binding antagonist or a PD-1 binding antagonist. In some embodiments, the PD-1 binding antagonist is an anti-PD-1 antagonist antibody. In some embodiments, the anti-PD-1 antagonist antibody is nivolumab (MDX-1106), pembrolizumab (MK-3475), or AMP-224. In some embodiments, the PD-L1 binding antagonist is an anti-PD-L1 antagonist antibody (e.g., atezolizumab (MPDL3280A), MSB0010718C, MDX-1105, or MED14736). In some embodiments, the anti-PD-L1 antagonist antibody is atezolizumab. In some embodiments, the anti-PD-L1 antagonist antibody comprises the following HVRs: an HVR-H1 sequence comprising the amino acid sequence of GFTFSDSWIH (SEQ ID NO: 20); an HVR-H2 sequence comprising the amino acid sequence of AWISPYGGSTYYADSVKG (SEQ ID NO: 21); an HVR-H3 sequence comprising the amino acid sequence of RHWPGGFDY (SEQ ID NO: 22); an HVR-L1 sequence comprising the amino acid sequence of RASQDVSTAVA (SEQ ID NO: 23); an HVR-L2 sequence comprising the amino acid sequence of SASFLYS (SEQ ID NO: 24); and an HVR-L3 sequence comprising the amino acid sequence of QQYLYHPAT (SEQ ID NO: 25). In some embodiments, the anti-PD-L1 antagonist antibody comprises: (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b). In some embodiments, the anti-PD-L1 antagonist antibody comprises: a VH domain comprising the amino acid sequence of SEQ ID NO: 26; and a VL domain comprising the amino acid sequence of SEQ ID NO: 27. In some embodiments, the anti-PD-L1 antagonist antibody is a monoclonal antibody. In some embodiments, the anti-PD-L1 antagonist antibody is a humanized antibody. In some embodiments, the anti-PD-L1 antagonist antibody is a full-length antibody.

In some embodiments, the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments. In some embodiments, the anti-PD-L1 antagonist antibody is an IgG class antibody (e.g., an IgG1 subclass antibody).

In some embodiments, the taxane is paclitaxel or nab-paclitaxel, or a pharmaceutically acceptable salt thereof. In some embodiments, the taxane is paclitaxel, or a pharmaceutically acceptable salt thereof. In some embodiments, the platinum agent is cisplatin or carboplatin, or a pharmaceutically acceptable salt thereof. In some embodiments, the platinum agent is cisplatin, or a pharmaceutically acceptable salt thereof.

In some embodiments, the method comprises administering to the subject or population of subjects the PD-1 axis binding antagonist before the anti-TIGIT antagonist antibody. In some embodiments, the method comprises a first observation period following administration of the PD-1 axis binding antagonist and a second observation period following administration of the anti-TIGIT antagonist antibody. In some embodiments, the first observation period and the second observation period are each between about 30 minutes to about 60 minutes in length.

In some embodiments, the method comprises administering to the subject or population of subjects the anti-TIGIT antagonist antibody before the PD-1 axis binding antagonist. In some embodiments, the method comprises a first observation period following administration of the anti-TIGIT antagonist antibody and a second observation period following administration of the PD-1 axis binding antagonist. In some embodiments, the first observation period and the second observation period are each between about 30 minutes to about 60 minutes in length.

In some embodiments, the method comprises administering to the subject or population of subjects the anti-TIGIT antagonist antibody and the PD-1 axis binding antagonist simultaneously.

In some embodiments, the anti-TIGIT antagonist antibody and the PD-1 axis binding antagonist are administered before the taxane and/or the platinum agent. In some embodiments, the method comprises administering to the subject or population of subjects the taxane before the platinum agent. In some embodiments, the method comprises a third observation period following administration of the taxane and a fourth observation period following administration of the platinum agent. In some embodiments, the third observation period and the fourth observation period are each between about 30 minutes to about 60 minutes in length.

In some embodiments, the method comprises administering to the subject or population of subjects the anti-TIGIT antagonist antibody, the PD-1 axis binding antagonist, the taxane, and the platinum agent intravenously. In some embodiments, the method comprises administering to the subject or population of subjects the anti-TIGIT antagonist antibody by intravenous infusion over 60±10 minutes. In some embodiments, the method comprises administering to the subject or population of subjects the PD-1 axis binding antagonist by intravenous infusion over 60±15 minutes. In some embodiments, the method comprises administering to the subject or population of subjects the taxane by intravenous infusion over 3 hours±30 minutes. In some embodiments, the method comprises administering to the subject or population of subjects the platinum agent by intravenous infusion over 1-4 hours. In some embodiments, the anti-TIGIT antagonist antibody is administered subcutaneously. In some embodiments, the PD-1 axis binding antagonist is administered subcutaneously. In some embodiments, the anti-TIGIT antagonist antibody and the PD-1 axis binding antagonist are administered subcutaneously.

In some embodiments of any of the preceding methods, the length of each of the one or more dosing cycles is 21 days. In some embodiments, the anti-TIGIT antagonist antibody, PD-1 axis binding antagonist, the taxane, and the platinum agent are administered in each of four to eight initial (induction phase) dosing cycles (e.g., four to six induction phase dosing cycles, six to eight induction phase dosing cycles, or five to seven induction phase dosing cycles, e.g., four induction phase dosing cycles, five induction phase dosing cycles, six induction phase dosing cycles, seven induction phase dosing cycles, or eight induction phase dosing cycles). In some embodiments, the anti-TIGIT antagonist antibody, PD-1 axis binding antagonist, the taxane, and the platinum agent are administered in each of six induction phase dosing cycles.

In some embodiments, the anti-TIGIT antagonist antibody and the PD-1 axis binding antagonist are further administered in one or more additional (maintenance phase) dosing cycles following the induction phase dosing cycles. In some embodiments, the taxane and the platinum agent are omitted from each of the one or more maintenance phase dosing cycles. In some embodiments, the length of each of the induction phase dosing cycles and/or the one or more maintenance phase dosing cycles is 21 days.

In some embodiments, an ESCC tumor sample obtained from the subject or population of subjects has been determined to have a detectable expression level of PD-L1 (e.g., a detectable protein expression level of PD-L1 or a detectable nucleic acid expression level of PD-L1). In some embodiments, the detectable protein expression level of PD-L1 has been determined by an IHC assay. In some embodiments, the IHC assay uses anti-PD-L1 antibody SP263, 22C3, SP142, or 28-8. In some embodiments, the IHC assay uses anti-PD-L1 antibody SP263. In some embodiments, the IHC assay is the Ventana SP263 Companion Diagnostic (CDx) assay. In some embodiments, the ESCC tumor sample has been determined to have a tumor and tumor-associated immune cell (TIC) score of greater than, or equal to, 1%. In some embodiments, the TIC score is greater than, or equal to, 10%. In some embodiments, the ESCC tumor sample has been determined to have a TIC score of less than 10%. In some embodiments, the TIC score is greater than, or equal to, 10% and less than 50%. In some embodiments, the ESCC tumor sample has been determined to have a TIC score greater than, or equal to, 10%, as determined using the anti-PD-L1 antibody SP263 as part of the Ventana SP263 IHC assay (companion CDx assay), and the PD-1 axis binding antagonist administered in combination with the anti-TIGIT antagonist antibody, the taxane, and the platinum agent (e.g., tiragolumab, paclitaxel, and cisplatin) is atezolizumab.

In some embodiments, the IHC assay uses the anti-PD-L1 antibody 22C3 (e.g., as part of the pharmDx 22C3 IHC assay). In some embodiments, the ESCC tumor sample has been determined to have a CPS of greater than, or equal to, 10. In some embodiments, the ESCC tumor sample has been determined to have a TPS of greater than, or equal to, 1%. In some embodiments, the ESCC tumor sample has been determined to have a TPS of greater than, or equal to, 50%. In some embodiments, the IHC assay uses the anti-PD-L1 antibody SP142 (e.g., as part of the Ventana SP142 IHC assay). In some embodiments, the IHC assay uses the anti-PD-L1 antibody 28-8 (e.g., as part of the pharmDx 28-8 IHC assay). For example, in some embodiments, the ESCC tumor sample has been determined to have a CPS of greater than, or equal to, 10, as determined using the anti-PDL1 antibody 22C3 as part of the pharmDx22C3 IHC assay, and the PD-1 axis binding antagonist administered in combination with the anti-TIGIT antagonist antibody, the taxane, and the platinum agent (e.g., tiragolumab, paclitaxel, and cisplatin) is pembrolizumab. In some embodiments, the ESCC tumor sample has been determined to have a CPS of greater than, or equal to, 10, as determined using the anti-PDL1 antibody 22C3 as part of the pharmDx22C3 IHC assay, and the PD-1 axis binding antagonist administered in combination with the anti-TIGIT antagonist antibody, the taxane, and the platinum agent (e.g., tiragolumab, paclitaxel, and cisplatin) is atezolizumab.

In some embodiments, the detectable expression level of PD-L1 is a detectable nucleic acid expression level of PD-L1 (e.g., as determined by RNA-seq, RT-qPCR, qPCR, multiplex qPCR or RT-qPCR, microarray analysis, SAGE, MassARRAY technique, ISH, or a combination thereof). In some embodiments, the advanced ESCC is a locally advanced ESCC. In some embodiments, the advanced ESCC is a recurrent or metastatic ESCC. In some embodiments, the advanced ESCC is an unresectable ESCC.

In some embodiments, the treatment results in a progression-free survival (PFS) of about 8 months or more. In some embodiments, the treatment results in an increase in a PFS of the subject or population of subjects as compared to treatment with the taxane and the platinum agent, without the PD-1 axis binding antagonist and the anti-TIGIT antagonist antibody. In some embodiments, the treatment extends the PFS of the subject or population of subjects by at least about 2 months or about 4 months. In some embodiments, the increase in PFS is about 2 months or more (e.g. about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more). In some embodiments, the treatment results in a median PFS of the population of subjects of about 6 months to about 10 months. In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median PFS of at least about 6 months or more (e.g., about 6-7 months, about 7-8 months, about 8-10 months, or more, e.g., about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

In some embodiments, the treatment results in an overall survival (OS) of about 18 months or more. In some embodiments, the treatment results in an increase in OS of the subject or population of subjects as compared to treatment with the taxane and the platinum agent, without the PD-1 axis binding antagonist and the anti-TIGIT antagonist antibody. In some embodiments, the treatment extends the OS of the subject or population of subjects by at least about 4 months or about 6 months. In some embodiments, the increase in OS is about 4 months or more. In some embodiments, the increase in OS is about 6 months or more. In some embodiments, the increase in OS is about 2 months or more (e.g. about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more). In some embodiments, the treatment results in a median OS of the population of subjects of about 14 months to about 20 months. In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median OS of at least about 14 months or more (e.g., about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

In some embodiments, the treatment results in an increase in duration of objective response (DOR) in the subject or population of subjects as compared to treatment with the taxane and the platinum agent, without the PD-1 axis binding antagonist and the anti-TIGIT antagonist antibody. In some embodiments, the increase in DOR is about 2 months or more (e.g. about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more). In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median DOR of at least about 2 months or more (e.g., about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

In some embodiments, the treatment results in a complete response or a partial response.

In one aspect, provided herein is a method for treating a subject having an advanced ESCC, the method comprising administering to the subject one or more dosing cycles of tiragolumab at a fixed dose of about 30 mg to about 1200 mg every three weeks, atezolizumab at a fixed dose of about 80 mg to about 1600 mg every three weeks, paclitaxel at a dose of about 100-250 mg/m² every three weeks, and cisplatin at a dose of about 20-200 mg/m² every three weeks, wherein the subject has received no prior systemic treatment for the advanced ESCC. In some embodiments, the tiragolumab is administered at a fixed dose of about 600 mg every three weeks, the atezolizumab is administered at a fixed dose of about 1200 mg every three weeks, the paclitaxel is administered at a dose of about 175 mg/m² every three weeks, and the cisplatin is administered at a dose of about 60-80 mg/m² every three weeks.

In one aspect, provided herein is a method for treating a subject having an advanced ESCC, the method comprising administering to the subject one or more dosing cycles of tiragolumab at a fixed dose of about 300 mg to about 800 mg every two weeks, atezolizumab at a fixed dose of about 200 mg to about 1200 mg every two weeks, paclitaxel, and cisplatin, wherein the subject has received no prior systemic treatment for the advanced ESCC. In some embodiments, the tiragolumab is administered at a fixed dose of about 420 mg every two weeks, and the atezolizumab is administered at a fixed dose of about 840 mg every two weeks. In some embodiments, the paclitaxel and/or cisplatin are administered every two weeks.

In one aspect, the invention provides a method for treating a subject having an advanced ESCC, the method comprising administering to the subject one or more dosing cycles of tiragolumab at a fixed dose of about 700 mg to about 1000 mg every four weeks, atezolizumab at a fixed dose of about 400 mg to about 2000 mg every four weeks, paclitaxel, and cisplatin, wherein the subject has received no prior systemic treatment for the advanced ESCC. In some embodiments, the tiragolumab is administered at a fixed dose of about 840 mg every four weeks, and the atezolizumab is administered at a fixed dose of about 1680 mg every four weeks. In some embodiments, the paclitaxel and/or cisplatin are administered every four weeks.

In one aspect, the invention provides a method for treating a subject having an advanced ESCC, the method comprising administering to the subject: (i) six induction phase dosing cycles of tiragolumab at a fixed dose of about 30 mg to about 1200 mg every three weeks, atezolizumab at a fixed dose of about 80 mg to about 1600 mg every three weeks, paclitaxel at a dose of about 100-250 mg/m² every three weeks, and cisplatin at a dose of about 20-200 mg/m² every three weeks; and (ii) one or more maintenance phase dosing cycles of tiragolumab at a fixed dose of about 30 mg to about 1200 mg every three weeks and atezolizumab at a fixed dose of about 80 mg to about 1600 mg every three weeks, wherein the paclitaxel and the cisplatin are omitted from each of the one or more maintenance phase dosing cycles, wherein the subject has received no prior systemic treatment for the advanced ESCC. In some embodiments, (i) in the six induction phase dosing cycles, the tiragolumab is administered at a fixed dose of about 600 mg every three weeks, the atezolizumab is administered at a fixed dose of about 1200 mg every three weeks, the paclitaxel is administered at a dose of about 175 mg/m² every three weeks, and the cisplatin is administered at a dose of about 60-80 mg/m² every three weeks; and (ii) in the one or more maintenance phase dosing cycles, the tiragolumab is administered at a fixed dose of about 600 mg every three weeks and the atezolizumab is administered at a fixed dose of about 1200 mg every three weeks. In some embodiments, the subject has received no prior treatment for non-advanced ESCC. In some embodiments, the subject has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., a chemoradiotherapy or chemotherapy administered with curative intent or in an adjuvant or neoadjuvant setting).

In some embodiments, an ESCC tumor sample obtained from the subject has been determined to have a TIC score of greater than, or equal to 10%, as determined by an IHC assay using anti-PD-L1 antibody SP263. In some embodiments, an ESCC tumor sample obtained from the subject has been determined to have a TIC score of less than 10%, as determined by an IHC assay using anti-PD-L1 antibody SP263. In some embodiments, the advanced ESCC is a locally advanced ESCC, an unresectable ESCC, an unresectable locally advanced ESCC, an unresectable recurrent ESCC, or a recurrent or metastatic ESCC.

In some embodiments, the subject is a human.

In another aspect, the invention provides a kit comprising an anti-TIGIT antagonist antibody for use in combination with a PD-1 axis binding antagonist, a taxane, and a platinum agent for treating a subject having an advanced ESCC according to any of the previous methods for treating a subject having an advanced ESCC. In some embodiments, the kit further comprises the PD-1 axis binding antagonist. In some embodiments, the anti-TIGIT antagonist antibody is tiragolumab and the PD-1 axis binding antagonist is atezolizumab.

In another aspect, provided herein is a kit comprising a PD-1 axis binding antagonist for use in combination with an anti-TIGIT antagonist antibody, a taxane, and a platinum agent for treating a subject having an advanced ESCC according to any of the previous methods for treating a subject having an advanced ESCC. In some embodiments, the kit further comprises the anti-TIGIT antagonist antibody. In some embodiments, the anti-TIGIT antagonist antibody is tiragolumab and the PD-1 axis binding antagonist is atezolizumab.

In another aspect, the invention provides an anti-TIGIT antagonist antibody, PD-1 axis binding antagonist, taxane, and platinum agent for use in a method of treating a subject having an advanced ESCC, wherein the method is according to any of the previous methods for treating a subject having an advanced ESCC.

In another aspect, the invention provides use of an anti-TIGIT antagonist antibody in the manufacture of a medicament for treating a subject having an advanced ESCC in combination with a PD-1 axis binding antagonist, a taxane, and a platinum agent, wherein the treatment is according to any of the previous methods for treating a subject having an advanced ESCC. In some embodiments, the anti-TIGIT antagonist antibody and the PD-1 axis binding antagonist are formulated separately. In other embodiments, the anti-TIGIT antagonist antibody and the PD-1 axis binding antagonist are formulated together.

In another aspect, the invention provides use of a PD-1 axis binding antagonist in the manufacture of a medicament for treating a subject having an advanced ESCC in combination with an anti-TIGIT antagonist antibody, a taxane, and a platinum agent, wherein the treatment is according to any of the previous methods for treating a subject having an advanced ESCC. In some embodiments, the anti-TIGIT antagonist antibody and the PD-1 axis binding antagonist are formulated separately. In other embodiments, the anti-TIGIT antagonist antibody and the PD-1 axis binding antagonist are formulated together.

### BRIEF DESCRIPTION OF THE DRAWINGS

FIG. 1 is a flow chart of a phase III study schema for a second-line (2L) ESCC therapy. SCLN—supraclavicular lymph node; CDx=companion diagnostic; ECOG PS=Eastern Cooperative Oncology Group Performance Status; Q3W every three weeks; TIC=tumor and tumor-associated immune cell. PD-L1 expression is assessed by a central laboratory using the investigational Ventana PD-L1 (SP263) CDx Assay.

FIG. 2 is a flow chart of a phase III trial schema for a first-line (1 L) advanced ESCC therapy. Atezo+Tira+PC=treatment with atezolizumab, tiragolumab, paclitaxel, and cisplatin; Placebo+PC=treatment with atezolizumab placebo, tiragolumab placebo, paclitaxel, and cisplatin; R=randomization.

FIG. 3 is a diagram showing the objective response rate (ORR) (complete response/partial response (CR/PR); stable disease/progressive disease (SD/PD); or not evaluable (NE)) in patients from the CITYSCAPE trial having low or high PD-L1 TPS as assessed by the pharmDx 22C3 IHC assay (high TPS≥50%; low TPS 1-49%) or low or high PD-L1 tumor content (TC) as assessed by the CE-IVD VENTANA SP263 IHC assay (high TC≥50%; low TC 1-49%).

FIG. 4A is a bar graph showing the response rate (95% confidence interval (CI)) for patients from the CITYSCAPE trial having a TPS≥1% as measured using the 22C3 IHC assay.

FIG. 4B is a bar graph showing the response rate (95% CI) for patients from the CITYSCAPE trial having a TC≥1% as measured using the SP263 IHC assay (and TPS≥1% as measured using the 22C3 IHC assay).

FIG. 5A is a graph showing progression-free survival (percent) for patients from the CITYSCAPE trial who were treated with tiragolumab and atezolizumab (tira+atezo) or placebo+atezo and had a TPS≥1% as measured using the 22C3 IHC assay. The inset table shows median PFS in months (mo) and hazard ratio (HR).

FIG. 5B is a graph showing progression-free survival (percent) for patients from the CITYSCAPE trial who were treated with tiragolumab and atezolizumab (tira+atezo) or placebo+atezo and had a TC≥1% as measured using the SP263 IHC assay (and TPS≥1% as measured using the 22C3 IHC assay). The inset table shows median PFS in months and HR.

FIG. 6A is a bar graph showing the response rate (95% confidence interval (CI)) for patients from the CITYSCAPE trial having a TPS≥50% as measured using the 22C3 IHC assay.

FIG. 6B is a bar graph showing the response rate (95% CI) for patients from the CITYSCAPE trial having a TC≥50% as measured using the SP263 IHC assay.

FIG. 7A is a graph showing progression-free survival (percent) for patients from the CITYSCAPE trial who were treated with tiragolumab and atezolizumab (tira+atezo) or placebo+atezo and had a TPS≥50% as measured using the 22C3 IHC assay. The inset table shows median PFS in months and HR.

FIG. 7B is a graph showing progression-free survival (percent) for patients from the CITYSCAPE trial who were treated with tiragolumab and atezolizumab (tira+atezo) or placebo+atezo and had a TC≥50% as measured using the SP263 IHC assay. The inset table shows median PFS in months and HR.

### DETAILED DESCRIPTION

The present invention involves methods of treating a subject or population of subjects having esophageal cancer, e.g., esophageal squamous cell carcinoma (ESCC), by administering a combination of an anti-TIGIT antagonist antibody (e.g., an anti-TIGIT antagonist antibody as disclosed herein, e.g., tiragolumab) and a PD-1 axis binding antagonist (e.g., an anti-PD-L1 antagonist antibody (e.g., atezolizumab)). In some aspects, the invention involves methods of treating a subject or population of subjects that has previously received definitive chemoradiation treatment for esophageal cancer, e.g., ESCC (e.g., as a second-line (2L) treatment). In some aspects, the invention involves methods of treating a subject or population of subjects that has an advanced ESCC, wherein the subject or population of subjects has received no prior systemic treatment for ESCC (e.g., as a first-line (1L) treatment).

The invention is based, in part, on the discovery that immunotherapies including an anti-TIGIT antibody in combination with a PD-1 axis binding antagonist (e.g., an anti-programmed death ligand-1 (PD-L1) antibody or an anti-programmed death-1 (PD-1) antibody) can be useful in the treatment of esophageal squamous cell carcinoma (ESCC) (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), e.g., in subjects or populations of subjects that have previously received definitive chemoradiation treatment for ESCC.

Another basis for the present invention is the development of a combination treatment for a subject or population of subjects having advanced ESCC (e.g., locally advanced ESCC, unresectable ESCC, locally advanced unresectable ESCC, or recurrent or metastatic ESCC), e.g., Stage II ESCC (e.g., Stage IIB or Stage IIC), Stage III ESCC, or Stage IV ESCC (e.g., a Stage IVA ESCC or a Stage IVB ESCC with supraclavicular lymph node metastases only)). In some instances, the subject or population of subjects received no prior systemic treatment for the advanced ESCC. In some instances, surgery is unsuitable for the subject or population of subjects. Such treatment includes an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody (e.g., atezolizumab)), a taxane (e.g., paclitaxel), and a platinum agent (e.g., cisplatin).

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein.

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, F1139873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

The term “PD-L1” or “Programmed Cell Death Ligand 1” refers herein to any native PD-L1 from any vertebrate source, including mammals such as primates (e.g., humans) and rodents (e.g., mice and rats), unless otherwise indicated. PD-L1 is also known in the art as CD274 molecule, CD274 antigen, B7 homolog 1, PDCD1 Ligand 1, PDCD1 LG1, PDCD1 L1, B7H1, PDL1, programmed death ligand 1, B7-H1, and B7-H. The term also encompasses naturally occurring variants of PD-L1, e.g., splice variants, or allelic variants. The amino acid sequence of an exemplary human PD-L1 may be found under UniProt Accession Number Q9NZQ7 (SEQ ID NO: 32).

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, target cell killing). As used herein, a PD-1 axis binding antagonist includes a PD-1 binding antagonist, a PD-L1 binding antagonist, and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is pembrolizumab (formerly lambrolizumab (MK-3475)) described herein. In another specific aspect, a PD-1 binding antagonist is AMP-224 described herein.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is atezolizumab described herein (e.g., MPDL3280A). In another specific aspect, an anti-PD-L1 antibody is MDX-1105 described herein. In still another specific aspect, an anti-PD-L1 antibody is MED14736 described herein.

As used herein, the term “atezolizumab” refers to anti-PD-L1 antagonist antibody having the International Nonproprietary Names for Pharmaceutical Substances (INN) List 112 (WHO Drug Information, Vol. 28, No. 4, 2014, p. 488), or the CAS Registry Number 1380723-44-3.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one embodiment, the extent of binding of an anti-TIGIT antagonist antibody to an unrelated, non-TIGIT protein is less than about 10% of the binding of the antibody to TIGIT as measured, e.g., by a radioimmunoassay (RIA). In certain embodiments, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g. from 10⁻⁸M to 10⁻¹³ M, e.g., from 10⁻⁹M to 10⁻¹³ M). In certain embodiments, an anti-TIGIT antagonist antibody binds to an epitope of TIGIT that is conserved among TIGIT from different species or an epitope on TIGIT that allows for cross-species reactivity. In one embodiment, the anti-TIGIT antagonist antibody is tiragolumab.

As used herein, the term “tiragolumab” refers to an anti-TIGIT antagonist antibody having the International Nonproprietary Names for Pharmaceutical Substances (INN) List 117 (WHO Drug Information, Vol. 31, No. 2, 2017, p. 343), or the CAS Registry Number 1918185-84-8. Tiragolumab is also interchangeably referred to as “RO7092284.”

As used herein, “administering” is meant a method of giving a dosage of a compound (e.g., an anti-TIGIT antagonist antibody, a PD-1 axis binding antagonist (e.g., an anti-PD-L1 antibody), a taxane, and/or a platinum agent) or a composition (e.g., a pharmaceutical composition, e.g., a pharmaceutical composition including an anti-TIGIT antibody, a PD-1 axis binding antagonist (e.g., an anti-PD-L1 antibody), a taxane, and/or a platinum agent to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

As used herein, to be “administered with curative intent” refers to administration of a treatment in a dose and frequency (including a single administration) intended to achieve a complete response in the subject.

As used herein, “systemic treatment” refers to a treatment that travels through the bloodstream and is capable of contacting multiple organ systems upon a single administration. The term “systemic treatment” is well understood by those skilled in the art and is equivalent to systemic therapy.

A “fixed” or “flat” dose of a therapeutic agent (e.g., an anti-TIGIT antagonist antibody or a PD-1 axis binding antagonist (e.g., an anti-PD-L1 antibody) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in conjunction with” refers to administration of one treatment modality in addition to another treatment modality. As such, “in conjunction with” refers to administration of one treatment modality before, during, or after administration of the other treatment modality to the individual.

A “disorder” or “disease” is any condition that would benefit from treatment including, but not limited to, disorders that are associated with some degree of abnormal cell proliferation, e.g., cancer, e.g., esophageal cancer, e.g., esophageal squamous cell carcinoma (ESCC) (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)).

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to esophageal cancer, e.g., esophageal squamous cell carcinoma (ESCC), (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)). Additional examples of cancer are gastric cancer or stomach cancer, including gastrointestinal cancer, gastrointestinal stromal cancer, or gastroesophageal junction cancer. colon cancer; rectal cancer; colorectal cancer; cancer of the peritoneum; hepatocellular cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2+ breast cancer and triple-negative breast cancer (TNBC), which are estrogen receptors (ER−), progesterone receptors (PR−), and HER2 (HER2−) negative); endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., ESCC, (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only))). Examples of anti-cancer therapeutic agents include, but are limited to, e.g., immunomodulatory agents (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), an anti-TIGIT antagonist antibody, or a PD-1 axis binding antagonist (e.g., an anti-PD-L1 antibody), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-TIGIT antagonist antibody or a PD-1 axis binding antagonist (e.g., anti-PD-L1 antibody), or a composition (e.g., pharmaceutical composition) thereof, is at least the minimum amount required to achieve the desired therapeutic result, such as a measurable increase in overall survival or progression-free survival of a particular disease or disorder (e.g., cancer, e.g., ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only))). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, symptomatic skeletal-related events (SSE), reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), reduction in pain as measured by, e.g., the 10-point pain severity (measured at its worst) numerical rating scale (NRS), and/or reduction in symptoms associated with lung cancer per the health-related quality of life (HRQoL) questionnaire as assessed by symptoms in lung cancer (SILC) scale (e.g., time to deterioration (TTD) in cough dyspenea and chest pain), increasing the quality of life of those suffering from the disease, decreasing the dose of other medications required to treat the disease, enhancing effect of another medication such as via targeting, delaying the progression of the disease (e.g. progression-free survival or radiographic progression-free survival (rPFS); delay of unequivocal clinical progression (e.g., cancer-related pain progression, symptomatic skeletal-related event, deterioration in Eastern Cooperative Group Oncology Group (ECOG) Performance Status (PS) (e.g., how the disease affects the daily living abilities of the patient), and/or initiation of next systemic anti-cancer therapy), and/or delaying time to lung-specific antigen progression), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “advanced esophageal squamous cell carcinoma” or “advanced ESCC” refers to an ESCC of stage II or greater, according to the America Joint Committee on Cancer/Union for International Cancer Control, 8^{th} Edition. See e.g., Rice et al. Ann. Cardiothorac. Surg. 2017, 6(2):119-130. In some instances, the advanced ESCC is a Stage II ESCC (e.g., a Stage IIA ESCC or a Stage IIB ESCC). In some instances, the advanced ESCC is a Stage III ESCC (e.g., a Stage IIIA ESCC or a Stage IIIB ESCC). In some instances, the advanced ESCC is a Stage IV ESCC (e.g., a Stage IVA ESCC or a Stage IVB ESCC (e.g., a Stage IVB ESCC with SCLN metastases)).

As used herein, a “subject having an advanced ESCC for whom surgery is unsuitable” refers to a subject having an advanced ESCC for whom surgery (e.g., surgical resection of the ESCC) is not an option. For example, the advanced ESCC may be unresectable.

As used herein, the term “non-advanced esophageal squamous cell carcinoma” or “non-advanced ESCC” refers to an ESCC less than Stage II (e.g., Stage 0 or Stage I (e.g., stage IA or stage IB), according to the America Joint Committee on Cancer/Union for International Cancer Control, 8^{th} Edition. See e.g., Rice et al. Ann. Cardiothorac. Surg. 2017, 6(2):119-130.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with a TIGIT and/or PD-L1 antagonist (e.g., anti-TIGIT antagonist antibodies and/or anti-PD-L1 antibodies).

“Individual response” or “response” can be assessed using any endpoint indicating a benefit to the subject, including, without limitation, (1) inhibition, to some extent, of disease progression (e.g., progression of cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)); (6) increase or extend in the length of survival, including overall survival and progression-free survival; and/or (9) decreased mortality at a given point of time following treatment.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, or death from any cause within 30 days of the last dose of a treatment, whichever occurs first.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0×length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) does not get worse. Progression-free survival may include the amount of time patients have experienced a complete response or a partial response, as well as the amount of time patients have experienced stable disease.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “delaying progression” of a disorder or disease means to defer, hinder, slow, retard, stabilize, and/or postpone development of the disease or disorder (e.g., cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), the presence or size of metastases, or the size of the primary tumor.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

As used herein, the “Ventana SP263 IHC assay” (also referred to herein as the Ventana SP263 CDx assay) is conducted according to the Ventana PD-L1 (SP263) Assay package insert (Tucson, Ariz.: Ventana Medical Systems, Inc.), which is incorporated herein by reference in its entirety.

As used herein, the “Ventana SP142 IHC assay” is conducted according to the Ventana PD-L1 (SP142) Assay package insert (Tucson, Ariz.: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, Calif.: Dako, Agilent Pathology Solutions), which is incorporated herein by reference in its entirety.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

The term “biomarker,” as used herein, refers to an indicator, e.g., predictive, diagnostic, and/or prognostic, which can be detected in a sample. The biomarker may serve as an indicator of a particular subtype of a disease or disorder (e.g., cancer, e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only))) characterized by certain, molecular, pathological, histological, and/or clinical features. In some embodiments, a biomarker is a gene. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers. In some embodiments, the biomarker is PD-L1.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four CH domains for μ and ε isotypes. Each L chain has at the N-terminus, a variable domain (VL) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′-fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994)

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRII) subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., lmmunomethods 4: 25-34 (1994); and de Haas et al., J. Lab. Clin. Med. 126: 330-41 (1995). Other FcRs, including those to be identified in the future, are encompassed by the term “FcR” herein.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, lgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., TIGIT or PD-L1). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” is meant a mammal, including, but not limited to, a human or non-human mammal, such as a bovine, equine, canine, ovine, or feline. In some embodiments, the subject is a human. Patients are also subjects herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample obtained from a subject of interest that would be expected or is known to contain the cellular and/or molecular entity that is to be characterized. In some embodiments, the sample is a tumor tissue sample (e.g., an ESCC tumor sample, e.g., an advanced ESCC tumor sample (e.g., a locally advanced ESCC tumor sample), an unresectable ESCC tumor sample (e.g., a locally advanced unresectable ESCC tumor tissue sample), a recurrent or metastatic ESCC tumor tissue sample, a Stage II ESCC tumor tissue sample, a Stage III ESCC tumor tissue sample, or a Stage IV ESCC tumor tissue sample (e.g., a Stage IVA ESCC tumor tissue sample or a Stage IVB ESCC tumor tissue sample)). Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a diseased tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

### III. SECOND-LINE ESCC THERAPIES

The invention is based, in part, on the discovery that immunotherapies including an anti-TIGIT antagonist antibody in combination with a PD-1 axis binding antagonist (e.g., an anti-programmed death ligand-1 (PD-L1) antibody or an anti-programmed death-1 (PD-1) antibody) can be useful in the treatment of esophageal squamous cell carcinoma (ESCC) (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) in subjects or populations of subjects that have previously received definitive chemoradiation treatment for ESCC. In some instances, the definitive chemoradiation treatment was completed no more than 89 days prior to administration with the anti-TIGIT antagonist antibody or the PD-1 axis binding antagonist (e.g., no more than 88, no more than 87, no more than 86, no more than 85, or no more than 84 days prior to administration with the anti-TIGIT antagonist antibody or the PD-1 axis binding antagonist, e.g., within twelve weeks and five days before administration with the anti-TIGIT antagonist antibody or the PD-1 axis binding antagonist, within twelve weeks before administration with the anti-TIGIT antagonist antibody or the PD-1 axis binding antagonist, within eleven weeks before administration with the anti-TIGIT antagonist antibody or the PD-1 axis binding antagonist, within ten weeks before administration with the anti-TIGIT antagonist antibody or the PD-1 axis binding antagonist, within nine weeks before administration with the anti-TIGIT antagonist antibody or the PD-1 axis binding antagonist, within eight weeks before administration with the anti-TIGIT antagonist antibody or the PD-1 axis binding antagonist, within seven weeks before administration with the anti-TIGIT antagonist antibody or the PD-1 axis binding antagonist, within six weeks before administration with the anti-TIGIT antagonist antibody or the PD-1 axis binding antagonist, within five weeks before administration with the anti-TIGIT antagonist antibody or the PD-1 axis binding antagonist, within four weeks before administration with the anti-TIGIT antagonist antibody or the PD-1 axis binding antagonist, within three weeks before administration with the anti-TIGIT antagonist antibody or the PD-1 axis binding antagonist, within two weeks before administration with the anti-TIGIT antagonist antibody or the PD-1 axis binding antagonist, or within one week before administration with the anti-TIGIT antagonist antibody or the PD-1 axis binding antagonist). In some instances, the definitive chemoradiation treatment was completed no more than 84 days prior to administration with the anti-TIGIT antagonist antibody or the PD-1 axis binding antagonist. In some instances, the definitive chemoradiation treatment was completed no more than 47 days prior to administration with the anti-TIGIT antagonist antibody or the PD-1 axis binding antagonist (e.g., no more than 46, no more than 45, no more than 44, no more than 43, or no more than 42 days prior to administration with the anti-TIGIT antagonist antibody or the PD-1 axis binding antagonist. In some instances, the definitive chemoradiation treatment was completed no more than 42 days prior to administration with the anti-TIGIT antagonist antibody or the PD-1 axis binding antagonist. In some instances, the definitive chemoradiation treatment received by the subject or population of subjects comprises at least two cycles of chemotherapy (e.g., platinum-based chemotherapy) and radiation therapy without evidence of radiographic disease progression. In some instances of any of the methods described herein, no chemotherapy is administered to the subject or population of subjects during the one or more dosing cycles. In some instances, the subject or population of subjects has not been treated previously with cancer immunotherapy. In some instances, the subject or population of subjects has completed a previous cancer immunotherapy for ESCC. In some instances, the anti-TIGIT antagonist antibody (e.g., anti-TIGIT antagonist antibody as disclosed herein, e.g., tiragolumab) and the PD-1 axis binding antagonist (e.g., an anti-PD-L1 antagonist antibody, e.g., atezolizumab) are administered every two weeks (e.g., on Days 1 and 15 of each 28-day dosing cycle), every three weeks (e.g., on Day 1 of each 21-day dosing cycle), or every four weeks (e.g., on Day 1 of each 28-day dosing cycle).

In one aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 30 mg to about 1200 mg every three weeks (e.g., about 30 mg to about 600 mg every three weeks, e.g., about 600 mg every three weeks)) and a PD-1 axis binding antagonist (e.g., at a fixed dose of about 80 mg to about 1600 mg every three weeks (e.g., about 800 mg to about 1400 mg every three weeks, e.g., about 1200 mg every three weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 30 mg to about 1200 mg every three weeks (e.g., about 30 mg to about 600 mg every three weeks, e.g., about 600 mg every three weeks)) and a PD-1 axis binding antagonist (e.g., at a fixed dose of about 80 mg to about 1600 mg every three weeks (e.g., about 800 mg to about 1400 mg every three weeks, e.g., about 1200 mg every three weeks)), wherein the subject or population of subjects previously received definitive chemoradiation treatment (e.g., definitive concurrent chemoradiation treatment) for ESCC.

In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)) and a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)) and a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)), wherein the subject or population of subjects previously received definitive chemoradiation treatment (e.g., definitive concurrent chemoradiation treatment) for ESCC.

In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks) and a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)). In another aspect, the invention provides a method for treating a subject or population of subjects having an ESCC (e.g., unresectable locally advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks) and a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)), wherein the subject or population of subjects previously received definitive chemoradiation treatment (e.g., definitive concurrent chemoradiation treatment) for ESCC. In some instances, the method involves administering to the subject or population of subjects one or more dosing cycles of an anti-TIGIT antagonist antibody (e.g., tiragolumab) at a fixed dose of about 30 mg to about 1200 mg (e.g., about 600 mg) every three weeks and a PD-1 axis binding antagonist (e.g., atezolizumab) at a fixed dose of about 200 mg to about 1200 mg (e.g., about 840 mg) every two weeks. In some instances, the method involves administering to the subject or population of subjects one or more dosing cycles of an anti-TIGIT antagonist antibody (e.g., tiragolumab) at a fixed dose of about 30 mg to about 1200 mg (e.g., about 600 mg) every three weeks and a PD-1 axis binding antagonist (e.g., atezolizumab) at a fixed dose of about 400 mg to about 2000 mg (e.g., about 1680 mg) every four weeks. In some instances, the method involves administering to the subject or population of subjects one or more dosing cycles of an anti-TIGIT antagonist antibody (e.g., tiragolumab) at a fixed dose of about 300 mg to about 800 mg (e.g., about 420 mg) every two weeks and a PD-1 axis binding antagonist (e.g., atezolizumab) at a fixed dose of about 80 mg to about 1600 mg (e.g., about 1200 mg) every three weeks. In some instances, the method involves administering to the subject or population of subjects one or more dosing cycles of an anti-TIGIT antagonist antibody (e.g., tiragolumab) at a fixed dose of about 300 mg to about 800 mg (e.g., about 420 mg) every two weeks and a PD-1 axis binding antagonist (e.g., atezolizumab) at a fixed dose of about 400 mg to about 2000 mg (e.g., about 1680 mg) every four weeks. In some instances, the method involves administering to the subject or population of subjects one or more dosing cycles of an anti-TIGIT antagonist antibody (e.g., tiragolumab) at a fixed dose of about 700 mg to about 1000 mg (e.g., about 840 mg) every four weeks and a PD-1 axis binding antagonist (e.g., atezolizumab) at a fixed dose of about 200 mg to about 1200 mg (e.g., about 840 mg) every two weeks. In some instances, the method involves administering to the subject or population of subjects one or more dosing cycles of an anti-TIGIT antagonist antibody (e.g., tiragolumab) at a fixed dose of about 700 mg to about 1000 mg (e.g., about 840 mg) every four weeks and a PD-1 axis binding antagonist (e.g., atezolizumab) at a fixed dose of about 80 mg to about 1600 mg (e.g., about 1200 mg) every three weeks.

In some embodiments, the subject or population of subjects has experienced disease progression or unacceptable toxicity as a result of the previous therapy (e.g., previous definitive chemoradiation therapy).

Therapeutic Methods for Second-Line ESCC Therapies

The therapeutic methods and uses of the invention described herein include, in one aspect, administering one or more dosing cycles to a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC. The one or more dosing cycles include an effective amount of an anti-TIGIT antagonist antibody (e.g., an anti-TIGIT antagonist antibody as disclosed herein, e.g., tiragolumab) and an effective amount of a PD-1 axis binding antagonist (e.g., an anti-PD-L1 antagonist antibody (e.g., atezolizumab)).

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W). In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab)) may be reduced as compared to a standard dose of the anti-TIGIT antagonist antibody administered as a monotherapy.

In some instances, the effective amount of the anti-TIGIT antagonist antibody (e.g., an anti-TIGIT antagonist antibody as disclosed herein, e.g., tiragolumab) is a fixed dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a fixed dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks).

In some instances, the effective amount of the anti-TIGIT antagonist antibody (e.g., an anti-TIGIT antagonist antibody as disclosed herein, e.g., tiragolumab) is a fixed dose of between about 200 mg to about 2000 mg (e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every four weeks (Q4W). In some instances, the effective amount of anti-TIGIT antagonist antibody (e.g., an anti-TIGIT antagonist antibody as disclosed herein, e.g., tiragolumab) is a fixed dose of about 840 mg every four weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every four weeks).

In some instances, the effective amount of the PD-1 axis binding antagonist (e.g., an anti-PD-L1 antagonist antibody (e.g., atezolizumab)) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some embodiments, the effective amount of the PD-1 axis binding antagonist is atezolizumab at a fixed dose of about 1200 mg every three weeks. In some embodiments, the effective amount of the PD-1 axis binding antagonist is pembrolizumab at a fixed dose of about 200 mg every three weeks or, alternatively, pembrolizumab at a fixed dose of about 400 mg every six weeks.

In some instances, the fixed dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., an anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., an anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of PD-1 axis binding antagonist (e.g., an anti-PD-L1 antagonist antibody (e.g., atezolizumab)) is a dose of about 15 mg/kg administered every three weeks. In some instances, the dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., anti-PD-L1 antagonist antibody (e.g., atezolizumab)) is a fixed dose of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist is atezolizumab at a fixed dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some embodiments, the effective amount of the PD-1 axis binding antagonist is avelumab at a fixed dose of about 800 mg every two weeks. In some embodiments, the effective amount of the PD-1 axis binding antagonist is nivolumab at a fixed dose of about 240 mg every two weeks.

In some instances, the effective amount of the PD-1 axis binding antagonist (e.g., anti-PD-L1 antagonist antibody (e.g., atezolizumab)) is a fixed dose of between about 500 mg to about 3000 mg (e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab)) is a fixed dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680±0.5 mg, e.g., 1680 mg every four weeks). In some embodiments, the effective amount of the PD-1 axis binding antagonist is nivolumab at a fixed dose of about 480 mg every four weeks.

In any of the methods and uses of the invention, the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab)) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, 17 dosing cycles are administered. In some instances, the dosing cycles of the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab)) continue until there is a loss of clinical benefit (e.g., confirmed disease progression, drug resistance, death, or unacceptable toxicity). In some instances, the length of each dosing cycle is about 15 to 24 days (e.g., 15 days, 16 days, 17 days, 18 days, 19 days, 20 days, 21 days, 22 days, 23 days, or 24 days). In some instances, the length of each dosing cycle is about 21 days. In some instances, the length of each dosing cycle is about 80 to 88 days (e.g., 80 days, 81 days, 82 days, 83 days, 84 days, 85 days, 86 days, 87 days, or 88 days). In some instances, the length of each dosing cycle is about 84 days. In some instances, the length of each dosing cycle is about 38 to 46 days (e.g., 38 days, 39 days, 40 days, 41 days, 42 days, 43 days, 44 days, 45 days, or 46 days). In some instances, the length of each dosing cycle is about 42 days. In some instances, the length of each dosing cycle is about 24 to 32 days (e.g., 24 days, 25 days, 26 days, 27 days, 28 days, 29 days, 30 days, 31 days, or 32 days). In some instances, the length of each dosing cycle is about 28 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks). For example, the anti-TIGIT antagonist antibody (e.g., an anti-TIGIT antagonist antibody as disclosed herein, e.g., tiragolumab) is administered intravenously at a fixed dose of about 840 mg on Day 1 of each 28-day cycle (i.e., at a fixed dose of about 840 mg every four weeks). In some instances, the anti-TIGIT antagonist antibody (e.g., an anti-TIGIT antagonist antibody as disclosed herein, e.g., tiragolumab) is administered on about Day 1 (e.g., Day 1±3 days) and Day 22 (e.g., Day 22±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 and Day 22 of each 42-day cycle (i.e., at a fixed dose of about 600 mg every three weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1, Day 22, Day 43, and Day 64 of each 84-day cycle (i.e., at a fixed dose of about 600 mg every three weeks). In some instances, the anti-TIGIT antagonist antibody (e.g., an anti-TIGIT antagonist antibody as disclosed herein, e.g., tiragolumab) is administered on about Day 1 (e.g., Day 1±3 days) and about Day 15 (e.g., Day 15±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 420 mg on Day 1 and Day 15 of each 28-day cycle (i.e., at a fixed dose of about 420 mg every two weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 15 (e.g., Day 15±3 days), and Day 29 (e.g., Day 29±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 420 mg on Day 1, Day 15, and Day 29 of each 42-day cycle (i.e., at a fixed dose of about 420 mg every two weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 29 (e.g., Day 29±3 days), and Day 57 (e.g., Day 57±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 840 mg on Day 1, Day 29, and Day 56 of each 84-day cycle (i.e., at a fixed dose of about 840 mg every four weeks). Similarly, in some instances, the PD-1 axis binding antagonist (e.g., an anti-PD-L1 antagonist antibody (e.g., atezolizumab)) is administered on about Day 1 (e.g., Day 1±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1 of each 21-day cycle (i.e., at a fixed dose of about 1200 mg every three weeks). For example, the PD-1 axis binding antagonist (e.g., an anti-PD-L1 antagonist antibody (e.g., atezolizumab)) is administered intravenously at a fixed dose of about 1680 mg on Day 1 of each 28-day cycle (i.e., at a fixed dose of about 1680 mg every four weeks). In some instances, the PD-1 axis binding antagonist (e.g., an anti-PD-L1 antagonist antibody (e.g., atezolizumab)) is administered on about Day 1 (e.g., Day 1±3 days) and Day 22 (e.g., Day 22±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1 and Day 22 of each 42-day cycle (i.e., at a fixed dose of about 1200 mg every three weeks). In some instances, the PD-1 axis binding antagonist (e.g., an anti-PD-L1 antagonist antibody (e.g., atezolizumab)) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1, Day 22, Day 43, and Day 64 of each 84-day cycle (i.e., at a fixed dose of about 1200 mg every three weeks). In some instances, the PD-1 axis binding antagonist (e.g., an anti-PD-L1 antagonist antibody (e.g., atezolizumab)) is administered on about Day 1 (e.g., Day 1±3 days) and about Day 15 (e.g., Day 15±3 days) of each dosing cycle. For example, the PD-1 axis binding antagonist (e.g., an anti-PD-L1 antagonist antibody (e.g., atezolizumab)) is administered intravenously at a fixed dose of about 840 mg on Day 1 and Day 15 of each 28-day cycle (i.e., at a fixed dose of about 840 mg every two weeks). In some instances, the PD-1 axis binding antagonist (e.g., an anti-PD-L1 antagonist antibody (e.g., atezolizumab)) is administered on about Day 1 (e.g., Day 1±3 days), Day 15 (e.g., Day 15±3 days), and Day 29 (e.g., Day 29±3 days) of each dosing cycle. For example, the PD-1 axis binding antagonist (e.g., an anti-PD-L1 antagonist antibody (e.g., atezolizumab)) is administered intravenously at a fixed dose of about 840 mg on Day 1, Day 15, and Day 29 of each 42-day cycle (i.e., at a fixed dose of about 840 mg every two weeks). In some instances, the PD-1 axis binding antagonist (e.g., an anti-PD-L1 antagonist antibody (e.g., atezolizumab)) is administered on about Day 1 (e.g., Day 1±3 days), Day 29 (e.g., Day 29±3 days), and Day 57 (e.g., Day 57±3 days) of each dosing cycle. For example, the PD-1 axis binding antagonist (e.g., an anti-PD-L1 antagonist antibody (e.g., atezolizumab)) is administered intravenously at a fixed dose of about 1680 mg on Day 1, Day 29, and Day 56 of each 84-day cycle (i.e., at a fixed dose of about 1680 mg every four weeks). In some instances, both the anti-TIGIT antagonist antibody (e.g., an anti-TIGIT antagonist antibody as disclosed herein, e.g., tiragolumab) and the PD-1 axis binding antagonist (e.g., an anti-PD-L1 antagonist antibody (e.g., atezolizumab)) are administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks), and the PD-1 axis binding antagonist (e.g., an anti-PD-L1 antagonist antibody (e.g., atezolizumab)) is administered intravenously at a fixed dose of about 1200 mg on Day 1 of each 21-day cycle (i.e., at a fixed dose of about 1200 mg every three weeks).

In some instances, the anti-TIGIT antagonist antibody (e.g., an anti-TIGIT antagonist antibody as disclosed herein, e.g., tiragolumab) is administered to the subject by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., an anti-PD-L1 antagonist antibody (e.g., atezolizumab)) is administered to the subject by intravenous infusion over about 60±15 minutes (e.g. about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject before the PD-1 axis binding antagonist (e.g., an anti-PD-L1 antagonist antibody (e.g., atezolizumab)). In some instances, for example, following administration of the anti-TIGIT antagonist antibody and before administration of the PD-1 axis binding antagonist, the method includes an intervening first observation period. In some instances, the method further includes a second observation period following administration of the PD-1 axis binding antagonist (e.g., an anti-PD-L1 antagonist antibody (e.g., atezolizumab)). In some instances, the method includes both a first observation period following administration of the anti-TIGIT antagonist antibody and second observation period following administration of the PD-1 axis binding antagonist. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and PD-1 axis binding antagonist during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and PD-1 axis binding antagonist during the first and second observation periods, respectively.

In other instances, the PD-1 axis binding antagonist (e.g., an anti-PD-L1 antagonist antibody (e.g., atezolizumab)) is administered to the subject before the anti-TIGIT antagonist antibody (e.g., an anti-TIGIT antagonist antibody as disclosed herein, e.g., tiragolumab). In some instances, for example, following administration of the PD-1 axis binding antagonist and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the PD-1 axis binding antagonist and a second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist and anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the anti-PD-L1 antagonist antibody (e.g., atezolizumab) are administered to the subject simultaneously. In some instances, for example, following administration of the anti-TIGIT antagonist antibody and the PD-1 axis binding antagonist, the method includes an observation period. In some instances, the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the PD-1 axis binding antagonist and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the PD-1 axis binding antagonist and anti-TIGIT antagonist antibody during the observation period.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of tiragolumab at a fixed dose of 600 mg every three weeks and atezolizumab at a fixed dose of 1200 mg every three weeks.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of an anti-TIGIT antagonist antibody at a fixed dose of 600 mg every three weeks and atezolizumab at a fixed dose of 840 mg every two weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of tiragolumab at a fixed dose of 600 mg every three weeks and atezolizumab at a fixed dose of 840 mg every two weeks.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of an anti-TIGIT antagonist antibody at a fixed dose of 600 mg every three weeks and atezolizumab at a fixed dose of 1680 mg every four weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of tiragolumab at a fixed dose of 600 mg every three weeks and atezolizumab at a fixed dose of 1680 mg every four weeks.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of an anti-TIGIT antagonist antibody at a fixed dose of 420 mg every two weeks and atezolizumab at a fixed dose of 1200 mg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of tiragolumab at a fixed dose of 420 mg every two weeks and atezolizumab at a fixed dose of 1200 mg every three weeks.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of an anti-TIGIT antagonist antibody at a fixed dose of 420 mg every two weeks and atezolizumab at a fixed dose of 1680 mg every four weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, by administering to the subject or population of subjects one or more dosing cycles of tiragolumab at a fixed dose of 420 mg every two weeks and atezolizumab at a fixed dose of 1680 mg every four weeks.

In another aspect, the invention provides an anti-TIGIT antagonist antibody (e.g., an anti-TIGIT antagonist antibody disclosed herein, e.g., tiragolumab) and PD-1 axis binding antagonist (e.g., an anti-PD-L1 antagonist antibody (e.g., atezolizumab)) for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of an effective amount of an anti-TIGIT antagonist antibody (e.g., tiragolumab) and an effective amount of a PD-1 axis binding antagonist (e.g., a PD-1 axis binding antagonist (e.g., an anti-PD-L1 antagonist antibody (e.g., atezolizumab))) (e.g., according to any of the methods described herein).

In another aspect, the invention provides uses of an anti-TIGIT antagonist antibody (e.g., an anti-TIGIT antagonist antibody disclosed herein, e.g., tiragolumab) and PD-1 axis binding antagonist (e.g., an anti-PD-L1 antagonist antibody (e.g., atezolizumab)) in the manufacture or preparation of a medicament for use in any of the methods described herein.

In another aspect, the invention provides uses of an anti-TIGIT antagonist antibody in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and a PD-1 axis binding antagonist (e.g., an anti-PD-L1 antagonist antibody (e.g., atezolizumab)), and wherein the medicament is formulated for administration of an effective amount of the anti-TIGIT antagonist antibody (e.g., tiragolumab) and an effective amount of the PD-1 axis binding antagonist (e.g., an anti-PD-L1 antagonist antibody (e.g., atezolizumab)) according to any of the methods described herein.

In another aspect, the invention provides uses of a PD-1 axis binding antagonist (e.g., an anti-PD-L1 antagonist antibody (e.g., atezolizumab)) and an anti-TIGIT antagonist antibody (e.g., an anti-TIGIT antagonist antibody disclosed herein, e.g., tiragolumab) in the manufacture or preparation of a medicament for use in any of the methods described herein.

In another aspect, the invention provides uses of a PD-1 axis binding antagonist (e.g., an anti-PD-L1 antagonist antibody (e.g., atezolizumab)) in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antagonist antibody (e.g., an anti-TIGIT antagonist antibody disclosed herein, e.g., tiragolumab), and wherein the medicament is formulated for administration of an effective amount of an effective amount of the PD-1 axis binding antagonist (e.g., an anti-PD-L1 antagonist antibody (e.g., atezolizumab)) and an effective amount of the anti-TIGIT antagonist antibody (e.g., tiragolumab) according to any of the methods described herein.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 1200 mg every three weeks and the anti-TIGIT antagonist antibody is to be administered at a fixed dose of 600 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 600 mg every three weeks and atezolizumab at a fixed dose of 1200 mg every three weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 600 mg every three weeks and atezolizumab is to be administered at a fixed dose of 1200 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 1200 mg every three weeks and tiragolumab is to be administered at a fixed dose of 600 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 840 mg every two weeks and the anti-TIGIT antagonist antibody is to be administered at a fixed dose of 600 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 600 mg every three weeks and atezolizumab at a fixed dose of 840 mg every two weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 600 mg every three weeks and atezolizumab is to be administered at a fixed dose of 840 mg every two weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 840 mg every two weeks and tiragolumab is to be administered at a fixed dose of 600 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 1680 mg every four weeks and the anti-TIGIT antagonist antibody is to be administered at a fixed dose of 600 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 600 mg every three weeks and atezolizumab at a fixed dose of 1680 mg every four weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 600 mg every three weeks and atezolizumab is to be administered at a fixed dose of 1680 mg every four weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 1680 mg every four weeks and tiragolumab is to be administered at a fixed dose of 600 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 1200 mg every three weeks and the anti-TIGIT antagonist antibody is to be administered at a fixed dose of 420 mg every two weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 420 mg every two weeks and atezolizumab at a fixed dose of 1200 mg every three weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 420 mg every two weeks and atezolizumab is to be administered at a fixed dose of 1200 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 1200 mg every three weeks and tiragolumab is to be administered at a fixed dose of 420 mg every two weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects or more dosing cycles of the medicament and an anti-TIGIT antibody, wherein the medicament is formulated for administration of atezolizumab at a fixed dose of 1680 mg every four weeks and the anti-TIGIT antagonist antibody is to be administered at a fixed dose of 420 mg every two weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 420 mg every two weeks and atezolizumab at a fixed dose of 1680 mg every four weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 420 mg every two weeks and atezolizumab is to be administered at a fixed dose of 1680 mg every four weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 1680 mg every four weeks and tiragolumab is to be administered at a fixed dose of 420 mg every two weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 1200 mg every three weeks and the anti-TIGIT antagonist antibody is to be administered at a fixed dose of 840 mg every four weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 840 mg every four weeks and atezolizumab at a fixed dose of 1200 mg every three weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable

ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 840 mg every four weeks and atezolizumab is to be administered at a fixed dose of 1200 mg every three weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 1200 mg every three weeks and tiragolumab is to be administered at a fixed dose of 840 mg every four weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and an anti-TIGIT antibody, wherein the medicament is formulated for administration of atezolizumab at a fixed dose of 840 mg every two weeks and the anti-TIGIT antagonist antibody is to be administered at a fixed dose of 840 mg every four weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament, wherein the medicament is formulated for administration of tiragolumab at a fixed dose of 840 mg every four weeks and atezolizumab at a fixed dose of 840 mg every two weeks.

In another aspect, the invention provides uses of tiragolumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and atezolizumab, wherein the medicament is formulated for administration of tiragolumab at a fixed dose of 840 mg every four weeks and atezolizumab is to be administered at a fixed dose of 840 mg every two weeks.

In another aspect, the invention provides uses of atezolizumab in the manufacture of a medicament for use in a method of treating a subject or population of subjects having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)), wherein the subject or population of subjects has previously received definitive chemoradiation treatment for ESCC, wherein the method comprises administering to the subject or population of subjects one or more dosing cycles of the medicament and tiragolumab, wherein the medicament is formulated for administration of atezolizumab at a fixed dose of 840 mg every two weeks and tiragolumab is to be administered at a fixed dose of 840 mg every four weeks.

A. PD-L1 Selection

In some instances of any of the methods, uses, or compositions for use described herein, the subject has a PD-L1 selected ESCC tumor (e.g., an ESCC tumor with a detectable expression level (e.g., protein expression level or nucleic acid expression level) of PD-L1. In some instances, the PD-L1 selected tumor is an ESCC tumor that has been determined to have a PD-L1-positive tumor associated immune cell (TIC) score of at least 1% (e.g., at least 10%) by an immunohistochemical (IHC) assay. In some instances, the TIC score is from 1% to 99% (e.g., from 2% to 98%, from 3% to 97%, from 4% to 96%, from 5% to 95%, from 10% to 90%, from 15% to 85%, from 20% to 80%, or from 25% to 75%, e.g., from 1% to 10% (e.g., from 1% to 5% (e.g., from 1% to 2%, from 2% to 3%, from 3% to 4%, or from 4% to 5%) or from 5% to 10% (e.g., from 5% to 6%, from 6% to 7%, from 7% to 8%, from 8% to 9%, or from 9% to 10%)), from 10% to 20% (e.g., from 10% to 15% (e.g., from 10% to 11%, from 11% to 12%, from 12% to 13%, from 13% to 14%, or from 14% to 15%) or from 15% to 20% (e.g., from 15% to 16%, from 16% to 17%, from 17% to 18%, from 18% to 19%, or from 19% to 20%)), or greater than 20%). In some instances, the TIC score is less than 10% (e.g., from 1% to 10%, from 2% to 10%, from 3% to 10%, from 4% to 10%, from 5% to 10%, from 6% to 10%, from 7% to 10%, from 8% to 10%, or from 9% to 10%). In some instances, the TIC score is less than 20% (e.g., from 1% to 20%, from 2% to 20%, from 3% to 20%, from 4% to 20%, from 5% to 20%, from 6% to 20%, from 7% to 20%, from 8% to 20%, from 9% to 20%, from 10% to 20%, from 11% to 20%, from 12% to 20%, from 13% to 20%, from 14% to 20%, from 15% to 20%, from 16% to 20%, from 17% to 20%, from 18% to 20%, or from 19% to 20%).

In some instances, the IHC assay is the pharmDX 22C3 assay and the ESCC tumor sample has been determined to have a combined positive score (CPS) of greater than, or equal to, 10 (e.g., greater than, or equal to, 15; greater than, or equal to, 20; greater than, or equal to, 25; greater than, or equal to, 30; greater than, or equal to, 40; greater than, or equal to, 45; or greater than, or equal to, 50). In some embodiments, the ESCC tumor sample has been determined to have a tumor proportion score (TPS) of greater than, or equal to, 1%. In some embodiments, the ESCC tumor sample has been determined to have a TPS of greater than, or equal to, 50%.

In some instances, the IHC assay uses the anti-PD-L1 antibody SP142 or 28-8. In some instances, the IHC assay uses anti-PD-L1 antibody SP142 (e.g., Ventana SP142 IHC assay). In some instances, the IHC assay uses anti-PD-L1 antibody 28-8 (e.g., pharmDx 28-8 IHC assay).

In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

B. Responses to Second-Line Therapies

In some embodiments of any of the methods described herein, a subject's or population of subjects' response to the therapy can be characterized by one or more measures. In some embodiments, the treatment results in a complete response or a partial response.

In some instances, the treatment results in an increase in progression-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. For example, the treatment with the PD-1 axis binding antagonist and the anti-TIGIT antagonist antibody may result in an increase in progression-free survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some embodiments, the treatment results in an increase in PFS of the subject or population of subjects as compared to treatment without the anti-TIGIT antagonist antibody and without the PD-1 axis binding antagonist. In some embodiments, the treatment extends the PFS of the subject or population of subjects by at least about 4 months or about 8 months. In some embodiments, the increase in PFS is about 4 months or more (e.g., about 4.5 months or more, about 5.0 months or more, about 5.5. months or more, about 6.0 months or more, about 6.5 months or more, about 7.0 months or more, about 7.5 months or more, about 8.0 months or more, about 8.5 months or more, about 9.0 months or more, about 9.5 months or more, about 10 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in PFS is about 8 months or more (e.g., about 8.5 months or more, about 9 months or more, about 9.5 months or more, about 10 months or more, about 10.5 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in PFS is 4-8 months (e.g., about 4 months, about 4.5 months, about 5 months, about 5.5. months, about 6 months, about 6.5 months, about 7 months, about 7.5 months, or about 8 months). In some embodiments, the treatment results in a median PFS of the population of subjects of about 15 months to about 23 months. In some embodiments, administration of the anti-TIGIT antagonist antibody (e.g., tiragolumab) and the PD-1 axis binding antagonist (e.g., atezolizumab) to a plurality of subjects results in a median PFS of at least about 15 months (e.g., about 15.5 months, about 16 months, about 16.5 months, about 17 months, about 17.5 months, about 18 months, or about 18.5 months) after the start of treatment with the anti-TIGIT antagonist antibody (e.g., tiragolumab) and the PD-1 axis binding antagonist (e.g., atezolizumab). In some embodiments, administration of the anti-TIGIT antagonist antibody (e.g., tiragolumab) and the PD-1 axis binding antagonist (e.g., atezolizumab) to a plurality of subjects results in a median PFS of at least about 19 months (e.g., about 19.5 months, about 20 months, about 20.5 months, about 21 months, about 21.5 months, about 22 months, about 22.5 months, about 23 months, about 23.5 months, about 24 months, about 25 months, about 26 months, about 27 months, about 28 months about 29 months, about 30 months, about 31 months, about 32 months, about 33 months, about 34 months, about 35 months, about 36 months, or more) after the start of treatment with the anti-TIGIT antagonist antibody (e.g., tiragolumab) and the PD-1 axis binding antagonist (e.g., atezolizumab). In some embodiments, administration of the anti-TIGIT antagonist antibody (e.g., tiragolumab) and the PD-1 axis binding antagonist (e.g., atezolizumab) to a plurality of subjects results in a median PFS between 19 months and 60 months (e.g., between 20 and 60 months, between 25 and 60 months, between 30 and 60 months, between 35 and 60 months, between 40 and 60 months, between 45 and 60 months, between 50 and 60 months, or between 55 and 60 months) after the start of treatment with the anti-TIGIT antagonist antibody (e.g., tiragolumab) and the PD-1 axis binding antagonist (e.g., atezolizumab).

In some instances, the treatment results in an increase in overall survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. For example, the treatment with the PD-1 axis binding antagonist and the anti-TIGIT antagonist antibody may result in an increase in overall survival of the subject or population of subjects, e.g., as compared to treatment with the PD-1 axis binding antagonist without the anti-TIGIT antagonist antibody or as compared to treatment with the anti-TIGIT antagonist antibody without the PD-1 axis binding antagonist. In some embodiments, the treatment results in an increase in OS of the subject or population of subjects as compared to treatment without the anti-TIGIT antagonist antibody and without the PD-1 axis binding antagonist. In some embodiments, the treatment extends the OS of the subject or population of subjects by at least about 7 months or about 12 months. In some embodiments, the increase in OS is about 7 months or more (e.g., about 7.0 months or more, about 7.5 months or more, about 8.0 months or more, about 8.5 months or more, about 9.0 months or more, about 9.5 months or more, about 10 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in OS is about 12 months or more (e.g., about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in OS is 4-6 months (e.g., about 4 months, about 4.5 months, about 5 months, about 5.5. months, or about 6 months). In some embodiments, the treatment results in a median OS of the population of subjects of about 24 months to about 36 months. In some embodiments, administration of the anti-TIGIT antagonist antibody (e.g., tiragolumab) and the PD-1 axis binding antagonist (e.g., atezolizumab) to a plurality of subjects results in a median OS of at least about 24 months (e.g., about 24.5 months, about 25 months, about 25.5 months, about 26 months, about 26.5 months, about 27 months, about 27.5 months, about 28 months, about 28.5 months, about 29 months, about 29.5 months, about 30 months, or about 30.5 months) after the start of treatment with the anti-TIGIT antagonist antibody (e.g., tiragolumab) and the PD-1 axis binding antagonist (e.g., atezolizumab). In some embodiments, administration of the anti-TIGIT antagonist antibody (e.g., tiragolumab) and the PD-1 axis binding antagonist (e.g., atezolizumab) to a plurality of subjects results in a median OS of at least about 31 months (e.g., about 31.5 months, about 32 months, about 32.5 months, about 33 months, about 33.5 months, about 34 months, about 34.5 months, about 35 months, about 35.5 months, about 36 months, about 36.5 months, about 37 months, about 37.5 months, about 38 months, about 38.5 months, about 39 months, about 39.5 months, about 40 months, or more) after the start of treatment with the anti-TIGIT antagonist antibody (e.g., tiragolumab) and the PD-1 axis binding antagonist (e.g., atezolizumab). In some embodiments, administration of the anti-TIGIT antagonist antibody (e.g., tiragolumab) and the PD-1 axis binding antagonist (e.g., atezolizumab) to a plurality of subjects results in a median OS between 31 months and 60 months (e.g., between 32 and 60 months, between 33 and 60 months, between 34 and 60 months, between 35 and 60 months, between 36 and 60 months, between 37 and 60 months, between 38 and 60 months, between 39 and 60 months, between 40 and 60 months, between 41 and 60 months, between 42 and 60 months, between 43 and 60 months, between 44 and 60 months, between 45 and 60 months, between 46 and 60 months, between 47 and 60 months, between 48 and 60 months, between 49 and 60 months, between 50 and 60 months, between 51 and 60 months, between 52 and 60 months, between 53 and 60 months, between 54 and 60 months, between 55 and 60 months, between 56 and 60 months, between 57 and 60 months, between 58 and 60 months, or between 59 and 60 months) after the start of treatment with the anti-TIGIT antagonist antibody (e.g., tiragolumab) and the PD-1 axis binding antagonist (e.g., atezolizumab). In some instances, the treatment results in an increase in duration of objective response (DOR) in the subject or population of subjects as compared to treatment with the PD-1 axis binding antagonist without the anti-TIGIT antagonist antibody or as compared to treatment with the anti-TIGIT antagonist antibody without the PD-1 axis binding antagonist. In some instances, the treatment results in an increase in DOR in the subject or population of subjects as compared to treatment without the anti-TIGIT antagonist antibody and without the PD-1 axis binding antagonist. In some embodiments, the treatment results in an increase in DOR in the subject or population of subjects as compared to treatment without the anti-TIGIT antagonist antibody and without the PD-1 axis binding antagonist. In some embodiments, the increase in DOR is about 4 months, about 5 months, about 6 months, about 7 months, about 8 months, about 9 months, about 10 months, about 11 months, about 12 months, about 12 months, about 13 months, about 14 months, about 15 months, about 16 months, about 17 months, about 18 months, about 19 months, about 20 months, about 21 months, about 22 months, about 23 months, about 24 months, or more. In some embodiments, administration of the anti-TIGIT antagonist antibody (e.g., tiragolumab) and the PD-1 axis binding antagonist (e.g., atezolizumab) to a plurality of subjects results in a median DOR of at least about 4 months or more (e.g., about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months or more) after the start of treatment with the anti-TIGIT antagonist antibody (e.g., tiragolumab) and the PD-1 axis binding antagonist (e.g., atezolizumab).

Progression-free survival of the subject or population of subjects can be measured according to RECIST v1.1 criteria, as described in Eisenhauer et al., Eur. J. Cancer. 2009, 45:228-47. In some embodiments, PFS is measured as the period of time from the start of treatment to the first occurrence of disease progression as determined by RECIST v1.1 criteria. In some embodiments, PFS is measured as the time from the start of treatment to the time of death.

Exemplary Anti-TIGIT Antagonist Antibodies and PD-1 Axis Binding Antagonists for Second-Line Therapies

Exemplary anti-TIGIT antagonist antibodies and PD-1 axis binding antagonists (e.g., anti-PD-L1 antibodies) useful for treating a subject or population of subjects (e.g., a human) having ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) in accordance with the methods, uses, and compositions for use of the invention are described herein. In particular, the following exemplary anti-TIGIT antagonist antibodies and PD-1 axis binding antagonists (e.g., anti-PD-L1 antibodies), can be used to treat subjects who have previously received definitive chemoradiation treatment for ESCC.

A. Anti-TIGIT Antagonist Antibodies

The invention provides anti-TIGIT antagonist antibodies useful for treating ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) in a subject (e.g., a human).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, anti-TIGIT antagonist antibodies may include (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 17)
EVQLQQSGPGLVKPSQTLSLTCAISGDSVSSNSAAWNWIRQSP
SRGLEWLGKTYYRFKVVYSDYAVSVKGRITINPDTSKNQFSLQ
LNSVTPEDTAVFYCTRESTTYDLLAGPFDYVVGQGTLVTVSS

or an amino acid sequence having at least 90% sequence identity (e.g., at least 91%, 92%, 93%, 94%, 95%, 96%, 97%, 98%, or 99% sequence identity) to, or the sequence of,

(SEQ ID NO: 18)
QVQLQQSGPGLVKPSQTLSLTCAISGDSVSSNSAAWNWIRQSP
SRGLEWLGKTYYRFKVVYSDYAVSVKGRITINPDTSKNQFSLQ
LNSVTPEDTAVFYCTRESTTYDLLAGPFDYVVGQGTLVTVSS;

and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAVVYQQKPGQPPNLLIYWASTRESGVPDRFS GSGSGTDFTLTISSLQAEDVAVYYCQQYYSTPFTFGPGTKVEIK (SEQ ID NO: 19). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 17 and a VL domain comprising the amino acid sequence of SEQ ID NO: 19. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 18 and a VL domain comprising the amino acid sequence of SEQ ID NO: 19.

In some instances, the anti-TIGIT antagonist antibody includes a heavy chain and a light chain sequence, wherein: (a) the heavy chain comprises the amino acid sequence:

(SEQ ID NO: 33)
EVQLQQSGPGLVKPSQTLSLTCAISGDSVSSNSAAWNWIR
QSPSRGLEWLGKTYYRFKVVYSDYAVSVKGRITINPDTSK
NQFSLQLNSVTPEDTAVFYCTRESTTYDLLAGPFDYVVGQ
GTLVTVSSASTKGPSVFPLAPSSKSTSGGTAALGCLVKDY
FPEPVTVSWNSGALTSGVHTFPAVLQSSGLYSLSSVVTVP
SSSLGTQTYICNVNHKPSNTKVDKKVEPKSCDKTHTCPPC
PAPELLGGPSVFLFPPKPKDTLMISRTPEVTCVVVDVSHE
DPEVKFNWYVDGVEVHNAKTKPREEQYNSTYRVVSVLTVL
HQDWLNGKEYKCKVSNKALPAPIEKTISKAKGQPREPQVY
TLPPSREEMTKNQVSLTCLVKGFYPSDIAVEWESNGQPEN
NYKTTPPVLDSDGSFFLYSKLTVDKSRWQQGNVFSCSVMH
EALHNHYTQKSLSLSPGK;

and (b) the light chain comprises the amino acid sequence:

(SEQ ID NO: 34)
DIVMTQSPDSLAVSLGERATINCKSSQTVLYSSNNKKYLA
VVYQQKPGQPPNLLIYWASTRESGVPDRFSGSGSGTDFTL
TISSLQAEDVAVYYCQQYYSTPFTFGPGTKVEIKRTVAAP
SVFIFPPSDEQLKSGTASVVCLLNNFYPREAKVQWKVDNA
LQSGNSQESVTEQDSKDSTYSLSSTLTLSKADYEKHKVYA
CEVTHQGLSSPVTKSFNRGEC.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of XiVQLQQSGPGLVKPSQTLSLTCAISGDSVS (SEQ ID NO: 11), wherein Xi is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), ora combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14. In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization. In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antagonist antibody is an Fc fusion protein.

In some embodiments, the anti-TIGIT antibody is selected from the group consisting of tiragolumab (MTIG7192A, RG6058 or R07092284), vibostolimab (MK-7684), ASP8374 (PTZ-201), EOS884448 (EOS-448), SEA-TGT (SGN-TGT)), BGB-A1217, BMS-986207 (ONO-4686), COM902 (CGEN-15137), 161939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or R07092284), vibostolimab (MK-7684), ASP8374 (PTZ-201), EOS-448, and SEA-TGT (SGN-TGT). The anti-TIGIT antibody may be tiragolumab (MTIG7192A, RG6058 or R07092284).

Non-limiting examples of anti-TIGIT antibodies that are useful for the methods disclosed herein, and methods for making thereof are described in PCT Pub. Nos. WO2018183889A1, WO2019129261A1, WO2016106302A9, WO2018033798A1, WO2020020281A1, WO2019023504A1, WO2017152088A1, WO2016028656A1, WO2017030823A2, WO2018204405A1, WO2019152574A1, and WO2020041541A2; U.S. Pat. Nos. 10,189,902, 10,213,505, 10,124,061, 10,537,633, and 10,618,958; and U.S. Pub. Nos. 2020/0095324, 2019/0112375, 2018/0371083, and 2020/0062859, each of which is incorporated herein by reference in its entirety. Additional non-limiting examples of anti-TIGIT antibodies, useful for the methods of disclosed herein, and methods for making thereof are described in PCT Pub. Nos. WO2018204363A1, WO2018047139A1, WO2019175799A2, WO2018022946A1, WO2015143343A2, WO2018218056A1, WO2019232484A1, WO2019079777A1, WO2018128939A1, WO2017196867A1, WO2019154415A1, WO2019062832A1, WO2018234793A3, WO2018102536A1, WO2019137548A1, WO2019129221A1, WO2018102746A1, WO2018160704A9, WO2020041541A2, WO2019094637A9, WO2017037707A1, WO2019168382A1, WO2006124667A3, WO2017021526A1, WO2017184619A2, WO2017048824A1, WO2019032619A9, WO2018157162A1, WO2020176718A1, WO2020047329A1, WO2020047329A1, WO2018220446A9; U.S. Pat. Nos. 9,617,338, 9,567,399, 10,604,576, and 9,994,637; and Pub. Nos. US 2018/0355040, US 2019/0175654, US 2019/0040154, US 2019/0382477, US 2019/0010246, US 2020/0164071, US 2020/0131267, US 2019/0338032, US 2019/0330351, US 2019/0202917, US 2019/0284269, US 2018/0155422, US 2020/0040082, US 2019/0263909, US 2018/0185480, US 2019/0375843, US 2017/0037133, US 2019/0077869, US 2019/0367579, US 2020/0222503, US 2020/0283496, CN109734806A, and CN110818795A, each of which is incorporated herein by reference in its entirety.

The anti-TIGIT antibodies useful in the methods disclosed herein include ASP8374 (PTZ-201), BGB-A1217, BMS-986207 (ONO-4686), COM902 (CGEN-15137), M6223, 161939, EOS-448, domvanalimab (A6154), vibostolimab (MK-7684), and SEA-TGT (SGN-TGT). Additional anti-TIGIT antibodies useful in the methods disclosed herein include AGEN1307; AGEN1777; antibody clones pab2197 and pab2196 (Agenus Inc.); antibody clones TBB8, TDC8, 3TB3, 5TB10, and D1Y1A (Anhui Anke Biotechnology Group Co. Ltd.), antibody clones MAB1, MAB2, MAB3, MAB4, MAB5, MAB6, MAB 7, MAB8, MAB9, MAB 10, MAB 11, MAB 12, MAB13, MAB 14, MAB 15, MAB 16, MAB 17, MAB 18, MAB19, MAB20, MAB21 (Astellas Pharma/Potenza Therapeutics), antibody clones hu1217-1-1 and hu1217-2-2 (BeiGene), antibody clones 4D4 and 19G (Brigham & Women's Hospital), antibody clones 11G11, 10D7, 15A6, 22G2, TIGIT G2a, and TIGIT G1 D265A, including such antibodies with modified heavy chain constant regions (Bristol-Myers Squibb); antibody clones 10A7, CPA.9.086, CPA.9.083.H4(S241P), CPA.9.086.H4(5241P), CHA.9.547.7.H4(S241P) and CHA.9.547.13.H4(S241P) (Compugen); anti-PVRIG/anti-TIGIT bispecific antibodies (Compugen), antibody clones 315293, 328189, 350426, 326504, and 331672 (Fred Hutchinson Cancer Research Center); antibody clones T-01, T-02, T-03, T-04, T-05, T-06, T-07, T-08, T-09, and T-10 (Gensun BioPharma Inc.); antibody clones 1H6, 2611, 3A10, 4A5, 4A9, 4H5, 6A2, 6B7, 7F4, 8E1, 8G3, 9F4, 9G6, 10C1, 10F10, 11G4, 1267, 12C8, 15E9, 16C11, 16D6, and 16E10 (Hefei Ruida Immunological Drugs Research Institute Co. Ltd.); antibody clones h3C5H1, h3C5H2, h3C5H3, h3C5H4, h3C5H3-1, h3C5H3-2, h3C5H3-3, h3C5L1, and h3C5L2 (IGM Biosciences Inc.); antibody clones 90D9, 101E1, 116H8, 118A12, 131A12, 14366, 167F7, 221F11, 222H4, 327C9, 342A9, 344F2, 349H6, and 350D10 (1-Mab Biopharma); antibody clones ADI-27238, ADI-30263, ADI-30267, ADI-30268, ADI-27243, ADI-30302, ADI-30336, ADI-27278, ADI-30193, ADI-30296, ADI-27291, ADI-30283, ADI-30286, ADI-30288, AD127297, ADI-30272, ADI-30278, ADI-27301, ADI-30306, and ADI-30311 (Innovent Biologics, Inc.); antibody clones 26518, 29478, 26452, 29487, 29489, 31282, 26486, 29494, 29499, 26521, 29513, 26493, 29520, 29523, 29527, 31288, 32919, 32931, 26432, and 32959 (iTeos Therapeutics); antibody clones m1707, m1708, m1709, m1710, m1711, h1707, h1708, h1709, h1710, and h1711 (Jiangsu Hengrui Medicine Co. Ltd.); antibody clones TIG1, TIG2, and TIG3 (JN Biosciences LLC); antibody clones (e.g., KY01, KY02, KY03, KY04, KY05, KY06, KY07, KY08, KY09, KY10, K11, K12, K13, K14, K15, K16, K17, K18, K19, K₂O, K21, K22, K23 Kymab TIGIT (Antibody 2), and Tool TIGIT (Antibody 4) (Kymab Limited); bispecific antibodies 1D05/in-house anti-TIGIT with 1D05 (anti-PD-L1) Native variable domain and Kymab TIGIT antigen binding site (ABS) domain (Bispecific 1), In-house anti-TIGIT/1D05 with Kymab TIGIT Native variable domain and 1D05 ABS domain (Bispecific 2), Tool anti-TIGIT/Tool anti-PD-L1 with Toon anti-TIGIT Native variable domain and Tool anti-PD-L1 ABS domain (Bispecific 3), Tool anti-PD-L1/Tool anti-TIGIT with Tool anti-PD-L1 Native variable domain and Tool anti-TIGIT ABS domain (Bispecific 4) (Kymab Limited); antibody clones and clone variants 14D7, 26B10, Hu14D7, Hu26B10, 14A6, Hu14A6, 28H5, 31C6, Hu31C6, 25G10, MBS43, 37D10, 18G10, 11A11, c18G10, and LB155.14A6.G2.A8 (Merck); etigilimab (OMP-313M32) (Mereo BioPharma); antibody clones 64G1E9B4, 100C4E7D11, 83G5H11C12, 92E9D4B4, 104G12E12G2, 121C2F1065, 128E3F10F3F2, 70A11A8E6, 11D8E124A, 16F10H12C11, 8F2D8E7, 48B5G4E12, 139E2C2D2, 128E3G7F5, AS19584, AS19852, AS19858, AS19886, AS19887, AS19888, AS20160, AS19584VH26, AS19584VH29, AS19584VH30, AS19584VH31, AS19886VH5, AS19886VH8, AS19886VH9, AS19886VH10, AS19886VH19, AS19886VH20, AS19584VH28-Fc, AS19886VH5-Fc, AS19886VH8-Fc, AS19584-Fc, and AS19886-Fc (Nanjing Legend Biotechnology Co. Ltd.); antibody clones ARE clones: Ab58, Ab69, Ab75, Ab133, Ab177, Ab122, Ab86, Ab180, Ab83, Ab26, Ab20, Ab147, Ab12, Ab66, Ab176, Ab96, Ab123, Ab109, Ab149, Ab34, Ab61, Ab64, Ab105, Ab108, Ab178, Ab166, Ab29, Ab135, Ab171, Ab194, Ab184, Ab164, Ab183, Ab158, Ab55, Ab136, Ab39, Ab159, Ab151, Ab139, Ab107, Ab36, Ab193, Ab115, Ab106, Ab13f8, Ab127, Ab165, Ab155, Ab19, Ab6, Ab187, Ab179, Ab65, Ab114, Ab102, Ab94, Ab163, Ab110, Ab80, Ab92, Ab117, Ab162, Ab121, Ab195, Ab84, Ab161, Ab198, Ab24, Ab98, Ab116, Ab174, Ab196, Ab51, Ab91, Ab185, Ab23, Ab7, Ab95, Ab100, Ab140, Ab145, Ab150, Ab168, Ab54, Ab77, Ab43, Ab160, Ab82, Ab189, Ab17, Ab103, Ab18, Ab130, Ab132, Ab134, Ab144; ARG Clones: Ab2, Ab47, Ab49, Ab31, Ab53, Ab40, Ab5, Ab9, Ab48, Ab4, Ab10, Ab37, Ab33, Ab42, Ab45; ARV Clones: Ab44, Ab97, Ab81, Ab188, Ab186, Ab62, Ab57, Ab192, Ab73, Ab60, Ab28, Ab32, Ab78, Ab14, Ab152, Ab72, Ab137, Ab128, Ab169, Ab87, Ab74, Ab172, Ab153, Ab120, Ab13, Ab113, Ab16, Ab56, Ab129, Ab50, Ab90, Ab99, Ab3, Ab148, Ab124, Ab22, Ab41, Ab119, Ab157, Ab27, Ab15, Ab191, Ab190, Ab79, Ab181, Ab146, Ab167, Ab88, Ab199, Ab71, Ab85, Ab59, Ab141, Ab68, Ab143, Ab46, Ab197, Ab175, Ab156, Ab63, Ab11, Ab182, Ab89, Ab8, Ab101, Ab25, Ab154, Ab21, Ab111, Ab118, Ab173, Ab38, Ab76, Ab131, Ab1, Ab67, Ab70, Ab170, Ab30, Ab93, Ab142, Ab104, Ab112, Ab35, Ab126, and Ab125 (Rigel Pharmaceuticals, Inc.); CASC-674 (Seattle Genetics); antibody clones 2, 2C, 3, 5, 13, 13A, 13B, 13C, 13D, 14, 16, 16C, 16D, 16E, 18, 21, 22, 25, 25A, 25B, 25C, 25D, 25E, 27, 54, 13 IgG2a afucosylated, 13 hIgG1 wild-type, and 13 LALA-PG (Seattle Genetics); JS006 (Shanghai Junshi Biosciences Ltd.); anti-TIGIT Fc antibody and bispecific antibody PD1×TIGIT (Xencor), antibody clone VSIG9#1 (Vsig9.01) and 258-CS1#4 (#4) (Yissum Research Development Company of The Hebrew University Of Jerusalem Ltd.); YH29143 (Yuhan Co, Ltd.); antibody clones S02, S03, S04, S05, S06, S11, S12, S14, S19, S32, S39, S43, S62, S64, F01, F02, F03, F04, 32D7, 101H3, 10A7, and 1F4 (Yuhan Co, Ltd.); anti-zB7R1 clones 318.4.1.1 (E9310), 318.28.2.1 (E9296), 318.39.1.1 (E9311), 318.59.3.1 (E9400), and 318.77.1.10 (ZymoGenetics, Inc).

In some embodiments, the anti-TIGIT antibody is selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT). ASP874 (PTZ-201) is an anti-TIGIT monoclonal antibody described in PCT Pub. No. WO2018183889A1 and US Pub. No. 2020/0095324. BGB-A1217 is an anti-TIGIT antibody as described in PCT Pub. No. WO2019129261A1. BMS-986207 (ONO-4686) is an anti-TIGIT antibody as described in PCT Pub. No. WO2016106302A9, U.S. Pat. No. 10,189,902 and US Pub. No. 2019/0112375. COM902 (CGEN-15137) is an anti-TIGIT antibody as described in PCT Pub. No. WO2018033798A1 and U.S. Pat. Nos. 10,213,505 and 10,124,061. IBI939 is an anti-TIGIT antibody as described in PCT Pub. No. WO2020020281A1. EOS884448 (EOS-448) is an anti-TIGIT antibody described in PCT Pub. No. WO2019023504A1. Domvanalimab (AB154) is an anti-TIGIT monoclonal antibody as described in PCT Pub. No. WO2017152088A1 and U.S. Pat. No. 10,537,633. Vibostolimab (MK-7684) is an anti-TIGIT antibody described in PCT Pub. Nos. WO2016028656A1, WO2017030823A2, WO2018204405A1, and/or WO2019152574A1, U.S. Pat. No. 10,618,958, and US Pub. No. 2018/0371083. SEA-TGT (SGN-TGT) is an anti-TIGIT antibody as described in PCT Pub. No. WO2020041541A2 and US Pub. No. 2020/0062859.

In some embodiments, the anti-TIGIT antagonist antibody is tiragolumab (CAS Registry Number: 1918185-84-8). Tiragolumab (Genentech) is also known as MTIG7192A, RG6058 or R07092284. Tiragolumab is an anti-TIGIT antagonistic monoclonal antibody described in PCT Pub. No. WO2003072305A8, WO2004024068A3, WO2004024072A3, WO2009126688A2, WO2015009856A2, WO2016011264A1, WO2016109546A2, WO2017053748A2, and WO2019165434A1, and US Pub. Nos. 2017/0044256, 2017/0037127, 2017/0145093, 2017/260594, 2017/0088613, 2018/0186875, 2019/0119376 and U.S. Pat. Nos. 9,873,740B2, 10,626,174B2, 10,611,836B2, 9,499,596B2, 8,431,350B2, U.S. Ser. No. 10/047,158B2, and U.S. Ser. No. 10/017,572B2.

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT).

In some embodiments, an anti-TIGIT antagonist antibody (according to any of the embodiments described herein may incorporate any of the features, singly or in combination, as described in Section C below.

B. PD-1 Axis Binding Antagonists

Provided herein are methods for treating ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) in a subject or population of subjects (e.g., a human) comprising administering to the subject or population of subjects an effective amount of a PD-1 axis binding antagonist. PD-1 axis binding antagonists include PD-L1 binding antagonists (e.g., PD-L1 antagonist antibodies), PD-1 binding antagonists (e.g., PD-1 antagonist antibodies), and PD-2 binding antagonists (e.g., PD-L2 antagonist antibodies).

In some instances, the PD-1 axis binding antagonist is an PD-1 axis binding antagonist that inhibits the binding of PD-L1 to its binding partners. In a specific aspect, PD-L1 binding partners are PD-1 and/or B7-1. In some instances, the anti-PD-L1 antagonist antibody is capable of inhibiting binding between PD-L1 and PD-1 and/or between PD-L1 and B7-1.

In some instances, the PD-1 axis binding antagonist is an anti-PD-L1 antibody.

In some instances, the anti-PD-L1 antibody is atezolizumab (CAS Registry Number: 1422185-06-5). Atezolizumab (Genentech) is also known as MPDL3280A.

In some instances, the anti-PD-L1 antibody (e.g., atezolizumab) includes at least one, two, three, four, five, or six HVRs selected from: (a) an HVR-H1 sequence is GFTFSDSWIH (SEQ ID NO: 20); (b) an HVR-H2 sequence is AWISPYGGSTYYADSVKG (SEQ ID NO: 21); (c) an HVR-H3 sequence is RHWPGGFDY (SEQ ID NO: 22), (d) an HVR-L1 sequence is RASQDVSTAVA (SEQ ID NO: 23); (e) an HVR-L2 sequence is SASFLYS (SEQ ID NO: 24); and (f) an HVR-L3 sequence is QQYLYHPAT (SEQ ID NO: 25).

In some instances, the anti-PD-L1 antibody (e.g., atezolizumab) comprises a heavy chain and a light chain sequence, wherein: (a) the heavy chain variable (VH) region sequence comprises the amino acid sequence:

(SEQ ID NO: 26)
EVQLVESGGGLVQPGGSLRLSCAASGFTFSDSWIHVVVRQ
APGKGLEVVVAWISPYGGSTYYADSVKGRFTISADTSKNT
AYLQMNSLRAEDTAVYYCARRHWPGGFDYWGQGTLVTVSS;

and (b) the light chain variable (VL) region sequence comprises the amino acid sequence:

(SEQ ID NO: 27)
DIQMTQSPSSLSASVGDRVTITCRASQDVSTAVAVVYQQK
PGKAPKLLIYSASFLYSGVPSRFSGSGSGTDFTLTISSLQ
PEDFATYYCQQYLYHPATFGQGTKVEIKR.

In some instances, the anti-PD-L1 antibody (e.g., atezolizumab) comprises a heavy chain and a light chain sequence, wherein: (a) the heavy chain comprises the amino acid sequence:

(SEQ ID NO: 28)
EVQLVESGGGLVQPGGSLRLSCAASGFTFSDSWIHVVVRQ
APGKGLEWVAWISPYGGSTYYADSVKGRFTISADTSKNTA
YLQMNSLRAEDTAVYYCARRHWPGGFDYVVGQGTLVTVSS
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKFNW
YVDGVEVHNAKTKPREEQYASTYRVVSVLTVLHQDWLNGK
EYKCKVSNKALPAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPG;

and (b) the light chain comprises the amino acid sequence:

(SEQ ID NO: 29)
DIQMTQSPSSLSASVGDRVTITCRASQDVSTAVAVVYQQK
PGKAPKLLIYSASFLYSGVPSRFSGSGSGTDFTLTISSLQ
PEDFATYYCQQYLYHPATFGQGTKVEIKRTVAAPSVFIFP
PSDEQLKSGTASVVCLLNNFYPREAKVQWKVDNALQSGNS
QESVTEQDSKDSTYSLSSTLTLSKADYEKHKVYACEVTHQ
GLSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 26); (b) a VL domain comprising an amino acid sequence comprising having at least 95% sequence identity (e.g., at least 95%, 96%, 97%, 98%, or 99% sequence identity) to, or the sequence of (SEQ ID NO: 27); or (c) a VH domain as in (a) and a VL domain as in (b). In other instances, the anti-PD-L1 antagonist antibody is selected from YW243.55.570, MDX-1105, and MEDI4736 (durvalumab), and MSB0010718C (avelumab). Antibody YW243.55.570 is an anti-PD-L1 described in PCT Pub. No. WO 2010/077634. MDX-1105, also known as BMS-936559, is an anti-PD-L1 antibody described in PCT Pub. No. WO 2007/005874. MED14736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference. The anti-PD-L1 antagonist antibodies (e.g., atezolizumab) useful in this invention, including compositions containing such antibodies, may be used in combination with an anti-TIGIT antagonist antibody to treat ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)).

In some instances, the anti-PD-L1 antagonist antibody is a monoclonal antibody. In some instances, the anti-PD-L1 antagonist antibody is an antibody fragment selected from the group consisting of Fab, Fab′-SH, Fv, scFv, and (Fab′)₂ fragments. In some instances, the anti-PD-L1 antagonist antibody is a humanized antibody. In some instances, the anti-PD-L1 antagonist antibody is a human antibody. In some instances, the anti-PD-L1 antagonist antibody described herein binds to human PD-L1.

In some instances, the PD-1 axis binding antagonist is an anti-PD-1 antagonist antibody that inhibits the binding of PD-1 to its binding partner (e.g., PD-L1). In some instances, the anti-PD-1 antagonist antibody is capable of inhibiting binding between PD-L1 and PD-1.

In some instances, the PD-1 axis binding antagonist is an anti-PD-1 antibody. In some instances, the anti-PD-1 antibody is nivolumab (MDX-1106), pembrolizumab (formerly lambrolizumab (MK-3475)), or AMP-224.

In a further aspect, a PD-1 axis binding antagonist is a PD-1 axis binding antagonist antibody according to any of the above instances may incorporate any of the features, singly or in combination, as described in Section C below.

C. Antibody Formats and Properties

1. Antibody Affinity

In certain instances, an anti-TIGIT antagonist antibody and/or PD-1 axis binding antagonist antibody (e.g., anti-PD-L1 antagonist antibody) provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In one instance, K_{D} is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M-¹s-¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an anti-TIGIT antagonist antibody and/or PD-1 axis binding antagonist antibody (e.g., anti-PD-L1 antagonist antibody) provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain instances, an anti-TIGIT antagonist antibody and/or PD-1 axis binding antagonist antibody (e.g., anti-PD-L1 antagonist antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain instances, an anti-TIGIT antagonist antibody and/or PD-1 axis binding antagonist antibody (e.g., anti-PD-L1 antagonist antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-TIGIT antagonist antibody and/or PD-1 axis binding antagonist antibodies (e.g., anti-PD-L1 antagonist antibodies) of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody and/or PD-1 axis binding antagonist antibodies (e.g., anti-PD-L1 antagonist antibodies) or antibody fragments isolated from human antibody libraries are considered human antibodies or human antibody fragments herein.

6. Antibody Variants

In certain instances, amino acid sequence variants of the anti-TIGIT antagonist antibodies and/or PD-1 axis binding antagonist antibodies (e.g., anti-PD-L1 antagonist antibodies) of the invention are contemplated. As described in detail herein, anti-TIGIT antagonist antibodies and PD-1 axis binding antagonist antibodies (e.g., anti-PD-L1 antagonist antibodies) may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, anti-TIGIT antagonist antibody and/or PD-1 axis binding antagonist antibody (e.g., anti-PD-L1 antagonist antibody) variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

II. Glycosylation Variants

In certain instances, anti-TIGIT antagonist antibodies and/or PD-1 axis binding antagonist antibodies (e.g., anti-PD-L1 antagonist antibodies) of the invention can be altered to increase or decrease the extent to which the antibody is glycosylated. Addition or deletion of glycosylation sites to anti-TIGIT antagonist antibody and/or PD-1 axis binding antagonist antibody (e.g., anti-PD-L1 antagonist antibody) of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-TIGIT antagonist antibody and/or PD-1 axis binding antagonist antibody (e.g., anti-PD-L1 antagonist antibody) variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject or population of subjects in the context of a fractionated, dose-escalation dosing regimen an anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab)) variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-TIGIT antagonist antibody and/or PD-1 axis binding antagonist antibody (e.g., anti-PD-L1 antagonist antibody) variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain instances, one or more amino acid modifications are introduced into the Fc region of an anti-TIGIT antagonist (e.g., an anti-TIGIT antagonist antibody disclosed herein, e.g., tiragolumab) antibody and/or PD-1 axis binding antagonist antibody (e.g., anti-PD-L1 antagonist antibody (e.g., atezolizumab)) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an anti-TIGIT antagonist antibody and/or PD-1 axis binding antagonist antibody (e.g., anti-PD-L1 antagonist antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII, and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instance, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the anti-TIGIT antagonist antibody (e.g., an anti-TIGIT antagonist antibody disclosed herein, e.g., tiragolumab) and/or anti-PD-L1 antagonist antibody (e.g., atezolizumab) comprises an Fc region comprising an N297G mutation (EU numbering).

In some instances, the anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab)) comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3₁ and CH3₂ domains each comprise a protuberance or cavity, and wherein the protuberance or cavity in the CH3₁ domain is positionable in the cavity or protuberance, respectively, in the CH3₂ domain. In some instances, the CH3₁ and CH3₂ domains meet at an interface between said protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) is an IgG1 antibody.

IV. Cysteine Engineered Antibody Variants

In certain instances, it is desirable to create cysteine engineered anti-TIGIT antagonist antibodies and/or PD-1 axis binding antagonist antibodies (e.g., anti-PD-L1 antagonist antibodies), e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

V. Antibody Derivatives

In certain instances, an anti-TIGIT antagonist antibody of the invention (e.g., an anti-TIGIT antagonist antibody (e.g., tiragolumab) or a variant thereof) and/or PD-1 axis binding antagonist antibody (e.g., anti-PD-L1 antagonist antibody of the invention (e.g., atezolizumab or a variant thereof)) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-non proteinaceous moiety are killed.

Recombinant Production Methods

Anti-TIGIT antagonist antibodies (e.g., an anti-TIGIT antagonist antibody disclosed herein, e.g., tiragolumab) and/or PD-1 axis binding antagonist antibodies (e.g., anti-PD-L1 antagonist antibodies (e.g., atezolizumab)) of the invention may be produced using recombinant methods and compositions, for example, as described in U.S. Pat. No. 4,816,567, which is incorporated herein by reference in its entirety.

For recombinant production of an anti-TIGIT antagonist antibody and/or PD-1 axis binding antagonist antibody (e.g., anti-PD-L1 antagonist antibody), nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. Fora review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Immunoconjugates

The invention also provides immunoconjugates comprising an anti-TIGIT antagonist (e.g., an anti-TIGIT antagonist antibody as disclosed herein, e.g., tiragolumab) and/or PD-1 axis binding antagonist (e.g., anti-PD-L1 antagonist antibody (e.g., atezolizumab)) of the invention conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some instances, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-TIGIT antagonist antibody as described herein (e.g., tiragolumab) or a PD-1 axis binding antagonist (e.g., an anti-PD-L1 antagonist antibody (e.g., atezolizumab)) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-TIGIT antagonist antibody as described herein (e.g., tiragolumab) and/or a PD-1 axis binding antagonist (e.g., an anti-PD-L1 antagonist antibody) as described herein (e.g., atezolizumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

Pharmaceutical Compositions, Formulations, and Kits for Second-Line Therapies

Any of the anti-TIGIT antagonist antibodies and PD-1 axis binding antagonists described herein can be used in pharmaceutical compositions and formulations. Pharmaceutical compositions and formulations of an anti-TIGIT antagonist antibody and a PD-1 axis binding antagonist (e.g., an anti-PD-L1 antagonist antibody) can be prepared by mixing one, two, three, or all four agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

In another embodiment of the invention, a kit is provided comprising an anti-TIGIT antagonist antibody for use in combination with a PD-1 axis binding antagonist for treating a subject having an ESCC according to any of the methods described herein. In some instances, the kit further comprises the PD-1 axis binding antagonist.

In another embodiment, a kit comprises tiragolumab for use in combination with atezolizumab for treating a subject having an ESCC according to any of the methods described herein. In some embodiments, the kit further comprises atezolizumab.

Kits provided herein may include a PD-1 axis binding antagonist (e.g., atezolizumab) for use in combination with an anti-TIGIT antagonist antibody (e.g., tiragolumab) for treating a subject having an ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC or a Stage IVB ESCC with supraclavicular lymph node metastases only)) according to any of the methods described herein. In some embodiments, the kit further comprises tiragolumab. In some embodiments, the kit comprises tiragolumab and atezolizumab.

### IV. FIRST-LINE ESCC THERAPIES

In some aspects, the present invention involves treatments for a subject or population of subjects having advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC)). In some embodiments, the subject or population of subjects received no prior systemic treatment for advanced ESCC. In some embodiments, surgery is not suitable for the subject or population of subjects. The present treatments include a combination of an anti-TIGIT antagonist antibody (e.g., an anti-TIGIT antagonist antibody as disclosed herein, e.g., tiragolumab), a PD-1 axis binding antagonist (e.g., an anti-PD-L1 antagonist antibody (e.g., atezolizumab)), a taxane (e.g., paclitaxel), and a platinum agent (e.g., cisplatin). In some instances, the subject or population of subjects has received no prior systemic treatment for non-advanced ESCC. Alternatively, the subject or population of subjects has received prior treatment for non-advanced ESCC, and the prior treatment was completed at least six months before diagnosis of the advanced ESCC. For example, in some instances, the subject or population of subjects has received a prior chemoradiotherapy or a chemotherapy (e.g., chemoradiotherapy or a chemotherapy administered with curative intent or in an adjuvant or neoadjuvant setting) as treatment for non-advanced ESCC, which was completed at least six months before diagnosis of the advanced ESCC. In some instances, the prior treatment (e.g., chemoradiotherapy or a chemotherapy, e.g., chemoradiotherapy or a chemotherapy administered with curative intent or in an adjuvant or neoadjuvant setting) was completed at least eight months, at least ten months, at least one year, at least two years, at least three years, at least four years, or at least five years before diagnosis of the advanced ESCC. In some instances, the advanced ESCC is not suitable for definitive treatment (e.g., radiotherapy, chemoradiotherapy, and/or surgery).

In one aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 30 mg to about 1200 mg every three weeks (e.g., at a fixed dose of about 30 mg to about 800 mg every three weeks, e.g., at a fixed dose of about 600 mg every three weeks)), a PD-1 axis binding antagonist (e.g., at a fixed dose of about 80 mg to about 1600 mg every three weeks (e.g., at a fixed dose of about 800 mg to about 1400 mg, e.g., at a fixed dose of about 1200 mg)), a taxane, and a platinum agent. In some embodiments, surgery is unsuitable for the subject or population of subjects. In some embodiments, the subject or population of subjects has received no prior systemic treatment for advanced ESCC. In some embodiments, the subject or population of subjects has received no prior systemic treatment for non-advanced ESCC. In other embodiments, the subject or population of subjects has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., chemoradiotherapy or chemotherapy administered with curative intent or in an adjuvant or neoadjuvant setting). In some embodiments, the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks. In some embodiments, the anti-TIGIT antagonist antibody, PD-1 axis binding antagonist, taxane, and platinum agent are administered during an induction phase. In some embodiments, the induction phase comprises a 21-day cycle or less than one complete 21-day dosing cycle. In some embodiments, the induction phase comprises one to six (e.g., one, two, three, four, five, or six) 21-day cycles. In some embodiments, the induction phase comprises at least six 21-day cycles. In some embodiments, the anti-TIGIT antagonist antibody and the PD-1 axis binding antagonist are further administered post-induction, e.g., during a maintenance phase following the sixth 21-day cycle. In some embodiments, the maintenance phase begins immediately after the end of the induction phase. In some embodiments, the induction phase and the maintenance phase are separated by an interval of time. In some embodiments, the maintenance phase begins at least about 1, 2, 3, 4, 5, 6, 7, 8, 9, or 10 weeks after the end of the induction phase. In some embodiments, the taxane and the platinum agent are omitted from each of the one or more maintenance phase dosing cycles.

In another aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 300 mg to about 800 mg every two weeks (e.g., at a fixed dose of about 400 mg to about 500 mg every two weeks, e.g., at a fixed dose of about 420 mg every two weeks)), a PD-1 axis binding antagonist (e.g., at a fixed dose of about 200 mg to about 1200 mg every two weeks (e.g., at a fixed dose of about 800 mg to about 1000 mg every two weeks, e.g., at a fixed dose of about 840 mg every two weeks)), a taxane, and a platinum agent. In some embodiments, surgery is unsuitable for the subject or population of subjects. In some embodiments, the subject or population of subjects has received no prior systemic treatment for advanced ESCC. In some embodiments, the subject or population of subjects has received no prior systemic treatment for non-advanced ESCC. In other embodiments, the subject or population of subjects has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., chemoradiotherapy or chemotherapy administered with curative intent or in an adjuvant or neoadjuvant setting). In some embodiments, the anti-TIGIT antagonist antibody is administered at a fixed dose of about 420 mg every two weeks and the PD-1 axis binding antagonist is administered at a fixed dose of about 840 mg every two weeks. In some embodiments, the anti-TIGIT antagonist antibody, PD-1 axis binding antagonist, taxane, and platinum agent are administered during an induction phase. In some embodiments, the induction phase comprises a 28-day cycle or less than one complete 28-day dosing cycle. In some embodiments, the induction phase comprises one to six (e.g., one, two, three, four, five, or six) 28-day cycles. In some embodiments, the induction phase comprises at least six 28-day cycles. In some embodiments, the anti-TIGIT antagonist antibody and the PD-1 axis binding antagonist are further administered post-induction, e.g., during a maintenance phase following the sixth 28-day cycle. In some embodiments, the maintenance phase begins immediately after the end of the induction phase. In some embodiments, the induction phase and the maintenance phase are separated by an interval of time. In some embodiments, the maintenance phase begins at least about 1, 2, 3, 4, 5, 6, 7, 8, 9, or 10 weeks after the end of the induction phase. In some embodiments, the taxane and the platinum agent are omitted from each of the one or more maintenance phase dosing cycles. In some embodiments, the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

In another aspect, provided herein is a method for treating a subject or population of subjects having an esophageal squamous cell carcinoma (ESCC) (e.g., an advanced ESCC), the method comprising administering to the subject or population of subjects one or more dosing cycles of an anti-TIGIT antagonist antibody (e.g., at a fixed dose of about 700 mg to about 1000 mg every four weeks (e.g., at a fixed dose of about 800 mg to about 900 mg every four weeks, e.g., at a fixed dose of about 840 mg every four weeks), a PD-1 axis binding antagonist (e.g., at a fixed dose of about 400 mg to about 2000 mg every four weeks (e.g., at a fixed dose of about 1600 mg to about 1800 mg every four weeks, e.g., at a fixed dose of about 1680 mg every four weeks)), a taxane, and a platinum agent. In some embodiments, surgery is unsuitable for the subject or population of subjects. In some embodiments, the subject or population of subjects has received no prior systemic treatment for advanced ESCC. In some embodiments, the subject or population of subjects has received no prior systemic treatment for non-advanced ESCC. In other embodiments, the subject or population of subjects has received prior treatment for non-advanced ESCC, wherein the prior treatment for the non-advanced ESCC was completed at least six months before diagnosis of the advanced ESCC. In some embodiments, the prior treatment for the non-advanced ESCC comprises a chemoradiotherapy or a chemotherapy (e.g., chemoradiotherapy or chemotherapy administered with curative intent or in an adjuvant or neoadjuvant setting). In some embodiments, the anti-TIGIT antagonist antibody is administered at a fixed dose of about 840 mg every four weeks and the PD-1 axis binding antagonist is administered at a fixed dose of about 1680 mg every four weeks. In some embodiments, the anti-TIGIT antagonist antibody, PD-1 axis binding antagonist, taxane, and platinum agent are administered during an induction phase. In some embodiments, the induction phase comprises a 28-day cycle or less than one complete 28-day dosing cycle. In some embodiments, the induction phase comprises one to six (e.g., one, two, three, four, five, or six) 28-day cycles. In some embodiments, the induction phase comprises at least six 28-day cycles. In some embodiments, the anti-TIGIT antagonist antibody and the PD-1 axis binding antagonist are further administered post-induction, e.g., during a maintenance phase following the sixth 28-day cycle. In some embodiments, the maintenance phase begins immediately after the end of the induction phase. In some embodiments, the induction phase and the maintenance phase are separated by an interval of time. In some embodiments, the maintenance phase begins at least about 1, 2, 3, 4, 5, 6, 7, 8, 9, or 10 weeks after the end of the induction phase. In some embodiments, the taxane and the platinum agent are omitted from each of the one or more maintenance phase dosing cycles. In some embodiments, the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

In some embodiments, the taxane is administered once per week, once every two weeks, once every three weeks, twice every three weeks, once every four weeks, twice every four weeks, or three times every four weeks. In some embodiments, the platinum agent is administered once per week, once every two weeks, once every three weeks, twice every three weeks, once every four weeks, twice every four weeks, or three times every four weeks. In some embodiments, the taxane and the platinum agent are both administered once per week, once every two weeks, once every three weeks, twice every three weeks, once every four weeks, twice every four weeks, or three times every four weeks.

Therapeutic Methods for First-Line Therapies

The therapeutic methods and uses of the invention described herein include, in one aspect, administering one or more dosing cycles to a subject or population of subjects having an advanced ESCC (e.g., locally advanced ESCC, unresectable ESCC, locally advanced unresectable ESCC, or recurrent or metastatic ESCC) e.g., Stage II ESCC, Stage III ESCC, or Stage IV ESCC (e.g., a Stage IVA ESCC), wherein the subject or population of subjects has received no prior systemic treatment for advanced ESCC. The one or more dosing cycles include an effective amount of an anti-TIGIT antagonist antibody (e.g., an anti-TIGIT antagonist antibody as disclosed herein, e.g., tiragolumab), an effective amount of a PD-1 axis binding antagonist (e.g., an anti-PD-L1 antagonist antibody (e.g., atezolizumab)), an effective amount of a taxane (e.g., paclitaxel), and an effective amount of a platinum agent (e.g., cisplatin).

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, the fixed dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab)) may be reduced as compared to a standard dose of the anti-TIGIT antagonist antibody administered as a monotherapy.

In some instances, the effective amount of the anti-TIGIT antagonist antibody (e.g., an anti-TIGIT antagonist antibody as disclosed herein, e.g., tiragolumab) is a fixed dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a fixed dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks).

In some instances, the effective amount of the anti-TIGIT antagonist antibody (e.g., an anti-TIGIT antagonist antibody as disclosed herein, e.g., tiragolumab) is a fixed dose of between about 200 mg to about 2000 mg (e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every four weeks (Q4W). In some instances, the effective amount of anti-TIGIT antagonist antibody (e.g., an anti-TIGIT antagonist antibody as disclosed herein, e.g., tiragolumab) is a fixed dose of about 840 mg every four weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every four weeks).

In some instances, the effective amount of the PD-1 axis binding antagonist (e.g., an anti-PD-L1 antagonist antibody (e.g., atezolizumab)) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks (Q3W). In some instances, the effective amount of the PD-1 axis binding antagonist is atezolizumab at a fixed dose of about 1200 mg every three weeks. In some embodiments, the effective amount of the PD-1 axis binding antagonist is pembrolizumab at a fixed dose of about 200 mg every three weeks or, alternatively, pembrolizumab at a fixed dose of about 400 mg every six weeks.

In some instances, the fixed dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab, a taxane (e.g., paclitaxel), and/or a platinum agent (e.g., cisplatin)) may be reduced as compared to a standard dose of the PD-1 axis binding antagonist (e.g., an anti-PD-L1 antagonist antibody (e.g., atezolizumab)) administered as a monotherapy.

In some instances, the effective amount of the PD-1 axis binding antagonist (e.g., an anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., an anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of PD-1 axis binding antagonist (e.g., an anti-PD-L1 antagonist antibody (e.g., atezolizumab)) is a dose of about 15 mg/kg administered every three weeks. In some instances, the dose of the PD-1 axis binding antagonist (e.g., an anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab, a taxane (e.g., paclitaxel), and/or a platinum agent (e.g., cisplatin) may be reduced as compared to a standard dose of the PD-1 axis binding antagonist (e.g., an anti-PD-L1 antagonist antibody (e.g., atezolizumab) administered as a monotherapy.

In some instances, the effective amount of the PD-1 axis binding antagonist (e.g., anti-PD-L1 antagonist antibody (e.g., atezolizumab)) is a fixed dose of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist is atezolizumab at a fixed dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some embodiments, the effective amount of the PD-1 axis binding antagonist is avelumab at a fixed dose of about 800 mg every two weeks. In some embodiments, the effective amount of the PD-1 axis binding antagonist is nivolumab at a fixed dose of about 240 mg every two weeks.

In some instances, the effective amount of the PD-1 axis binding antagonist (e.g., anti-PD-L1 antagonist antibody (e.g., atezolizumab)) is a fixed dose of between about 500 mg to about 3000 mg (e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab)) is a fixed dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680±0.5 mg, e.g., 1680 mg every four weeks). In some embodiments, the effective amount of the PD-1 axis binding antagonist is nivolumab at a fixed dose of about 480 mg every four weeks.

In some instances, the effective amount of the taxane (e.g., paclitaxel or nab-paclitaxel (ABRAXANE®)) is about 25 to about 300 mg/m² every three weeks (e.g., about 100-250 mg/m² or about 150-200 mg/m², e.g., about 25 mg/m², about 50 mg/m², about 75 mg/m², about 100 mg/m², about 125 mg/m², about 150 mg/m², about 175 mg/m², about 200 mg/m², about 225 mg/m², about 250 mg/m², about 275 mg/m², or about 300 mg/m²), whether by one or more administrations, every three weeks. In some instances, the taxane is administered at a dose of about 175 mg/m² every three weeks. For example, in some instances, paclitaxel is administered at a dose from about 25 to about 300 mg/m² every three weeks (e.g., about 100-250 mg/m² or about 150-200 mg/m², e.g., about 25 mg/m², about 50 mg/m², about 75 mg/m², about 100 mg/m², about 125 mg/m², about 150 mg/m², about 175 mg/m², about 200 mg/m², about 225 mg/m², about 250 mg/m², about 275 mg/m², or about 300 mg/m²), whether by one or more administrations, every three weeks. In some instances, the paclitaxel is administered at a dose of about 175 mg/m² every three weeks.

In some instances, the effective amount of the platinum agent (e.g., cisplatin or carboplatin) is about 20-200 mg/m² every three weeks (e.g., about 40-120 mg/m², about 50-100 mg/m², or about 60-80 mg/m², e.g., about 25 mg/m², about 50 mg/m², about 60 mg/m², about 65 mg/m², about 70 mg/m², about 75 mg/m², about 80 mg/m², about 100 mg/m², about 125 mg/m², about 150 mg/m², about 175 mg/m², or about 200 mg/m²), whether by one or more administrations, every three weeks. In some instances, the platinum agent is administered at a dose from about 60-80 mg/m² every three weeks. For example, in some instances, cisplatin is administered at a dose from about 20-200 mg/m² every three weeks (e.g., about 40-120 mg/m², about 50-100 mg/m², or about 60-80 mg/m², e.g., about 25 mg/m², about 50 mg/m², about 60 mg/m², about 65 mg/m², about 70 mg/m², about 75 mg/m², about 80 mg/m², about 100 mg/m², about 125 mg/m², about 150 mg/m², about 175 mg/m², or about 200 mg/m²), whether by one or more administrations, every three weeks. In some instances, the platinum agent is administered at a dose from about 60-80 mg/m² every three weeks.

In some instances, the effective amount of the platinum agent (e.g., carboplatin or cisplatin) is a dose sufficient to achieve an AUC=6 mg/ml/min. In some instances, the effective amount of the platinum agent (e.g., carboplatin or cisplatin) is a dose sufficient to achieve an AUC=5 mg/ml/min.

AUC can be calculated using the Calvert formula (Calvert et al., J. Clin. Oncol. 1989, 7:1748-56):

Total dose (mg)=(target AUC)×(glomerular filtration rate [GFR]+25)

In some instances, the effective amount of the platinum agent (e.g., carboplatin or cisplatin) is 200 mg-1500 mg (e.g., 300 mg-1200 mg, 400 mg-1100 mg, or 500 mg-1000 mg, e.g., 300 mg-400 mg, 400 mg-500 mg, 500 mg-600 mg, 600 mg-700 mg, 700 mg-750 mg, 750 mg-800 mg, 800 mg-900 mg, 900 mg-1000 mg, 1000 mg-1100 mg, or 1100 mg-1200 mg, e.g., about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg). In some instances, the effective amount of the platinum agent (e.g., carboplatin or cisplatin) is about 500 mg-1000 mg (e.g., about 500 mg, about 600 mg, about 700 mg, about 800 mg, about 900 mg, or about 1000 mg).

In any of the methods and uses of the invention, the anti-TIGIT antagonist antibody (e.g., an anti-TIGIT antagonist antibody as disclosed herein, e.g., tiragolumab), the PD-1 axis binding antagonist (e.g., an anti-PD-L1 antagonist antibody (e.g., atezolizumab)), the taxane (e.g., paclitaxel), and the platinum agent (e.g., cisplatin) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab), the PD-1 axis binding antagonist (e.g., an anti-PD-L1 antagonist antibody (e.g., atezolizumab)), the taxane (e.g., paclitaxel), and the platinum agent (e.g., cisplatin) continue until there is a loss of clinical benefit (e.g., confirmed disease progression, drug resistance, death, or unacceptable toxicity). In some instances, the length of each dosing cycle is about 15 to 24 days (e.g., 15 days, 16 days, 17 days, 18 days, 19 days, 20 days, 21 days, 22 days, 23 days, or 24 days). In some instances, the length of each dosing cycle is about 21 days. In some instances, the length of each dosing cycle is about 80 to 88 days (e.g., 80 days, 81 days, 82 days, 83 days, 84 days, 85 days, 86 days, 87 days, or 88 days). In some instances, the length of each dosing cycle is about 84 days. In some instances, the length of each dosing cycle is about 38 to 46 days (e.g., 38 days, 39 days, 40 days, 41 days, 42 days, 43 days, 44 days, 45 days, or 46 days). In some instances, the length of each dosing cycle is about 42 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, in some instances, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks). In some instances, the anti-TIGIT antagonist antibody (e.g., an anti-TIGIT antagonist antibody as disclosed herein, e.g., tiragolumab) is administered on about Day 1 (e.g., Day 1±3 days) and Day 22 (e.g., Day 22±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 and Day 22 of each 42-day cycle (i.e., at a fixed dose of about 600 mg every three weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1, Day 22, Day 43, and Day 64 of each 84-day cycle (i.e., at a fixed dose of about 600 mg every three weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 15 (e.g., Day 15±3 days), and Day 29 (e.g., Day 29±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 420 mg on Day 1, Day 15, and Day 29 of each 42-day cycle (i.e., at a fixed dose of about 420 mg every two weeks). In some instances, the anti-TIGIT antagonist antibody (e.g., an anti-TIGIT antagonist antibody as disclosed herein, e.g., tiragolumab) is administered on about Day 1 (e.g., Day 1±3 days), Day 29 (e.g., Day 29±3 days), and Day 57 (e.g., Day 57±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 840 mg on Day 1, Day 29, and Day 56 of each 84-day cycle (i.e., at a fixed dose of about 840 mg every four weeks). Similarly, in some instances, the PD-1 axis binding antagonist (e.g., an anti-PD-L1 antagonist antibody (e.g., atezolizumab)) is administered on about Day 1 (e.g., Day 1±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1 of each 21-day cycle (i.e., at a fixed dose of about 1200 mg every three weeks). In some instances, the PD-1 axis binding antagonist (e.g., an anti-PD-L1 antagonist antibody (e.g., atezolizumab)) is administered on about Day 1 (e.g., Day 1±3 days) and Day 22 (e.g., Day 22±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1 and Day 22 of each 42-day cycle (i.e., at a fixed dose of about 1200 mg every three weeks). In some instances, the PD-1 axis binding antagonist (e.g., an anti-PD-L1 antagonist antibody (e.g., atezolizumab)) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the PD-1 axis binding antagonist (e.g., an anti-PD-L1 antagonist antibody (e.g., atezolizumab)) is administered intravenously at a fixed dose of about 1200 mg on Day 1, Day 22, Day 43, and Day 64 of each 84-day cycle (i.e., at a fixed dose of about 1200 mg every three weeks). In some instances, the PD-1 axis binding antagonist (e.g., an anti-PD-L1 antagonist antibody (e.g., atezolizumab)) is administered on about Day 1 (e.g., Day 1±3 days), Day 15 (e.g., Day 15±3 days), and Day 29 (e.g., Day 29±3 days) of each dosing cycle. For example, the PD-1 axis binding antagonist (e.g., an anti-PD-L1 antagonist antibody (e.g., atezolizumab)) is administered intravenously at a fixed dose of about 840 mg on Day 1, Day 15, and Day 29 of each 42-day cycle (i.e., at a fixed dose of about 840 mg every two weeks). In some instances, the PD-1 axis binding antagonist (e.g., an anti-PD-L1 antagonist antibody (e.g., atezolizumab)) is administered on about Day 1 (e.g., Day 1±3 days), Day 29 (e.g., Day 29±3 days), and Day 57 (e.g., Day 57±3 days) of each dosing cycle. For example, the PD-1 axis binding antagonist (e.g., an anti-PD-L1 antagonist antibody (e.g., atezolizumab)) is administered intravenously at a fixed dose of about 1680 mg on Day 1, Day 29, and Day 56 of each 84-day cycle (i.e., at a fixed dose of about 1680 mg every four weeks). In some instances, the taxane (e.g., paclitaxel) is administered on about Day 1 (e.g., Day 1±3 days) of each dosing cycle. For example, in some instances, the taxane (e.g., paclitaxel) is administered intravenously at a dose of about 175 mg/m² on Day 1 of each 21-day cycle (i.e., at a dose of about 175 mg/m² every three weeks). In some instances, the taxane (e.g., paclitaxel) is administered on about Day 1 (e.g., Day 1±3 days) and Day 22 (e.g., Day 22±3 days) of each dosing cycle. For example, the taxane (e.g., paclitaxel) is administered intravenously at a dose of about 175 mg/m² on Day 1 and Day 22 of each 42-day cycle (i.e., at a dose of about 175 mg/m² every three weeks). In some instances, the taxane (e.g., paclitaxel) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the taxane (e.g., paclitaxel) is administered intravenously at a dose of about 175 mg/m² on Day 1, Day 22, Day 43, and Day 64 of each 84-day cycle (i.e., at a dose of about 175 mg/m² every three weeks). In some instances, the platinum agent (e.g., cisplatin) is administered on about Day 1 (e.g., Day 1±3 days) of each dosing cycle. For example, in some instances, the platinum agent (e.g., cisplatin) is administered intravenously at a dose of about 60-80 mg/m² on Day 1 of each 21-day cycle (i.e., at a dose of about 60-80 mg/m² every three weeks). In some instances, the platinum agent (e.g., cisplatin) is administered on about Day 1 (e.g., Day 1±3 days) and Day 22 (e.g., Day 22±3 days) of each dosing cycle. For example, the platinum agent (e.g., cisplatin) is administered intravenously at a dose of about 60-80 mg/m² on Day 1 and Day 22 of each 42-day cycle (i.e., at a dose of about 60-80 mg/m² mg every three weeks). In some instances, the platinum agent (e.g., cisplatin) is administered on about Day 1 (e.g., Day 1±3 days), Day 22 (e.g., Day 22±3 days), Day 43 (e.g., Day 43±3 days), and Day 64 (e.g., Day 64±3 days) of each dosing cycle. For example, the platinum agent (e.g., cisplatin) is administered intravenously at a dose of about 60-80 mg/m² on Day 1, Day 22, Day 43, and Day 64 of each 84-day cycle (i.e., at a dose of about 60-80 mg/m² every three weeks). In some instances, the anti-TIGIT antagonist antibody (e.g., an anti-TIGIT antagonist antibody as disclosed herein, e.g., tiragolumab), the PD-1 axis binding antagonist (e.g., an anti-PD-L1 antagonist antibody (e.g., atezolizumab)), the taxane (e.g., paclitaxel), and the platinum agent (e.g., cisplatin) are administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks), the PD-1 axis binding antagonist (e.g., an anti-PD-L1 antagonist antibody (e.g., atezolizumab)) is administered intravenously at a fixed dose of about 1200 mg on Day 1 of each 21-day cycle (i.e., at a fixed dose of about 1200 mg every three weeks), the taxane (e.g., paclitaxel) is administered intravenously at a dose of about 175 mg/m² on Day 1 of each 21-day cycle (i.e., at a dose of about 175 mg/m² every three weeks), and the platinum agent (e.g., cisplatin) is administered intravenously at a dose of about 60-80 mg/m² on Day 1 of each 21-day cycle (i.e., at a dose of about 60-80 mg/m² every three weeks).

In some instances, the anti-TIGIT antagonist antibody (e.g., an anti-TIGIT antagonist antibody as disclosed herein, e.g., tiragolumab) is administered to the subject by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., an anti-PD-L1 antagonist antibody (e.g., atezolizumab)) is administered to the subject by intravenous infusion over about 60±15 minutes (e.g. about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes). In some instances, the taxane (e.g., paclitaxel) is administered to the subject by intravenous infusion over about three hours±30 minutes (e.g., about 150 minutes, about 155 minutes, about 160 minutes, about 165 minutes, about 170 minutes, about 175 minutes, about 180 minutes, about 185 minutes, about 190 minutes, about 195 minutes, about 200 minutes, about 205 minutes, or about 210 minutes). In some instances, the platinum agent (e.g., cisplatin) is administered to the subject by intravenous infusion over about one to four hours (e.g., about two to three hours, e.g., about one hour, about two hours, about three hours, or about four hours, e.g., about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, about 190 minutes, about 200 minutes, about 210 minutes, about 220 minutes, about 230 minutes, or about 240 minutes).

In some instances, the anti-TIGIT antagonist antibody (e.g., an anti-TIGIT antagonist antibody as disclosed herein, e.g., tiragolumab) is administered to the subject before the PD-1 axis binding antagonist (e.g., an anti-PD-L1 antagonist antibody (e.g., atezolizumab)). In some instances, for example, following administration of the anti-TIGIT antagonist antibody and before administration of the PD-1 axis binding antagonist, the method includes an intervening first observation period. In some instances, the method further includes a second observation period following administration of the PD-1 axis binding antagonist (e.g., an anti-PD-L1 antagonist antibody (e.g., atezolizumab)). In some instances, the method includes both a first observation period following administration of the anti-TIGIT antagonist antibody and second observation period following administration of the PD-1 axis binding antagonist. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and PD-1 axis binding antagonist during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and PD-1 axis binding antagonist during the first and second observation periods, respectively.

In other instances, the PD-1 axis binding antagonist (e.g., an anti-PD-L1 antagonist antibody (e.g., atezolizumab)) is administered to the subject before the anti-TIGIT antagonist antibody (e.g., an anti-TIGIT antagonist antibody as disclosed herein, e.g., tiragolumab). In some instances, for example, following administration of the PD-1 axis binding antagonist and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the PD-1 axis binding antagonist and a second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist and anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist and anti-TIGIT antagonist antibody during the first and second observation periods, respectively.

In some instances, the anti-TIGIT antagonist antibody (e.g., an anti-TIGIT antagonist antibody as disclosed herein, e.g., tiragolumab) and the anti-PD-L1 antagonist antibody (e.g., atezolizumab) are administered to the subject simultaneously. In some instances, for example, following administration of the anti-TIGIT antagonist antibody and the PD-1 axis binding antagonist the method includes an observation period. In some instances, the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the PD-1 axis binding antagonist and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the PD-1 axis binding antagonist and anti-TIGIT antagonist antibody during the observation period.

In some instances, the taxane (e.g., paclitaxel) and the platinum agent (e.g., cisplatin) are administered after the anti-TIGIT antagonist antibody and the PD-1 axis binding antagonist. In some instances, the taxane (e.g., paclitaxel) is administered to the subject before the platinum agent (e.g., cisplatin). In some instances, for example, following administration of the taxane and before administration of the platinum agent, the method includes an intervening third observation period. In some instances, the method further includes a fourth observation period following administration of the platinum agent. In some instances, the method includes both a third observation period following administration of the taxane and fourth observation period following administration of the platinum agent. In some instances, the third and fourth observation periods are each between about 30 minutes to about 60 minutes in length. In instances in which the third and fourth observation periods are each about 60 minutes in length, the method may include recording the subject's vital signs (e.g., pulse rate, respiratory rate, blood pressure, and temperature) at about 30±10 minutes after administration of the taxane and the platinum agent during the third and fourth observation periods, respectively. In instances in which the third and fourth observation periods are each about 30 minutes in length, the method may include recording the subject's vital signs (e.g., pulse rate, respiratory rate, blood pressure, and temperature) at about 15±10 minutes after administration of the taxane and platinum agent during the first and second observation periods, respectively.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of an anti-TIGIT antagonist antibody at a fixed dose of 600 mg every three weeks, atezolizumab at a fixed dose of 1200 mg every three weeks, paclitaxel at a dose of 175 mg/m² every three weeks, and cisplatin at a dose from 60-80 mg/m² every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of tiragolumab at a fixed dose of 600 mg every three weeks, atezolizumab at a fixed dose of 1200 mg every three weeks, paclitaxel at a dose of 175 mg/m² every three weeks, and cisplatin at a dose from 60-80 mg/m² every three weeks.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of an anti-TIGIT antagonist antibody at a fixed dose of 600 mg every three weeks, atezolizumab at a fixed dose of 840 mg every two weeks, paclitaxel at a dose of 175 mg/m² every three weeks, and cisplatin at a dose from 60-80 mg/m² every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of tiragolumab at a fixed dose of 600 mg every three weeks, atezolizumab at a fixed dose of 840 mg every two weeks, paclitaxel at a dose of 175 mg/m² every three weeks, and cisplatin at a dose from 60-80 mg/m² every three weeks.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of an anti-TIGIT antagonist antibody at a fixed dose of 600 mg every three weeks, atezolizumab at a fixed dose of 1680 mg every four weeks, paclitaxel at a dose of 175 mg/m² every three weeks, and cisplatin at a dose from 60-80 mg/m² every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of tiragolumab at a fixed dose of 600 mg every three weeks, atezolizumab at a fixed dose of 1680 mg every four weeks, paclitaxel at a dose of 175 mg/m² every three weeks, and cisplatin at a dose from 60-80 mg/m² every three weeks.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of an anti-TIGIT antagonist antibody at a fixed dose of 420 mg every two weeks, atezolizumab at a fixed dose of 1200 mg every three weeks, paclitaxel at a dose of 175 mg/m² every three weeks, and cisplatin at a dose from 60-80 mg/m² every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of tiragolumab at a fixed dose of 420 mg every two weeks, atezolizumab at a fixed dose of 1200 mg every three weeks, paclitaxel at a dose of 175 mg/m² every three weeks, and cisplatin at a dose from 60-80 mg/m² every three weeks.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of an anti-TIGIT antagonist antibody at a fixed dose of 420 mg every two weeks, atezolizumab at a fixed dose of 1680 mg every four weeks, paclitaxel at a dose of 175 mg/m² every three weeks, and cisplatin at a dose from 60-80 mg/m² every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, by administering to the subject or population of subjects one or more dosing cycles of tiragolumab at a fixed dose of 420 mg every two weeks, atezolizumab at a fixed dose of 1680 mg every four weeks, paclitaxel at a dose of 175 mg/m² every three weeks, and cisplatin at a dose from 60-80 mg/m² every three weeks.

In another aspect, the invention provides an anti-TIGIT antagonist antibody (e.g., an anti-TIGIT antagonist antibody disclosed herein, e.g., tiragolumab) and PD-1 axis binding antagonist (e.g., an anti-PD-L1 antagonist antibody (e.g., atezolizumab)) for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of an effective amount of an anti-TIGIT antagonist antibody (e.g., tiragolumab), an effective amount of a PD-1 axis binding antagonist (e.g., a PD-1 axis binding antagonist (e.g., an anti-PD-L1 antagonist antibody (e.g., atezolizumab))), an effective amount of a taxane (e.g., paclitaxel), and an effective amount of a platinum agent (e.g., cisplatin) (e.g., according to any of the methods described herein).

In another aspect, the invention provides uses of an anti-TIGIT antagonist antibody (e.g., an anti-TIGIT antagonist antibody disclosed herein, e.g., tiragolumab), a PD-1 axis binding antagonist (e.g., an anti-PD-L1 antagonist antibody (e.g., atezolizumab)), a taxane (e.g., paclitaxel), and a platinum agent (e.g., cisplatin) in the manufacture or preparation of a medicament for use in any of the methods described herein.

In another aspect, the invention provides uses of an anti-TIGIT antagonist antibody in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and a PD-1 axis binding antagonist (e.g., an anti-PD-L1 antagonist antibody (e.g., atezolizumab)), a taxane (e.g., paclitaxel), and a platinum agent (e.g., cisplatin), and wherein the medicament is formulated for administration of an effective amount of the anti-TIGIT antagonist antibody (e.g., tiragolumab), an effective amount of the PD-1 axis binding antagonist (e.g., an anti-PD-L1 antagonist antibody (e.g., atezolizumab)), an effective amount of the taxane (e.g., paclitaxel), and an effective amount of the platinum agent (e.g., cisplatin), according to any of the methods described herein.

In another aspect, the invention provides uses of a PD-1 axis binding antagonist (e.g., an anti-PD-L1 antagonist antibody (e.g., atezolizumab)), an anti-TIGIT antagonist antibody (e.g., an anti-TIGIT antagonist antibody disclosed herein, e.g., tiragolumab), a taxane (e.g., paclitaxel), and a platinum agent (e.g., cisplatin) in the manufacture or preparation of a medicament for use in any of the methods described herein.

In another aspect, the invention provides uses of a PD-1 axis binding antagonist (e.g., an anti-PD-L1 antagonist antibody (e.g., atezolizumab)) in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antagonist antibody (e.g., an anti-TIGIT antagonist antibody disclosed herein, e.g., tiragolumab), a taxane (e.g., paclitaxel), and a platinum agent (e.g., cisplatin), and wherein the medicament is formulated for administration of an effective amount of an effective amount of the PD-1 axis binding antagonist (e.g., an anti-PD-L1 antagonist antibody (e.g., atezolizumab)), an effective amount of the anti-TIGIT antagonist antibody (e.g., tiragolumab), an effective amount of the taxane (e.g., paclitaxel), and an effective amount of the platinum agent (e.g., cisplatin), according to any of the methods described herein.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 1200 mg every three weeks, the anti-TIGIT antagonist antibody is to be administered at a fixed dose of 600 mg every three weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 600 mg every three weeks, atezolizumab at a fixed dose of 1200 mg every three weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, atezolizumab, paclitaxel, and cisplatin, wherein the medicament is formulated for administration of tiragolumab at a fixed dose of 600 mg every three weeks, atezolizumab is to be administered at a fixed dose of 1200 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, tiragolumab, paclitaxel, and cisplatin, wherein the medicament is formulated for administration of atezolizumab at a fixed dose of 1200 mg every three weeks, tiragolumab is to be administered at a fixed dose of 600 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 840 mg every two weeks, the anti-TIGIT antagonist antibody is to be administered at a fixed dose of 600 mg every three weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 600 mg every three weeks, atezolizumab at a fixed dose of 840 mg every two weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 600 mg every three weeks, atezolizumab is to be administered at a fixed dose of 840 mg every two weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 840 mg every two weeks and tiragolumab is to be administered at a fixed dose of 600 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 1680 mg every four weeks, the anti-TIGIT antagonist antibody is to be administered at a fixed dose of 600 mg every three weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 600 mg every three weeks, atezolizumab at a fixed dose of 1680 mg every four weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 600 mg every three weeks, atezolizumab is to be administered at a fixed dose of 1680 mg every four weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 1680 mg every four weeks and tiragolumab is to be administered at a fixed dose of 600 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 1200 mg every three weeks, the anti-TIGIT antagonist antibody is to be administered at a fixed dose of 420 mg every two weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 420 mg every two weeks, atezolizumab at a fixed dose of 1200 mg every three weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 420 mg every two weeks, atezolizumab is to be administered at a fixed dose of 1200 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 1200 mg every three weeks and tiragolumab is to be administered at a fixed dose of 420 mg every two weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 1680 mg every four weeks, the anti-TIGIT antagonist antibody is to be administered at a fixed dose of 420 mg every two weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 420 mg every two weeks, atezolizumab at a fixed dose of 1680 mg every four weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 420 mg every two weeks, atezolizumab is to be administered at a fixed dose of 1680 mg every four weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 1680 mg every four weeks and tiragolumab is to be administered at a fixed dose of 420 mg every two weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 1200 mg every three weeks, the anti-TIGIT antagonist antibody is to be administered at a fixed dose of 840 mg every four weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 840 mg every four weeks, atezolizumab at a fixed dose of 1200 mg every three weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 840 mg every four weeks, atezolizumab is to be administered at a fixed dose of 1200 mg every three weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 1200 mg every three weeks and tiragolumab is to be administered at a fixed dose of 840 mg every four weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and an anti-TIGIT antibody, a taxane, and a platinum agent, wherein the medicament is formulated for administration of atezolizumab at a fixed dose of 840 mg every two weeks, the anti-TIGIT antagonist antibody is to be administered at a fixed dose of 840 mg every four weeks, the taxane is to be administered at a dose of about 175 mg/m² every three weeks, and the platinum agent is to be administered at a dose from 60-80 mg/m² every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament, wherein the medicament is formulated for administration of tiragolumab at a fixed dose of 840 mg every four weeks, atezolizumab at a fixed dose of 840 mg every two weeks, paclitaxel at a dose of about 175 mg/m² every three weeks, and cisplatin at a dose from 60-80 mg/m² every three weeks.

In another aspect, the invention provides uses of tiragolumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and atezolizumab, wherein the medicament is formulated for administration of tiragolumab at a fixed dose of 840 mg every four weeks, atezolizumab is to be administered at a fixed dose of 840 mg every two weeks, paclitaxel is to be administered at a dose of about 175 mg/m² every three weeks, and cisplatin is to be administered at a dose from 60-80 mg/m² every three weeks.

In another aspect, the invention provides uses of atezolizumab in the manufacture of a medicament for use in a method of treating a subject or population of subjects having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC), wherein the subject or population of subjects has received no prior systemic treatment for advanced ESCC, wherein the method comprises administering to the subject or population of subjects one or more dosing cycles of the medicament and tiragolumab, wherein the medicament is formulated for administration of atezolizumab at a fixed dose of 840 mg every two weeks and tiragolumab is to be administered at a fixed dose of 840 mg every four weeks, paclitaxel is to be administered at a dose of about 175 mg/m² every three weeks, and cisplatin is to be administered at a dose from 60-80 mg/m² every three weeks.

A. PD-L1 Selection

In some instances of any of the methods, uses, or compositions for use described herein, the subject or population of subjects has a PD-L1 selected ESCC tumor (e.g., an ESCC tumor with a detectable expression level (e.g., protein expression level or nucleic acid expression level) of PD-L1. In some instances, the PD-L1 selected tumor is an ESCC tumor that has been determined to have a PD-L1-positive tumor associated immune cell (TIC) score of at least 1% (e.g., at least 10%) by an immunohistochemical (IHC) assay. In some instances, the TIC score is from 1% to 99% (e.g., from 2% to 98%, from 3% to 97%, from 4% to 96%, from 5% to 95%, from 10% to 90%, from 15% to 85%, from 20% to 80%, or from 25% to 75%, e.g., from 1% to 10% (e.g., from 1% to 5% (e.g., from 1% to 2%, from 2% to 3%, from 3% to 4%, or from 4% to 5%) or from 5% to 10% (e.g., from 5% to 6%, from 6% to 7%, from 7% to 8%, from 8% to 9%, or from 9% to 10%)), from 10% to 20% (e.g., from 10% to 15% (e.g., from 10% to 11%, from 11% to 12%, from 12% to 13%, from 13% to 14%, or from 14% to 15%) or from 15% to 20% (e.g., from 15% to 16%, from 16% to 17%, from 17% to 18%, from 18% to 19%, or from 19% to 20%)), or greater than 20%). In some instances, the TIC score is less than 10% (e.g., from 1% to 10%, from 2% to 10%, from 3% to 10%, from 4% to 10%, from 5% to 10%, from 6% to 10%, from 7% to 10%, from 8% to 10%, or from 9% to 10%). In some instances, the TIC score is less than 20% (e.g., from 1% to 20%, from 2% to 20%, from 3% to 20%, from 4% to 20%, from 5% to 20%, from 6% to 20%, from 7% to 20%, from 8% to 20%, from 9% to 20%, from 10% to 20%, from 11% to 20%, from 12% to 20%, from 13% to 20%, from 14% to 20%, from 15% to 20%, from 16% to 20%, from 17% to 20%, from 18% to 20%, or from 19% to 20%).

In some instances, the IHC assay is the pharmDX 22C3 assay and the ESCC tumor sample has been determined to have a combined positive score (CPS) of greater than, or equal to, 10 (e.g., greater than, or equal to, 15; greater than, or equal to, 20; greater than, or equal to, 25; greater than, or equal to, 30; greater than, or equal to, 40; greater than, or equal to, 45; or greater than, or equal to, 50). In some embodiments, the ESCC tumor sample has been determined to have a TPS of greater than, or equal to, 1%. In some embodiments, the ESCC tumor sample has been determined to have a TPS of greater than, or equal to, 50%.

In some instances, the IHC assay uses the anti-PD-L1 antibody SP142 or 28-8. In some instances, the IHC assay uses anti-PD-L1 antibody SP142 (e.g., Ventana SP142 IHC assay). In some instances, the IHC assay uses anti-PD-L1 antibody 28-8 (e.g., pharmDx 28-8 IHC assay).

In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

B. Responses to First-Line Therapies

In some embodiments of any of the methods described herein, a subject or population of subjects' response to the therapy can be characterized by one or more measures. In some embodiments, the treatment results in a complete response or a partial response.

In some instances, the treatment results in an increase in progression-free survival of the subject, e.g., as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). For example, the treatment with the PD-1 axis binding antagonist and the anti-TIGIT antagonist antibody may result in an increase in progression-free survival of the subject, e.g., as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). In some embodiments, the treatment extends the PFS of the subject or population of subjects by at least about 2 months or about 4 months. In some embodiments, the increase in PFS is about 3.7 months or more (e.g., about 4.0 months or more, about 4.5 months or more, about 5.0 months or more, about 5.5. months or more, about 6.0 months or more, about 6.5 months or more, about 7.0 months or more, about 7.5 months or more, about 8.0 months or more, about 8.5 months or more, about 9.0 months or more, about 9.5 months or more, about 10 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in PFS is about 6 months or more (e.g., about 6.5 months or more, about 7 months or more, about 7.5 months or more, about 8 months or more, about 8.5 months or more, about 9 months or more, about 9.5 months or more, about 10 months or more, about 10.5 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in PFS is 2-4 months (e.g., about 2 months, about 2.5 months, about 3 months, about 3.5. months, or about 4 months). In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median PFS of at least about 8 months (e.g., about 8.5 months, about 9 months, about 9.5 months, about 10 months, about 10.5 months, about 11 months, about 11.5 months, about 12 months, about 12.5 months, about 13 months, about 14 months, about 15 months, about 16 months, about 17 months, about 18 months, about 19 months, about 20 months, about 21 months, about 22 months, about 23 months, about 24 months, about 25 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin). In some embodiments, the treatment results in a median PFS of the population of subjects of about 6 months to about 10 months. In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median PFS between 8 months and 60 months (e.g., between 9 and 60 months, between 10 and 60 months, between 11 and 60 months, between 12 and 60 months, between 13 and 60 months, between 14 and 60 months, between 15 and 60 months, between 16 and 60 months, between 17 and 60 months, between 18 and 60 months, between 19 and 60 months, between 20 and 60 months, between 25 and 60 months, between 30 and 60 months, between 35 and 60 months, between 40 and 60 months, between 45 and 60 months, between 50 and 60 months, or between 55 and 60 months) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

In some instances, the treatment results in an increase in overall survival of the subject or population of subjects, e.g., as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). For example, the treatment with the PD-1 axis binding antagonist and the anti-TIGIT antagonist antibody may result in an increase in overall survival of the subject or population of subjects, e.g., as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). In some embodiments, the treatment extends the OS of the subject or population of subjects by at least about 4 months or about 6 months. In some embodiments, the increase in OS is about 4.1 months or more (e.g., about 4.5 months or more, about 5.0 months or more, about 5.5. months or more, about 6.0 months or more, about 6.5 months or more, about 7.0 months or more, about 7.5 months or more, about 8.0 months or more, about 8.5 months or more, about 9.0 months or more, about 9.5 months or more, about 10 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in OS is about 6 months or more (e.g., about 6.5 months or more, about 7 months or more, about 7.5 months or more, about 8 months or more, about 8.5 months or more, about 9 months or more, about 9.5 months or more, about 10 months or more, about 10.5 months or more, about 11 months or more, about 11.5 months or more, about 12 months or more, about 12.5 months or more, about 13 months or more, about 13.5 months or more, about 14 months or more, about 14.5 months or more, about 15 months or more, about 15.5 months or more, about 16 months or more, about 16.5 months or more, about 17 months or more, about 17.5 months or more, about 18 months or more, about 18.5 months or more, about 19 months or more, about 19.5 months or more, or about 20 months or more). In some embodiments, the increase in OS is 4-6 months (e.g., about 4 months, about 4.5 months, about 5 months, about 5.5. months, or about 6 months). In some embodiments, the treatment results in a median OS of the population of subjects of about 14 months to about 20 months. In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median OS of at least about 14 months (e.g., about 14.5 months, about 15 months, about 15.5 months, about 16 months, about 16.5 months, about 17 months, or about 17.5 months) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin). In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median OS of at least about 14 months (e.g., about 14.5 months, about 15 months, about 15.5 months, about 16 months, about 16.5 months, about 17 months, about 17.5 months, about 18 months, about 18.5 months, about 19 months, about 19.5 months, about 20 months, about 20.5 months, about 21 months, about 21.5 months, about 22 months, about 22.5 months, about 23 months, about 23.5 months, about 24 months, about 24.5 months, about 25 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin). In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median OS between 18 months and 60 months (e.g., between 19 and 60 months, between 20 and 60 months, between 25 and 60 months, between 30 and 60 months, between 35 and 60 months, between 40 and 60 months, between 45 and 60 months, between 50 and 60 months, or between 55 and 60 months) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

In some instances, the treatment results in an increase in duration of objective response (DOR) in the subject or population of subjects as compared to treatment with the PD-1 axis binding antagonist without the anti-TIGIT antagonist antibody or as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). In some instances, the treatment results in an increase in DOR in the subject or population of subjects as compared to treatment with the taxane (e.g., paclitaxel) and the platinum agent (e.g., cisplatin), without the PD-1 axis binding antagonist (e.g., atezolizumab) and the anti-TIGIT antagonist antibody (e.g., tiragolumab). In some embodiments, the increase in DOR is about 2 months or more (e.g. about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more). In some embodiments, administration of the anti-TIGIT antagonist antibody (e.g., tiragolumab), the PD-1 axis binding antagonist (e.g., atezolizumab), the taxane (e.g., paclitaxel), and the platinum agent (e.g., cisplatin) to a plurality of subjects results in a median DOR of at least about 2 months or more (e.g., about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, about 12 months, about 12.5 months, about 13 months, about 13.5 months, about 14 months, about 14.5 months, about 15 months, about 15.5 months, about 16 months, about 16.5 months, about 17 months, about 17.5 months, about 18 months, about 18.5 months, about 19 months, about 19.5 months, about 20 months, or more) after the start of treatment with the anti-TIGIT antagonist antibody (e.g., tiragolumab), the PD-1 axis binding antagonist (e.g., atezolizumab), the taxane (e.g., paclitaxel), and the platinum agent (e.g., cisplatin).

Progression-free survival of the subject or population of subjects can be measured according to RECIST v1.1 criteria, as described in Eisenhauer et al., Eur. J. Cancer. 2009, 45:228-47. In some embodiments, PFS is measured as the period of time from the start of treatment to the first occurrence of disease progression as determined by RECIST v1.1 criteria. In some embodiments, PFS is measured as the time from the start of treatment to the time of death.

Exemplary Anti-TIGIT Antagonist Antibodies, PD-1 Axis Binding Antagonists, Taxanes, and Platinum Agents for First-Line Therapies

Exemplary anti-TIGIT antagonist antibodies, PD-1 axis binding antagonists (e.g., anti-PD-L1 antibodies), taxanes, and platinum agents useful for treating a subject (e.g., a human) having advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC) in accordance with the methods, uses, and compositions for use of the invention are described herein. In particular, the following exemplary anti-TIGIT antagonist antibodies, PD-1 axis binding antagonists (e.g., anti-PD-L1 antibodies), taxanes, and platinum agents can be used to treat subjects who have received no prior systemic treatment for advanced ESCC.

A. Anti-TIGIT Antagonist Antibodies

The invention provides anti-TIGIT antagonist antibodies useful for treating advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC) in a subject (e.g., a human).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, anti-TIGIT antagonist antibodies may include (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 17)
EVQLQQSGPGLVKPSQTLSLTCAISGDSVSSNSAAWNWIR
QSPSRGLEWLGKTYYRFKVVYSDYAVSVKGRITINPDTSK
NQFSLQLNSVTPEDTAVFYCTRESTTYDLLAGPFDYVVGQ
GTLVTVSS

or an amino acid sequence having at least 90% sequence identity (e.g., at least 91%, 92%, 93%, 94%, 95%, 96%, 97%, 98%, or 99% sequence identity) to, or the sequence of,

(SEQ ID NO: 18)
QVQLQQSGPGLVKPSQTLSLTCAISGDSVSSNSAAWNWIR
QSPSRGLEWLGKTYYRFKVVYSDYAVSVKGRITINPDTSK
NQFSLQLNSVTPEDTAVFYCTRESTTYDLLAGPFDYVVGQ
GTLVTVSS;

and/or a VL domain comprising an amino acid sequence having at least 90% sequence identity (e.g., at least 91%, 92%, 93%, 94%, 95%, 96%, 97%, 98%, or 99% sequence identity) to, or the sequence of,

(SEQ ID NO: 19)
DIVMTQSPDSLAVSLGERATINCKSSQTVLYSSNNKKYLA
VVYQQKPGQPPNLLIYWASTRESGVPDRFSGSGSGTDFTL
TISSLQAEDVAVYYCQQYYSTPFTFGPGTKVEIK.

In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 17 and a VL domain comprising the amino acid sequence of SEQ ID NO: 19. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 18 and a VL domain comprising the amino acid sequence of SEQ ID NO: 19.

In some instances, the anti-TIGIT antagonist antibody includes a heavy chain and a light chain sequence, wherein: (a) the heavy chain comprises the amino acid sequence:

(SEQ ID NO: 33)
EVQLQQSGPGLVKPSQTLSLTCAISGDSVSSNSAAWNWIR
QSPSRGLEWLGKTYYRFKVVYSDYAVSVKGRITINPDTSK
NQFSLQLNSVTPEDTAVFYCTRESTTYDLLAGPFDYVVGQ
GTLVTVSSASTKGPSVFPLAPSSKSTSGGTAALGCLVKDY
FPEPVTVSWNSGALTSGVHTFPAVLQSSGLYSLSSVVTVP
SSSLGTQTYICNVNHKPSNTKVDKKVEPKSCDKTHTCPPC
PAPELLGGPSVFLFPPKPKDTLMISRTPEVTCVVVDVSHE
DPEVKFNVVYVDGVEVHNAKTKPREEQYNSTYRVVSVLTV
LHQDWLNGKEYKCKVSNKALPAPIEKTISKAKGQPREPQV
YTLPPSREEMTKNQVSLTCLVKGFYPSDIAVEWESNGQPE
NNYKTTPPVLDSDGSFFLYSKLTVDKSRWQQGNVFSCSVM
HEALHNHYTQKSLSLSPGK;

and (b) the light chain comprises the amino acid sequence:

(SEQ ID NO: 34)
DIVMTQSPDSLAVSLGERATINCKSSQTVLYSSNNKKYLA
VVYQQKPGQPPNLLIYWASTRESGVPDRFSGSGSGTDFTL
TISSLQAEDVAVYYCQQYYSTPFTFGPGTKVEIKRTVAAP
SVFIFPPSDEQLKSGTASVVCLLNNFYPREAKVQWKVDNA
LQSGNSQESVTEQDSKDSTYSLSSTLTLSKADYEKHKVYA
CEVTHQGLSSPVTKSFNRGEC.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of XiVQLQQSGPGLVKPSQTLSLTCAISGDSVS (SEQ ID NO: 11), wherein Xi is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), ora combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14. In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization. In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In some aspects, the anti-TIGIT antagonist antibody is an antibody having intact Fc-mediated effector function (e.g., tiragolumab, vibostolimab, etigilimab, EO5084448, or TJ-T6) or enhanced effector function (e.g., SGN-TGT).

In other aspects, the anti-TIGIT antagonist antibody is an antibody that lacks Fc-mediated effector function (e.g., domvanalimab, BMS-986207, ASP8374, or COM902).

In some aspects, the anti-TIGIT antagonist antibody is an IgG1 class antibody, e.g., tiragolumab, vibostolimab, domvanalimab, BMS-986207, etigilimab, BGB-A1217, SGN-TGT, EOS084448 (EOS-448), TJ-T6, or AB308.

In other aspects, the anti-TIGIT antagonist antibody is an IgG4 class antibody, e.g., ASP8374 or COM902.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or R07092284), vibostolimab (MK-7684), ASP8374 (PTZ-201), EO5884448 (EOS-448), SEA-TGT (SGN-TGT)), BGB-A1217, BMS-986207 (ONO-4686), COM902 (CGEN-15137), 161939, domvanalimab (AB154), M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or RO7092284), vibostolimab (MK-7684), ASP8374 (PTZ-201), EOS-448, and SEA-TGT (SGN-TGT). The anti-TIGIT antibody may be tiragolumab (MTIG7192A, RG6058 or R07092284).

Non-limiting examples of anti-TIGIT antibodies that are useful for the methods disclosed herein, and methods for making thereof are described in PCT Pub. Nos. WO2018183889A1, WO2019129261A1, WO2016106302A9, WO2018033798A1, WO2020020281A1, WO2019023504A1, WO2017152088A1, WO2016028656A1, WO2017030823A2, WO2018204405A1, WO2019152574A1, and WO2020041541A2; U.S. Pat. Nos. 10,189,902, 10,213,505, 10,124,061, 10,537,633, and 10,618,958; and U.S. Pub. Nos. 2020/0095324, 2019/0112375, 2018/0371083, and 2020/0062859, each of which is incorporated herein by reference in its entirety. Additional non-limiting examples of anti-TIGIT antibodies, useful for the methods of disclosed herein, and methods for making thereof are described in PCT Pub. Nos. WO2018204363A1, WO2018047139A1, WO2019175799A2, WO2018022946A1, WO2015143343A2, WO2018218056A1, WO2019232484A1, WO2019079777A1, WO2018128939A1, WO2017196867A1, WO2019154415A1, WO2019062832A1, WO2018234793A3, WO2018102536A1, WO2019137548A1, WO2019129221A1, WO2018102746A1, WO2018160704A9, WO2020041541A2, WO2019094637A9, WO2017037707A1, WO2019168382A1, WO2006124667A3, WO2017021526A1, WO2017184619A2, WO2017048824A1, WO2019032619A9, WO2018157162A1, WO2020176718A1, WO2020047329A1, WO2020047329A1, WO2018220446A9; U.S. Pat. Nos. 9,617,338, 9,567,399, 10,604,576, and 9,994,637; and Pub. Nos. US 2018/0355040, US 2019/0175654, US 2019/0040154, US 2019/0382477, US 2019/0010246, US 2020/0164071, US 2020/0131267, US 2019/0338032, US 2019/0330351, US 2019/0202917, US 2019/0284269, US 2018/0155422, US 2020/0040082, US 2019/0263909, US 2018/0185480, US 2019/0375843, US 2017/0037133, US 2019/0077869, US 2019/0367579, US 2020/0222503, US 2020/0283496, CN109734806A, and CN110818795A, each of which is incorporated herein by reference in its entirety.

The anti-TIGIT antibodies useful in the methods disclosed herein include ASP8374 (PTZ-201), BGB-A1217, BMS-986207 (ONO-4686), COM902 (CGEN-15137), M6223,161939, EOS-448, domvanalimab (AB154), vibostolimab (MK-7684), and SEA-TGT (SGN-TGT). Additional TIGIT binding molecules, including anti-TIGIT antibodies, useful in the methods disclosed herein include AGEN1307; AGEN1777; antibody clones pab2197 and pab2196 (Agenus Inc.); antibody clones TBB8, TDC8, 3TB3, 5TB10, and D1Y1A (Anhui Anke Biotechnology Group Co. Ltd.), antibody clones MAB1, MAB2, MAB3, MAB4, MAB5, MAB6, MAB 7, MAB8, MAB9, MAB 10, MAB 11, MAB 12, MAB13, MAB 14, MAB 15, MAB 16, MAB 17, MAB 18, MAB19, MAB20, MAB21 (Astellas Pharma/Potenza Therapeutics), antibody clones hu1217-1-1 and hu1217-2-2 (BeiGene), antibody clones 4D4 and 19G (Brigham & Women's Hospital), antibody clones 11G11, 10D7, 15A6, 22G2, TIGIT G2a, and TIGIT G1 D265A, including such antibodies with modified heavy chain constant regions (Bristol-Myers Squibb); antibody clones 10A7, CPA.9.086, CPA.9.083.H4(S241P), CPA.9.086.H4(5241P), CHA.9.547.7.H4(S241P) and CHA.9.547.13.H4(S241P) (Compugen); anti-PVRIG/anti-TIGIT bispecific antibodies (Compugen), antibody clones 315293, 328189, 350426, 326504, and 331672 (Fred Hutchinson Cancer Research Center); antibody clones T-01, T-02, T-03, T-04, T-05, T-06, T-07, T-08, T-09, and T-10 (Gensun BioPharma Inc.); antibody clones 1H6, 2611, 3A10, 4A5, 4A9, 4H5, 6A2, 6B7, 7F4, 8E1, 8G3, 9F4, 9G6, 10C1, 10F10, 11G4, 1267, 12C8, 15E9, 16C11, 16D6, and 16E10 (Hefei Ruida Immunological Drugs Research Institute Co. Ltd.); antibody clones h3C5H1, h3C5H2, h3C5H3, h3C5H4, h3C5H3-1, h3C5H3-2, h3C5H3-3, h3C5L1, and h3C5L2 (IGM Biosciences Inc.); antibody clones 90D9, 101E1, 116H8, 118A12, 131A12, 143B6, 167F7, 221F11, 222H4, 327C9, 342A9, 344F2, 349H6, and 350D10 (I-Mab Biopharma); antibody clones ADI-27238, ADI-30263, ADI-30267, ADI-30268, ADI-27243, ADI-30302, ADI-30336, ADI-27278, ADI-30193, ADI-30296, ADI-27291, ADI-30283, ADI-30286, ADI-30288, AD127297, ADI-30272, ADI-30278, ADI-27301, ADI-30306, and ADI-30311 (Innovent Biologics, Inc.); antibody clones 26518, 29478, 26452, 29487, 29489, 31282, 26486, 29494, 29499, 26521, 29513, 26493, 29520, 29523, 29527, 31288, 32919, 32931, 26432, and 32959 (iTeos Therapeutics); antibody clones m1707, m1708, m1709, m1710, m1711, h1707, h1708, h1709, h1710, and h1711 (Jiangsu Hengrui Medicine Co. Ltd.); antibody clones TIG1, TIG2, and TIG3 (JN Biosciences LLC); antibody clones (e.g., KY01, KY02, KY03, KY04, KY05, KY06, KY07, KY08, KY09, KY10, K11, K12, K13, K14, K15, K16, K17, K18, K19, K₂O, K21, K22, K23 Kymab TIGIT (Antibody 2), and Tool TIGIT (Antibody 4) (Kymab Limited); bispecific antibodies 1D05/in-house anti-TIGIT with 1D05 (anti-PD-L1) Native variable domain and Kymab TIGIT antigen binding site (ABS) domain (Bispecific 1), In-house anti-TIGIT/1D05 with Kymab TIGIT Native variable domain and 1D05 ABS domain (Bispecific 2), Tool anti-TIGIT/Tool anti-PD-L1 with Toon anti-TIGIT Native variable domain and Tool anti-PD-L1 ABS domain (Bispecific 3), Tool anti-PD-L1/Tool anti-TIGIT with Tool anti-PD-L1 Native variable domain and Tool anti-TIGIT ABS domain (Bispecific 4) (Kymab Limited); antibody clones and clone variants 14D7, 26B10, Hu14D7, Hu26B10, 14A6, Hu14A6, 28H5, 3106, Hu31C6, 25G10, MBS43, 37D10, 18G10, 11A11, c18G10, and LB155.14A6.G2.A8 (Merck); etigilimab (OMP-313M32) (Mereo BioPharma); antibody clones 64G1E9B4, 100C4E7D11, 83G5H11C12, 92E9D4B4, 104G12E12G2, 121C2F10B5, 128E3F10F3F2, 70A11A8E6, 11D8E124A, 16F10H12C11, 8F2D8E7, 48B5G4E12, 139E2C2D2, 128E3G7F5, AS19584, AS19852, AS19858, AS19886, AS19887, AS19888, AS20160, AS19584VH26, AS19584VH29, AS19584VH30, AS19584VH31, AS19886VH5, AS19886VH8, AS19886VH9, AS19886VH10, AS19886VH19, AS19886VH20, AS19584VH28-Fc, AS19886VH5-Fc, AS19886VH8-Fc, AS19584-Fc, and AS19886-Fc (Nanjing Legend Biotechnology Co. Ltd.); antibody clones ARE clones: Ab58, Ab69, Ab75, Ab133, Ab177, Ab122, Ab86, Ab180, Ab83, Ab26, Ab20, Ab147, Ab12, Ab66, Ab176, Ab96, Ab123, Ab109, Ab149, Ab34, Ab61, Ab64, Ab105, Ab108, Ab178, Ab166, Ab29, Ab135, Ab171, Ab194, Ab184, Ab164, Ab183, Ab158, Ab55, Ab136, Ab39, Ab159, Ab151, Ab139, Ab107, Ab36, Ab193, Ab115, Ab106, Ab13f8, Ab127, Ab165, Ab155, Ab19, Ab6, Ab187, Ab179, Ab65, Ab114, Ab102, Ab94, Ab163, Ab110, Ab80, Ab92, Ab117, Ab162, Ab121, Ab195, Ab84, Ab161, Ab198, Ab24, Ab98, Ab116, Ab174, Ab196, Ab51, Ab91, Ab185, Ab23, Ab7, Ab95, Ab100, Ab140, Ab145, Ab150, Ab168, Ab54, Ab77, Ab43, Ab160, Ab82, Ab189, Ab17, Ab103, Ab18, Ab130, Ab132, Ab134, Ab144; ARG Clones: Ab2, Ab47, Ab49, Ab31, Ab53, Ab40, Abs, Ab9, Ab48, Ab4, Ab10, Ab37, Ab33, Ab42, Ab45; ARV Clones: Ab44, Ab97, Ab81, Ab188, Ab186, Ab62, Ab57, Ab192, Ab73, Ab60, Ab28, Ab32, Ab78, Ab14, Ab152, Ab72, Ab137, Ab128, Ab169, Ab87, Ab74, Ab172, Ab153, Ab120, Ab13, Ab113, Ab16, Ab56, Ab129, Ab50, Ab90, Ab99, Ab3, Ab148, Ab124, Ab22, Ab41, Ab119, Ab157, Ab27, Ab15, Ab191, Ab190, Ab79, Ab181, Ab146, Ab167, Ab88, Ab199, Ab71, Ab85, Ab59, Ab141, Ab68, Ab143, Ab46, Ab197, Ab175, Ab156, Ab63, Ab11, Ab182, Ab89, Ab8, Ab101, Ab25, Ab154, Ab21, Ab111, Ab118, Ab173, Ab38, Ab76, Ab131, Ab1, Ab67, Ab70, Ab170, Ab30, Ab93, Ab142, Ab104, Ab112, Ab35, Ab126, and Ab125 (Rigel Pharmaceuticals, Inc.); CASC-674 (Seattle Genetics); antibody clones 2, 2C, 3, 5, 13, 13A, 13B, 13C, 13D, 14, 16, 16C, 16D, 16E, 18, 21, 22, 25, 25A, 25B, 25C, 25D, 25E, 27, 54, 13 IgG2a afucosylated, 13 hIgG1 wild-type, and 13 LALA-PG (Seattle Genetics); JS006 (Shanghai Junshi Biosciences Ltd.); anti-TIGIT Fc antibody and bispecific antibody PD1×TIGIT (Xencor), antibody clone VSIG9#1 (Vsig9.01) and 258-CS1#4 (#4) (Yissum Research Development Company of The Hebrew University Of Jerusalem Ltd.); YH29143 (Yuhan Co, Ltd.); antibody clones S02, S03, S04, S05, S06, S11, S12, S14, S19, S32, S39, S43, S62, S64, F01, F02, F03, F04, 32D7, 101H3, 10A7, and 1F4 (Yuhan Co, Ltd.); anti-zB7R1 clones 318.4.1.1 (E9310), 318.28.2.1 (E9296), 318.39.1.1 (E9311), 318.59.3.1 (E9400), and 318.77.1.10 (ZymoGenetics, Inc).

In some embodiments, the anti-TIGIT antibody is selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT). ASP874 (PTZ-201) is an anti-TIGIT monoclonal antibody described in PCT Pub. No. WO2018183889A1 and US Pub. No. 2020/0095324. BGB-A1217 is an anti-TIGIT antibody as described in PCT Pub. No. WO2019129261A1. BMS-986207 (ONO-4686) is an anti-TIGIT antibody as described in PCT Pub. No. WO2016106302A9, U.S. Pat. No. 10,189,902 and US Pub. No. 2019/0112375. COM902 (CGEN-15137) is an anti-TIGIT antibody as described in PCT Pub. No. WO2018033798A1 and U.S. Pat. Nos. 10,213,505 and 10,124,061. IBI939 is an anti-TIGIT antibody as described in PCT Pub. No. WO2020020281A1. EOS884448 (EOS-448) is an anti-TIGIT antibody described in PCT Pub. No. WO2019023504A1. Domvanalimab (AB154) is an anti-TIGIT monoclonal antibody as described in PCT Pub. No. WO2017152088A1 and U.S. Pat. No. 10,537,633. Vibostolimab (MK-7684) is an anti-TIGIT antibody described in PCT Pub. Nos. WO2016028656A1, WO2017030823A2, WO2018204405A1, and/or WO2019152574A1, U.S. Pat. No. 10,618,958, and US Pub. No. 2018/0371083. SEA-TGT (SGN-TGT) is an anti-TIGIT antibody as described in PCT Pub. No. WO2020041541A2 and US Pub. No. 2020/0062859.

In some embodiments, the anti-TIGIT antagonist antibody is tiragolumab (CAS Registry Number: 1918185-84-8). Tiragolumab (Genentech) is also known as MTIG7192A, RG6058 or R07092284. Tiragolumab is an anti-TIGIT antagonistic monoclonal antibody described in PCT Pub. No. WO2003072305A8, WO2004024068A3, WO2004024072A3, WO2009126688A2, WO2015009856A2, WO2016011264A1, WO2016109546A2, WO2017053748A2, and WO2019165434A1, and US Pub. Nos. 2017/0044256, 2017/0037127, 2017/0145093, 2017/260594, 2017/0088613, 2018/0186875, 2019/0119376 and U.S. Pat. Nos. 9,873,740B2, 10,626,174B2, 10,611,836B2, 9,499,596B2, 8,431,350B2, 1,004,7158B2, and 1,001,7572B2.

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, ASP8374 (PTZ-201), BGB-A1217, BMS-986207 (ONO-4686), COM902 (CGEN-15137), M6223,161939, EOS884448 (EOS-448), domvanalimab (AB154), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, ASP8374 (PTZ-201), BGB-A1217, BMS-986207 (ONO-4686), COM902 (CGEN-15137), M6223, 161939, EOS884448 (EOS-448), domvanalimab (AB154), vibostolimab (MK-7684), and SEA-TGT (SGN-TGT).

In some embodiments, an anti-TIGIT antagonist antibody (according to any of the embodiments described herein may incorporate any of the features, singly or in combination, as described in Section C below.

B. PD-1 Axis Binding Antagonists

Provided herein are methods for treating advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC) in a subject (e.g., a human) comprising administering to the subject an effective amount of a PD-1 axis binding antagonist. PD-1 axis binding antagonists include PD-L1 binding antagonists (e.g., PD-L1 antagonist antibodies), PD-1 binding antagonists (e.g., PD-1 antagonist antibodies), and PD-2 binding antagonists (e.g., PD-L2 antagonist antibodies).

In some instances, the PD-1 axis binding antagonist is an PD-1 axis binding antagonist that inhibits the binding of PD-L1 to its binding partners. In a specific aspect, PD-L1 binding partners are PD-1 and/or B7-1. In some instances, the anti-PD-L1 antagonist antibody is capable of inhibiting binding between PD-L1 and PD-1 and/or between PD-L1 and B7-1.

In some instances, the PD-1 axis binding antagonist is an anti-PD-L1 antibody.

In some instances, the anti-PD-L1 antibody is atezolizumab (CAS Registry Number: 1422185-06-5). Atezolizumab (Genentech) is also known as MPDL3280A.

In some instances, the anti-PD-L1 antibody (e.g., atezolizumab) includes at least one, two, three, four, five, or six HVRs selected from: (a) an HVR-H1 sequence is GFTFSDSWIH (SEQ ID NO: 20); (b) an HVR-H2 sequence is AWISPYGGSTYYADSVKG (SEQ ID NO: 21); (c) an HVR-H3 sequence is RHWPGGFDY (SEQ ID NO: 22), (d) an HVR-L1 sequence is RASQDVSTAVA (SEQ ID NO: 23); (e) an HVR-L2 sequence is SASFLYS (SEQ ID NO: 24); and (f) an HVR-L3 sequence is QQYLYHPAT (SEQ ID NO: 25).

In some instances, the anti-PD-L1 antibody (e.g., atezolizumab) comprises a heavy chain and a light chain sequence, wherein: (a) the heavy chain variable (VH) region sequence comprises the amino acid sequence:

(SEQ ID NO: 26)
EVQLVESGGGLVQPGGSLRLSCAASGFTFSDSWIHVVVRQ
APGKGLEVVVAWISPYGGSTYYADSVKGRFTISADTSKNT
AYLQMNSLRAEDTAVYYCARRHWPGGFDYWGQGTLVTVSS;

and (b) the light chain variable (VL) region sequence comprises the amino acid sequence:

(SEQ ID NO: 27)
DIQMTQSPSSLSASVGDRVTITCRASQDVSTAVAVVYQQK
PGKAPKLLIYSASFLYSGVPSRFSGSGSGTDFTLTISSLQ
PEDFATYYCQQYLYHPATFGQGTKVEIKR.

In some instances, the anti-PD-L1 antibody (e.g., atezolizumab) comprises a heavy chain and a light chain sequence, wherein: (a) the heavy chain comprises the amino acid sequence:

(SEQ ID NO: 28)
EVQLVESGGGLVQPGGSLRLSCAASGFTFSDSWIHVVVRQ
APGKGLEVVVAWISPYGGSTYYADSVKGRFTISADTSKNT
AYLQMNSLRAEDTAVYYCARRHWPGGFDYVVGQGTLVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKFN
WYVDGVEVHNAKTKPREEQYASTYRVVSVLTVLHQDWLNG
KEYKCKVSNKALPAPIEKTISKAKGQPREPQVYTLPPSRE
EMTKNQVSLTCLVKGFYPSDIAVEWESNGQPENNYKTTPP
VLDSDGSFFLYSKLTVDKSRWQQGNVFSCSVMHEALHNHY
TQKSLSLSPG;

and (b) the light chain comprises the amino acid sequence:

(SEQ ID NO: 29)
DIQMTQSPSSLSASVGDRVTITCRASQDVSTAVAVVYQQK
PGKAPKLLIYSASFLYSGVPSRFSGSGSGTDFTLTISSLQ
PEDFATYYCQQYLYHPATFGQGTKVEIKRTVAAPSVFIFP
PSDEQLKSGTASVVCLLNNFYPREAKVQWKVDNALQSGNS
QESVTEQDSKDSTYSLSSTLTLSKADYEKHKVYACEVTHQ
GLSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 26); (b) a VL domain comprising an amino acid sequence comprising having at least 95% sequence identity (e.g., at least 95%, 96%, 97%, 98%, or 99% sequence identity) to, or the sequence of (SEQ ID NO: 27); or (c) a VH domain as in (a) and a VL domain as in (b). In other instances, the anti-PD-L1 antagonist antibody is selected from YW243.55.570, MDX-1105, and MED14736 (durvalumab), and MSB0010718C (avelumab). Antibody YW243.55.570 is an anti-PD-L1 described in PCT Pub. No. WO 2010/077634. MDX-1105, also known as BMS-936559, is an anti-PD-L1 antibody described in PCT Pub. No. WO 2007/005874. MED14736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference. The anti-PD-L1 antagonist antibodies (e.g., atezolizumab) useful in this invention, including compositions containing such antibodies, may be used in combination with an anti-TIGIT antagonist antibody to treat ESCC (e.g., advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC)).

In some instances, the anti-PD-L1 antagonist antibody is a monoclonal antibody. In some instances, the anti-PD-L1 antagonist antibody is an antibody fragment selected from the group consisting of Fab, Fab′-SH, Fv, scFv, and (Fab′)₂ fragments. In some instances, the anti-PD-L1 antagonist antibody is a humanized antibody. In some instances, the anti-PD-L1 antagonist antibody is a human antibody. In some instances, the anti-PD-L1 antagonist antibody described herein binds to human PD-L1.

In some instances, the PD-1 axis binding antagonist is an anti-PD-1 antagonist antibody that inhibits the binding of PD-1 to its binding partner (e.g., PD-L1). In some instances, the anti-PD-1 antagonist antibody is capable of inhibiting binding between PD-L1 and PD-1.

In some instances, the PD-1 axis binding antagonist is an anti-PD-1 antibody. In some instances, the anti-PD-1 antibody is nivolumab (MDX-1106), pembrolizumab (formerly lambrolizumab (MK-3475)), or AMP-224.

In a further aspect, a PD-1 axis binding antagonist is a PD-1 axis binding antagonist antibody according to any of the above instances may incorporate any of the features, singly or in combination, as described in Section C below.

C. Antibody Formats and Properties

1. Antibody Affinity

In certain instances, an anti-TIGIT antagonist antibody and/or PD-1 axis binding antagonist antibody (e.g., anti-PD-L1 antagonist antibody) provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In one instance, K_{D} is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M-¹s-¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an anti-TIGIT antagonist antibody and/or PD-1 axis binding antagonist antibody (e.g., anti-PD-L1 antagonist antibody) provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain instances, an anti-TIGIT antagonist antibody and/or PD-1 axis binding antagonist antibody (e.g., anti-PD-L1 antagonist antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain instances, an anti-TIGIT antagonist antibody and/or PD-1 axis binding antagonist antibody (e.g., anti-PD-L1 antagonist antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-TIGIT antagonist antibody and/or PD-1 axis binding antagonist antibodies (e.g., anti-PD-L1 antagonist antibodies) of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody and/or PD-1 axis binding antagonist antibodies (e.g., anti-PD-L1 antagonist antibodies) or antibody fragments isolated from human antibody libraries are considered human antibodies or human antibody fragments herein.

6. Antibody Variants

In certain instances, amino acid sequence variants of the anti-TIGIT antagonist antibodies and/or PD-1 axis binding antagonist antibodies (e.g., anti-PD-L1 antagonist antibodies) of the invention are contemplated. As described in detail herein, anti-TIGIT antagonist antibodies and PD-1 axis binding antagonist antibodies (e.g., anti-PD-L1 antagonist antibodies) may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, anti-TIGIT antagonist antibody and/or PD-1 axis binding antagonist antibody (e.g., anti-PD-L1 antagonist antibody) variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 2
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

II. Glycosylation Variants

In certain instances, anti-TIGIT antagonist antibodies and/or PD-1 axis binding antagonist antibodies (e.g., anti-PD-L1 antagonist antibodies) of the invention can be altered to increase or decrease the extent to which the antibody is glycosylated. Addition or deletion of glycosylation sites to anti-TIGIT antagonist antibody and/or PD-1 axis binding antagonist antibody (e.g., anti-PD-L1 antagonist antibody) of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-TIGIT antagonist antibody and/or PD-1 axis binding antagonist antibody (e.g., anti-PD-L1 antagonist antibody) variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject in the context of a fractionated, dose-escalation dosing regimen an anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab)) variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-TIGIT antagonist antibody and/or PD-1 axis binding antagonist antibody (e.g., anti-PD-L1 antagonist antibody) variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain instances, one or more amino acid modifications are introduced into the Fc region of an anti-TIGIT antagonist (e.g., an anti-TIGIT antagonist antibody disclosed herein, e.g., tiragolumab) antibody and/or PD-1 axis binding antagonist antibody (e.g., anti-PD-L1 antagonist antibody (e.g., atezolizumab)) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an anti-TIGIT antagonist antibody and/or PD-1 axis binding antagonist antibody (e.g., anti-PD-L1 antagonist antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII, and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Intl. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instance, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the anti-TIGIT antagonist antibody (e.g., an anti-TIGIT antagonist antibody disclosed herein, e.g., tiragolumab) and/or anti-PD-L1 antagonist antibody (e.g., atezolizumab) comprises an Fc region comprising an N297G mutation (EU numbering).

In some instances, the anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab)) comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3₁ and CH3₂ domains each comprise a protuberance or cavity, and wherein the protuberance or cavity in the CH3₁ domain is positionable in the cavity or protuberance, respectively, in the CH3₂ domain. In some instances, the CH3₁ and CH3₂ domains meet at an interface between said protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) is an IgG1 antibody.

IV. Cysteine Engineered Antibody Variants

In certain instances, it is desirable to create cysteine engineered anti-TIGIT antagonist antibodies and/or PD-1 axis binding antagonist antibodies (e.g., anti-PD-L1 antagonist antibodies), e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

V. Antibody derivatives

In certain instances, an anti-TIGIT antagonist antibody of the invention (e.g., an anti-TIGIT antagonist antibody (e.g., tiragolumab) or a variant thereof) and/or PD-1 axis binding antagonist antibody (e.g., anti-PD-L1 antagonist antibody of the invention (e.g., atezolizumab or a variant thereof)) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-non proteinaceous moiety are killed.

Recombinant Production Methods

Anti-TIGIT antagonist antibodies (e.g., an anti-TIGIT antagonist antibody disclosed herein, e.g., tiragolumab) and/or PD-1 axis binding antagonist antibodies (e.g., anti-PD-L1 antagonist antibodies (e.g., atezolizumab)) of the invention may be produced using recombinant methods and compositions, for example, as described in U.S. Pat. No. 4,816,567, which is incorporated herein by reference in its entirety.

For recombinant production of an anti-TIGIT antagonist antibody and/or PD-1 axis binding antagonist antibody (e.g., anti-PD-L1 antagonist antibody), nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Immunoconjugates

The invention also provides immunoconjugates comprising an anti-TIGIT antagonist (e.g., an anti-TIGIT antagonist antibody as disclosed herein, e.g., tiragolumab) and/or PD-1 axis binding antagonist (e.g., anti-PD-L1 antagonist antibody (e.g., atezolizumab)) of the invention conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some instances, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-TIGIT antagonist antibody as described herein (e.g., tiragolumab) or a PD-1 axis binding antagonist (e.g., an anti-PD-L1 antagonist antibody (e.g., atezolizumab)) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-TIGIT antagonist antibody as described herein (e.g., tiragolumab) and/or a PD-1 axis binding antagonist (e.g., an anti-PD-L1 antagonist antibody) as described herein (e.g., atezolizumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

D. Taxanes

Taxanes are chemotherapeutic agents that may bind to tubulin, promoting microtubule assembly and stabilization and/or prevent microtubule depolymerization. Examplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. Other taxanes included herein are taxoid 10-deacetylbaccatin III and/or derivatives thereof. In some embodiments, the taxane is an albumin-coated nanoparticle (e.g., nano-albumin bound (nab)-paclitaxel, i.e., ABRAXANE® and/or nab-docetaxel, ABI-008). In some embodiments, the taxane is nab-paclitaxel (ABRAXANE®). In some embodiments, the taxane is formulated in CREMAPHOR® (e.g., TAXOL®) and/or in Tween such as polysorbate 80 (e.g., TAXOTERE®). In some embodiments, the taxane is liposome-encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of CREMAPHOR and/or Tween-such as TOCOSOL® paclitaxel).

In some instances, paclitaxel is administered as part of the methods of the present invention. Paclitaxel may have the following structure:

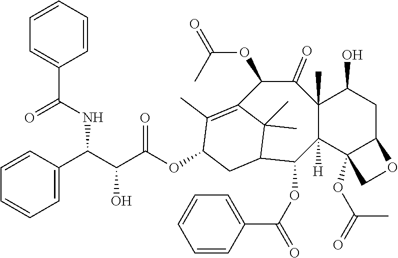 [SMILES: CC(=O)O[C@H]1C(=O)[C@@]2(C)[C@H]([C@H](OC(=O)c3ccccc3)C3C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In some instances, the methods include administration of nano-albumin bound (nab)-paclitaxel.

A skilled artisan will appreciate that any of the aforementioned taxanes can be administered in various forms, such as salt forms, which are contemplated as part of the present invention.

E. Platinum Agents

Platinum agents include an organic compound which contains platinum as an integral part of the molecule. Typically platinum-based chemotherapeutic agents are coordination complexes of platinum. agents include, but are not limited to, cisplatin, carboplatin, and oxaliplatin.

Platinum agents (such as cisplatin, carboplatin, oxaliplatin, and staraplatin) are widely used antitumor drugs that cause crosslinking of DNA as monoadduct, interstrand crosslinks, intrastrand crosslinks or DNA protein crosslinks. Platinum agents typically act on the adjacent N-7 position of guanine, forming a 1, 2 intrastrand crosslink (Poklar et al. (1996). Proc. Natl. Acad. Sci. U.S.A. 93 (15): 7606-11; Rudd et al. (1995). Cancer Chemother. Pharmacol. 35 (4): 323-6). The resultant crosslinking inhibits DNA repair and/or DNA synthesis in cancer cells.

An exemplary platinum agent used in the methods described herein is cisplatin, which has the following structure:

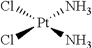

A skilled artisan will appreciate that any of the aforementioned platinum agents can be administered in various forms, such as salt forms, which are contemplated as part of the present invention.

Pharmaceutical Compositions, Formulations, And Kits for First-Line Therapies

Any of the anti-TIGIT antagonist antibodies, PD-1 axis binding antagonists, taxanes, and platinum agents described herein can be used in pharmaceutical compositions and formulations. Pharmaceutical compositions and formulations of an anti-TIGIT antagonist antibody, a PD-1 axis binding antagonist (e.g., an anti-PD-L1 antagonist antibody), a taxane, and a platinum agent can be prepared by mixing one, two, three, or all four agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

In another embodiment of the invention, a kit is provided comprising an anti-TIGIT antagonist antibody for use in combination with a PD-1 axis binding antagonist, a taxane, and a platinum agent for treating a subject having an advanced ESCC according to any of the methods described herein. In some instances, the kit further comprises the PD-1 axis binding antagonist, the taxane, and/or the platinum agent.

In another embodiment, a kit comprises tiragolumab for use in combination with atezolizumab, paclitaxel, and cisplatin for treating a subject having an advanced ESCC according to any of the methods described herein. In some embodiments, the kit further comprises atezolizumab, paclitaxel, and/or cisplatin.

Kits provided herein may include a PD-1 axis binding antagonist (e.g., atezolizumab) for use in combination with an anti-TIGIT antagonist antibody (e.g., tiragolumab), a taxane (e.g., paclitaxel), and/or a platinum agent (e.g., cisplatin) for treating a subject having an advanced ESCC (e.g., locally advanced ESCC, unresectable ESCC, locally advanced unresectable ESCC, or recurrent or metastatic ESCC), e.g., Stage II ESCC, Stage III ESCC, or Stage IV ESCC (e.g., a Stage IVA ESCC) according to any of the methods described herein. In some embodiments, the kit further comprises tiragolumab, paclitaxel, and/or cisplatin. In some embodiments, the kit comprises tiragolumab and atezolizumab. In some embodiments, the kit comprises tiragolumab, atezolizumab, paclitaxel, and cisplatin.

### V. EXAMPLES

The following are examples of the methods of the invention. It is understood that various other embodiments may be practiced, given the general description provided above.

### Example 1. A Phase III, Randomized, Double-Blind, Placebo-Controlled Study of Atezolizumab with or without Tiragolumab in Patients with Unresectable Locally Advanced Esophageal Squamous Cell Carcinoma

The present example describes a Phase III study (YO42137) evaluating the efficacy and safety of tiragolumab plus atezolizumab compared with placebo in patients with unresectable esophageal squamous cell carcinoma (or those who are unable or unwilling to undergo surgery) and whose cancer has not progressed following definitive concurrent chemoradiotherapy. The purpose of this study is to test the hypothesis that tiragolumab plus atezolizumab prolongs the duration of investigator-assessed PFS and/or OS relative to placebo in the ITT population.

Study Design

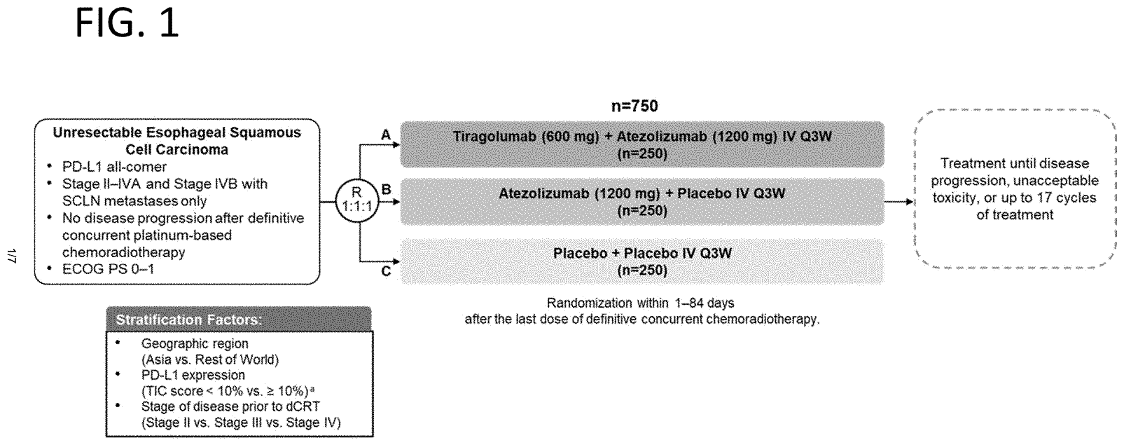

Described below are the details of a randomized, Phase III, global, multicenter, double-blinded, placebo-controlled study designed to evaluate the safety and efficacy of tiragolumab in combination with atezolizumab compared with placebo in patients with unresectable locally advanced esophageal squamous cell carcinoma (or those who are unable or unwilling to undergo surgery) and who have completed definitive concurrent chemoradiation therapy. FIG. 1 presents an overview of the study design.

This study enrolls approximately 750 patients randomized in a 1:1:1 ratio to one of three treatment arms:
Arm A: tiragolumab+atezolizumab
Arm B: tiragolumab placebo+atezolizumab
Arm C: tiragolumab placebo+atezolizumab placebo Randomization is stratified according to the following stratification factors:
Geographic region (Asia vs. Rest of World)
PD-L1 expression (tumor and tumor-associated immune cell (TIC) score<10% vs. ≥10%), as assessed by a central laboratory using the investigational Ventana PD-L1 (SP263) Companion Diagnostic (CDx) Assay
Stage of disease prior to definitive chemoradiotherapy (Stage II vs. Stage III vs. Stage IVA)

Patients receive either tiragolumab plus atezolizumab (Arm A), placebo plus atezolizumab (Arm B), or double placebo (Arm C).

In Arm A, patients receive atezolizumab at a fixed dose of 1200 mg administered by IV infusion every 3 weeks (Q3W) on Day 1 of each 21-day cycle, followed by tiragolumab at a fixed dose of 600 mg administered by IV infusion Q3W on Day 1 of each 21-day cycle for up to 17 cycles.

Tumor assessments continue regardless of whether treatment has been delayed, until radiographic disease progression per RECIST v1.1, withdrawal of consent, death, or study termination, whichever occurs first. Tumor assessments are to continue according to schedule in patients who discontinue treatment for reasons other than radiographic disease progression per RECIST v1.1, even if they start new anti-cancer therapy, until consent is withdrawn, death or the study is terminated, whichever occurs first.

For equivocal findings of radiographic progression (e.g., very small and uncertain new lesions; cystic changes or necrosis in existing lesions), a confirmatory scan must be performed again within 4-6 weeks. If at the next scheduled assessment, progression is confirmed, the date of progression recorded should be the earlier date when progression was suspected.

Following treatment discontinuation, information on survival and subsequent anti-cancer therapies is collected until death, loss to follow-up, withdrawal of consent, or study termination, whichever occurs first.

Patients undergo patient-reported outcome (PRO) assessments at specified timepoints during treatment and for up to 1 year after treatment discontinuation.

Safety assessment includes the incidence, nature, and severity of adverse events and laboratory abnormalities graded per the National Cancer Institute Common Terminology Criteria for Adverse Events, Version 5.0 (NCI CTCAE v5.0). Severity for CRS is also graded according to the American Society for Transplantation and Cellular Therapy consensus grading scale. Laboratory safety assessments include the regular monitoring of hematology and blood chemistry.

Serum samples are collected to monitor tiragolumab and atezolizumab pharmacokinetics and to detect the presence of antibodies to tiragolumab and atezolizumab.

Patient samples, including archival and fresh tumor tissue, serum, plasma, and blood samples, are also collected for exploratory biomarker assessments.

Study Treatment Dosage and Administration

Patients receive either tiragolumab plus atezolizumab (Arm A), placebo plus atezolizumab (Arm B), or double placebo (Arm C).

In Arm A, patients receive atezolizumab at a fixed dose of 1200 mg administered by IV infusion every 3 weeks (Q3W) on Day 1 of each 21-day cycle, followed by tiragolumab at a fixed dose of 600 mg administered by IV infusion Q3W on Day 1 of each 21-day cycle for up to 17 cycles.

In Arm B, patients receive atezolizumab at a fixed dose of 1200 mg administered by IV infusion Q3W on Day 1 of each 21-day cycle, followed by placebo administered by IV infusion Q3W on Day 1 of each 21-day cycle for up to 17 cycles.

In Arm C, patients receive placebo administered by IV infusion Q3W on Day 1 of each 21-day cycle in two consecutive administrations for up to 17 cycles.

Treatment should continue until unacceptable toxicity or radiographic progression per investigator-assessed Response Evaluation Criteria in Solid Tumors, Version 1.1 (RECIST v1.1), or up to 17 cycles of treatment, whichever occurs first. Patients undergo tumor assessments at scheduled intervals during the study. Additional scans are performed as clinically indicated.

Atezolizumab/Placebo

Atezolizumab/placebo is administered by IV infusion at a fixed dose of 1200 mg on Day 1 of each 21-day cycle. The atezolizumab/placebo dose is fixed and is not dependent on body weight.

No dose modification for atezolizumab is allowed.

Atezolizumab/placebo infusions are administered per the instructions outlined in Table 3.

TABLE 3
Administration of First and Subsequent Atezolizumab and Tiragolumab/Placebo Infusions

| | First infusion | Subsequent infusion |
|---|---|---|
| Atezolizumab/placebo | No premedication is permitted prior | If the patient experienced an IRR |
| infusion | to the atezolizumab/placebo infusion, | with any previous infusion of |
| | Vital signs (pulse rate, respiratory | atezolizumab/placebo, |
| | rate, blood pressure, and | premedication with an antihistamine |
| | temperature) are recorded within 60 | and/or antipyretic may be |
| | minutes prior to starting the infusion, | administered for subsequent doses |
| | Atezolizumab/placebo is infused | at the discretion of the investigator. |
| | over 60 (±15) minutes. | Vital signs are recorded within 60 |
| | If clinically indicated, vital signs are | minutes prior to the infusion. |
| | recorded every 15 (±5) minutes | Atezolizumab/placebo is infused |
| | during the infusion. | over 30 (±10) minutes if the previous |
| | Patients are informed about the | infusion was tolerated without an |
| | possibility of delayed post-infusion | IRR or 60 (±15) minutes if the |
| | symptoms and instructed to contact | patient experienced an IRR with the |
| | their study physician if they develop | previous infusion. |
| | such symptoms. | Vital signs are recorded during the |
| | | infusion if clinically indicated or if the |
| | | patient experienced an IRR with the |
| | | previous infusion. |
| Observation period | After the infusion of | If the patient tolerated the previous |
| after infusion of | atezolizumab/placebo, the patient | atezolizumab/placebo infusion well |
| atezolizumab/placebo | begins a 60-minute observation | without infusion-associated adverse |
| | period. | events, the observation period after |
| | Vital signs (pulse rate, respiratory | the next and following infusions may |
| | rate, blood pressure, and | be reduced to 30 minutes. |
| | temperature) are recorded at 30 | If the patient experienced infusion- |
| | (±10) minutes after the infusion of | associated adverse events in the |
| | atezolizumab/placebo. | previous infusion, the observation |
| | Patients are informed about the | period should be 60 minutes. |
| | possibility of delayed post-infusion | If clinically indicated, vital signs are |
| | symptoms and instructed to contact | recorded at 30 (±10) minutes after |
| | their study physician if they develop | the infusion of atezolizumab/placebo. |
| | such symptoms. | |
| Tiragolumab/placebo | No premedication is permitted prior | If the patient experienced an IRR |
| infusion | to tiragolumab/placebo infusion, | during any previous infusion of |
| | Vital signs (pulse rate, respiratory | tiragolumab/placebo, premedication |
| | rate, blood pressure, and | with an antihistamine and/or anti- |
| | temperature) are recorded within 60 | pyretic may be administered for |
| | minutes before starting the infusion | subsequent doses at the discretion |
| | of tiragolumab/placebo. | of the investigator. |
| | Tiragolumab/placebo is infused | Vital signs are recorded within 60 |
| | over 60 (±15) minutes. | minutes prior to the |
| | Vital signs are recorded every 15 | tiragolumab/placebo infusion. |
| | (±5) minutes during the infusion. | Tiragolumab/placebo should be |
| | | infused over 30 (±10) minutes if the |
| | | previous infusion was tolerated |
| | | without an infusion-related reaction, |
| | | or 60 (±15) minutes if the patient |
| | | experienced an infusion-related |
| | | reaction with the previous infusion. |
| | | Vital signs are recorded during and |
| | | after the infusion if clinically |
| | | indicated. |
| Observation period | After the infusion of | If the patient tolerated the previous |
| after infusion of | tiragolumab/placebo, the patient | infusion of tiragolumab/placebo well |
| tiragolumab/placebo | begins a 60-minute observation | without infusion-associated adverse |
| | period. | events, the observation period is |
| | Vital signs are recorded at 30 | reduced to 30 minutes. |
| | (±10) minutes after the infusion of | If the patient experienced an |
| | tiragolumab/placebo. | infusion-associated adverse event in |
| | Patients are informed about the | the previous infusion, the |
| | possibility of delayed post-infusion | observation period should be 60 |
| | symptoms and are instructed to | minutes. |
| | contact their study physician if they | If clinically indicated, vital signs are |
| | develop such symptoms. | recorded at 30 (±10) minutes after |
| | | the infusion of tiragolumab/placebo. |
| | | Patients are informed about the |
| | | possibility of delayed post-infusion |
| | | symptoms and will be instructed to |
| | | contact their study physician if they |
| | | develop such symptoms. |

Tiragolumab/Placebo

Following the administration of atezolizumab/placebo and an observation period (see Table 3), patients receive 600 mg tiragolumab/placebo administered by IV infusion on Day 1 of each 21-day cycle. The tiragolumab/placebo dose is fixed and is not dependent on body weight.

No dose modification for tiragolumab/placebo is allowed.

Tiragolumab/placebo infusions are administered per the instruction outlined in Table 3.

Atezolizumab/Placebo and Tiragolumab/Placebo

The following rules apply as long as neither atezolizumab/placebo nor tiragolumab/placebo has been permanently discontinued:
Treatment cycles normally begin with dosing of atezolizumab/placebo and tiragolumab/placebo on Day 1 of each 21-day cycle. If either study drug is delayed for a related toxicity, it is recommended that the other study drug is also delayed since the safety profiles for atezolizumab and tiragolumab are similar; however, a cycle may begin with the administration of the other study drug if considered appropriate at the discretion of the investigator.
In case of delays in dosing of one study drug for drug-related toxicity while the other study drug is given as planned, it is recommended that the study drug being delayed will be administered at the next scheduled infusion (i.e., at the next scheduled 21-day cycle).

Treatment Interruption

Study treatment may be temporarily suspended as appropriate for management of toxicity. On the basis of the available characterization of mechanism of action, tiragolumab may cause adverse events similar to but independent of atezolizumab, may exacerbate the frequency or severity of atezolizumab-related adverse events, or may have non-overlapping toxicities with atezolizumab. Because these scenarios may not be distinguished from one another in the clinical setting, immune-mediated adverse events should generally be attributed to both study drugs, and dose interruptions or treatment discontinuation in response to immune-mediated adverse events should be applied to both tiragolumab/placebo and atezolizumab/placebo.

Tiragolumab/placebo and atezolizumab/placebo may be held for a maximum of approximately 12 weeks (approximately four cycles). If tiragolumab/placebo is interrupted for more than approximately 12 weeks for any reason, the patient will have to permanently discontinue tiragolumab/placebo treatment but may continue atezolizumab/placebo if there is no contraindication and after discussion with the Medical Monitor to determine whether the toxicity is considered related to tiragolumab/placebo and/or to the combination with atezolizumab/placebo. Continued dosing with single-agent atezolizumab/placebo administered to patients Q3W requires that all other study eligibility criteria continue to be met.

An exception can be made if in the judgment of the investigator, the patient is likely to derive clinical benefit from resuming tiragolumab/placebo after a hold >12 weeks. In this case, tiragolumab/placebo may be restarted with the approval of the Medical Monitor.

If atezolizumab/placebo is interrupted for approximately >12 weeks (or approximately four cycles), the patient will have to permanently discontinue atezolizumab/placebo. However, if, in the judgment of the investigator, the patient is likely to derive clinical benefit from atezolizumab/placebo after a hold of approximately >12 weeks, atezolizumab/placebo may be restarted with the approval of the Medical Monitor.

If a patient must be tapered off steroids used to treat adverse events, atezolizumab/placebo may be withheld for additional time beyond approximately 12 weeks from the last dose, and tiragolumab/placebo may be withheld for an additional time beyond approximately 12 weeks from the last dose until steroids are discontinued, or until steroids are reduced to prednisone dose (or dose equivalent) ≤10 mg/day. The acceptable length of interruption will depend on an agreement between the investigator and the Medical Monitor. Dose interruptions for reason(s) other than toxicity, such as surgical procedures, may be allowed with Medical Monitor approval.

After both tiragolumab/placebo and atezolizumab/placebo have been permanently discontinued, the patient is monitored for safety and efficacy.

Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the treatment discontinuation visit.

Permitted Therapy

Patients are permitted to use the following therapies during the study:
Oral contraceptives with a failure rate of <1% per year
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

Corticosteroids, Immunosuppressive Medications, and TNFα Inhibitors

Systemic corticosteroids, immunosuppressive medications, and TNF-α inhibitors may attenuate potential beneficial immunologic effects of treatment with atezolizumab. Therefore, in situations in which systemic corticosteroids, immunosuppressive medications, or TNF-α inhibitors would be routinely administered, alternatives, including antihistamines, should be considered. If the alternatives are not feasible, systemic corticosteroids, immunosuppressive medications, and TNF-α inhibitors may be administered at the discretion of the investigator.

Systemic corticosteroids are recommended, at the discretion of the investigator, for the treatment of specific adverse events when associated with atezolizumab therapy.

Herbal Therapies

Concomitant use of herbal therapies is not recommended because their pharmacokinetics, safety profiles, and potential drug-drug interactions are generally unknown. However, herbal therapies not intended for the treatment of cancer may be used during the study at the discretion of the investigator.

Prohibited Therapy

Use of the following concomitant therapies is prohibited as described below:
Concomitant therapy intended for the treatment of cancer, whether health authority-approved or experimental, is prohibited for various time periods prior to starting study treatment, depending on the agent, and during study treatment, until disease progression is documented and the patient has discontinued study treatment, with the exception of palliative radiotherapy under certain circumstances.
Investigational therapy is prohibited within 28 days prior to initiation of study treatment and during study treatment.
Live, attenuated vaccines (e.g., FLUMIST®) are prohibited within 4 weeks prior to initiation of study treatment, during study treatment, for 5 months after the final dose of atezolizumab/placebo, and or 90 days after the final dose of tiragolumab/placebo, whichever is later.
Systemic immunostimulatory agents (including, but not limited to, interferons and IL-2) are prohibited within 4 weeks or 5 drug-elimination half-lives (whichever is longer) prior to initiation of study treatment and during study treatment because these agents could potentially increase the risk for autoimmune conditions when given in combination with atezolizumab.

Objectives and Efficacy Endpoints

This study evaluates the efficacy and safety of tiragolumab plus atezolizumab compared with placebo in patients with locally advanced esophageal squamous cell carcinoma (or those who are unable or unwilling to undergo surgery) and who have completed definitive concurrent chemoradiation therapy. Specific objectives and corresponding endpoints for the study are outlined in Table 4.

TABLE 4
Objectives and Corresponding Endpoints

| | Corresponding Endpoints |
|---|---|
| Primary Efficacy Objective | |
| To evaluate the efficacy of tira + atezo compared | PFS, defined as the time from randomization to |
| with double placebo | the first occurrence of disease progression or |
| | death from any cause (whichever occurs first), as |
| | determined by the investigator according to |
| | RECIST v1.1 |
| | OS, defined as the time from randomization to |
| | death from any cause |
| To evaluate the efficacy of tira + atezo compared | OS, defined as the time from randomization to |
| with double placebo | death from any cause |
| Secondary Efficacy Objective | |
| To evaluate the efficacy of placebo + atezo | PFS, defined as the time from randomization to |
| compared with double placebo | the first occurrence of disease progression or |
| | death from any cause (whichever occurs first), as |
| | determined by the investigator according to |
| | RECIST v1.1 |
| To evaluate the efficacy of tira + atezo versus | PFS, defined as the time from randomization to |
| placebo + atezo to demonstrate the contribution | the first occurrence of disease progression or |
| of tiragolumab | death from any cause (whichever occurs first), as |
| | determined by the investigator according to |
| | RECIST v1.1 |
| | OS, defined as the time from randomization to |
| | death from any cause |
| To evaluate the efficacy of tira + atezo and | IRF-assessed PFS, defined as the time from |
| placebo + atezo compared with double placebo | randomization to the first occurrence of disease |
| | progression or death from any cause (whichever |
| | occurs first), as determined by an IRF according |
| | to RECIST v1.1 |
| To evaluate the efficacy of tira + atezo versus | Confirmed ORR, defined as the proportion of |
| placebo + atezo to demonstrate the contribution | patients with a CR or PR on two consecutive |
| of tiragolumab | occasions ≥4 weeks apart among patients who |
| | have remaining baseline disease (i.e., non-target |
| | lesions) after chemoradiation therapy, as |
| | determined by the investigator according to |
| | RECIST v1.1 |
| | Confirmed ORR as determined by an IRF |
| | according to RECIST v1.1 |
| | DOR, defined as the time from the first |
| | occurrence of a confirmed objective response to |
| | the first occurrence of disease progression or |
| | death from any cause (whichever occurs first), as |
| | determined by the investigator according to |
| | RECIST v1.1 |
| | DOR as determined by an IRF according to |
| | RECIST v1.1 |
| | Proportion of patients with clinically meaningful |
| | changes in physical functioning, role functioning, |
| | GHS/QoL, and dysphagia, as measured by the |
| | respective scales of the EORTC QLQ-C30 and |
| | the EORTC QLQ-OES18 |
| Exploratory Efficacy Objective | |
| To evaluate the efficacy of tira + atezo and | PFS rates at 12 months and 18 months, defined |
| placebo + atezo compared with double placebo | as the proportion of patients who have not |
| To evaluate the efficacy of tira + atezo versus | experienced disease progression or death from |
| placebo + atezo to demonstrate the contribution | any cause at 12 months or 18 months, as |
| of tiragolumab | determined by the investigator according to |
| | RECIST v1.1 |
| | PFS rates at 12 months and 18 months as |
| | determined by an IRF according to RECIST v1.1 |
| | OS rates at 12 months and 24 months, defined as |
| | the proportion of patients who have not |
| | experienced death from any cause at 12 months |
| | or 24 months, respectively |
| | Mean scores and mean change from baseline in |
| | scores (by cycle) in all scales of the EORTC QLQ- |
| | C30 and EORTC QLQ-OES18 |
| Safety Objective | |
| To evaluate the safety and tolerability of tira + | Incidence and severity of adverse events |
| atezo and placebo + atezo compared with double | Severity for all events will be graded according |
| placebo | to NCI CTCAE v5.0, and severity for CRS will also |
| | be graded according to the ASTCT consensus |
| | grading scale. |
| Pharmacokinetic Objective | |
| To characterize the pharmacokinetics of | Serum concentration of tiragolumab and |
| tiragolumab and atezolizumab | atezolizumab at specified timepoints |
| Immunogenicity Objective | Corresponding Endpoint |
| To evaluate the immune response to tiragolumab | Prevalence of ADAs to tiragolumab at baseline |
| and atezolizumab | and incidence of ADAs to tiragolumab during the |
| | study |
| | Prevalence of ADAs to atezolizumab at baseline |
| | and incidence of ADAs to atezolizumab during the |
| | study |
| Exploratory Immunogenicity Objective | |
| To evaluate potential effects of ADAs | Relationship between tiragolumab and |
| | atezolizumab ADA status and efficacy, safety, or |
| | PK endpoints |
| Exploratory Biomarker Objective | |
| To identify and/or evaluate biomarkers that are | Relationship between biomarkers in tumor tissue |
| associated with progression to a more severe | and blood and efficacy, safety, PK, |
| disease state (i.e., prognostic biomarkers), are | immunogenicity, or other biomarker endpoints |
| associated with acquired resistance to study | |
| treatment, can provide evidence of study | |
| treatment activity (i.e., pharmacodynamic | |
| biomarkers), or can increase the knowledge and | |
| understanding of disease biology and drug safety | |
| Exploratory Health Status Utility Objectives | |
| To evaluate health status utility scores of patients | Mean change from baseline in the index-based |
| treated with Atezo + Tira and placebo compared | and VAS scores of the EQ-5D-5L |
| with double placebo | |

ADA = anti-drug antibody; atezo = atezolizumab; ASTCT = American Society for Transplantation and Cellular Therapy; CR = complete response; DOR = duration of response; EORTC = European Organisation for Research and Treatment of Cancer; EQ-5D-5L = EuroQol 5-Dimension, 5-Level Questionnaire; GHS/QoL = global health status and quality of life; IRF = independent review facility; NCI CTCAE v5.0 = National Cancer Institute Common Terminology Criteria for Adverse Events, Version 5.0; ORR = objective response rate; OS = overall survival; PFS = progression-free survival; PK = pharmacokinetic; PR = partial response; QLQ-C30 = Quality of Life-Core 30 Questionnaire; QLQ-OE518 = Quality of Life-Esophageal Cancer, Module 18 Questionnaire; RECIST v1.1 = Response Evaluation Criteria in Solid Tumors, Version 1.1; SD = stable disease; tira = tiragolumab; VAS = visual analog scale.
a The PK, immunogenicity, and/or exploratory biomarker objective(s) and related sample collections will not be applicable if approval by the local regulatory and/or required decision bodies are not obtained.
Note:
Irradiated lesions are usually not considered measurable per RECIST v1.1 (unless there has been demonstrated progression in the lesion). Therefore, patients in this study (whose cancers have not progressed following definitive concurrent chemoradiation therapy prior to randomization) should not have target lesions at baseline.

Efficacy Analysis

In this study, the co-primary efficacy endpoints are investigator-assessed PFS (supported by a secondary analysis of independent review facility (IRF)-assessed PFS) and OS. This study tests the hypothesis that treatment with tiragolumab plus atezolizumab will prolong PFS and OS compared with placebo alone.

PFS as an endpoint can reflect tumor growth and can be assessed before the determination of a survival benefit. Additionally, its determination is not generally confounded by subsequent therapies, which is particularly relevant in the locally advanced disease setting. In addition, data from several tumor types have suggested a strong correlation between PFS and OS, thus supporting PFS as a robust surrogate predictor of OS and clinical benefit (Adunlin et al., Breast Cancer Res Treat, 154:591-608 (2015); Dabbous et al., Ann Oncol, 28 Suppl. 3:iii77 (2017)). As such, PFS as a co-primary endpoint may enable an earlier indication of benefit and more quickly make this new treatment combination available to patients.

Improvement in OS is generally accepted as the best measure of clinical benefit for patients with advanced cancers and is an endpoint that is objective and easily measured. Recent data also suggest that OS may be a more sensitive endpoint for CIT (Fehrenbacher et al., Lancet, 387:1837-46 (2016)). For these reasons, OS is also a co-primary endpoint in this study.

Investigator-Assessed Progression-Free Survival

Investigator-assessed PFS is defined as the time from randomization to the first occurrence of disease progression or death from any cause (whichever occurs first), as determined by the investigator according to RECIST v1.1. Patients who have not experienced disease progression or death at the time of analysis are censored at the time of the last tumor assessment. Patients with no post-baseline tumor assessment are censored at the date of randomization.

The two-sided log-rank test, stratified by geographic region (Asia vs. Rest of World), PD-L1 (SP263) expression (TIC<10% vs. ≥10%) and stage of disease prior to definitive concurrent chemoradiotherapy (Stage II vs. Stage III vs. Stage IV), is used as the primary analysis to compare PFS between treatment arms. The results from the unstratified log-rank test are also provided as a sensitivity analysis to check the robustness of the results of the stratified log-rank test.

The stratified Cox proportional-hazards model is used to estimate the HR and its 95% Cl. The stratification factors are the same as those used for the primary stratified log-rank test. The unstratified HR is also provided.

Kaplan-Meier methodology is used to estimate the median PFS for each treatment arm, and Kaplan-Meier curves are constructed to provide a visual description of the difference between treatment arms. The Brookmeyer-Crowley methodology is used to construct the 95% CI for the median PFS for each treatment arm.

In order to assess the homogeneity of the treatment effect with respect to the co-primary efficacy endpoint of PFS across subgroups defined by demographics (e.g., age, sex and race/ethnicity) and baseline characteristics (e.g., ECOG Performance Status, PD-L1 expression), forest plots (including the estimated HRs) are provided.

Overall Survival

OS is defined as the time from randomization to death from any cause. Patients who are not reported as having died at the time of analysis are censored at the last date they were known to be alive. Patients with no post-baseline survival information are censored at the date of randomization.

Methods for the OS analysis are similar to those described for investigator-assessed PFS. A group sequential design is used for testing OS to account for the interim analyses.

There are two interim analyses planned for OS. The first interim analysis of OS is conducted at the time of the primary PFS analysis, which is expected to take place at approximately 34 months after the first patient is randomized. It is anticipated that at this time, approximately 212 OS events will have occurred in Arm A and Arm C. The second interim analysis of OS is performed when approximately 240 OS events have been observed in Arm A and Arm C, which is expected to occur at approximately 40 months after the first patient is randomized. The interim analyses is conducted by the Sponsor. The final analysis of OS occurs when approximately 280 OS events have been observed in Arm A and Arm C. The analysis timing of OS between Arm B vs. Arm C and between Arm A vs. Arm B is the same as for OS between Arm A vs. Arm C. Given the testing hierarchy, if OS between Arm A vs. Arm C is not statistically significant at an interim or final analysis, OS between Arm B vs. Arm C and Arm A vs. Arm B will not be formally tested at that time, but the comparisons will be performed descriptively to characterize the individual contribution of atezolizumab and tiragolumab. In this case, the trial will continue to the next planned analysis timing for OS hierarchical testing.

If fewer than 175 OS events have occurred in Arm A and Arm C (<35% of 500 patients) at the time of the primary PFS analysis, the first interim OS analysis is delayed until 212 OS events have occurred. An administrative α of 0.000001 (negligible impact on overall type I error rate) is spent on the OS hypothesis at the time of the primary PFS analysis.

A group sequential design is used to account for the conduct of the interim analyses and control the two-sided type I error for the OS endpoints in the testing hierarchy. The stopping boundaries for each of the OS interim and final analyses are computed with use of the Lan-DeMets α-spending function that approximates the O'Brien-Fleming boundary, respectively. The timing of the interim and final analyses as well as the projected events rate for the co-primary endpoints are shown in Table 5. The corresponding a stopping boundaries and MDD HR are also shown based on the planned number of PFS/OS events at each PFS/OS analysis. The MDD HR for each PFS/OS endpoint at final analysis is considered both clinically meaningful and achievable. The actual boundaries are calculated at the time of PFS/OS analysis based on the observed information fraction, i.e., actual number of events observed at time of analysis over the total planned target number of events in the ITT population.

TABLE 5
Analysis Timing and Stopping Boundaries for Interim and Final Analyses of the Co-
Primary Endpoints

| | Number of Events (Event/Patient Ratio) and Stopping Boundary: HR | | |
|---|---|---|---|
| | (Two-Sided P-Value) | | |
| Planned Analysis of | Analysis Timing 1 | Analysis Timing 2 | Analysis Timing 3 |
| Co-Primary Endpoints | (34 m from FPI) | (40 m from FPI) | (48 m from FPI) |
| Investigator-assessed | 287 (57%) MDD HR | NA | NA |
| PFS in A vs. C | ≤0.774 (p ≤ 0.0300) | | |
| OS in A vs. C | 212 (42%) MDD HR | 240 (48%) MDD HR | 280 (56%) MDD HR |
| (if α = 0.02) | ≤0.686 (p ≤ 0.0061) | ≤0.714 (p ≤ 0.0090) | ≤0.751 (p ≤ 0.0165) |
| OS in A vs. C | 212 (42%) MDD HR | 240 (48%) MDD HR | 280 (56%) MDD HR |
| (if α = 0.05) | ≤0.726 (p ≤ 0.0200) | ≤0.749 (p ≤ 0.0253) | ≤0.782 (p ≤ 0.0400) |
| OS in B vs. C | 222 (44%) MDD HR | 251 (50%) MDD HR | 292 (58%) MDD HR |
| (if α = 0.02) | ≤0.693 (p ≤ 0.0063) | ≤0.720 (p ≤ 0.0091) | ≤0.755 (p ≤ 0.0164) |
| OS in B vs. C | 222 (44%) MDD HR | 251 (50%) MDD HR | 292 (58%) MDD HR |
| (if α = 0.05) | ≤0.732 (p ≤ 0.0203) | ≤0.754 (p ≤ 0.0255) | ≤0.786 (p ≤ 0.0399) |
| OS in A vs. B | 192 (38%) MDD HR | 219 (44%) MDD HR | 258 (52%) MDD HR |
| (if α = 0.02) | ≤0.671 (p ≤ 0.0057) | ≤0.702 (p ≤ 0.0087) | ≤0.742 (p ≤ 0.0166) |
| OS in A vs. B | 192 (38%) MDD HR | 219 (44%) MDD HR | 258 (52%) MDD HR |
| (if α = 0.05) | ≤0.712 (p ≤ 0.0187) | ≤0.738 (p ≤ 0.0247) | ≤0.775 (p ≤ 0.0403) |

IRF-Assessed Progression-Free Survival

IRF-assessed PFS is defined as the time from randomization to the first occurrence of disease progression or death from any cause (whichever occurs first), as determined by an IRF according to RECIST v1.1. Patients who have not experienced disease progression or death at the time of analysis are censored at the time of the last tumor assessment. Patients with no post-baseline tumor assessment are censored at the date of randomization.

Methods for the IRF-assessed PFS analysis are similar to those described for investigator-assessed PFS.

Investigator-Assessed Objective Response Rate

An objective response per investigator is defined as a complete response (CR) or PR as determined by the investigator according to RECIST v1.1. Patients not meeting these criteria, including patients without any post-baseline tumor assessment, are considered non-responders. Confirmed ORR is defined as the proportion of patients who achieved an objective response on two consecutive occasions 4 weeks apart. The analysis population for ORR is all patients in the ITT population with measurable disease at baseline.

The two-sided Cochran-Mantel-Haenszel test, stratified by geographic region (Asia) vs. Rest of World), PD-L1 (SP263) expression (TIC<10% vs. 10%) and stage of disease prior to definitive concurrent chemoradiotherapy (Stage II vs. Stage III vs. Stage IV), is used to compare ORR between treatment arms. ORR is calculated for each treatment arm and the difference in ORR between treatment arms is computed. The 95% CI for ORR for each arm is derived using the Clopper-Pearson method (Clopper and Pearson, Biometrika, 26:404-13 (1934)). The 95% CI for difference in ORR is computed by normal approximation.

IRF-Assessed Objective Response Rate

ORR analyses are performed separately based on IRF-assessed tumor response according to RECIST v1.1. The analysis methods are similar to those described for investigator-assessed ORR.

Duration of Response

Duration of response (DOR) is assessed in patients who achieved an objective response. DOR is defined as the time from the first occurrence of a confirmed objective response (CR or PR, whichever status is recorded first) to the first occurrence of disease progression or death from any cause, whichever occurs first. Patients whose cancers have not progressed and who have not died at the time of analysis will be censored at the date of last tumor assessment. If no tumor assessments are performed after the date of the first occurrence of an objective response, DOR will be censored at the date of the first occurrence of the response. The analysis of DOR is based on a nonrandomized subset of patients (specifically, patients who achieved an objective response); therefore, comparisons between treatment arms will be made for descriptive purposes only.

DOR analyses are performed separately based on investigator- and IRF-assessed tumor response. The analysis methods are similar to those described for PFS.

Proportion of Patients with Clinically Meaningful Changes in Functioning, Quality of Life, and Dysphagia

The proportion of patients with clinically meaningful changes (improved, deteriorated, remained stable) in physical functioning, role functioning, GHS/QoL, and dysphagia, as measured by the respective scales of the EORTC QLQ-C30 and the EORTC QLQ-OES18, are summarized by treatment arm. Previously published minimally important differences are used to identify clinically meaningful changes (e.g., Osoba et al., J Clin Oncol, 16:139-44 (1998); Cocks et al., J Clin Oncol, 29:89-96 (2011)).

Progression-Free Survival Rate at Landmark Timepoints

The PFS rate at 12 months and 18 months are estimated based on investigator- and IRF-assessed tumor response separately using Kaplan-Meier methodology for each treatment arm and the 95% Cls calculated using the standard error derived from Greenwood's formula. The 95% CI for the difference in PFS rates between treatment arms are estimated using the normal approximation method.

Overall Survival Rate at Landmark Timepoints

The OS rate at 12 months and 24 months are estimated using the same methods as those described for PFS rate.

Patient-Reported Outcomes

Completion rates and reasons for missing data are summarized for the EORTC QLQC30 and EORTC QLQ-OES18 questionnaires at each cycle by treatment arm.

Visit mean summary and change from baseline analyses are performed for all scales of the EORTC QLQ-C30 and EORTC QLQ-OES18. Summary statistics (number of patients, mean, standard deviation, median, minimum, maximum, 95% CI) of linearly transformed scores (per the EORTC scoring manual) are calculated at all assessment timepoints for each study arm.

Biomarkers

In this study, archival tissue samples obtained prior to definitive chemoradiation therapy are collected from all patients and tested for PD-L1 expression by a central laboratory during the screening period. The study enrolls an all-comers population with respect to PD-L1 status; however, patients are stratified by PD-L1 expression (TIC score <10% vs. ≥10%), as assessed by a central laboratory using the investigational Ventana PD-L1 (SP263) CDx Assay. The U.S. FDA granted approval of pembrolizumab for the treatment patients with recurrent locally advanced or metastatic squamous cell carcinoma of the esophagus with disease progression after one or more prior lines of systemic therapy whose tumors are PD-L1 positive, as determined using the DAKO 22C3 PD-L1 immunohistochemistry (IHC) assay in CPS≥10. In an esophageal cancer cross-assay evaluation, PD-L1 positive cases identified using the SP263 PD-L1 TIC 10% are very similar to those identified with 22C3 CPS≥10.

Archival, pre-treatment, on-treatment, and/or post-treatment biopsy tumor specimens obtained from patients are used for exploratory analysis of other tumor-based biomarkers, which may include, but are not limited to, PD-L1/PD-1 immunobiology, TIGIT immunobiology, tumor immunobiology, mechanisms of resistance, or tumor types or subtypes, and tumor mutational burden. The evaluation of biomarkers may help to identify which patients may potentially benefit most from tiragolumab plus atezolizumab and may help to guide future development of novel therapeutic and diagnostic options.

DNA and/or RNA extraction and analysis may be performed to enable next-generation sequencing (NGS) and to evaluate expression of genes to assess their association with efficacy and/or to identify selected somatic mutations and disease pathways to increase the understanding of disease pathobiology.

Blood samples are collected at baseline and during the study to evaluate changes in surrogate biomarkers. Correlations between these biomarkers and safety and efficacy endpoints are explored to identify blood-based biomarkers that might predict which patients are more likely to benefit from atezolizumab.

Tissue samples are collected for DNA extraction to enable whole exome sequencing (WES) to identify variants that are predictive of response to study drug, are associated with progression to a more severe disease state, are associated with susceptibility to developing adverse events, can lead to improved adverse event monitoring or investigation, or can increase the knowledge and understanding of disease biology and drug safety. Genomics is increasingly informing researchers' understanding of disease pathobiology. WES provides a comprehensive characterization of the exome, and, along with clinical data collected in this study, may increase the opportunity for developing new therapeutic approaches or new methods for monitoring efficacy and safety or predicting which patients are more likely to respond to a drug or develop adverse events. Data are analyzed in the context of this study but may also be explored in aggregate with data from other studies. The availability of a larger dataset will assist in identification and characterization of important biomarkers and pathways to support future drug development.

Samples for the following laboratory tests are sent to the study site's local laboratory for analysis:
Hematology: WBC count, RBC count, hemoglobin, hematocrit, platelet count, and differential count (neutrophils, eosinophils, basophils, monocytes, lymphocytes)
Chemistry panel (serum or plasma): bicarbonate or total carbon dioxide (if considered standard of care for the region), sodium, potassium, chloride, glucose, BUN or urea, creatinine, total protein, albumin, phosphate, calcium, total bilirubin, ALP, ALT, AST, and LDH
Coagulation: INR, and aPTT
Thyroid function testing: thyroid-stimulating hormone, free triiodothyronine (T3) (or total T3 for sites where free T3 is not performed), and free thyroxine (also known as T4)
EBV serology, as outlined below:
   EBV VCA IgM
   EBV VCA IgG or Epstein-Barr nuclear antigen IgG
   if clinically indicated: EBV PCR
HIV serology
HBV serology: hepatitis B surface antigen (HBsAg), hepatitis B surface antibody (HBsAb), and total hepatitis B core antibody (HBcAb) for all patients; HBV DNA for patients with a positive HBsAg and patients with negative HBsAg and HBsAb tests and a positive total HBcAb test
HCV serology: HCV antibody and (if HCV antibody test is positive) HCV RNA
Pregnancy test

All women of childbearing potential will have a serum pregnancy test at screening. During the study, urine pregnancy tests will be performed on Day 1 of every cycle, and after study treatment is discontinued, pregnancy tests will be performed at the treatment discontinuation visit, and also at either 90 days after the final dose of tiragolumab/placebo or 5 months after the final dose of atezolizumab/placebo, whichever is later. If a urine pregnancy test is positive, it must be confirmed by a serum pregnancy test.

A woman is considered to be of childbearing potential if she is postmenarchal, has not reached a postmenopausal state 12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis).
Urinalysis (pH, specific gravity, glucose, protein, ketones, and blood); dipstick permitted

Samples (blood and tumor) for the following laboratory tests are sent to one or several central laboratories or to the Sponsor or a designee for analysis:
C-reactive protein
PK assays
   Serum samples are obtained for measurement of tiragolumab and/or atezolizumab concentrations using validated immunoassays.
ADA assays
   Serum samples are obtained for measurement of ADAs to tiragolumab and/or to atezolizumab using validated assays.
Exploratory biomarker assays
   Blood samples are obtained for biomarker evaluation (including but not limited to biomarkers that are related to esophageal or tumor immune biology) from all eligible patients at visits. Samples may be processed to obtain plasma, serum, and/or peripheral blood mononuclear cells (PBMCs) and their derivatives (e.g., RNA and DNA).
Auto-antibody assays
   Serum samples collected for the assessment of PK, ADAs, or biomarkers at baseline on Day 1 of Cycle 1 prior to the first dose of study treatment, may be used for auto-antibody testing if an immune-mediated adverse event develops in a patient that would warrant such an assessment.

Archival tissue samples collected prior to definitive concurrent chemoradiation therapy are analyzed for PD-L1 expression through the use of the investigational Ventana PD-L1 (SP263) CDx Assay for stratification purposes (TIC<10% vs. 10%) and (optional) fresh tissue sample will be collected for exploratory research on other biomarkers and biomarker development.
Tumor tissue should be of good quality based on total and viable tumor content. Samples must contain a minimum of 50 viable tumor cells that preserve cellular context and tissue architecture regardless of needle gauge or retrieval method. Fine-needle aspiration, brushing, cell pellets from pleural effusion, and lavage samples are not acceptable. For core-needle biopsy specimens, at least three cores should be submitted for evaluation.
Archival tumor tissue samples obtained outside of this study for central assessment of PD-L1 results and other biomarker analyses will be collected from all patients (paraffin blocks are preferred; or at least 15 unstained serial slides are acceptable). The availability of archival tumor tissue must be confirmed prior to study entry. The patient may still be eligible upon discussion with the Medical Monitor if <15 unstained, serial slides can be provided. Fine-needle aspirates, cell pellets from effusions or ascites, and lavage samples do not satisfy the requirement for archival tissue.
If adequate tissue from distinct timepoints, priority should be given to the tissue most recently collected.
Patients having additional tissue samples from procedures performed at different times during this study are requested (but not required) to also submit these samples for central testing. Tissue samples will be obtained at multiple times for individual patients will greatly contribute to understanding an improved understanding of the mechanism of action of the treatment and disease biology. This will not be applicable if approval by the local regulatory and/or required decision bodies are not obtained. For patients who agree to optional biopsies, tissue samples for biopsy may be collected per investigator discretion, either on-treatment or at progression. Optional biopsies should consist of core-needle biopsies (at least 3 cores, preferred) for deep tumor tissue or organs or excisional, incisional, punch, or forceps biopsies for cutaneous, subcutaneous, or mucosal lesions. This will not be applicable if approval by the local regulatory and/or required decision bodies are not obtained.

Exploratory biomarker analyses may be performed in an effort to understand the association of these markers (e.g., TIGIT status) with study treatment efficacy. The efficacy outcomes may be explored in a population of patients whose tumors have high TIGIT expression, as determined by IHC and/or RNA analysis. Exploratory analysis of WGS data may be conducted in the context of this study.

Exploratory biomarker research may include, but will not be limited to, analysis of genes or gene signatures associated with tumor immunobiology, PD-L1, lymphocyte subpopulations, T-cell receptor repertoire, or cytokines associated with T-cell activation. Research may involve extraction of DNA, cell-free DNA, or RNA; analysis of mutations, single nucleotide polymorphisms, and other genomic variants; and genomic profiling through use of NGS of a comprehensive panel of genes. DNA extracted from blood may be compared with DNA extracted from tissue to identify somatic variants by distinguishing germline variants from somatic variants NGS methods may include WES of tissue and blood samples, but WES of blood samples will be performed only at participating sites.

Use of Investigational Ventana PD-L1 (SP263) CDx Assay

The investigational Ventana PD-L1 (SP263) CDx Assay is intended for the qualitative immunohistochemical assessment of the programmed death ligand (PD-L1) protein in FFPE esophageal squamous cell carcinoma tissue with PD-L1 (SP263) rabbit monoclonal antibody, using the BenchMark ULTRA staining platform with OptiView DAB IHC detection kit. TIC Score of <10% vs. 10% of PD-L1 expression is used for interpretation of the IHC analysis results.

PD-L1 expression is determined using the investigational Ventana PD-L1 (SP263) CDx Assay. TIC score of <10% vs. ≥10% of PD-L1 expression is used as one of the factors for patient stratification from archival specimen pre-treatment tissue samples collected prior to the initiation of definitive concurrent chemoradiotherapy.

Patients with histologically or cytologically confirmed diagnosis of unresectable locally advanced esophageal squamous cell carcinoma who have not progressed following definitive concurrent chemoradiotherapy regardless of PD-L1 expression are eligible for enrollment into the trial. Prevalence of PD-L1 expression using SP263-based TIC scoring was investigated in an internal procured set of 669 esophageal squamous cell carcinoma tissues, of which 94% (627 of 669) were PD-L1 positive (TIC 1%) and 41% (274 of 669) had a TIC 10%, the proposed stratification cutoff for this study.

Archival tumor tissue (recommended to be less than 6 months old) collected prior to definitive concurrent chemoradiation therapy is used to determine the baseline PD-L1 status. The likelihood of a fresh pre-treatment tumor biopsy collected prior to definitive concurrent chemoradiation therapy solely for PD-L1 testing is pretty small and will be at the investigator's discretion.

Since archival tumor specimens are mainly used for PD-L1 testing and that the results of PD-L1 testing will be used for patient stratification in an all-comers trial, there is no risk to health, safety, or welfare of the patient with regard to use of the investigational Ventana PD-L1 (SP263) CDx Assay.

Patients may undergo additional optional biopsies for exploratory biomarker research, including PD-L1 assessment by the investigational Ventana PD-L1 (SP263) CDx Assay to determine PD-L1 expression levels. For patients who agree to optional biopsies, tissue samples for biopsy may be collected per investigator discretion, either pre-treatment, on-treatment, or at disease progression, and sampling will be performed according to standard of care.

Patient Eligibility

Inclusion Criteria

Patients must meet the following criteria for study entry:
Signed Informed Consent Form
Age≥18 years at time of signing Informed Consent Form
Ability to comply with the study protocol
ECOG Performance Status of 0 or 1
Histologically or cytologically confirmed diagnosis of squamous cell carcinoma of the esophagus
Stage II-IVA per American Joint Committee on Cancer/Union for International Cancer Control, 8th edition, unresectable locally advanced disease (medically or surgery is declined)
   Patients are not expected to undergo tumor resection during the course of the study.
   Ineligibility for curative surgery must be based on the documented opinion of the qualified medical, surgical or radiation oncologist.
   Stage IVB patients diagnosed with cervical or upper thoracic esophageal squamous cell carcinoma with supraclavicular lymph node metastases only and are deemed suitable for definitive concurrent chemoradiation therapy in the opinion of the treating physician, multidisciplinary team or tumor board are eligible.
Definitive concurrent chemoradiation treatment according to regional oncology guidelines for esophageal cancer, with the following criteria:
   Patients with inoperable cancer must have received at least 2 cycles of platinum-based chemotherapy and radiation therapy consistent with definitive treatment (50-64 Gy) without evidence of radiographic disease progression per RECIST v1.1, as documented by comparison of scans (pre- and post-definitive concurrent chemoradiotherapy) prior to randomization. Patients with cervical esophageal squamous cell carcinoma may receive higher radiation dose (50-66 Gy), as per local oncology guidelines.
   Randomization into the study must occur within 1-84 days after the last dose of definitive concurrent chemoradiotherapy.
Representative archival formalin-fixed, paraffin-embedded (FFPE) tumor specimens <6 months old, collected prior to initiation of definitive chemoradiotherapy (either an archival specimen or fresh pre-treatment tissue prior to initiation of definitive concurrent chemoradiation therapy) in paraffin blocks (preferred) or ≥15 unstained slides containing freshly cut, serial sections
   Patients with <15 unstained slides available at baseline may be eligible upon discussion with the Medical Monitor. Patients with archival tumor specimens months old available at baseline may be eligible upon discussion with the Medical Monitor if recent biopsy is not clinically feasible.
   Tumor tissue should be of good quality based on total and viable tumor content and must be evaluated for PD-L1 (SP263) expression prior to randomization.
   Patients whose tumor tissue is not evaluable for PD-L1 expression are not eligible.
   For the purpose of stratification, the PD-L1 score of the patient's tumor will be the highest PD-L1 TIC score among all samples tested from a single patient prior to stratification, if multiple samples are submitted.
   Acceptable samples include core needle biopsies for deep tumor tissue or excisional, incisional, punch, or forceps biopsies for cutaneous, subcutaneous, or mucosal lesions.
   FFPE tumor specimens in paraffin blocks are preferred. Fine-needle aspiration, brushing, cell pellet from effusions, and lavage samples are not acceptable.
Adequate hematologic and end-organ function, defined by the following laboratory test results, obtained after the last dose of chemotherapy within 14 days prior to initiation of study treatment:
   ANC≥1.2×10⁹/L (1200/μL) without granulocyte colony-stimulating factor support
   Lymphocyte count ≥0.5×10⁹/L (500/4)
   Platelet count ≥100×10⁹/L (100,000/4) without transfusion
   Hemoglobin ≥90 g/L (9 g/dL)
   Patients may be transfused to meet this criterion.
   AST, ALT, and ALP≤2.5×upper limit of normal (ULN)
   Total bilirubin ≤1.5×ULN with the following exception:
   Patients with known Gilbert disease: total bilirubin 3×ULN
   Creatinine ≤1.5×ULN
   Albumin ≥25 g/L (2.5 g/dL)
   For patients not receiving therapeutic anticoagulation: INR and aPTT≤1.5×ULN
For patients receiving therapeutic anticoagulation: stable anticoagulant regimen
Negative HIV test at screening
   Patients without hepatitis B virus (HBV) infection or for patients with a positive hepatitis B surface antigen (HBsAg) test and/or a positive total hepatitis B core antibody (HBcAb) test in the absence of a positive hepatitis B surface antibody (HBsAb) test at screening: HBV DNA <500 IU/mL.
      Patients with detectable HBV DNA should be managed per institutional guidelines. Initiation of anti-HBV therapy should be 14 days prior to initiation of study treatment, and patients should be willing to continue anti-HBV therapy for the duration of study treatment, and longer per institutional guidelines.
Negative hepatitis C virus (HCV) antibody test at screening, or positive HCV antibody test followed by a negative HCV RNA test at screening The HCV RNA test is performed only for patients who have a positive HCV antibody test.
For women of childbearing potential: agreement to remain abstinent (refrain from heterosexual intercourse) or use contraception, as defined below:
Women must remain abstinent or use contraceptive methods with a failure rate of <1% per year during the treatment period, for 5 months after the final dose of atezolizumab/placebo, and for 90 days after the final dose of tiragolumab/placebo, whichever is later.
A woman is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
Examples of contraceptive methods with a failure rate of <1% per year include bilateral tubal ligation, male sterilization, hormonal contraceptives that inhibit ovulation, hormone-releasing intrauterine devices, and copper intrauterine devices.
The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For men: agreement to remain abstinent (refrain from heterosexual intercourse) or use a condom, and agreement to refrain from donating sperm, as defined below:
With a female partner of childbearing potential or pregnant female partner, men must remain abstinent or use a condom during the treatment period and for 90 days after the final dose of tiragolumab/placebo to avoid exposing the embryo. Men must refrain from donating sperm during this same period.
The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of preventing drug exposure. If required per local guidelines or regulations, information about the reliability of abstinence is described in the local Informed Consent Form.

Exclusion Criteria

Patients who meet any of the following criteria are excluded from study entry:
Prior treatment with CD137 agonists or immune checkpoint blockade therapies, including anti-CTLA-4, anti-PD-1, anti-PD-L1 and anti-TIGIT therapeutic antibodies
Any unresolved toxicity of NCI CTCAE Grade 2 from the prior chemoradiation therapy Patients with irreversible and manageable hearing loss are eligible.
Evidence of complete esophageal obstruction not amenable to treatment
Histology consistent with small cell esophageal carcinoma, esophageal adenocarcinoma, or mixed carcinoma
Grade ≥2 peripheral neuropathy as defined by NCI CTCAE v5.0 criteria
High risk for developing esophageal fistula by clinical assessment or imaging, such as prior history or associated symptoms of esophageal fistula, or primary tumor invasion of the great vessels or trachea
Prior esophagectomy
Positive Epstein-Barr virus (EBV) viral capsid antigen IgM test at screening
An EBV polymerase chain reaction (PCR) test should be performed as clinically indicated to screen for active infection or suspected chronic active infection. Patients with a positive EBV PCR test are excluded.
Uncontrolled tumor-related pain
Patients requiring pain medication must be on a stable regimen at study entry.
Uncontrolled pleural effusion, pericardial effusion, or ascites requiring recurrent drainage procedures (once monthly or more frequently) Patients with indwelling catheters (e.g., PleurX®) are allowed.
Uncontrolled or symptomatic hypercalcemia (ionized calcium >1.5 mmol/L, calcium >12 mg/dL, or corrected calcium >ULN)
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
Patients with a history of autoimmune-related hypothyroidism who are on thyroid-replacement hormone are eligible for the study.
Patients with controlled Type 1 diabetes mellitus who are on an insulin regimen are eligible for the study.
Patients with eczema, psoriasis, lichen simplex chronicus, or vitiligo with dermatologic manifestations only (e.g., patients with psoriatic arthritis are excluded) are eligible for the study provided all of following conditions are met:
   Rash must cover <10% of body surface area
   Disease is well controlled at baseline and requires only low-potency topical corticosteroids
   No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan
History of radiation pneumonitis in the radiation field (fibrosis) is permitted.
Active tuberculosis
Significant cardiovascular disease (such as New York Heart Association Class II or greater cardiac disease, myocardial infarction, or cerebrovascular accident) within 3 months prior to initiation of study treatment, unstable arrhythmia, or unstable angina
Patients with known coronary artery disease, congestive heart failure not meeting the above criteria, or left ventricular ejection fraction <50% must be on a stable medical regimen that is optimized in the opinion of the treating physician, in consultation with a cardiologist if appropriate
Major surgical procedure, other than for diagnosis, within 4 weeks prior to initiation of definitive concurrent chemoradiation
History of malignancy other than esophageal cancer within 2 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer
Patients who received endoscopic mucosal resection or dissection for superficial mucosal cancers other than ESCC within 2 years prior to screening are eligible for the study.
Patients with illness or conditions that interfere with their capacity to understand, follow, and/or comply with study procedures
Severe infection within 4 weeks prior to randomization, including, but not limited to, hospitalization for complications of infection, bacteremia, or severe pneumonia, or any active infection that, in the opinion of the investigator, could impact patient safety.
Treatment with therapeutic oral or IV antibiotics within 2 weeks prior to randomization Patients receiving prophylactic antibiotics (e.g., to prevent a urinary tract infection or chronic obstructive pulmonary disease exacerbation) are eligible for the study.
Prior allogeneic stem cell or solid organ transplantation
Any other disease, metabolic dysfunction, physical examination finding, or clinical laboratory finding that contraindicates the use of an investigational drug, may affect the interpretation of the results, or may render the patient at high risk from treatment complications
Treatment with a live, attenuated vaccine (e.g., FLUMIST®) within 4 weeks prior to initiation of study treatment, or anticipation of need for such a vaccine during study treatment, within 5 months after the last dose of atezolizumab/placebo or 90 days after the last dose of tiragolumab/placebo, whichever occurs later.
Patients must not receive live, attenuated influenza vaccines (e.g., FluMist) within 4 weeks prior to randomization, during treatment, and for 5 months following the last dose of study treatment.
Treatment with any other investigational agent, including EGFR inhibitors, with therapeutic intent for esophageal cancer prior to randomization
Treatment with systemic immunostimulatory agents (including, but not limited to, interferon and interleukin-2 (IL-2)) within 4 weeks or 5 drug-elimination half-lives (whichever is longer) prior to randomization
Treatment with systemic immunosuppressive medication (including, but not limited to, corticosteroids, cyclophosphamide, azathioprine, methotrexate, thalidomide, and anti-TNF-α agents) within 2 weeks prior randomization or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study after Medical Monitor confirmation has been obtained.
   Patients who received mineralocorticoids (e.g., fludrocortisone), corticosteroids for chronic obstructive pulmonary disease (COPD) or asthma, or low-dose corticosteroids for orthostatic hypotension or adrenal insufficiency are eligible for the study.
History of severe allergic anaphylactic reactions to chimeric or humanized antibodies or fusion proteins
Known hypersensitivity to Chinese hamster ovary cell products or to any component of the tiragolumab or atezolizumab formulation
Pregnancy or breastfeeding, or intention of becoming pregnant during study treatment, within 5 months after the final dose of atezolizumab, or within 90 days after the final dose of tiragolumab, whichever occurs later.
Women of childbearing potential must have a negative serum pregnancy test result within 14 days prior to randomization.

### Example 2. A Phase III, Randomized, Multicenter, Double-Blind Study Designed to Evaluate the Efficacy and Safety of Atezolizumab Plus Tiragolumab in Combination with Paclitaxel and Cisplatin Compared with Atezolizumab Placebo Plus Tiragolumab Placebo in Combination with Paclitaxel and Cisplatin as First-Line Treatment in Patients with Unresectable Locally Advanced, Unresectable Recurrent, or Metastatic Esophageal Squamous Cell Carcinoma

The present example describes a randomized, Phase III, multicenter, double-blinded study (YO42138) designed to evaluate whether atezolizumab plus tiragolumab in combination with paclitaxel and cisplatin (Atezo+Tira+PC) as first-line treatment in patients with unresectable locally advanced, unresectable recurrent, or metastatic esophageal squamous cell carcinoma (ESCC) is safe and effective compared with atezolizumab placebo plus tiragolumab placebo in combination with paclitaxel and cisplatin (Placebo+PC).

Study Design

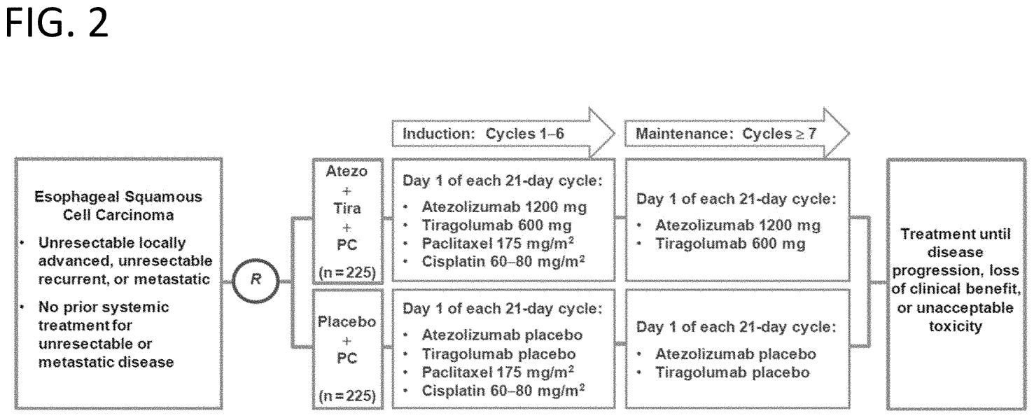

Described below are the details of a randomized, Phase III, multicenter, double-blinded study for evaluating the safety and efficacy of Atezo+Tira+PC compared with Placebo+PC in patients with unresectable locally advanced, unresectable recurrent, or metastatic ESCC. FIG. 2 illustrates the study design.

Eligible patients are randomized in a 1:1 ratio to Atezo+Tira+PC or Placebo+PC and stratified by PD-L1 expression as assessed by a central laboratory through use of the investigational Ventana PD-L1 (SP263) CDx Assay (tumor and tumor-associated immune cell (TIC) score <10% vs. TIC score ≥10%), previous curative treatment consisting of either esophagectomy or chemoradiotherapy (yes vs. no), and Eastern Cooperative Oncology Group (ECOG) Performance Status (0 vs. 1). Patients receive treatment as outlined in Table 6.

TABLE 6
Study Treatment Arms

| | Dose, Route, and Regimen | |
|---|---|---|
| | (Drugs Listed in Order of Administration) | |
| | Induction: Cycles 1-6 | Maintenance: Cycles ≥ 7 |
| Treatment Arm | (21-Day Cycles) | (21-Day Cycles) |
| Atezo + Tira + PC | Atezolizumab 1200 mg IV on Day 1 | Atezolizumab 1200 mg IV on Day 1 |
| | Tiragolumab 600 mg IV on Day 1 | Tiragolumab 600 IV mg on Day 1 |
| | Paclitaxel 175 mg/m² IV on Day 1 | |
| | Cisplatin 60-80 mg/m² IV on Day 1 ^{a} | |
| Placebo + PC | Atezolizumab placebo IV on Day 1 | Atezolizumab placebo IV on Day 1 |
| | Tiragolumab placebo IV on Day 1 | Tiragolumab placebo IV on Day 1 |
| | Paclitaxel 175 mg/m² IV on Day 1 | |
| | Cisplatin 60-80 mg/m 2 IV on Day 1 ^{a} | |

^{a} Cisplatin dose should be consistent with manufacturer and institutional standards.

Patients receive study treatment until unacceptable toxicity or loss of clinical benefit as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune-cell infiltration in the setting of a T-cell response (termed pseudoprogression) with cancer immunotherapy, radiographic progression per Response Evaluation Criteria in Solid Tumors, Version 1.1 (RECIST v1.1) may not be indicative of true disease progression. In the absence of unacceptable toxicity, patients who meet criteria for disease progression per RECIST v1.1 while receiving study treatment are permitted to continue treatment if they meet all of the following criteria:
Evidence of clinical benefit, as determined by the investigator following a review of all available data
Absence of symptoms and signs (including laboratory values, such as new or worsening hypercalcemia) indicating unequivocal progression of disease
Absence of decline in ECOG Performance Status that can be attributed to disease progression
Absence of tumor progression at critical anatomical sites (e.g., leptomeningeal disease) that cannot be managed by protocol-allowed medical interventions

Patients are closely monitored for adverse events throughout the study, and adverse events are graded according to the National Cancer Institute Common Terminology Criteria for Adverse Events, Version 5.0. Laboratory safety assessments include regular monitoring of hematology and blood chemistry. Patients undergo tumor assessments every 6-9 weeks. Patients undergo patient-reported outcome (PRO) assessments at specified timepoints during treatment and for up to 1 year after treatment discontinuation.

Serum samples are collected for pharmacokinetic (PK) and immunogenicity analyses. Peripheral blood mononuclear cell (PBMC) and archival or newly collected tumor tissue samples are collected for determination of PD-L1 expression and/or exploratory biomarker research.

Following treatment discontinuation, information on survival follow-up and new anti-cancer therapy is collected until death (unless the patient withdraws consent or the Sponsor terminates the study).

Study Treatment Dosage and Administration

Patients receive study treatment until unacceptable toxicity or loss of clinical benefit as determined by the investigator after an integrated assessment of radiographic and biochemical data, local biopsy results (if available), and clinical status. The treatment regimens are summarized in Table 7.

Atezolizumab/Placebo

Atezolizumab 1200 mg or matching placebo (referred to as “atezolizumab” hereafter) is administered by IV infusion on Day 1 of each 21-day cycle. Atezolizumab infusions are administered per the instructions outlined in Table 7.

TABLE 7
Administration of First and Subsequent Atezolizumab Infusions

| First Infusion | Subsequent Infusions |
|---|---|
| No premedication is permitted prior to the | If the patient experienced an infusion-related |
| atezolizumab infusion. | reaction with any previous infusion, |
| Vital signs (pulse rate, respiratory rate, blood | premedication with antihistamines, anti- |
| pressure, and temperature) should be | pyretics, and/or analgesics may be |
| measured within 60 minutes prior to the | administered for subsequent doses at the |
| infusion. | discretion of the investigator. |
| Atezolizumab should be infused over 60 (±15) | Vital signs should be measured within |
| minutes. | 60 minutes prior to the infusion. |
| If clinically indicated, vital signs should be | Atezolizumab should be infused over |
| measured every 15 (±5) minutes during the | 30 (±10) minutes if the previous infusion was |
| infusion and at 30 (±10) minutes after the | tolerated without an infusion-related reaction, |
| infusion. | or 60 (±15) minutes if the patient |
| Patients should be informed about the | experienced an infusion-related reaction with |
| possibility of delayed post-infusion symptoms | the previous infusion. |
| and instructed to contact their study physician | If the patient experienced an infusion-related |
| if they develop such symptoms. | reaction with the previous infusion or if |
| | clinically indicated, vital signs should be |
| | measured during the infusion and at |
| | 30 (±10) minutes after the infusion. |

Tiragolumab/Placebo

Tiragolumab 600 mg or matching placebo (referred to as “tiragolumab” hereafter) will be administered by IV infusion on Day 1 of each 21-day cycle. On Day 1 of Cycle 1, tiragolumab will be administered 60 minutes after completion of the atezolizumab infusion. The interval between subsequent infusions will be 30 minutes if the previous atezolizumab infusion was tolerated without an infusion-related reactions (IRR) or 60 minutes if the patient experienced an IRR with the previous atezolizumab infusion. Tiragolumab infusions will be administered per the instructions outlined in Table 8.

TABLE 8
Administration of First and Subsequent Tiragolumab Infusions

| First Infusion | Subsequent Infusions |
|---|---|
| No premedication is permitted prior to the | If the patient experienced an infusion-related |
| tiragolumab infusion. | reaction with any previous infusion, premedication |
| Vital signs (pulse rate, respiratory rate, | with antihistamines, anti-pyretics, and/or analgesics |
| blood pressure, and temperature) should | may be administered for subsequent doses at the |
| be recorded within 60 minutes prior to the | discretion of the investigator. |
| infusion. | Vital signs should be recorded within 60 minutes |
| Tiragolumab should be infused over | prior to the infusion. |
| 60 (±10) minutes. | Tiragolumab should be infused over |
| Vital signs should be recorded every | 30 (±10) minutes if the previous infusion was |
| 15 (±5) minutes during the infusion and at | tolerated without an infusion-related reaction, or |
| 30 (±10) minutes after the infusion. | 60 (±10) minutes if the patient experienced an |
| Patients should be observed for 60 minutes | infusion-related reaction with the previous infusion. |
| after completion of the tiragolumab infusion. | Patients should be observed for 30 minutes after |
| Patients will be informed about the | completion of the tiragolumab infusion if the |
| possibility of delayed post-infusion | previous infusion was tolerated without an infusion- |
| symptoms and will be instructed to contact | related reaction, or 60 minutes after completion of |
| the study physician if they develop such | the tiragolumab infusion if the patient experienced |
| symptoms. | an infusion-related reaction with the previous |
| | infusion. |
| | If the patient experienced an infusion-related |
| | reaction with the previous infusion or if clinically |
| | indicated, vital signs should be recorded during the |
| | infusion and at 15 (±10) minutes after the infusion. |

Paclitaxel and Cisplatin

On Day 1 of Cycles 1-6 (21-day cycles), patients receive paclitaxel 175 mg/m², administered by IV infusion over 3 hours (±30) minutes, followed by cisplatin 60-80 mg/m² (dose should be consistent with manufacturer and institutional standards), administered by IV infusion over 2-3 hours (±60 minutes). On Day 1 of Cycle 1, paclitaxel is administered 60 minutes after completion of the tiragolumab infusion to allow for observation after tiragolumab administration. The interval between subsequent infusions is 30 minutes if the previous tiragolumab infusion was tolerated without an IRR or 60 minutes if the patient experienced an IRR with the previous tiragolumab infusion.

Patients should receive anti-emetics and IV hydration according to institutional standards and manufacturers instructions for paclitaxel and cisplatin. Because of the immunomodulatory effects of corticosteroids, premedication with corticosteroids should be minimized to the extent that is clinically feasible.

Dose Modifications

There are no dose modifications for atezolizumab or tiragolumab in this study.

For management of drug-related toxicities, the dose of paclitaxel and the dose of cisplatin may be reduced by up to two times, as outlined in Table 9. If the dose of one chemotherapy drug is reduced because of a toxicity considered to be solely related to that drug, there is no need to reduce the dose of the other chemotherapy drug.

TABLE 9
Recommended Dose Reduction for Paclitaxel and Cisplatin

| | | First Dose | Second Dose |
|---|---|---|---|
| | Initial Dose | Reduction | Reduction |
| Paclitaxel | 175 mg/m² | 135 mg/m² | 90 mg/m² |
| Cisplatin | 60-80 mg/m² | 75% of previous | 75% of previous |
| | | dose | dose |

If further dose reduction is indicated for cisplatin and/or paclitaxel after two dose reductions, that drug (or both drugs, if applicable) should be discontinued, but the patient may continue other study treatments at the investigator's discretion. After dose reduction, the dose may be escalated during subsequent administrations at the investigators discretion.

Suggested recommendations for cisplatin dose reductions for renal impairment are provided in Table 10.

TABLE 10
Recommended Cisplatin Dose Reductions for Renal Impairment

| | Creatinine Clearance (mL/min) | | |
|---|---|---|---|
| | >50 to <60 | >40 to ≤50 | ≤40 |
| Cisplatin dose | 75% of previous | 75% of previous | Permanently |
| | dose | dose | discontinue |

Treatment Interruption

Study treatment may be temporarily suspended as appropriate for management of toxicity. On the basis of the available characterization of mechanism of action, tiragolumab may cause adverse events similar to but independent of atezolizumab, may exacerbate the frequency or severity of atezolizumab-related adverse events, or may have non-overlapping toxicities with atezolizumab. Because these scenarios may not be distinguished from one another in the clinical setting, immune-mediated adverse events should generally be attributed to both study drugs, and dose interruptions or treatment discontinuation in response to immune-mediated adverse events should be applied to both atezolizumab and tiragolumab.

Atezolizumab and/or tiragolumab may be temporarily suspended for up to 12 weeks (approximately four cycles). If corticosteroids are initiated for treatment of the toxicity, they must be tapered over 1 month to equivalent of 10 mg/day oral prednisone or equivalent before drug can be resumed. If atezolizumab or tiragolumab is withheld for >12 weeks, the patient will be discontinued from that drug. However, the drug may be withheld for >12 weeks to allow for patients to taper off corticosteroids prior to resuming treatment. Atezolizumab or tiragolumab can be resumed after being withheld for >12 weeks if the Medical Monitor agrees that the patient is likely to derive clinical benefit.

On the basis of the available characterization of mechanism of action, tiragolumab may cause adverse events similar to, but independent of, atezolizumab. Tiragolumab may also exacerbate the frequency or severity of atezolizumab-related adverse events or may have non-overlapping toxicities with atezolizumab. Because these scenarios may not be distinguishable from each other in the clinical setting, immune-mediated adverse events should generally be attributed to both agents, and dose interruptions or treatment discontinuation in response to immune-mediated adverse events should be applied to both tiragolumab and atezolizumab.

Paclitaxel and/or cisplatin treatment may be temporarily suspended in patients experiencing toxicity considered to be related to study treatment. If paclitaxel or cisplatin have been withheld for >6 weeks because of toxicity, the patient should be discontinued from both chemotherapy agents. However, paclitaxel or cisplatin can be resumed after being withheld for >6 weeks if the Medical Monitor agrees that the patient is likely to derive clinical benefit.

If one or more study treatments is interrupted, subsequent cycles should be restarted such that the study treatment infusions remain synchronized.

If atezolizumab is discontinued, tiragolumab should also be discontinued, but paclitaxel and cisplatin may be continued if the patient is likely to derive clinical benefit, as determined by the investigator. If paclitaxel, cisplatin, or tiragolumab is discontinued, the other drugs can be continued if the patient is likely to derive clinical benefit, as determined by the investigator.

Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the treatment discontinuation visit.

Permitted Therapy

Patients are permitted to use the following therapies during the study:
Oral contraceptives with a failure rate of <1% per year
Hormone-replacement therapy
Prophylactic or therapeutic anticoagulation therapy (such as warfarin at a stable dose or low-molecular-weight heparin)
Inactivated influenza vaccinations
Megestrol acetate administered as an appetite stimulant
Mineralocorticoids (e.g., fludrocortisone)
Corticosteroids administered for chronic obstructive pulmonary disease or asthma
Low-dose corticosteroids administered for orthostatic hypotension or adrenocortical insufficiency
Palliative radiotherapy (e.g., treatment of known bony metastases or symptomatic relief of pain) as outlined below:

For patients without documentation of progression of disease, investigators are strongly encouraged to maximize supportive care for symptomatic management and avoid radiotherapy that will interfere with the assessment of target lesions.

Treatment with tiragolumab and atezolizumab may be continued during palliative radiotherapy.

Premedication with antihistamines, anti-pyretics, and/or analgesics may be administered for the second and subsequent atezolizumab and tiragolumab infusions only, at the discretion of the investigator.

In general, investigators should manage a patient's care (including preexisting conditions) with supportive therapies other than those defined as cautionary or prohibited therapies as clinically indicated, per local standard practice. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

Corticosteroids and TNFα Inhibitors

Systemic corticosteroids and TNF-α inhibitors may attenuate potential beneficial immunologic effects of treatment with atezolizumab. Therefore, in situations in which systemic corticosteroids or TNF-α inhibitors would be routinely administered, alternatives, including antihistamines, should be considered. If the alternatives are not feasible, systemic corticosteroids and TNF-α inhibitors may be administered at the discretion of the investigator.

Systemic corticosteroids are recommended, at the discretion of the investigator, for the treatment of specific adverse events when associated with atezolizumab therapy.

Herbal Therapies

Concomitant use of herbal therapies is not recommended because their pharmacokinetics, safety profiles, and potential drug-drug interactions are generally unknown. However, herbal therapies not intended for the treatment of cancer may be used during the study at the discretion of the investigator.

Prohibited Therapy

Use of the following concomitant therapies is prohibited as described below:
Concomitant therapy intended for the treatment of cancer, whether health authority-approved or experimental, is prohibited for various time periods prior to starting study treatment, depending on the agent, and during study treatment, until disease progression is documented and the patient has discontinued study treatment, with the exception of palliative radiotherapy under certain circumstances.
Investigational therapy is prohibited within 28 days prior to initiation of study treatment and during study treatment.
Live, attenuated vaccines (e.g., FLUMIST®) are prohibited within 4 weeks prior to initiation of study treatment, during atezolizumab treatment, and for 5 months after the final dose of atezolizumab.
Systemic immunostimulatory agents (including, but not limited to, interferons and IL-2) are prohibited within 4 weeks or 5 drug-elimination half-lives (whichever is longer) prior to initiation of study treatment and during study treatment because these agents could potentially increase the risk for autoimmune conditions when given in combination with atezolizumab.

Systemic immunosuppressive medications (including, but not limited to, cyclophosphamide, azathioprine, methotrexate, and thalidomide) are prohibited during study treatment because these agents could potentially alter the efficacy and safety of atezolizumab.

Objectives and Efficacy Endpoints

This study evaluates the efficacy and safety of atezolizumab plus tiragolumab in combination with paclitaxel and cisplatin (Atezo+Tira+PC) compared with atezolizumab placebo plus tiragolumab placebo in combination with paclitaxel and cisplatin (Placebo+PC) as first line treatment in patients with unresectable locally advanced, unresectable recurrent, or metastatic ESCC. Specific objectives and corresponding endpoints for the study are outlined in Table 11.

TABLE 11
Objectives and Corresponding Endpoints

| | Corresponding Endpoints |
|---|---|
| Primary Efficacy Objective | |
| To evaluate the efficacy of Atezo + Tira + PC | OS, defined as the time from randomization to |
| compared with Placebo + PC | death from any cause |
| | PFS, defined as the time from randomization to |
| | the first occurrence of disease progression or |
| | death from any cause (whichever occurs first), as |
| | determined by the investigator according to |
| | RECIST v1.1 |
| To evaluate the efficacy of Atezo + Tira + PC | Confirmed ORR, defined as the proportion of |
| compared with Placebo + PC | patients with a CR or PR on two consecutive |
| | occasions ≥4 weeks apart, as determined by the |
| | investigator according to RECIST v1.1 |
| | DOR, defined as the time from the first |
| | occurrence of a confirmed objective response to |
| | the first occurrence of disease progression or |
| | death from any cause (whichever occurs first), as |
| | determined by the investigator according to |
| | RECIST v1.1 |
| To evaluate the efficacy of Atezo + Tira + PC | TTCD in patient-reported physical functioning, |
| compared with Placebo + PC | role functioning, and GHS/QoL as measured by |
| | the respective scales of the EORTC QLQ-C30 |
| | and defined as the time from randomization to first |
| | deterioration (decrease from baseline of ≥10 |
| | points) that is either maintained for two |
| | consecutive assessments or followed by death |
| | from any cause within 3 weeks |
| | TTCD in patient-reported dysphagia as measured |
| | by the dysphagia scale of the EORTC |
| | QLQ-OES18 and defined as the time from |
| | randomization to first deterioration (increase from |
| | baseline of ≥10 points) that is either maintained |
| | for two consecutive assessments or followed by |
| | death from any cause within 3 weeks |
| Exploratory Efficacy Objective | |
| To evaluate the efficacy of Atezo + Tira + PC | TTP, defined as the time from randomization to |
| compared with Placebo + PC | the first occurrence of disease progression, as |
| | determined by the investigator according to |
| | RECIST v1.1 |
| | Mean scores and mean change from baseline |
| | scores in all scales of the EORTC QLQ-C30 and |
| | the EORTC QLQ-OES18 |
| | Proportion of patients with clinically meaningful |
| | changes in physical functioning, role functioning, |
| | GHS/QoL, and dysphagia as measured by the |
| | respective scales of the EORTC QLQ-C30 and |
| | the EORTC QLQ-OES18 |
| Safety Objective | |
| To evaluate the safety of Atezo + Tira + PC | Incidence and severity of adverse events, with |
| compared with Placebo + PC | severity determined according to NCI CTCAE |
| | v5.0 |
| | Change from baseline in targeted vital signs |
| | Change from baseline in targeted clinical |
| | laboratory test results |
| Pharmacokinetic Objective | |
| To characterize the PK profiles of tiragolumab | Serum concentration of tiragolumab and |
| and atezolizumab when given in combination with | atezolizumab at specified timepoints |
| paclitaxel and cisplatin | |
| Immunogenicity Objective | |
| To evaluate the immune response to tiragolumab | Prevalence of ADAs to tiragolumab at baseline |
| and atezolizumab when given in combination with | and incidence of ADAs to tiragolumab during the |
| paclitaxel and cisplatin | study |
| | Prevalence of ADAs to atezolizumab at baseline |
| | and incidence of ADAs to atezolizumab during the |
| | study |
| Exploratory Immunogenicity Objective | |
| To evaluate potential effects of ADAs | Relationship between tiragolumab and |
| | atezolizumab ADA status and efficacy, safety, or |
| | PK endpoints |
| Biomarker Objective | |
| To identify and/or evaluate biomarkers that are | Relationship between biomarkers in PBMCs and |
| associated with progression to a more severe | tumor tissue and efficacy, safety, PK, |
| disease state (i.e., prognostic biomarkers), are | immunogenicity, or other biomarker endpoints |
| associated with acquired resistance to study | |
| treatment, can provide evidence of study | |
| treatment activity (i.e., pharmacodynamic | |
| biomarkers), or can increase the knowledge and | |
| understanding of disease biology and drug safety | |
| Health Status Utility Objectives | |
| To evaluate health status utility scores of patients | Mean change from baseline in the index-based |
| treated with Atezo + Tira + PC compared with | and VAS scores of the EQ-5D-5L |
| Placebo + PC | |

ADA = anti-drug antibody; Atezo + Tira + PC = treatment with atezolizumab, tiragolumab, paclitaxel, and cisplatin; CR = complete response; DOR = duration of response; EORTC = European Organisation for Research and Treatment of Cancer; EQ-5D-5L = EuroQol 5-Dimension Questionnaire, 5-level version; GHS/QoL = global health status and quality of life; NCI CTCAE v5.0 = National Cancer Institute Common Terminology Criteria for Adverse Events, Version 5.0; ORR = objective response rate; OS = overall survival; PBMCs = peripheral blood mononuclear cell; PFS = progression-free survival; PR = partial response; PK = pharmacokinetic; Placebo + PC = treatment with atezolizumab placebo, tiragolumab placebo, paclitaxel, and cisplatin; QLQ-C30 = quality-of-life questionnaire for cancer; QLQ-OE518 = quality-of-life questionnaire for esophageal cancer; RECIST v1.1 = Response Evaluation Criteria in Solid Tumors, Version 1.1; TTCD = time to confirmed deterioration; TTP = time to progression; VAS = visual analog scale.

Efficacy Analysis

In this study, the co-primary efficacy endpoints are investigator-assessed PFS and OS. This study tests the hypothesis that treatment with Atezo+Tira+PC will prolong PFS and OS compared with treatment with Placebo+PC.

PFS as an endpoint can reflect tumor growth and can be assessed before the determination of a survival benefit; additionally, its determination is not generally confounded by subsequent therapies. Whether an improvement in PFS represents a direct clinical benefit or a surrogate for clinical benefit depends on the magnitude of the effect and the benefit-risk profile of the new treatment compared with available therapies (FDA 2007; European Medicines Agency 2012).

Improvement in OS is generally accepted as the best measure of clinical benefit for patients with advanced/unresectable or metastatic esophageal cancer. Recent data also suggest that OS may be a more sensitive endpoint for cancer immunotherapy than PFS.

Unless otherwise specified, efficacy analyses are performed in the ITT population, defined as all randomized patients regardless of whether they receive any study treatment, with patients grouped according to the treatment assigned at randomization.

Overall Survival

OS is defined as the time from randomization to death from any cause. Patients who are not reported as having died at the time of analysis are censored at the last date they were known to be alive. Patients with no post-baseline survival information are censored at the date of randomization.

The sample size of the study was determined on the basis of the number of deaths (OS events) required in the ITT population to demonstrate efficacy in terms of OS. To detect an improvement in OS through use of a log-rank test at a two-sided significance level of 0.04, approximately 267 OS events (59% of 450 patients) will be required at the final OS analysis to achieve an overall 80% power, assuming a target HR of 0.70. The minimum detectable difference (MDD) is an OS HR of 0.775 (median OS improvement of 4.1 months, from 14 months in the Placebo+PC arm to 18.1 months in the Atezo+Tira+PC arm). The final analysis of OS is expected to occur at approximately 34 months after the first patient is randomized. The calculation of sample size and estimates of the OS analysis timeline are based on the following assumptions:
Patient randomization in a 1:1 ratio to Atezo+Tira+PC or Placebo+PC
One-piece exponential distribution for OS in each arm
Median OS of 14 months in the Placebo+PC arm and 20 months in the Atezo+Tira+PC arm (increase of 6 months, corresponding to a target HR of 0.70)
Annual dropout rate of 5% for OS in each arm
One planned OS interim analysis at the time of the primary PFS analysis (number of deaths estimated to be 176), using the O'Brien-Fleming stopping boundaries approximated by the Lan-DeMets α-spending function
Recruitment of 450 patients will take place over approximately 16 months

One interim analysis of OS is performed at the time of the primary PFS analysis, which is estimated to occur at approximately 23 months after the first patient is randomized. It is anticipated that at this time, approximately 176 OS events (39% of 450 patients) will have occurred.

If fewer than 135 OS events (<30% of 450 patients) have occurred at the time of the primary PFS analysis, the interim OS analysis is delayed until 176 OS events have occurred. An administrative α of 0.000001 (negligible impact on overall type I error rate) is spent on the OS hypothesis at the time of the primary PFS analysis.

A group sequential design is used to account for the conduct of the interim analysis and control the type I error for OS. The Lan-DeMets α-spending function is used to approximate the O'Brien-Fleming stopping boundaries for the OS interim and final analyses. Table 12 shows the projected timing and stopping boundaries based on the number of OS events required at each OS analysis; actual boundaries based on the observed information fraction (i.e., actual number of events observed at time of analysis over the total planned target number of events in the ITT population) are calculated at the time of each OS analysis.

TABLE 12
Analysis Timing and Stopping Boundaries for OS Analyses

| | | | | Stopping Boundary: | |
|---|---|---|---|---|---|
| | | | | HR (Two-Sided p-Value) | |
| | Planned | Number of | Estimated | α Recycled to | α Not Recycled |
| Planned | Information | Events | Analysis Timing | OS | to OS |
| Analysis | Fraction | (Deaths) | from FPI ^{a} | (OS α = 0.05) | (OS α = 0.04) |
| OS interim | 66% | 176 ^{b} | 23 months | MDD | MDD HR ≤ 0.672 |
| analysis | | | | HR ≤ 0.683 | (p ≤ 0.008) |
| | | | | (p ≤ 0.012) | |
| OS final | 100% | 267 | 34 months | MDD | MDD HR ≤ 0.775 |
| analysis | | | | HR ≤ 0.784 | (p ≤ 0.037) |
| | | | | (p ≤ 0.046) | |

FPI= first patient in; HR = hazard ratio; MDD = minimum detectable difference; OS = overall survival; PFS = progression-free survival.
Note:
MDD HR is estimated on the basis of proportional hazards assumption.
^{a} Analysis timing is estimated on the basis of protocol assumptions. Actual timing depends on the exact time when the required events have accrued.
^{b} The OS interim analysis will be conducted when approximately 293 PFS events have occurred. It is anticipated that approximately 176 OS events will have occurred at the time of the primary PFS analysis.

Progression-Free Survival

Investigator-assessed PFS is defined as the time from randomization to the first occurrence of disease progression or death from any cause (whichever occurs first), as determined by the investigator according to RECIST v1.1. Patients who have not experienced disease progression or death at the time of analysis are censored at the time of the last tumor assessment. Patients with no post-baseline tumor assessment are censored at the date of randomization.

The primary analysis of investigator-assessed PFS takes place when approximately 293 PFS events have been observed in the ITT population (65% of 450 patients). This provides an overall 93.5% power to detect a target HR of 0.62 for PFS using a log-rank test at a two-sided significance level of 0.01. The MDD is a PFS HR of 0.740 (median PFS improvement of 2.1 months, from 6 months in the Placebo+PC arm to 8.1 months in the Atezo+Tira+PC arm). The primary analysis of PFS is expected to occur at approximately 23 months after the first patient is randomized. The estimates are based on the following assumptions:
Patient randomization in a 1:1 ratio to Atezo+Tira+PC or Placebo+PC
One-piece exponential distribution for PFS in each arm
Median PFS of 6 months in the Placebo+PC arm and 9.7 months in the Atezo+Tira+PC arm (increase of 3.7 months, corresponding to a target HR of 0.62)
Annual dropout rate of 5% for PFS in each arm
Recruitment of 450 patients will take place over approximately 16 months

Objective Response Rate

An objective response is defined as a complete response (CR) or partial response (PR) as determined by the investigator according to RECIST v1.1. Patients not meeting these criteria, including patients without any postbaseline tumor assessment, will be considered non-responders. Confirmed ORR is defined as the proportion of patients who achieved an objective response on two consecutive occasions ≥4 weeks apart. The analysis population for ORR is all patients in the ITT population with measurable disease at baseline.

The two-sided Cochran-Mantel-Haenszel test, stratified by PD-L1 expression (TIC score <10% vs. TIC score ≥10%), previous curative treatment (yes vs. no), and ECOG Performance Status (0 vs. 1), is used to compare ORR between the two treatment arms. ORR is calculated for each treatment arm, and the difference in ORR between treatment arms is computed. The 95% CI for ORR for each arm is derived through use of the Clopper-Pearson method (Clopper and Pearson, Biometrika, 26:404-13 (1934)). The 95% CI for difference in ORR is computed by normal approximation.

Duration of Response

Duration of response (DOR) is assessed in patients who achieved an objective response, as determined by the investigator according to RECIST v1.1. DOR is defined as the time from the first occurrence of a confirmed objective response (CR or PR, whichever status is recorded first) to the first occurrence of disease progression or death from any cause, whichever occurs first. Patients who have not progressed and who have not died at the time of analysis are censored at the date of the last tumor assessment. If no tumor assessments are performed after the date of the first occurrence of a documented CR or PR, DOR is censored at the date of the first occurrence of a documented CR or PR. The analysis of DOR is based on a non-randomized subset of patients (specifically, patients who achieved an objective response); therefore, comparisons between treatment arms are made for descriptive purposes only.

The analysis methods are similar to those described for OS.

Time to Confirmed Deterioration

Time to confirmed deterioration (TTCD) in patient-reported physical functioning, role functioning, and GHS/QoL, as measured by the respective scales of the EORTC QLQ-C30, is defined as the time from randomization to first deterioration (decrease from baseline of 10 points) that is maintained for two consecutive assessments or followed by death from any cause within 3 weeks.

TTCD in patient-reported dysphagia, as measured by the dysphagia scale of the EORTC QLQ-OES18, is defined as the time from randomization to first deterioration (increase from baseline of 10 points) that is maintained for two consecutive assessments or followed by death from any cause within 3 weeks.

The analysis methods are similar to those described for OS.

Time to Progression

Time to progression is defined as the time from randomization to the first occurrence of disease progression, as determined by the investigator according to RECIST v1.1. Patients who have not experienced disease progression at the time of analysis are censored at the date of the last tumor assessment. Patients with no postbaseline tumor assessment are censored at the date of randomization. The analysis methods will be similar to those described for OS.

Patient-Reported Outcomes

Completion rates and reasons for missing data are summarized for the EORTC QLQ-C30 and EORTC QLQ-OES18 questionnaires at each cycle by treatment arm.

Visit mean summary and change from baseline analyses are performed for all scales of the EORTC QLQ-C30 and EORTC QLQ-OES18. Summary statistics (e.g., number of patients, mean, standard deviation, median, minimum, maximum, 95% CI) of linearly transformed scores (per the EORTC scoring manual) are calculated at all assessment timepoints for each study arm.

The proportion of patients with clinically meaningful changes (improved, deteriorated, remained stable) in physical functioning, role functioning, GHS/QoL, and dysphagia, as measured by the respective scales of the EORTC QLQ-C30 and the EORTC QLQ-OES18, are summarized by treatment arm. Previously published minimally important differences are used to identify clinically meaningful changes (e.g., Osoba et al., J Clin Oncol, 16:139-44 (1998); Cocks et al., J Clin Oncol, 29:89-96 (2011)).

Biomarkers

In the current study, archival or baseline tumor specimens are collected from patients and tested for PD-L1 expression by a central laboratory during the screening period. In addition to the assessment of PD-L1 status, other exploratory biomarkers, such as potential predictive and prognostic biomarkers related to the clinical benefit of tiragolumab and atezolizumab, tumor immunobiology, mechanisms of resistance, or tumor type, may be analyzed.

Blood samples are collected at baseline and during the study to evaluate changes in surrogate biomarkers. Changes in biomarkers associated with T cell activation and lymphocyte subpopulations may provide evidence of biologic activity of tiragolumab and atezolizumab in humans. Correlations between these biomarkers and safety and efficacy endpoints are explored to identify blood-based biomarkers that might predict which patients are more likely to benefit from tiragolumab and atezolizumab.

Exploratory biomarker research may include, but is not limited to, analysis of genes or gene signatures associated with tumor immunobiology, PD-L1, TIGIT, lymphocyte subpopulations, T-cell receptor repertoire, or genes associated with T-cell activation. Research may involve extraction of DNA to enable T cell-receptor sequencing (PBMCs only) or extraction of RNA.

Samples for the following laboratory tests will be sent to the study site's local laboratory for analysis:
Hematology: WBC count, RBC count, hemoglobin, hematocrit, platelet count, and differential count (neutrophils, eosinophils, basophils, monocytes, lymphocytes)
Chemistry panel (serum or plasma): bicarbonate or total carbon dioxide (if considered standard of care for the region), sodium, potassium, chloride, glucose, BUN or urea, creatinine, total protein, albumin, phosphate, calcium, total bilirubin, ALP, ALT, AST, and LDH
Coagulation: INR, and aPTT
Thyroid function testing: thyroid-stimulating hormone, free triiodothyronine (T3) (or total T3 for sites where free T3 is not performed), and free thyroxine (also known as T4)
Pregnancy test

All women of childbearing potential will have a serum pregnancy test at screening. Urine pregnancy tests will be performed at specified subsequent visits. If a urine pregnancy test is positive, it must be confirmed by a serum pregnancy test.

A woman is considered to be of childbearing potential if she is postmenarchal, has not reached a postmenopausal state 12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis).
Urinalysis (pH, specific gravity, glucose, protein, ketones, and blood); dipstick permitted

Samples for the following laboratory tests are sent to the study site's local laboratory or to a central laboratory for analysis:
HIV serology
EBV serology, as outlined below:
   EBV VCA IgM
   EBV VCA IgG or Epstein-Barr nuclear antigen IgG
   EBV PCR (only if clinically indicated)
HBV serology: hepatitis B surface antigen (HBsAg), hepatitis B surface antibody (HBsAb), and total hepatitis B core antibody (HBcAb) for all patients; HBV DNA for patients with a positive HBsAg and patients with negative HBsAg and HBsAb tests and a positive total HBcAb test
HCV serology: HCV antibody and (if HCV antibody test is positive) HCV RNA

If a patient has a positive HCV antibody test at screening, an HCV RNA test must also be performed to determine if the patient has an HCV infection.
C-reactive protein

The following samples are sent to one or several central laboratories or to the Sponsor or a designee for analysis:
Serum samples for tiragolumab and atezolizumab PK analysis through use of validated assays

All patients undergo sparse PK sample collection.
Serum samples for assessment of ADAs to tiragolumab and atezolizumab through use of validated assays
PBMC samples for exploratory research on biomarkers and biomarker assay development
Archival or newly collected tumor tissue sample obtained at baseline for determination of PD-L1 expression through use of the investigational Ventana PD-L1 (SP263) CDx Assay for patient stratification purposes and for exploratory research on biomarkers and biomarker assay development

A representative FFPE tumor specimen in a paraffin block (preferred) or at least 12 slides containing unstained, freshly cut, serial sections (5 slides for determination of PD-L1 expression and 7 slides for exploratory biomarker research and biomarker assay development) should be submitted along with an associated pathology report prior to randomization. If only 8-11 slides are available, the patient may still be eligible for the study, after Medical Monitor confirmation has been obtained.

Tumor tissue should be of good quality based on total and viable tumor content. Samples must contain a minimum of 50 viable tumor cells that preserve cellular context and tissue architecture regardless of needle gauge or retrieval method. Samples collected via resection, core-needle biopsy (at least three cores, embedded in a single paraffin block), or excisional, incisional, punch, or forceps biopsy are acceptable. Fine-needle aspiration (defined as samples that do not preserve tissue architecture and yield cell suspension and/or smears), brushing, cell pellets from pleural effusion, and lavage samples are not acceptable. Tumor tissue from bone metastases that have been decalcified is not acceptable.

If archival tumor tissue is unavailable or is determined to be unsuitable for required testing, a pretreatment tumor biopsy is required. A pretreatment tumor biopsy may also be performed if a patient's archival tissue test results do not meet eligibility criteria.

Patient Eligibility

Inclusion Criteria Patients must meet the following criteria for study entry:
Signed Informed Consent Form
Age ≥18 years at time of signing Informed Consent Form
Ability to comply with the study protocol
Histologically confirmed ESCC
   Patients with tumors of mixed histology (i.e., squamous and non-squamous) are eligible if the major histological component appears to be squamous, except if small-cell elements are present.
Unresectable locally advanced, unresectable recurrent, or metastatic disease (i.e., advanced disease, not suitable for definitive treatment such as radiotherapy, chemoradiotherapy, and/or surgery) that meets the following criteria:
   No prior systemic treatment for advanced disease
   For patients receiving prior chemoradiotherapy or chemotherapy for non-advanced ESCC: treatment must have been given with curative intent or in the adjuvant or neoadjuvant setting, with an interval of at least 6 months between the final treatment and the diagnosis of advanced disease
Measurable disease per RECIST v1.1
   Previously irradiated lesions can be considered as measurable disease only if progressive disease has been unequivocally documented at that site since radiation.
Availability of a representative tumor specimen that is suitable for determination of PD-L1 expression, as assessed by a central laboratory through use of the investigational Ventana PD-L1 (SP263) CDx Assay, and for exploratory biomarker research and biomarker assay development
   A formalin-fixed paraffin-embedded (FFPE) tumor specimen in a paraffin block (preferred) or at least 12 slides containing unstained, freshly cut, serial sections (5 slides for determination of PD-L1 expression and 7 slides for exploratory biomarker research and biomarker assay development) should be submitted along with an associated pathology report prior to randomization. If only 8-10 slides are available, the patient may still be eligible for the study, after Medical Monitor confirmation has been obtained. If archival tumor tissue is unavailable or is determined to be unsuitable for required testing, tumor tissue must be obtained from a biopsy performed at screening.
ECOG Performance Status of 0 or 1
Body mass index ≥13 kg/m²
Life expectancy ≥3 months
Adequate hematologic and end-organ function, defined by the following laboratory test results, obtained within 14 days prior to initiation of study treatment:
   ANC ≥1.5×10⁹/L (1500/4) without granulocyte colony-stimulating factor support
   Lymphocyte count ≥0.5×10⁹/L (500/4)
   Platelet count ≥100×10⁹/L (100,000/μL) without transfusion
   Hemoglobin ≥90 g/L (9 g/dL)
      Patients may be transfused to meet this criterion.
   AST, ALT, and ALP 2.5×upper limit of normal (ULN), with the following exceptions:
      Patients with documented liver metastases: AST and ALT≤5×ULN
      Patients with documented liver or bone metastases: ALP≤5×ULN
   Total bilirubin ≤1.5×ULN with the following exception:
      Patients with known Gilbert disease: total bilirubin 3×ULN
   Creatinine ≤1.5×ULN and creatinine clearance ≥60 mL/min (calculated through use of the Cockcroft-Gault formula)
   Albumin ≥25 g/L (2.5 g/dL) without transfusion
   For patients not receiving therapeutic anticoagulation: INR and aPTT≤1.5×ULN
For patients receiving therapeutic anticoagulation: stable anticoagulant regimen
Negative HIV test at screening
Negative for Epstein-Barr virus (EBV) at screening
   Patients must have a negative EBV viral capsid antigen (VCA) IgM test at screening to be eligible for the study. If clinically indicated, patients will undergo an EBV polymerase chain reaction (PCR) test at screening and must have a negative PCR test to be eligible for the study.
Patients with hepatitis B virus (HBV): HBV DNA <500 IU/mL (or 2500 copies/mL) at screening Patients with detectable hepatitis B surface antigen or detectable HBV DNA should be managed per institutional guidelines. Patients receiving anti-viral medication must have initiated treatment at least 2 weeks prior to randomization and should continue treatment for at least 6 months after the final dose of study treatment.
Negative hepatitis C virus (HCV) antibody test at screening, or positive HCV antibody test followed by a negative HCV RNA test at screening
   The HCV RNA test will be performed only for patients who have a positive HCV antibody test.
For women of childbearing potential: agreement to remain abstinent (refrain from heterosexual intercourse) or use contraception, and agreement to refrain from donating eggs, as defined below:
   Women must remain abstinent or use contraceptive methods with a failure rate of <1% per year during the treatment period and for 90 days after the final dose of tiragolumab, for 5 months after the final dose of atezolizumab, and for 6 months after the final dose of paclitaxel or cisplatin. Women must refrain from donating eggs during this same period.
   A woman is considered to be of childbearing potential if she is postmenarchal, has not reached a postmenopausal state 12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For men: agreement to remain abstinent (refrain from heterosexual intercourse) or use contraceptive methods, and agreement to refrain from donating sperm, as defined below:
   With a female partner of childbearing potential who is not pregnant, men must remain abstinent or use a condom plus an additional contraceptive method that together result in a failure rate of <1% per year during the treatment period and for 6 months after the final dose of paclitaxel or cisplatin. In the event of chemotherapy discontinuation, men who continue to receive tiragolumab for more than 6 months after the final chemotherapy dose must remain abstinent or use a condom until 90 days after the final dose of tiragolumab. Men must refrain from donating sperm during this same period.
   With a pregnant female partner, men must remain abstinent or use a condom during the treatment period and for 90 days after the final dose of tiragolumab and 6 months after the final dose of paclitaxel or cisplatin to avoid exposing the embryo.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.

Exclusion Criteria

Patients who meet any of the following criteria are excluded from study entry:
Palliative radiation treatment for ESCC within 4 weeks prior to initiation of study treatment
Evidence of fistula (either esophageal/bronchial or esophageal/aorta)
Evidence of complete esophageal obstruction not amenable to treatment
Symptomatic, untreated, or actively progressing CNS metastases
   Asymptomatic patients with treated CNS lesions are eligible, provided that all of the following criteria are met:
      Measurable disease, per RECIST v1.1, must be present outside the CNS.
      The patient has no history of intracranial hemorrhage or spinal cord hemorrhage.
      The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
      The patient has no ongoing requirement for corticosteroids as therapy for CNS disease. Anti-convulsant therapy at a stable dose is permitted.
      Metastases are limited to the cerebellum or the supratentorial region (i.e., no metastases to the midbrain, pons, medulla, or spinal cord).
      There is no evidence of interim progression between completion of CNS-directed therapy and initiation of study treatment.
   Asymptomatic patients with CNS metastases newly detected at screening are eligible for the study after receiving radiotherapy or surgery, with no need to repeat the screening brain scan.
Uncontrolled tumor-related pain
   Patients requiring pain medication must be on a stable regimen at study entry.
   Symptomatic lesions (e.g., bone metastases or metastases causing nerve impingement) amenable to palliative radiotherapy should be treated prior to randomization. Patients should be recovered from the effects of radiation. There is no required minimum recovery period. Asymptomatic metastatic lesions that would likely cause functional deficits or intractable pain with further growth (e.g., epidural metastasis that is not currently associated with spinal cord compression) should be considered for loco-regional therapy if appropriate prior to randomization.
Uncontrolled pleural effusion, pericardial effusion, or ascites requiring recurrent drainage procedures (once monthly or more frequently)
   Patients with indwelling catheters (e.g., PLEURX®) are allowed.
Uncontrolled or symptomatic hypercalcemia (ionized calcium >1.5 mmol/L, calcium >12 mg/dL, or corrected calcium >ULN)
History of leptomeningeal disease
Active or history of autoimmune disease or immune deficiency, including, but not limited to, myasthenia gravis, myositis, autoimmune hepatitis, systemic lupus erythematosus, rheumatoid arthritis, inflammatory bowel disease, anti-phospholipid antibody syndrome, Wegener granulomatosis, Sjögren syndrome, Guillain-Barré syndrome, or multiple sclerosis, with the following exceptions:
   Patients with a history of autoimmune-related hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study provided all of following conditions are met:
      Rash must cover <10% of body surface area
      Disease is well controlled at baseline and requires only low-potency topical corticosteroids
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months
History of idiopathic pulmonary fibrosis, organizing pneumonia (e.g., bronchiolitis obliterans), drug-induced pneumonitis, or idiopathic pneumonitis, or evidence of active pneumonitis on screening chest computed tomography (CT) scan
   History of radiation pneumonitis in the radiation field (fibrosis) is permitted.
Severe chronic or active infection within 4 weeks prior to initiation of study treatment, including, but not limited to, hospitalization for complications of infection, bacteremia, or severe pneumonia
Treatment with therapeutic oral or IV antibiotics within 2 weeks prior to initiation of study treatment
   Patients receiving prophylactic antibiotics (e.g., to prevent a urinary tract infection or chronic obstructive pulmonary disease exacerbation) are eligible for the study.
Active tuberculosis
Major surgical procedure, other than for diagnosis, within 4 weeks prior to initiation of study treatment, or anticipation of need for a major surgical procedure during the study
Any of the following cardiovascular risk factors:
   Cardiac chest pain, defined as moderate pain that limits instrumental activities of daily living, within 28 days prior to initiation of study treatment
   Symptomatic pulmonary embolism within 28 days prior to initiation of study treatment
   Acute myocardial infarction within 6 months prior to initiation of study treatment
   Heart failure meeting New York Heart Association Classification III or IV within 6 months prior to initiation of study treatment
   Grade 2 ventricular arrhythmia within 6 months prior to initiation of study treatment
   Cerebrovascular accident within 6 months prior to initiation of study treatment
   Uncontrolled hypertension, defined as systolic pressure 160 mmHg or diastolic pressure 100 mmHg despite anti-hypertensive medications, within 28 days prior to initiation of study treatment
   Episode of syncope or seizure within 28 days prior to initiation of study treatment
History of severe allergic anaphylactic reactions to chimeric or humanized antibodies or fusion proteins
History of malignancy within 2 years prior to screening, with the exception of the cancer under investigation in this study and malignancies with a negligible risk of metastasis or death (e.g., 5-year overall survival (OS) rate >90%), such as adequately treated carcinoma in situ of the cervix, non-melanoma skin carcinoma, localized prostate cancer, ductal carcinoma in situ, or
Stage I uterine cancer
Grade ≥2 peripheral neuropathy at screening
Uncontrolled diabetes or Grade 2 abnormalities in potassium, sodium, or corrected calcium despite standard medical management within 14 days prior to initiation of study treatment
Any other disease, medical condition, metabolic dysfunction, alcohol or drug abuse or dependence, physical examination finding, clinical laboratory finding that contraindicates the use of an investigational drug, may affect the interpretation of the results, or may render the patient at high risk from treatment complications
Poor peripheral venous access
Prior treatment with CD137 agonists, T-cell co-stimulating, or immune checkpoint blockade therapies, including anti-CTLA-4, anti-PD-1, anti-PD-L1, and anti-TIGIT therapeutic antibodies
Treatment with systemic immunostimulatory agents (including, but not limited to, interferon and interleukin 2) within 4 weeks or 5 drug-elimination half-lives (whichever is longer) prior to initiation of study treatment
Treatment with chemotherapy, immunotherapy (e.g., interleukin, interferon, thymosin), or any investigational therapy within 14 days or 5 drug-elimination half-lives (whichever is longer) prior to initiation of study treatment
Treatment with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) may be eligible for the study, after discussion with the Medical Monitor
   Patients who received mineralocorticoids (e.g., fludrocortisone), corticosteroids for chronic obstructive pulmonary disease or asthma, or low-dose corticosteroids for orthostatic hypotension or adrenal insufficiency are eligible for the study
Prior allogeneic stem cell or solid organ transplantation
Treatment with a live, attenuated vaccine within 4 weeks prior to initiation of study treatment, or anticipation of need for such a vaccine during study treatment or within 5 months after the final dose of study treatment
Concurrent participation in another therapeutic clinical trial
Known hypersensitivity to Chinese hamster ovary cell products or to any component of the atezolizumab formulation
Known allergy or hypersensitivity to any component of the tiragolumab formulation
Pregnant or breastfeeding
   Women of childbearing potential must have a negative serum pregnancy test result within 14 days prior to initiation of study treatment.

### Example 3. Results of a Phase Ib Study of the Anti-TIGIT Antibody Tiragolumab in Combination with Atezolizumab in Patients with Metastatic Esophageal Cancer

A Phase Ib dose-escalation and dose-expansion study (GO30103, NCT02794571) showed that tiragolumab (tira) combined with atezolizumab (atezo) was safe and tolerable, and activity was seen in an expansion cohort of patients with PD-L1-positive non-small cell lung cancer (Bendell et al., Phase Ia/Ib dose-escalation study of the anti-TIGIT antibody tiragolumab as a single agent and in combination with atezolizumab in patients with advanced solid tumors. In: Proceedings of the 111th Annual Meeting of the American Association for Cancer Research; 2020 Jun. 22-24. Philadelphia (Pa.): AACR; 2020. Abstract CT302).

Preliminary safety and antitumor activity of tira+atezo in patients with metastatic PD-L1-positive esophageal cancer not previously treated with cancer immunotherapy were assessed.

Methods

Patients enrolled in this Phase Ib expansion cohort had metastatic esophageal cancers of squamous or adenocarcinoma histology, who had progressed on available therapies. Patients had ECOG PS 0-1, had not been treated previously with cancer immunotherapy, and were enrolled from the US, EU, and Asia. PD-L1 expression of the esophageal tumors was determined by central immunohistochemistry review. Patients received tira 400 mg or 600 mg IV every 3 weeks (Q3W)+atezo 1200 mg IV Q3W until disease progression, intolerable toxicity, or patient/investigator decision. The safety, tolerability, and preliminary antitumor activity of tira+atezo were evaluated at a data cut-off date of 2 Dec. 2019.

Results

19 patients with metastatic esophageal cancers of squamous or adenocarcinoma histologies were treated: median age was 62 years, ECOG 0 in 5 patients (26%) and ECOG 1 in 14 patients (74%), 14 patients (74%) had received prior therapies, 6 patients (32%) were from Asia and 13 (68%) were from US/EU. Treatment-related AEs (TRAEs), as assessed by investigators, occurred in 63% (12 patients), with Grade A in 5% (1 patient); there were no Grade 4 or 5 TRAEs. The most common AEs reported in 5% of patients were anemia (3 patients, 16%), cough (16%), dysphagia (16%), and pyrexia (16%). Of 16 evaluable patients with at least one tumor assessment, there were 4 confirmed partial responses (objective response rate of 25%), and a disease control rate of 50%, with 2 patients still on study at 1+ years.

### Conclusions

Tira combined with atezo was well-tolerated and had an acceptable safety profile in patients with metastatic esophageal cancers. Preliminary antitumor activity was observed in a cohort of patients with metastatic esophageal cancers not previously treated with immunotherapy.

### Example 4. PD-L1 as a Predictive Biomarker for Tiragolumab+Atezolizumab Treatment

In the Phase Ib study (G030103; NCT02794571), tiragolumab was well-tolerated as monotherapy and in combination with atezolizumab in multiple solid tumor types. In the randomized Phase II CITYSCAPE study in 1L NSCLC (NCT03563716), clinically meaningful improvements were seen in ORR and PFS with tiragolumab+atezolizumab vs placebo+atezolizumab in the intention-to-treat (ITT) population. A greater magnitude of improvement was seen in the PD-L1 tumor proportion score (TPS) ≥50% subgroup (as assessed using the PharmDx 22C3 IHC assay; data cut-off December 2019; median follow-up 10.9 months).

The value of PD-L1 as a predictive biomarker for tiragolumab+atezolizumab treatment was found to be consistent across different PD-L1 assays. IHC was performed to evaluate PD-L1 protein expression for all available patient samples using the pharmDx 22C3 assay (ITT population) and the Conformité Européenne (European Conformity) in vitro diagnostic (CE-IVDO VENTANA SP263 IHC assay (biomarker evaluable population); the levels of PD-L1 expression were scored using the established algorithms for each assay. Baseline characteristics of the BEP and ITT populations were similar (Table 89).

TABLE 13
Administration of first and subsequent tiragolumab infusions

| | ITT | BEP (SP263)* |
|---|---|---|
| n, (%) | (N = 135) | (n = 113) |
| Age <65 years | 56 (41%) | 49 (43%) |
| Male | 87 (64%) | 75 (66%) |
| White | 82 (61%) | 70 (62%) |
| Asian | 41 (30%) | 32 (28%) |
| ECOG PS 0 | 39 (29%) | 29 (26%) |
| Never used tobacco | 14 (10%) | 13 (12%) |
| Non-squamous histology | 80 (59%) | 65 (58%) |
| PD-L1 TPS ≥50% | 58 (43%) | 50 (44%) |
| PD-L1 TPS 1-49% | 77 (57%) | 63 (56%) |
| PD-L1 SP263 TC ≥50% | 45 (33%) | 45 (40%) |
| PD-L1 SP263 TC <50% | 68 (50%) | 68 (60%) |
| PD-L1 5P263 TC missing | 22 (16%) | — |
| TIGIT IHC ≥5% | 49 (36%) | 43 (38%) |
| TIGIT IHC <5% | 56 (41%) | 54 (48%) |
| TIGIT IHC missing | 30 (22%) | 16 (14%) |

*Not all patients had available tissue sample for testing with the SP263 IHC assay.

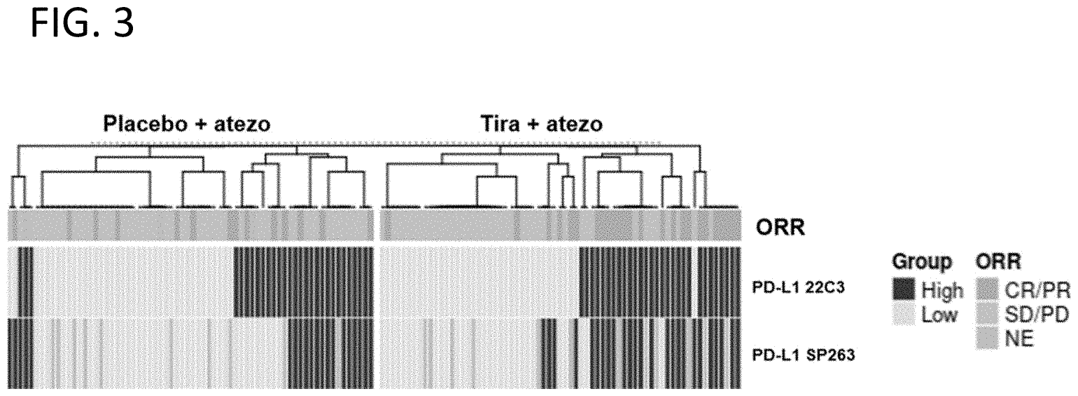

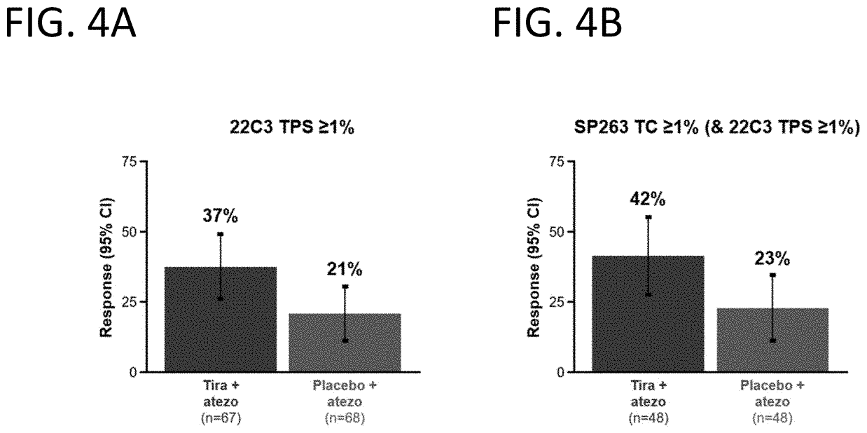

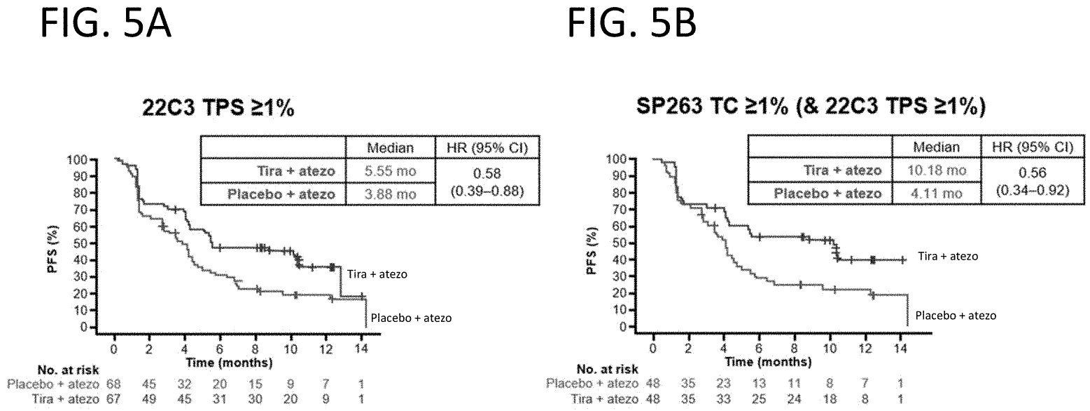

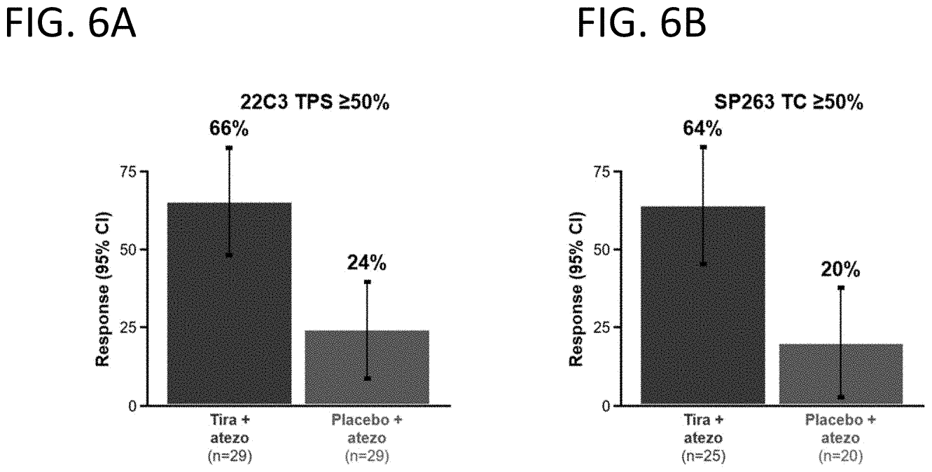

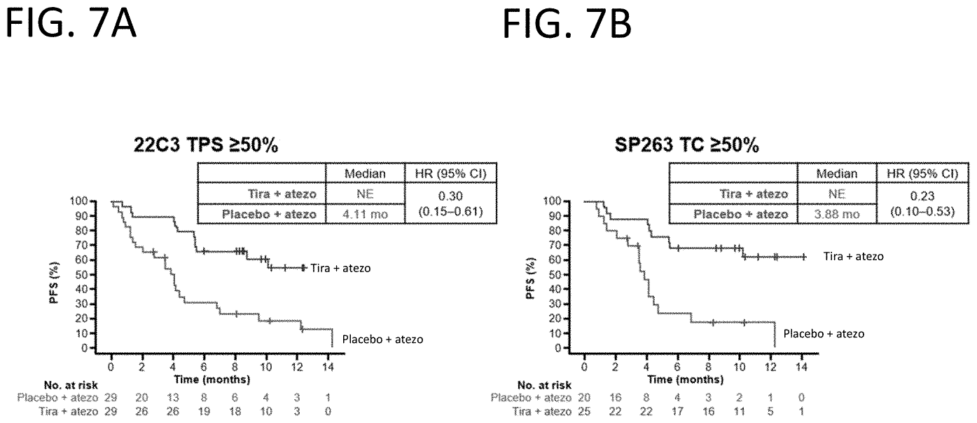

Prevalence of PD-L1 subgroups was comparable between the two IHC assays (FIG. 3). For the PD-L1 22C3 assay, high TPS was classified as TPS≥50%; low TPS was classified as TPS 1-49%. For the SP263 IHC assay, high TC was classified as TC≥50%; low TC was classified as TC 1-49%. Comparable ORR and PFS improvements with tiragolumab+atezolizumab vs. atezolizumab monotherapy were seen between the PD-L1-positive (TC≥1%) subgroup defined by SP263 (PFS HR 0.56, 95% CI: 0.34-0.92) and the PD-L1-positive (TPS≥1%) subgroup defined by 22C3 (FIGS. 4A, 4B, 5A, and 5B). Comparable ORR and PFS improvements with tiragolumab+atezolizumab vs. atezolizumab monotherapy were also seen between the PD-L1-high (TC≥50%) subgroup defined by SP263 (PFS HR 0.23, 95% CI: 0.10-0.53) and the PD-L1-high (TPS≥50%) subgroup defined by 22C3 (FIGS. 6A, 6B, 7A, and 7B).

High PD-L1 expression, assessed either by 22C3 or SP263 IHC, may be an important predictive biomarker for tiragolumab+atezolizumab combination therapy.

### VI. OTHER EMBODIMENTS

Some embodiments of the technology described herein can be defined according to any of the following numbered embodiments:

1. A method for treating a subject or population of subjects having an esophageal squamous cell carcinoma (ESCC), the method comprising administering to the subject or population of subjects one or more dosing cycles of an anti-TIGIT antagonist antibody and a PD-1 axis binding antagonist, wherein the subject or population of subjects previously received definitive chemoradiation treatment (e.g., definitive concurrent chemoradiation treatment) for ESCC.

2. The method of embodiment 1, wherein the definitive chemoradiation treatment was completed no more than 89 days prior to administration with the anti-TIGIT antagonist antibody or the PD-1 axis binding antagonist.

3. The method of embodiment 1 or 2, wherein the definitive chemoradiation treatment comprises at least two cycles of platinum-based chemotherapy and radiation therapy without evidence of radiographic disease progression.

4. The method of any one of embodiments 1-3, wherein no chemotherapy is administered to the subject or population of subjects during the one or more dosing cycles.

5. The method of any one of embodiments 1-4, wherein the anti-TIGIT antagonist antibody is administered at a fixed dose of about 30 mg to about 1200 mg every three weeks.

6. The method of any one of embodiments 1-5, wherein the anti-TIGIT antagonist antibody is administered at a fixed dose of about 30 mg to about 800 mg every three weeks.

7. The method of embodiment 6, wherein the anti-TIGIT antagonist antibody is administered at a fixed dose of about 600 mg every three weeks.

8. The method of any one of embodiments 1-7, wherein the PD-1 axis binding antagonist is administered at a fixed dose of about 80 mg to about 1600 mg every three weeks.

9. The method of any one of embodiments 1-8, wherein the PD-1 axis binding antagonist is administered at a fixed dose of about 800 mg to about 1400 mg every three weeks.

10. The method of embodiment 9, wherein the PD-1 axis binding antagonist is administered at a fixed dose of about 1200 mg every three weeks.

11. The method of embodiment 10, wherein the anti-TIGIT antagonist antibody is administered at a fixed dose of about 600 mg every three weeks and the PD-1 axis binding antagonist is administered at a fixed dose of about 1200 mg every three weeks.

12. The method of any one of embodiments 1-11, wherein the length of each of the one or more dosing cycles is 21 days.

13. The method of any one of embodiments 1-4, wherein the anti-TIGIT antagonist antibody is administered at a fixed dose of about 300 mg to about 800 mg every two weeks.

14. The method of embodiment 13, wherein the anti-TIGIT antagonist antibody is administered at a fixed dose of about 400 mg to about 500 mg every two weeks.

15. The method of embodiment 14, wherein the anti-TIGIT antagonist antibody is administered at a fixed dose of about 420 mg every two weeks.

16. The method of any one of embodiments 1˜4 and 13-15, wherein the PD-1 axis binding antagonist is administered at a fixed dose of about 200 mg to about 1200 mg every two weeks.

17. The method of embodiment 16, wherein the PD-1 axis binding antagonist is administered at a fixed dose of about 800 mg to about 1000 mg every two weeks.

18. The method of embodiment 17, wherein the PD-1 axis binding antagonist is administered at a fixed dose of about 840 mg every two weeks.

19. The method of embodiment 18, wherein the anti-TIGIT antagonist antibody is administered at a fixed dose of about 420 mg every two weeks and the PD-1 axis binding antagonist is administered at a fixed dose of about 840 mg every two weeks.

20. The method of any one of embodiments 1-4, wherein the anti-TIGIT antagonist antibody is administered at a fixed dose of about 700 mg to about 1000 mg every four weeks.

21. The method of embodiment 20, wherein the anti-TIGIT antagonist antibody is administered at a fixed dose of about 800 mg to about 900 mg every four weeks.

22. The method of embodiment 21, wherein the anti-TIGIT antagonist antibody is administered at a fixed dose of about 840 mg every four weeks.

23. The method of any one of embodiments 1˜4 and 20-22, wherein the PD-1 axis binding antagonist is administered at a fixed dose of about 400 mg to about 2000 mg every four weeks.

24. The method of embodiment 23, wherein the PD-1 axis binding antagonist is administered at a fixed dose of about 1600 mg to about 1800 mg every four weeks.

25. The method of embodiment 24, wherein the PD-1 axis binding antagonist is administered at a fixed dose of about 1680 mg every four weeks.

26. The method of embodiment 25, wherein the anti-TIGIT antagonist antibody is administered at a fixed dose of about 840 mg every four weeks and the PD-1 axis binding antagonist is administered at a fixed dose of about 1680 mg every four weeks.

27. The method of any one of embodiments 1-26, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

28. The method of embodiment 27, wherein the anti-TIGIT antagonist antibody further comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

29. The method of embodiment 27 or 28, wherein the anti-TIGIT antagonist antibody further comprises the following heavy chain variable region FRs:

an FR-H1 comprising the amino acid sequence of XiVQLQQSGPGLVKPSQTLSLTCAISGDSVS (SEQ ID NO: 11), wherein Xi is E or Q;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

30. The method of embodiment 29, wherein Xi is E.

31. The method of embodiment 29, wherein Xi is Q.

32. The method of any one of embodiments 27-31, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

33. The method of any one of embodiments 1-32, wherein the anti-TIGIT antagonist antibody comprises:

(a) a VH domain comprising the amino acid sequence of SEQ ID NO: 17 or 18; and

(b) a VL domain comprising the amino acid sequence of SEQ ID NO: 19.

34. The method of any one of embodiments 1-33, wherein the anti-TIGIT antagonist antibody is a monoclonal antibody.

35. The method of embodiment 34, wherein the anti-TIGIT antagonist antibody is a human antibody.

36. The method of any one of embodiments 1-35, wherein the anti-TIGIT antagonist antibody is a full-length antibody.

37. The method of any one of embodiments 1-26 and 34-36, wherein the anti-TIGIT antagonist antibody has intact Fc-mediated effector function.

38. The method of embodiment 37, wherein the anti-TIGIT antagonist antibody is tiragolumab, vibostolimab, etigilimab, EOS084448, SGN-TGT, or TJ-T6.

39. The method of any one of embodiments 1-30 and 32-38, wherein the anti-TIGIT antagonist antibody is tiragolumab.

40. The method of any one of embodiments 1-26 and 34-36, wherein the anti-TIGIT antagonist antibody has enhanced Fc-mediated effector function.

41. The method of any one of embodiments 1-26, 34-38, and 40, wherein the anti-TIGIT antagonist antibody is SGN-TGT.

42. The method of any one of embodiments 1-26 and 34-36, wherein the anti-TIGIT antagonist antibody does not have Fc-mediated effector function.

43. The method of any one of embodiments 1-26, 34-36, and 42, wherein the anti-TIGIT antagonist antibody is domvanalimab, BMS-986207, ASP8374, or COM902.

44. The method of any one of embodiments 1-35, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

45. The method of any one of embodiments 1-26 and 34-43, wherein the anti-TIGIT antagonist antibody is an IgG class antibody.

46. The method of embodiment 45, wherein the IgG class antibody is an IgG1 subclass antibody.

47. The method of embodiment 46, wherein the anti-TIGIT antagonist antibody is tiragolumab, vibostolimab, etigilimab, EOS084448, SGN-TGT, TJ-T6, BGB-A1217, AB308, domvanalimab, or BMS-986207.

48. The method of embodiment 47, wherein the anti-TIGIT antagonist antibody is tiragolumab.

49. The method of embodiment 45, wherein the IgG class antibody is an IgG4 subclass antibody.

50. The method of embodiment 49, wherein the anti-TIGIT antagonist antibody is ASP8374 or COM902.

51. The method of any one of embodiments 1-50, wherein the PD-1 axis binding antagonist is a PD-L1 binding antagonist or a PD-1 binding antagonist.

52. The method of embodiment 51, wherein the PD-L1 binding antagonist is an anti-PD-L1 antagonist antibody.

53. The method of any one of embodiments 1-52, wherein the anti-PD-L1 antagonist antibody is atezolizumab (MPDL3280A), MSB0010718C, MDX-1105, or MED14736.

54. The method of embodiment 53, wherein the anti-PD-L1 antagonist antibody is atezolizumab.

55. The method of embodiment 51, wherein the PD-1 binding antagonist is an anti-PD-1 antagonist antibody.

56. The method of embodiment 55, wherein the anti-PD-1 antagonist antibody is nivolumab (MDX-1106), pembrolizumab (MK-3475), or AMP-224.

57. The method of any one of embodiments 1-52, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

58. The method of embodiment 57, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

59. The method of any one of embodiments 1-58, wherein the anti-PD-L1 antagonist antibody comprises:

a VH domain comprising the amino acid sequence of SEQ ID NO: 26; and

a VL domain comprising the amino acid sequence of SEQ ID NO: 27.

60. The method of any one of embodiments 57-59, wherein the anti-PD-L1 antagonist antibody is a monoclonal antibody.

61. The method of embodiment 60, wherein the anti-PD-L1 antagonist antibody is a humanized antibody.

62. The method of embodiment 60 or 61, wherein the anti-PD-L1 antagonist antibody is a full-length antibody.

63. The method of any one of embodiments 57-61, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

64. The method of any one of embodiments 57-63, wherein the anti-PD-L1 antagonist antibody is an IgG class antibody.

65. The method of embodiment 64, wherein the IgG class antibody is an IgG1 subclass antibody.

66. The method of any one of embodiments 1-65, wherein the method comprises administering to the subject or population of subjects the anti-TIGIT antagonist antibody and the PD-1 axis binding antagonist on about Day 1 of each of the one or more dosing cycles.

67. The method of any one of embodiments 1-66, wherein the method comprises administering to the subject or population of subjects the PD-1 axis binding antagonist before the anti-TIGIT antagonist antibody.

68. The method of embodiment 67, wherein the method comprises a first observation period following administration of the PD-1 axis binding antagonist and a second observation period following administration of the anti-TIGIT antagonist antibody.

69. The method of embodiment 68, wherein the first observation period and the second observation period are each between about 30 minutes to about 60 minutes in length.

70. The method of any one of embodiments 1-66, wherein the method comprises administering to the subject or population of subjects the anti-TIGIT antagonist antibody before the PD-1 axis binding antagonist.

71. The method of embodiment 70, wherein the method comprises a first observation period following administration of the anti-TIGIT antagonist antibody and a second observation period following administration of the PD-1 axis binding antagonist.

72. The method of embodiment 71, wherein the first observation period and the second observation period are each between about 30 minutes to about 60 minutes in length.

73. The method of any one of embodiments 1-66, wherein the method comprises administering to the subject or population of subjects the anti-TIGIT antagonist antibody and the PD-1 axis binding antagonist simultaneously.

74. The method of any one of embodiments 1-73, wherein the method comprises administering to the subject or population of subjects the anti-TIGIT antagonist antibody and the PD-1 axis binding antagonist intravenously.

75. The method of embodiment 74, wherein the method comprises administering to the subject or population of subjects the anti-TIGIT antagonist antibody by intravenous infusion over 60±10 minutes.

76. The method of embodiment 74 or 75, wherein the method comprises administering to the subject or population of subjects the PD-1 axis binding antagonist by intravenous infusion over 60±15 minutes.

77. The method of any one of embodiments 1-76, wherein an ESCC tumor sample obtained from the subject or population of subjects has been determined to have a detectable expression level of PD-L1.

78. The method of embodiment 77, wherein the detectable expression level of PD-L1 is a detectable protein expression level of PD-L1.

79. The method of embodiment 78, wherein the detectable protein expression level of PD-L1 has been determined by an immunohistochemical (IHC) assay.

80. The method of embodiment 79, wherein the IHC assay uses anti-PD-L1 antibody SP263, 22C3, SP142, or 28-8.

81. The method of embodiment 80, wherein the IHC assay uses anti-PD-L1 antibody SP263.

82. The method of embodiment 81, wherein the IHC assay is the Ventana SP263 IHC assay.

83. The method of embodiment 82, wherein the ESCC tumor sample has been determined to have a tumor and tumor-associated immune cell (TIC) score of greater than, or equal to, 1%.

84. The method of embodiment 83, wherein the TIC score is greater than, or equal to, 10%.

85. The method of embodiment 82 or 83, wherein the ESCC tumor sample has been determined to have a TIC score of less than 10%.

86. The method of embodiment 84, wherein the TIC score is greater than, or equal to, 10% and less than 50%.

87. The method of embodiment 80, wherein the IHC assay uses the anti-PD-L1 antibody 22C3.

88. The method of embodiment 87, wherein the IHC assay is the pharmDx 22C3 IHC assay.

89. The method of embodiment 88, wherein the ESCC tumor sample has been determined to have a combined positive score (CPS) of greater than, or equal to, 10 or a tumor proportion score (TPS) of greater than, or equal to, 1%.

90. The method of embodiment 80, wherein the IHC assay uses the anti-PD-L1 antibody SP142.

91. The method of embodiment 90, wherein the IHC assay is the Ventana SP142 IHC assay.

92. The method of embodiment 80, wherein the IHC assay uses the anti-PD-L1 antibody 28-8.

93. The method of embodiment 92, wherein the IHC assay is the pharmDx 28-8 IHC assay.

94. The method of embodiment 77, wherein the detectable expression level of PD-L1 is a detectable nucleic acid expression level of PD-L1.

95. The method of embodiment 94, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

96. The method of any one of embodiments 1-95, wherein the ESCC is a locally advanced ESCC.

97. The method of any one of embodiments 1-96, wherein the ESCC is an unresectable ESCC.

98. The method of any one of embodiments 1-97, wherein the ESCC is a recurrent or metastatic ESCC.

99. The method of any one of embodiments 1-98, wherein the ESCC comprises a cervical esophageal tumor.

100. The method of any one of embodiments 1-99, wherein the ESCC is a Stage II ESCC, a Stage III ESCC, or a Stage IV ESCC, optionally wherein the Stage IV ESCC is a Stage IVA ESCC or a Stage IVB ESCC with supraclavicular lymph node metastases only.

101. The method of any one of embodiments 1-100, wherein the subject or population of subjects has not been treated previously with cancer immunotherapy.

102. The method of any one of embodiments 1-100, wherein the subject or population of subjects has completed a previous cancer immunotherapy for ESCC.

103. The method of any one of embodiments 1-102, wherein the treatment results in an increase in progression-free survival (PFS) of the subject or population of subjects as compared to treatment with the PD-1 axis binding antagonist without the anti-TIGIT antagonist antibody.

104. The method of any one of embodiments 1-103, wherein the treatment results in an increase in PFS of the subject or population of subjects as compared to treatment with the anti-TIGIT antagonist antibody without the PD-1 axis binding antagonist.

105. The method of any one of embodiments 1-104, wherein the treatment results in an increase in PFS of the subject or population of subjects as compared to treatment without the anti-TIGIT antagonist antibody and without the PD-1 axis binding antagonist.

106. The method of embodiment 105, wherein the treatment extends the PFS of the subject or population of subjects by at least about 4 months or about 8 months.

107. The method of embodiment 105, wherein the treatment results in a median PFS of the population of subjects of about 15 months to about 23 months.

108. The method of any one of embodiments 1-107, wherein the treatment results in an increase in overall survival (OS) of the subject or population of subjects as compared to treatment with the PD-1 axis binding antagonist without the anti-TIGIT antagonist antibody.

109. The method of any one of embodiments 1-107, wherein the treatment results in an increase in OS of the subject or population of subjects as compared to treatment with the anti-TIGIT antagonist antibody and without treatment with the PD-1 axis binding antagonist.

110. The method of any one of embodiments 1-107, wherein the treatment results in an increase in OS of the subject or population of subjects as compared to treatment without the anti-TIGIT antagonist antibody and without the PD-1 axis binding antagonist.

111. The method of embodiment 110, wherein the treatment extends the OS of the subject or population of subjects by at least about 7 months or about 12 months.

112. The method of embodiment 111, wherein the treatment results in a median OS of the population of subjects of about 24 months to about 36 months.

113. The method of any one of embodiments 1-112, wherein the treatment results in an increase in duration of objective response (DOR) in the subject or population of subjects as compared to treatment with the PD-1 axis binding antagonist without the anti-TIGIT antagonist antibody.

114. The method of any one of embodiments 1-112, wherein the treatment results in an increase in DOR in the subject or population of subjects as compared to treatment with the anti-TIGIT antagonist antibody without the PD-1 axis binding antagonist.

115. The method of any one of embodiments 1-112, wherein the treatment results in an increase in DOR in the subject or population of subjects as compared to treatment without the anti-TIGIT antagonist antibody and without the PD-1 axis binding antagonist.

116. The method of any one of embodiments 1-115, wherein the treatment results in a complete response or a partial response.

117. The method of any one of embodiments 1-116, wherein the method comprises administering to the subject or population of subjects at least five dosing cycles.

118. The method of embodiment 117, wherein the method comprises administering to the subject or population of subjects 17 dosing cycles.

119. A method for treating a subject having an ESCC, the method comprising administering to the subject one or more dosing cycles of tiragolumab at a fixed dose of about 30 mg to about 1200 mg every three weeks and atezolizumab at a fixed dose of about 80 mg to about 1600 mg every three weeks, wherein the subject previously received definitive chemoradiation treatment (e.g., definitive concurrent chemoradiation treatment) for ESCC.

120. The method of embodiment 119, wherein the tiragolumab is administered at a fixed dose of about 600 mg every three weeks and the atezolizumab is administered at a fixed dose of about 1200 mg every three weeks.

121. A method for treating a subject having an ESCC, the method comprising administering to the subject one or more dosing cycles of tiragolumab at a fixed dose of about 300 mg to about 800 mg every two weeks and atezolizumab at a fixed dose of about 200 mg to about 1200 mg every two weeks, wherein the subject previously received definitive chemoradiation treatment (e.g., definitive concurrent chemoradiation treatment) for ESCC.

122. The method of embodiment 121, wherein the tiragolumab is administered at a fixed dose of about 420 mg every two weeks and the atezolizumab is administered at a fixed dose of about 840 mg every two weeks.

123. A method for treating a subject having an ESCC, the method comprising administering to the subject one or more dosing cycles of tiragolumab at a fixed dose of about 700 mg to about 1000 mg every four weeks and atezolizumab at a fixed dose of about 400 mg to about 2000 mg every four weeks, wherein the subject previously received definitive chemoradiation treatment (e.g., definitive concurrent chemoradiation treatment) for ESCC.

124. The method of embodiment 123, wherein the tiragolumab is administered at a fixed dose of about 840 mg every four weeks and the atezolizumab is administered at a fixed dose of about 1680 mg every four weeks.

125. The method of any one of embodiments 119-124, wherein no chemotherapy is administered to the subject during the one or more dosing cycles.

126. The method of any one of embodiments 119-125, wherein an ESCC tumor sample obtained from the subject has been determined to have a TIC score of greater than, or equal to 10%, as determined by an IHC assay using anti-PD-L1 antibody SP263.

127. The method of any one of embodiments 119-126, wherein an ESCC tumor sample obtained from the subject has been determined to have a TIC score of less than 10%, as determined by an IHC assay using anti-PD-L1 antibody SP263.

128. The method of any one of embodiments 119-127, wherein the ESCC is a locally advanced ESCC, an unresectable ESCC, an unresectable locally advanced ESCC, a recurrent or metastatic ESCC, or an ESCC comprising a cervical esophageal tumor.

129. The method of any one of embodiments 119-128, wherein the ESCC is a Stage II ESCC, a Stage III ESCC, or a Stage IV ESCC.

130. The method of embodiment 129, wherein the Stage IV ESCC is a Stage IVA ESCC ora Stage IVB ESCC with supraclavicular lymph node metastases only.

131. The method of any one of embodiments 119-130, wherein the method comprises administering to the subject 17 dosing cycles.

132. The method of any one of embodiments 1-131, wherein the subject is a human.

133. A kit comprising an anti-TIGIT antagonist antibody for use in combination with a PD-1 axis binding antagonist for treating a subject having an ESCC according to the method of any one of embodiments 1-118.

134. The kit of embodiment 133, wherein the kit further comprises the PD-1 axis binding antagonist.

135. A kit comprising a PD-1 axis binding antagonist for use in combination with an anti-TIGIT antagonist antibody for treating a subject having an ESCC according to the method of any one of embodiments 1-118.

136. The kit of embodiment 135, wherein the kit further comprises anti-TIGIT antagonist antibody.

137. The kit of any one of embodiments 133-136, wherein the anti-TIGIT antagonist antibody is tiragolumab and the PD-1 axis binding antagonist is atezolizumab.

138. An anti-TIGIT antagonist antibody and a PD-1 axis binding antagonist for use in a method of treating a subject having an ESCC.

139. The anti-TIGIT antagonist antibody and PD-1 axis binding antagonist for use of embodiment

138, wherein the method comprises administering to the subject one or more dosing cycles of an anti-TIGIT antagonist antibody and a PD-1 axis binding antagonist, wherein the subject previously received definitive chemoradiation treatment (e.g., definitive concurrent chemoradiation treatment) for ESCC.

140. The anti-TIGIT antagonist antibody and PD-1 axis binding antagonist for use of embodiment 139, wherein the definitive chemoradiation treatment was completed no more than 89 days prior to administration with the anti-TIGIT antagonist antibody or the PD-1 axis binding antagonist.

141. The anti-TIGIT antagonist antibody and PD-1 axis binding antagonist for use of embodiment 139 or 140, wherein the definitive chemoradiation treatment comprises at least two cycles of platinum-based chemotherapy and radiation therapy without evidence of radiographic disease progression.

142. The anti-TIGIT antagonist antibody and PD-1 axis binding antagonist for use of any one of embodiments 139-141, wherein no chemotherapy is administered to the subject during the one or more dosing cycles.

143. The anti-TIGIT antagonist antibody and PD-1 axis binding antagonist for use of any one of embodiments 139-142, wherein the anti-TIGIT antagonist antibody is administered at a fixed dose of about 30 mg to about 1200 mg every three weeks.

144. The anti-TIGIT antagonist antibody and PD-1 axis binding antagonist for use of any one of embodiments 139-143, wherein the anti-TIGIT antagonist antibody is administered at a fixed dose of about 30 mg to about 800 mg every three weeks.

145. The anti-TIGIT antagonist antibody and PD-1 axis binding antagonist for use of embodiment 144, wherein the anti-TIGIT antagonist antibody is administered at a fixed dose of about 600 mg every three weeks.

146. The anti-TIGIT antagonist antibody and PD-1 axis binding antagonist for use of any one of embodiments 139-145, wherein the PD-1 axis binding antagonist is administered at a fixed dose of about 80 mg to about 1600 mg every three weeks.

147. The anti-TIGIT antagonist antibody and PD-1 axis binding antagonist for use of any one of embodiments 1-146, wherein the PD-1 axis binding antagonist is administered at a fixed dose of about 800 mg to about 1400 mg every three weeks.

148. The anti-TIGIT antagonist antibody and PD-1 axis binding antagonist for use of embodiment 147, wherein the PD-1 axis binding antagonist is administered at a fixed dose of about 1200 mg every three weeks.

149. The anti-TIGIT antagonist antibody and PD-1 axis binding antagonist for use of embodiment 148, wherein the anti-TIGIT antagonist antibody is administered at a fixed dose of about 600 mg every three weeks and the PD-1 axis binding antagonist is administered at a fixed dose of about 1200 mg every three weeks.

150. The anti-TIGIT antagonist antibody and PD-1 axis binding antagonist for use of any one of embodiments 139-148, wherein the length of each of the one or more dosing cycles is 21 days.

151. The anti-TIGIT antagonist antibody and PD-1 axis binding antagonist for use of any one of embodiments 139-142, wherein the anti-TIGIT antagonist antibody is administered at a fixed dose of about 300 mg to about 800 mg every two weeks.

152. The anti-TIGIT antagonist antibody and PD-1 axis binding antagonist for use of embodiment

151, wherein the anti-TIGIT antagonist antibody is administered at a fixed dose of about 400 mg to about 500 mg every two weeks.

153. The anti-TIGIT antagonist antibody and PD-1 axis binding antagonist for use of embodiment 152, wherein the anti-TIGIT antagonist antibody is administered at a fixed dose of about 420 mg every two weeks.

154. The anti-TIGIT antagonist antibody and PD-1 axis binding antagonist for use of any one of embodiments 139-142 and 151-153, wherein the PD-1 axis binding antagonist is administered at a fixed dose of about 200 mg to about 1200 mg every two weeks.

155. The anti-TIGIT antagonist antibody and PD-1 axis binding antagonist for use of embodiment 154, wherein the PD-1 axis binding antagonist is administered at a fixed dose of about 800 mg to about 1000 mg every two weeks.

156. The anti-TIGIT antagonist antibody and PD-1 axis binding antagonist for use of embodiment 155, wherein the PD-1 axis binding antagonist is administered at a fixed dose of about 840 mg every two weeks.

157. The anti-TIGIT antagonist antibody and PD-1 axis binding antagonist for use of embodiment 156, wherein the anti-TIGIT antagonist antibody is administered at a fixed dose of about 420 mg every two weeks and the PD-1 axis binding antagonist is administered at a fixed dose of about 840 mg every two weeks.

158. The anti-TIGIT antagonist antibody and PD-1 axis binding antagonist for use of any one of embodiments 139-142, wherein the anti-TIGIT antagonist antibody is administered at a fixed dose of about 700 mg to about 1000 mg every four weeks.

159. The anti-TIGIT antagonist antibody and PD-1 axis binding antagonist for use of embodiment 158, wherein the anti-TIGIT antagonist antibody is administered at a fixed dose of about 800 mg to about 900 mg every four weeks.

160. The anti-TIGIT antagonist antibody and PD-1 axis binding antagonist for use of embodiment 159, wherein the anti-TIGIT antagonist antibody is administered at a fixed dose of about 840 mg every four weeks.

161. The anti-TIGIT antagonist antibody and PD-1 axis binding antagonist for use of any one of embodiments 139-142 and 158-160, wherein the PD-1 axis binding antagonist is administered at a fixed dose of about 400 mg to about 2000 mg every four weeks.

162. The anti-TIGIT antagonist antibody and PD-1 axis binding antagonist for use of embodiment 161, wherein the PD-1 axis binding antagonist is administered at a fixed dose of about 1600 mg to about 1800 mg every four weeks.

163. The anti-TIGIT antagonist antibody and PD-1 axis binding antagonist for use of embodiment 162, wherein the PD-1 axis binding antagonist is administered at a fixed dose of about 1680 mg every four weeks.

164. The anti-TIGIT antagonist antibody and PD-1 axis binding antagonist for use of embodiment 163, wherein the anti-TIGIT antagonist antibody is administered at a fixed dose of about 840 mg every four weeks and the PD-1 axis binding antagonist is administered at a fixed dose of about 1680 mg every four weeks.

165. The anti-TIGIT antagonist antibody and PD-1 axis binding antagonist for use of any one of embodiments 1-164, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

166. The anti-TIGIT antagonist antibody and PD-1 axis binding antagonist for use of embodiment 165, wherein the anti-TIGIT antagonist antibody further comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

167. The anti-TIGIT antagonist antibody and PD-1 axis binding antagonist for use of embodiment 165 or 166, wherein the anti-TIGIT antagonist antibody further comprises the following heavy chain variable region FRs:

an FR-H1 comprising the amino acid sequence of XiVQLQQSGPGLVKPSQTLSLTCAISGDSVS (SEQ ID NO: 11), wherein Xi is E or Q;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14). 168. The anti-TIGIT antagonist antibody and PD-1 axis binding antagonist for use of embodiment 167, wherein Xi is E.

169. The anti-TIGIT antagonist antibody and PD-1 axis binding antagonist for use of embodiment 167, wherein Xi is Q.

170. The anti-TIGIT antagonist antibody and PD-1 axis binding antagonist for use of any one of embodiments 165-169, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b). 181. The anti-TIGIT antagonist antibody and PD-1 axis binding antagonist for use of any one of embodiments 139-170, wherein the anti-TIGIT antagonist antibody comprises:

(a) a VH domain comprising the amino acid sequence of SEQ ID NO: 17 or 18; and

(b) a VL domain comprising the amino acid sequence of SEQ ID NO: 19.

172. The anti-TIGIT antagonist antibody and PD-1 axis binding antagonist for use of any one of embodiments 139-171, wherein the anti-TIGIT antagonist antibody is a monoclonal antibody.

173. The anti-TIGIT antagonist antibody and PD-1 axis binding antagonist for use of embodiment 172, wherein the anti-TIGIT antagonist antibody is a human antibody.

174. The anti-TIGIT antagonist antibody and PD-1 axis binding antagonist for use of any one of embodiments 139-173, wherein the anti-TIGIT antagonist antibody is a full-length antibody.

175. The anti-TIGIT antagonist antibody and PD-1 axis binding antagonist for use of any one of embodiments 139-164 and 172-174, wherein the anti-TIGIT antagonist antibody has intact Fc-mediated effector function.

176. The anti-TIGIT antagonist antibody and PD-1 axis binding antagonist for use of embodiment 175, wherein the anti-TIGIT antagonist antibody is tiragolumab, vibostolimab, etigilimab, EOS084448, SGN-TGT, or TJ-T6.

177. The anti-TIGIT antagonist antibody and PD-1 axis binding antagonist for use of any one of embodiments 139-168 and 170-176, wherein the anti-TIGIT antagonist antibody is tiragolumab.

178. The anti-TIGIT antagonist antibody and PD-1 axis binding antagonist for use of any one of embodiments 139-164 and 172-174, wherein the anti-TIGIT antagonist antibody has enhanced Fc-mediated effector function.

179. The anti-TIGIT antagonist antibody and PD-1 axis binding antagonist for use of any one of embodiments 139-164, 172-176, and 178, wherein the anti-TIGIT antagonist antibody is SGN-TGT.

180. The anti-TIGIT antagonist antibody and PD-1 axis binding antagonist for use of any one of embodiments 139-164 and 172-174, wherein the anti-TIGIT antagonist antibody does not have Fc-mediated effector function.

181. The anti-TIGIT antagonist antibody and PD-1 axis binding antagonist for use of any one of embodiments 139-164, 172-174, and 180, wherein the anti-TIGIT antagonist antibody is domvanalimab, BMS-986207, ASP8374, or COM902.

182. The anti-TIGIT antagonist antibody and PD-1 axis binding antagonist for use of any one of embodiments 129-163, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

183. The anti-TIGIT antagonist antibody and PD-1 axis binding antagonist for use of any one of embodiments 139-164 and 172-181, wherein the anti-TIGIT antagonist antibody is an IgG class antibody.

184. The anti-TIGIT antagonist antibody and PD-1 axis binding antagonist for use of embodiment 183, wherein the IgG class antibody is an IgG1 subclass antibody.

185. The anti-TIGIT antagonist antibody and PD-1 axis binding antagonist for use of embodiment 184, wherein the anti-TIGIT antagonist antibody is tiragolumab, vibostolimab, etigilimab, EOS084448, SGN-TGT, TJ-T6, BGB-A1217, AB308, domvanalimab, or BMS-986207.

186. The anti-TIGIT antagonist antibody and PD-1 axis binding antagonist for use of embodiment 185, wherein the anti-TIGIT antagonist antibody is tiragolumab.

187. The anti-TIGIT antagonist antibody and PD-1 axis binding antagonist for use of embodiment 183, wherein the IgG class antibody is an IgG4 subclass antibody.

188. The anti-TIGIT antagonist antibody and PD-1 axis binding antagonist for use of embodiment 187, wherein the anti-TIGIT antagonist antibody is ASP8374 or COM902.

189. The anti-TIGIT antagonist antibody and PD-1 axis binding antagonist for use of any one of embodiments 139-188, wherein the PD-1 axis binding antagonist is a PD-L1 binding antagonist or a PD-1 binding antagonist.

190. The anti-TIGIT antagonist antibody and PD-1 axis binding antagonist for use of embodiment 189, wherein the PD-L1 binding antagonist is an anti-PD-L1 antagonist antibody.

191. The anti-TIGIT antagonist antibody and PD-1 axis binding antagonist for use of any one of embodiments 1-190, wherein the anti-PD-L1 antagonist antibody is atezolizumab (MPDL3280A), MSB0010718C, MDX-1105, or MED14736.

192. The anti-TIGIT antagonist antibody and PD-1 axis binding antagonist for use of embodiment 191, wherein the anti-PD-L1 antagonist antibody is atezolizumab.

193. The anti-TIGIT antagonist antibody and PD-1 axis binding antagonist for use of embodiment 189, wherein the PD-1 binding antagonist is an anti-PD-1 antagonist antibody.

194. The anti-TIGIT antagonist antibody and PD-1 axis binding antagonist for use of embodiment 193, wherein the anti-PD-1 antagonist antibody is nivolumab (MDX-1106), pembrolizumab (MK-3475), or AMP-224.

195. The anti-TIGIT antagonist antibody and PD-1 axis binding antagonist for use of any one of embodiments 139-190, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

196. The anti-TIGIT antagonist antibody and PD-1 axis binding antagonist for use of embodiment 195, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

197. The anti-TIGIT antagonist antibody and PD-1 axis binding antagonist for use of any one of embodiments 139-196, wherein the anti-PD-L1 antagonist antibody comprises:

a VH domain comprising the amino acid sequence of SEQ ID NO: 26; and

a VL domain comprising the amino acid sequence of SEQ ID NO: 27.

198. The anti-TIGIT antagonist antibody and PD-1 axis binding antagonist for use of any one of embodiments 195-197, wherein the anti-PD-L1 antagonist antibody is a monoclonal antibody.

199. The anti-TIGIT antagonist antibody and PD-1 axis binding antagonist for use of embodiment 198, wherein the anti-PD-L1 antagonist antibody is a humanized antibody.

200. The anti-TIGIT antagonist antibody and PD-1 axis binding antagonist for use of embodiment 198 or 199, wherein the anti-PD-L1 antagonist antibody is a full-length antibody.

201. The anti-TIGIT antagonist antibody and PD-1 axis binding antagonist for use of any one of embodiments 195-199, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

202. The anti-TIGIT antagonist antibody and PD-1 axis binding antagonist for use of any one of embodiments 195-201, wherein the anti-PD-L1 antagonist antibody is an IgG class antibody.

203. The anti-TIGIT antagonist antibody and PD-1 axis binding antagonist for use of embodiment 202, wherein the IgG class antibody is an IgG1 subclass antibody.

204. The anti-TIGIT antagonist antibody and PD-1 axis binding antagonist for use of any one of embodiments 139-203, wherein the method comprises administering to the subject the anti-TIGIT antagonist antibody and the PD-1 axis binding antagonist on about Day 1 of each of the one or more dosing cycles.

205. The anti-TIGIT antagonist antibody and PD-1 axis binding antagonist for use of any one of embodiments 139-204, wherein the method comprises administering to the subject the PD-1 axis binding antagonist before the anti-TIGIT antagonist antibody.

206. The anti-TIGIT antagonist antibody and PD-1 axis binding antagonist for use of embodiment 205, wherein the method comprises a first observation period following administration of the PD-1 axis binding antagonist and a second observation period following administration of the anti-TIGIT antagonist antibody.

207. The anti-TIGIT antagonist antibody and PD-1 axis binding antagonist for use of embodiment 206, wherein the first observation period and the second observation period are each between about 30 minutes to about 60 minutes in length.

208. The anti-TIGIT antagonist antibody and PD-1 axis binding antagonist for use of any one of embodiments 139-204, wherein the method comprises administering to the subject the anti-TIGIT antagonist antibody before the PD-1 axis binding antagonist.

209. The anti-TIGIT antagonist antibody and PD-1 axis binding antagonist for use of embodiment 208, wherein the method comprises a first observation period following administration of the anti-TIGIT antagonist antibody and a second observation period following administration of the PD-1 axis binding antagonist.

210. The anti-TIGIT antagonist antibody and PD-1 axis binding antagonist for use of embodiment 209, wherein the first observation period and the second observation period are each between about 30 minutes to about 60 minutes in length.

211. The anti-TIGIT antagonist antibody and PD-1 axis binding antagonist for use of any one of embodiments 139-204, wherein the method comprises administering to the subject the anti-TIGIT antagonist antibody and the PD-1 axis binding antagonist simultaneously.

212. The anti-TIGIT antagonist antibody and PD-1 axis binding antagonist for use of any one of embodiments 139-211, wherein the method comprises administering to the subject the anti-TIGIT antagonist antibody and the PD-1 axis binding antagonist intravenously.

213. The anti-TIGIT antagonist antibody and PD-1 axis binding antagonist for use of embodiment 212, wherein the method comprises administering to the subject the anti-TIGIT antagonist antibody by intravenous infusion over 60±10 minutes.

214. The anti-TIGIT antagonist antibody and PD-1 axis binding antagonist for use of embodiment 212 or 213, wherein the method comprises administering to the subject the PD-1 axis binding antagonist by intravenous infusion over 60±15 minutes.

215. The anti-TIGIT antagonist antibody and PD-1 axis binding antagonist for use of any one of embodiments 139-214, wherein an ESCC tumor sample obtained from the subject has been determined to have a detectable expression level of PD-L1.

216. The anti-TIGIT antagonist antibody and PD-1 axis binding antagonist for use of embodiment 215, wherein the detectable expression level of PD-L1 is a detectable protein expression level of PD-L1.

217. The anti-TIGIT antagonist antibody and PD-1 axis binding antagonist for use of embodiment 216, wherein the detectable protein expression level of PD-L1 has been determined by an immunohistochemical (IHC) assay.

218. The anti-TIGIT antagonist antibody and PD-1 axis binding antagonist for use of embodiment 217, wherein the IHC assay uses anti-PD-L1 antibody SP263, 22C3, SP142, or 28-8.

219. The anti-TIGIT antagonist antibody and PD-1 axis binding antagonist for use of embodiment 218, wherein the IHC assay uses anti-PD-L1 antibody SP263.

220. The anti-TIGIT antagonist antibody and PD-1 axis binding antagonist for use of embodiment 219, wherein the IHC assay is the Ventana SP263 IHC assay.

221. The anti-TIGIT antagonist antibody and PD-1 axis binding antagonist for use of embodiment 220, wherein the ESCC tumor sample has been determined to have a tumor and tumor-associated immune cell (TIC) score of greater than, or equal to, 1%.

222. The anti-TIGIT antagonist antibody and PD-1 axis binding antagonist for use of embodiment 221, wherein the TIC score is greater than, or equal to, 10%.

223. The anti-TIGIT antagonist antibody and PD-1 axis binding antagonist for use of embodiment 220 or 221, wherein the ESCC tumor sample has been determined to have a TIC score of less than 10%.

224. The anti-TIGIT antagonist antibody and PD-1 axis binding antagonist for use of embodiment 222, wherein the TIC score is greater than, or equal to, 10% and less than 50%.

225. The anti-TIGIT antagonist antibody and PD-1 axis binding antagonist for use of embodiment 218, wherein the IHC assay uses the anti-PD-L1 antibody 22C3.

226. The anti-TIGIT antagonist antibody and PD-1 axis binding antagonist for use of embodiment 225, wherein the IHC assay is the pharmDx 22C3 IHC assay.

227. The anti-TIGIT antagonist antibody and PD-1 axis binding antagonist for use of embodiment 226, wherein the ESCC tumor sample has been determined to have a combined positive score (CPS) of greater than, or equal to, 10 or a TPS of greater than, or equal to, 1%.

228. The anti-TIGIT antagonist antibody and PD-1 axis binding antagonist for use of embodiment 218, wherein the IHC assay uses the anti-PD-L1 antibody SP142.

229. The anti-TIGIT antagonist antibody and PD-1 axis binding antagonist for use of embodiment 228, wherein the IHC assay is the Ventana SP142 IHC assay.

230. The anti-TIGIT antagonist antibody and PD-1 axis binding antagonist for use of embodiment 218, wherein the IHC assay uses the anti-PD-L1 antibody 28-8.

231. The anti-TIGIT antagonist antibody and PD-1 axis binding antagonist for use of embodiment 230, wherein the IHC assay is the pharmDx 28-8 IHC assay.

232. The anti-TIGIT antagonist antibody and PD-1 axis binding antagonist for use of embodiment 215, wherein the detectable expression level of PD-L1 is a detectable nucleic acid expression level of PD-L1.

233. The anti-TIGIT antagonist antibody and PD-1 axis binding antagonist for use of embodiment 232, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

234. The anti-TIGIT antagonist antibody and PD-1 axis binding antagonist for use of any one of embodiments 139-233, wherein the ESCC is a locally advanced ESCC.

235. The anti-TIGIT antagonist antibody and PD-1 axis binding antagonist for use of any one of embodiments 139-234, wherein the ESCC is an unresectable ESCC.

236. The anti-TIGIT antagonist antibody and PD-1 axis binding antagonist for use of any one of embodiments 139-235, wherein the ESCC is a recurrent or metastatic ESCC.

237. The anti-TIGIT antagonist antibody and PD-1 axis binding antagonist for use of any one of embodiments 139-236, wherein the ESCC comprises a cervical esophageal tumor.

238. The anti-TIGIT antagonist antibody and PD-1 axis binding antagonist for use of any one of embodiments 139-237, wherein the ESCC is a Stage II ESCC, a Stage III ESCC, ora Stage IV ESCC, optionally wherein the Stage IV ESCC is a Stage IVA ESCC or a Stage IVB ESCC with supraclavicular lymph node metastases only.

239. The anti-TIGIT antagonist antibody and PD-1 axis binding antagonist for use of any one of embodiments 139-238, wherein the subject or population of subjects has not been treated previously with cancer immunotherapy.

240. The anti-TIGIT antagonist antibody and PD-1 axis binding antagonist for use of any one of embodiments 139-238, wherein the subject has completed a previous cancer immunotherapy for ESCC.

241. The anti-TIGIT antagonist antibody and PD-1 axis binding antagonist for use of any one of embodiments 139-240, wherein the treatment results in an increase in progression-free survival (PFS) of the subject as compared to treatment with the PD-1 axis binding antagonist without the anti-TIGIT antagonist antibody.

242. The anti-TIGIT antagonist antibody and PD-1 axis binding antagonist for use of any one of embodiments 139-241, wherein the treatment results in an increase in PFS of the subject as compared to treatment with the anti-TIGIT antagonist antibody without the PD-1 axis binding antagonist.

243. The anti-TIGIT antagonist antibody and PD-1 axis binding antagonist for use of any one of embodiments 139-242, wherein the treatment results in an increase in PFS of the subject as compared to treatment without the anti-TIGIT antagonist antibody and without the PD-1 axis binding antagonist.

234. The anti-TIGIT antagonist antibody and PD-1 axis binding antagonist for use of embodiment 243, wherein the treatment extends the PFS of the subject or population of subjects by at least about 4 months or about 8 months.

245. The anti-TIGIT antagonist antibody and PD-1 axis binding antagonist for use of embodiment 243, wherein the treatment results in a median PFS of the population of subjects of about 15 months to about 23 months.

246. The anti-TIGIT antagonist antibody and PD-1 axis binding antagonist for use of any one of embodiments 139-245, wherein the treatment results in an increase in overall survival (OS) of the subject as compared to treatment with the PD-1 axis binding antagonist without the anti-TIGIT antagonist antibody.

247. The anti-TIGIT antagonist antibody and PD-1 axis binding antagonist for use of any one of embodiments 139-245, wherein the treatment results in an increase in OS of the subject as compared to treatment with the anti-TIGIT antagonist antibody and without treatment with the PD-1 axis binding antagonist.

248. The anti-TIGIT antagonist antibody and PD-1 axis binding antagonist for use of any one of embodiments 139-245, wherein the treatment results in an increase in OS of the subject as compared to treatment without the anti-TIGIT antagonist antibody and without the PD-1 axis binding antagonist.

249. The anti-TIGIT antagonist antibody and PD-1 axis binding antagonist for use of embodiment 248, wherein the treatment extends the OS of the subject or population of subjects by at least about 7 months or about 12 months.

250. The anti-TIGIT antagonist antibody and PD-1 axis binding antagonist for use of embodiment 248, wherein the treatment results in a median OS of the population of subjects of about 24 months to about 36 months.

251. The anti-TIGIT antagonist antibody and PD-1 axis binding antagonist for use of any one of embodiments 139-250, wherein the treatment results in an increase in duration of objective response (DOR) in the subject as compared to treatment with the PD-1 axis binding antagonist without the anti-TIGIT antagonist antibody.

252. The anti-TIGIT antagonist antibody and PD-1 axis binding antagonist for use of any one of embodiments 139-251, wherein the treatment results in an increase in DOR in the subject as compared to treatment with the anti-TIGIT antagonist antibody without the PD-1 axis binding antagonist.

253. The anti-TIGIT antagonist antibody and PD-1 axis binding antagonist for use of any one of embodiments 139-250, wherein the treatment results in an increase in DOR in the subject as compared to treatment without the anti-TIGIT antagonist antibody and without the PD-1 axis binding antagonist.

254. The anti-TIGIT antagonist antibody and PD-1 axis binding antagonist for use of any one of embodiments 139-253, wherein the treatment results in a complete response or a partial response.

255. The anti-TIGIT antagonist antibody and PD-1 axis binding antagonist for use of any one of embodiments 139-254, wherein the method comprises administering to the subject at least five dosing cycles.

256. The anti-TIGIT antagonist antibody and PD-1 axis binding antagonist for use of embodiment 255, wherein the method comprises administering to the subject 17 dosing cycles.

257. Use of an anti-TIGIT antagonist antibody in the manufacture of a medicament for treating a subject having an ESCC in combination with a PD-1 axis binding antagonist, wherein the treatment is according to the method of any one of embodiments 1-118.

258. Use of a PD-1 axis binding antagonist in the manufacture of a medicament for treating a subject having an ESCC in combination with an anti-TIGIT antagonist antibody, wherein the treatment is according to the method of any one of embodiments 1-118. 259. The use of embodiment 257 or 258, wherein the anti-TIGIT antagonist antibody and the PD-1 axis binding antagonist are formulated separately.

260. The use of embodiment 257 or 258, wherein the anti-TIGIT antagonist antibody and the PD-1 axis binding antagonist are formulated together.

261. A method for treating a subject or population of subjects having an advanced esophageal squamous cell carcinoma (ESCC), the method comprising administering to the subject or population of subjects one or more dosing cycles of an anti-TIGIT antagonist antibody, a PD-1 axis binding antagonist, a taxane, and a platinum agent, wherein the subject or population of subjects has received no prior systemic treatment for advanced ESCC.

262. A method for treating a subject or population of subjects having an advanced ESCC for whom surgery is unsuitable, the method comprising administering to the subject or population of subjects one or more dosing cycles of an anti-TIGIT antagonist antibody, a PD-1 axis binding antagonist, a taxane, and a platinum agent.

263. The method of embodiment 262, wherein the subject or population of subjects has received no prior systemic treatment for advanced ESCC.

264. The method of any one of embodiments 261-263, wherein the subject or population of subjects has received no prior systemic treatment for non-advanced ESCC.

265. The method of any one of embodiments 261-263, wherein the subject or population of subjects has received prior treatment for non-advanced ESCC, wherein the prior treatment for the non-advanced ESCC was completed at least six months before diagnosis of the advanced ESCC.

266. The method of embodiment 265, wherein the prior treatment for the non-advanced ESCC comprises a chemoradiotherapy or a chemotherapy.

267. The method of embodiment 266, wherein the chemoradiotherapy or chemotherapy was administered with curative intent or in an adjuvant or neoadjuvant setting.

268. The method of any one of embodiments 261-267, wherein the anti-TIGIT antagonist antibody is administered at a fixed dose of about 30 mg to about 1200 mg every three weeks,

269. The method of embodiment 268, wherein the anti-TIGIT antagonist antibody is administered at a fixed dose of about 30 mg to about 800 mg every three weeks.

270. The method of embodiment 269, wherein the anti-TIGIT antagonist antibody is administered at a fixed dose of about 600 mg every three weeks.

271. The method of any one of embodiments 261-270, wherein the PD-1 axis binding antagonist is administered at a fixed dose of about 80 mg to about 1600 mg every three weeks

272. The method of embodiment 271, wherein the PD-1 axis binding antagonist is administered at a fixed dose of about 800 mg to about 1400 mg every three weeks.

273. The method of embodiment 272, wherein the PD-1 axis binding antagonist is administered at a fixed dose of about 1200 mg every three weeks.

274. The method of any one of embodiments 261-273, wherein the taxane is administered at a dose of about 100-250 mg/m² every three weeks

275. The method of embodiment 274, wherein the taxane is administered at a dose of 150-200 mg/m² every three weeks.

276. The method of embodiment 275, wherein the taxane is administered at a dose of about 175 mg/m² every three weeks.

277. The method of any one of embodiments 261-276, wherein the platinum agent is administered at a dose of about 20-200 mg/m² every three weeks

278. The method of embodiment 277, wherein the platinum agent is administered at a dose of about 40-120 mg/m² every three weeks.

279. The method of embodiment 278, wherein the platinum agent is administered at a dose of about 60-80 mg/m² every three weeks.

280. The method of embodiment 279, wherein the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks.

281. The method of any one of embodiments 261-280, wherein the length of each of the one or more dosing cycles is 21 days.

282. The method of any one of embodiments 261-281, wherein the anti-TIGIT antagonist antibody, PD-1 axis binding antagonist, the taxane, and the platinum agent are administered in each of 4-8 induction phase dosing cycles.

283. The method of embodiment 282, wherein the anti-TIGIT antagonist antibody, PD-1 axis binding antagonist, the taxane, and the platinum agent are administered in each of six induction phase dosing cycles.

284. The method of embodiment 282 or 283, wherein the anti-TIGIT antagonist antibody and the PD-1 axis binding antagonist are further administered in one or more maintenance phase dosing cycles following the induction phase dosing cycles.

285. The method of embodiment 284, wherein the taxane and the platinum agent are omitted from each of the one or more maintenance phase dosing cycles.

286. The method of any one of embodiments 282-285, wherein the length of each of the induction phase dosing cycles and/or the one or more maintenance phase dosing cycles is 21 days.

287. The method of any one of embodiments 261-267, wherein the anti-TIGIT antagonist antibody is administered at a fixed dose of about 300 mg to about 800 mg every two weeks.

288. The method of embodiment 287, wherein the anti-TIGIT antagonist antibody is administered at a fixed dose of about 400 mg to about 500 mg every two weeks.

289. The method of embodiment 288, wherein the anti-TIGIT antagonist antibody is administered at a fixed dose of about 420 mg every two weeks.

290. The method of any one of embodiments 261-267 and 287-289, wherein the PD-1 axis binding antagonist is administered at a fixed dose of about 200 mg to about 1200 mg every two weeks.

291. The method of embodiment 290, wherein the PD-1 axis binding antagonist is administered at a fixed dose of about 800 mg to about 1000 mg every two weeks.

292. The method of embodiment 291, wherein the PD-1 axis binding antagonist is administered at a fixed dose of about 840 mg every two weeks.

293. The method of embodiment 292, wherein the anti-TIGIT antagonist antibody is administered at a fixed dose of about 420 mg every two weeks and the PD-1 axis binding antagonist is administered at a fixed dose of about 840 mg every two weeks.

294. The method of any one of embodiments 287-293, wherein the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

295. The method of any one of embodiments 261-267, wherein the anti-TIGIT antagonist antibody is administered at a fixed dose of about 700 mg to about 1000 mg every four weeks.

296. The method of embodiment 295, wherein the anti-TIGIT antagonist antibody is administered at a fixed dose of about 800 mg to about 900 mg every four weeks.

297. The method of embodiment 296, wherein the anti-TIGIT antagonist antibody is administered at a fixed dose of about 840 mg every four weeks.

298. The method of any one of embodiments 261-267 and 295-297, wherein the PD-1 axis binding antagonist is administered at a fixed dose of about 400 mg to about 2000 mg every four weeks.

299. The method of embodiment 298, wherein the PD-1 axis binding antagonist is administered at a fixed dose of about 1600 mg to about 1800 mg every four weeks.

300. The method of embodiment 299, wherein the PD-1 axis binding antagonist is administered at a fixed dose of about 1680 mg every four weeks.

301. The method of embodiment 300, wherein the anti-TIGIT antagonist antibody is administered at a fixed dose of about 840 mg every four weeks and the PD-1 axis binding antagonist is administered at a fixed dose of about 1680 mg every four weeks.

302. The method of any one of embodiments 295-301, wherein the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

303. The method of any one of embodiments 287-302, wherein the taxane is administered once per week, once every two weeks, once every three weeks, twice every three weeks, once every four weeks, twice every four weeks, or three times every four weeks.

304. The method of any one of embodiments 287-303, wherein the platinum agent is administered once per week, once every two weeks, once every three weeks, twice every three weeks, once every four weeks, twice every four weeks, or three times every four weeks.

305. The method of any one of embodiments 261-304, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

306. The method of embodiment 305, wherein the anti-TIGIT antagonist antibody further comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

307. The method of embodiment 305 or 306, wherein the anti-TIGIT antagonist antibody further comprises the following heavy chain variable region FRs:

an FR-H1 comprising the amino acid sequence of XiVQLQQSGPGLVKPSQTLSLTCAISGDSVS (SEQ ID NO: 11), wherein Xi is E or Q;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

308. The method of embodiment 307, wherein Xi is E.

309. The method of embodiment 307, wherein Xi is Q.

310. The method of any one of embodiments 305-309, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

311. The method of any one of embodiments 261-310, wherein the anti-TIGIT antagonist antibody comprises:

(a) a VH domain comprising the amino acid sequence of SEQ ID NO: 17 or 18; and

(b) a VL domain comprising the amino acid sequence of SEQ ID NO: 19.

312. The method of any one of embodiments 261-311, wherein the anti-TIGIT antagonist antibody is a monoclonal antibody.

313. The method of embodiment 312, wherein the anti-TIGIT antagonist antibody is a human antibody.

314. The method of any one of embodiments 261-313, wherein the anti-TIGIT antagonist antibody is a full-length antibody.

315. The method of any one of embodiments 261-304 and 312-314, wherein the anti-TIGIT antagonist antibody has intact Fc-mediated effector function.

316. The method of embodiment 315, wherein the anti-TIGIT antagonist antibody is tiragolumab, vibostolimab, etigilimab, EOS084448, SGN-TGT, or TJ-T6.

317. The method of any one of embodiments 261-308 and 310-316, wherein the anti-TIGIT antagonist antibody is tiragolumab.

318. The method of any one of embodiments 261-304 and 312-314, wherein the anti-TIGIT antagonist antibody has enhanced Fc-mediated effector function.

319. The method of any one of embodiments 261-304, 312-314, and 318, wherein the anti-TIGIT antagonist antibody is SGN-TGT.

320. The method of any one of embodiments 261-304 and 312-314, wherein the anti-TIGIT antagonist antibody does not have Fc-mediated effector function.

321. The method of any one of embodiments 261-304, 312-314, and 320, wherein the anti-TIGIT antagonist antibody is domvanalimab, BMS-986207, ASP8374, or COM902.

322. The method of any one of embodiments 261-313, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

323. The method of any one of embodiments 261-304 and 312-321, wherein the anti-TIGIT antagonist antibody is an IgG class antibody.

324. The method of embodiment 323, wherein the IgG class antibody is an IgG1 subclass antibody.

325. The method of embodiment 324, wherein the anti-TIGIT antagonist antibody is tiragolumab, vibostolimab, etigilimab, EOS084448, SGN-TGT, TJ-T6, BGB-A1217, AB308, domvanalimab, or BMS-986207.

326. The method of embodiment 325, wherein the anti-TIGIT antagonist antibody is tiragolumab.

327. The method of embodiment 323, wherein the IgG class antibody is an IgG4 subclass antibody. 328. The method of embodiment 327, wherein the anti-TIGIT antagonist antibody is ASP8374 or COM902.

329. The method of any one of embodiments 261-328, wherein the PD-1 axis binding antagonist is a PD-L1 binding antagonist or a PD-1 binding antagonist.

330. The method of embodiment 329, wherein the PD-L1 binding antagonist is an anti-PD-L1 antagonist antibody.

331. The method of any one of embodiments 261-330, wherein the anti-PD-L1 antagonist antibody is atezolizumab (MPDL3280A), MSB0010718C, MDX-1105, or MED14736.

332. The method of embodiment 331, wherein the anti-PD-L1 antagonist antibody is atezolizumab.

333. The method of embodiment 329, wherein the PD-1 binding antagonist is an anti-PD-1 antagonist antibody.

334. The method of embodiment 333, wherein the anti-PD-1 antagonist antibody is nivolumab (MDX-1106), pembrolizumab (MK-3475), or AMP-224.

335. The method of any one of embodiments 261-330, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

336. The method of embodiment 335, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b). 337. The method of any one of embodiments 261-336, wherein the anti-PD-L1 antagonist antibody comprises:

a VH domain comprising the amino acid sequence of SEQ ID NO: 26; and

a VL domain comprising the amino acid sequence of SEQ ID NO: 27.

338. The method of any one of embodiments 335-337, wherein the anti-PD-L1 antagonist antibody is a monoclonal antibody.

339. The method of embodiment 338, wherein the anti-PD-L1 antagonist antibody is a humanized antibody.

340. The method of embodiment 338 or 339, wherein the anti-PD-L1 antagonist antibody is a full-length antibody.

341. The method of any one of embodiments 335-339, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

342. The method of any one of embodiments 335-339, wherein the anti-PD-L1 antagonist antibody is an IgG class antibody.

343. The method of embodiment 342, wherein the IgG class antibody is an IgG1 subclass antibody.

344. The method of any one of embodiments 261-343, wherein the taxane is paclitaxel or nab-paclitaxel.

345. The method of embodiment 344, wherein the taxane is paclitaxel.

346. The method of any one of embodiments 261-345, wherein the platinum agent is cisplatin or carboplatin.

347. The method of embodiment 346, wherein the platinum agent is cisplatin.

348. The method of any one of embodiments 261-347, wherein the method comprises administering to the subject or population of subjects the PD-1 axis binding antagonist before the anti-TIGIT antagonist antibody.

349. The method of embodiment 348, wherein the method comprises a first observation period following administration of the PD-1 axis binding antagonist and a second observation period following administration of the anti-TIGIT antagonist antibody.

350. The method of embodiment 349, wherein the first observation period and the second observation period are each between about 30 minutes to about 60 minutes in length.

351. The method of any one of embodiments 261-347, wherein the method comprises administering to the subject or population of subjects the anti-TIGIT antagonist antibody before the PD-1 axis binding antagonist.

352. The method of embodiment 351, wherein the method comprises a first observation period following administration of the anti-TIGIT antagonist antibody and a second observation period following administration of the PD-1 axis binding antagonist.

353. The method of embodiment 352, wherein the first observation period and the second observation period are each between about 30 minutes to about 60 minutes in length.

354. The method of any one of embodiments 261-347, wherein the method comprises administering to the subject or population of subjects the anti-TIGIT antagonist antibody and the PD-1 axis binding antagonist simultaneously.

355. The method of any one of embodiments 261-354, wherein the anti-TIGIT antagonist antibody and the PD-1 axis binding antagonist are administered before the taxane and/or the platinum agent.

356. The method of embodiment 355, wherein the method comprises administering to the subject or population of subjects the taxane before the platinum agent.

357. The method of embodiment 356, wherein the method comprises a third observation period following administration of the taxane and a fourth observation period following administration of the platinum agent.

358. The method of embodiment 357, wherein the third observation period and the fourth observation period are each between about 30 minutes to about 60 minutes in length.

359. The method of any one of embodiments 261-358, wherein the method comprises administering to the subject or population of subjects the anti-TIGIT antagonist antibody, the PD-1 axis binding antagonist, the taxane, and the platinum agent intravenously.

360. The method of embodiment 359, wherein the method comprises administering to the subject or population of subjects the anti-TIGIT antagonist antibody by intravenous infusion over 60±10 minutes.

361. The method of embodiment 359 or 360, wherein the method comprises administering to the subject or population of subjects the PD-1 axis binding antagonist by intravenous infusion over 60±15 minutes.

362. The method of any one of embodiments 359-361, wherein the method comprises administering to the subject or population of subjects the taxane by intravenous infusion over 3 hours±30 minutes.

363. The method of any one of embodiments 359-362, wherein the method comprises administering to the subject or population of subjects the platinum agent by intravenous infusion over 1-4 hours.

364. The method of any one of embodiments 261-363, wherein an ESCC tumor sample obtained from the subject or population of subjects has been determined to have a detectable expression level of PD-L1.

365. The method of embodiment 364, wherein the detectable expression level of PD-L1 is a detectable protein expression level of PD-L1.

366. The method of embodiment 365, wherein the detectable protein expression level of PD-L1 has been determined by an immunohistochemical (IHC) assay.

367. The method of embodiment 366, wherein the IHC assay uses anti-PD-L1 antibody SP263, 22C3, SP142, or 28-8.

368. The method of embodiment 367, wherein the IHC assay uses anti-PD-L1 antibody SP263.

369. The method of embodiment 368, wherein the IHC assay is the Ventana SP263 Companion Diagnostic (CDx) assay.

370. The method of embodiment 369, wherein the ESCC tumor sample has been determined to have a tumor and tumor-associated immune cell (TIC) score of greater than, or equal to, 1%.

371. The method of embodiment 370, wherein the TIC score is greater than, or equal to, 10%.

372. The method of embodiment 369 or 370, wherein the ESCC tumor sample has been determined to have a TIC score of less than 10%.

373. The method of embodiment 371, wherein the TIC score is greater than, or equal to, 10% and less than 50%.

374. The method of embodiment 367, wherein the IHC assay uses the anti-PD-L1 antibody 22C3.

375. The method of embodiment 374, wherein the IHC assay is the pharmDx 22C3 IHC assay.

376. The method of embodiment 375, wherein the ESCC tumor sample has been determined to have a combined positive score (CPS) of greater than, or equal to, 10 or a TPS of greater than, or equal to, 1%.

377. The method of embodiment 367, wherein the IHC assay uses the anti-PD-L1 antibody SP142.

378. The method of embodiment 377, wherein the IHC assay is the Ventana SP142 IHC assay.

379. The method of embodiment 367, wherein the IHC assay uses the anti-PD-L1 antibody 28-8.

380. The method of embodiment 379, wherein the IHC assay is the pharmDx 28-8 IHC assay.

381. The method of embodiment 364, wherein the detectable expression level of PD-L1 is a detectable nucleic acid expression level of PD-L1.

382. The method of embodiment 381, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

383. The method of any one of embodiments 261-382, wherein the advanced ESCC is a locally advanced ESCC.

384. The method of any one of embodiments 261-383, wherein the advanced ESCC is a recurrent or metastatic ESCC.

385. The method of any one of embodiments 261-384, wherein the advanced ESCC is an unresectable ESCC.

386. The method of any one of embodiments 261-385, wherein the treatment results in a progression-free survival (PFS) of about 8 months or more.

387. The method of any one of embodiments 261-386, wherein the treatment results in an increase in a PFS of the subject or population of subjects as compared to treatment with the taxane and the platinum agent, without the PD-1 axis binding antagonist and the anti-TIGIT antagonist antibody.

388. The method of embodiment 387, wherein the treatment extends the PFS of the subject or population of subjects by at least about 2 months or about 4 months.

389. The method of embodiment 387, wherein the increase in PFS is about 4 months or more. 390. The method of any one of embodiments 261-389, wherein the treatment results in a median PFS of the population of subjects of about 6 months to about 10 months.

391. The method of any one of embodiments 261-390, wherein the treatment results in an increase in OS of the subject or population of subjects as compared to treatment with the taxane and the platinum agent, without the PD-1 axis binding antagonist and the anti-TIGIT antagonist antibody.

392. The method of embodiment 391, wherein the treatment extends the OS of the subject or population of subjects by at least about 4 months or about 6 months.

393. The method of embodiment 391, wherein the treatment results in a median OS of the population of subjects of about 14 months to about 20 months.

394. The method of any one of embodiments 261-393, wherein the treatment results in an increase in duration of objective response (DOR) in the subject or population of subjects as compared to treatment with the taxane and the platinum agent, without the PD-1 axis binding antagonist and the anti-TIGIT antagonist antibody.

395. The method of any one of embodiments 261-394, wherein the treatment results in a complete response or a partial response.

396. A method for treating a subject having an advanced ESCC, the method comprising administering to the subject one or more dosing cycles of tiragolumab at a fixed dose of about 30 mg to about 1200 mg every three weeks, atezolizumab at a fixed dose of about 80 mg to about 1600 mg every three weeks, paclitaxel at a dose of about 100-250 mg/m² every three weeks, and cisplatin at a dose of about 20-200 mg/m² every three weeks, wherein the subject has received no prior systemic treatment for the advanced ESCC.

397. The method of embodiment 396, wherein the tiragolumab is administered at a fixed dose of about 600 mg every three weeks, the atezolizumab is administered at a fixed dose of about 1200 mg every three weeks, the paclitaxel is administered at a dose of about 175 mg/m² every three weeks, and the cisplatin is administered at a dose of about 60-80 mg/m² every three weeks.

398. A method for treating a subject having an advanced ESCC, the method comprising administering to the subject one or more dosing cycles of tiragolumab at a fixed dose of about 300 mg to about 800 mg every two weeks, atezolizumab at a fixed dose of about 200 mg to about 1200 mg every two weeks, paclitaxel, and cisplatin, wherein the subject has received no prior systemic treatment for the advanced ESCC.

399. The method of embodiment 398, wherein the tiragolumab is administered at a fixed dose of about 420 mg every two weeks, and the atezolizumab is administered at a fixed dose of about 840 mg every two weeks.

400. A method for treating a subject having an advanced ESCC, the method comprising administering to the subject one or more dosing cycles of tiragolumab at a fixed dose of about 700 mg to about 1000 mg every four weeks, atezolizumab at a fixed dose of about 400 mg to about 2000 mg every four weeks, paclitaxel, and cisplatin, wherein the subject has received no prior systemic treatment for the advanced ESCC.

401. The method of embodiment 400, wherein the tiragolumab is administered at a fixed dose of about 840 mg every four weeks, and the atezolizumab is administered at a fixed dose of about 1680 mg every four weeks.

402. A method for treating a subject having an advanced ESCC, the method comprising administering to the subject:

(i) six induction phase dosing cycles of tiragolumab at a fixed dose of about 30 mg to about 1200 mg every three weeks, atezolizumab at a fixed dose of about 80 mg to about 1600 mg every three weeks, paclitaxel at a dose of about 100-250 mg/m² every three weeks, and cisplatin at a dose of about 20-200 mg/m² every three weeks; and

(ii) one or more maintenance phase dosing cycles of tiragolumab at a fixed dose of about 30 mg to about 1200 mg every three weeks and atezolizumab at a fixed dose of about 80 mg to about 1600 mg every three weeks, wherein the paclitaxel and the cisplatin are omitted from each of the one or more maintenance phase dosing cycles,

wherein the subject has received no prior systemic treatment for the advanced ESCC.

403. The method of embodiment 402, wherein:

(i) in the six induction phase dosing cycles, the tiragolumab is administered at a fixed dose of about 600 mg every three weeks, the atezolizumab is administered at a fixed dose of about 1200 mg every three weeks, the paclitaxel is administered at a dose of about 175 mg/m² every three weeks, and the cisplatin is administered at a dose of about 60-80 mg/m² every three weeks; and

(ii) in the one or more maintenance phase dosing cycles, the tiragolumab is administered at a fixed dose of about 600 mg every three weeks and the atezolizumab is administered at a fixed dose of about 1200 mg every three weeks.

404. The method of any one of embodiments 396-403, wherein the subject has received no prior treatment for non-advanced ESCC.

405. The method of any one of embodiments 396-404, wherein the subject has received prior treatment for non-advanced ESCC, wherein the prior treatment for the non-advanced ESCC was completed at least six months before diagnosis of the advanced ESCC.

406. The method of embodiment 405, wherein the prior treatment for the non-advanced ESCC comprises a chemoradiotherapy or a chemotherapy.

407. The method of embodiment 406, wherein the chemoradiotherapy or chemotherapy was administered with curative intent or in an adjuvant or neoadjuvant setting.

408. The method of any one of embodiments 396-407, wherein an ESCC tumor sample obtained from the subject has been determined to have a TIC score of greater than, or equal to 10%, as determined by an IHC assay using anti-PD-L1 antibody SP263.

409. The method of any one of embodiments 396-407, wherein an ESCC tumor sample obtained from the subject has been determined to have a TIC score of less than 10%, as determined by an IHC assay using anti-PD-L1 antibody SP263.

410. The method of any one of embodiments 396-409, wherein the advanced ESCC is a locally advanced ESCC, an unresectable ESCC, an unresectable locally advanced ESCC, an unresectable recurrent ESCC, or a recurrent or metastatic ESCC.

411. The method of any one of embodiments 261-410, wherein the subject is a human.

412. A kit comprising an anti-TIGIT antagonist antibody for use in combination with a PD-1 axis binding antagonist, a taxane, and a platinum agent for treating a subject having an advanced ESCC according to the method of any one of embodiments 261-395.

413. The kit of embodiment 412, wherein the kit further comprises the PD-1 axis binding antagonist.

413. A kit comprising a PD-1 axis binding antagonist for use in combination with an anti-TIGIT antagonist antibody, a taxane, and a platinum agent for treating a subject having an advanced ESCC according to the method of any one of embodiments 261-413.

414. The kit of embodiment 413, wherein the kit further comprises the anti-TIGIT antagonist antibody.

415. The kit of any one of embodiments 412-414, wherein the anti-TIGIT antagonist antibody is tiragolumab and the PD-1 axis binding antagonist is atezolizumab.

416. An anti-TIGIT antagonist antibody, PD-1 axis binding antagonist, taxane, and platinum agent for use in a method of treating a subject having an advanced ESCC.

417. An anti-TIGIT antagonist antibody, PD-1 axis binding antagonist, taxane, and platinum agent for use in a method of treating a subject having an advanced ESCC, the method comprising administering to the subject one or more dosing cycles of an anti-TIGIT antagonist antibody, a PD-1 axis binding antagonist, a taxane, and a platinum agent, wherein the subject has received no prior systemic treatment for advanced ESCC.

418. An anti-TIGIT antagonist antibody, PD-1 axis binding antagonist, taxane, and platinum agent for use in a method of treating a subject having an advanced ESCC for whom surgery is unsuitable, the method comprising administering to the subject one or more dosing cycles of an anti-TIGIT antagonist antibody, a PD-1 axis binding antagonist, a taxane, and a platinum agent.

419. The anti-TIGIT antagonist antibody, PD-1 axis binding antagonist, taxane, and platinum agent for use of embodiment 418, wherein the subject has received no prior systemic treatment for advanced ESCC.

420. The anti-TIGIT antagonist antibody, PD-1 axis binding antagonist, taxane, and platinum agent for use of any one of embodiments 417-419, wherein the subject has received no prior systemic treatment for non-advanced ESCC.

421. The anti-TIGIT antagonist antibody, PD-1 axis binding antagonist, taxane, and platinum agent for use of any one of embodiments 417-419, wherein the subject has received prior treatment for non-advanced ESCC, wherein the prior treatment for the non-advanced ESCC was completed at least six months before diagnosis of the advanced ESCC.

422. The anti-TIGIT antagonist antibody, PD-1 axis binding antagonist, taxane, and platinum agent for use of embodiment 421, wherein the prior treatment for the non-advanced ESCC comprises a chemoradiotherapy or a chemotherapy.

423. The anti-TIGIT antagonist antibody, PD-1 axis binding antagonist, taxane, and platinum agent for use of embodiment 422, wherein the chemoradiotherapy or chemotherapy was administered with curative intent or in an adjuvant or neoadjuvant setting.

424. The anti-TIGIT antagonist antibody, PD-1 axis binding antagonist, taxane, and platinum agent for use of any one of embodiments 417-423, wherein the anti-TIGIT antagonist antibody is administered at a fixed dose of about 30 mg to about 1200 mg every three weeks,

425. The anti-TIGIT antagonist antibody, PD-1 axis binding antagonist, taxane, and platinum agent for use of embodiment 424, wherein the anti-TIGIT antagonist antibody is administered at a fixed dose of about 30 mg to about 800 mg every three weeks.

426. The anti-TIGIT antagonist antibody, PD-1 axis binding antagonist, taxane, and platinum agent for use of embodiment 425, wherein the anti-TIGIT antagonist antibody is administered at a fixed dose of about 600 mg every three weeks.

427. The anti-TIGIT antagonist antibody, PD-1 axis binding antagonist, taxane, and platinum agent for use of any one of embodiments 417-426, wherein the PD-1 axis binding antagonist is administered at a fixed dose of about 80 mg to about 1600 mg every three weeks

428. The anti-TIGIT antagonist antibody, PD-1 axis binding antagonist, taxane, and platinum agent for use of embodiment 427, wherein the PD-1 axis binding antagonist is administered at a fixed dose of about 800 mg to about 1400 mg every three weeks.

429. The anti-TIGIT antagonist antibody, PD-1 axis binding antagonist, taxane, and platinum agent for use of embodiment 428, wherein the PD-1 axis binding antagonist is administered at a fixed dose of about 1200 mg every three weeks.

430. The anti-TIGIT antagonist antibody, PD-1 axis binding antagonist, taxane, and platinum agent for use of any one of embodiments 417-429, wherein the taxane is administered at a dose of about 100-250 mg/m² every three weeks

431. The anti-TIGIT antagonist antibody, PD-1 axis binding antagonist, taxane, and platinum agent for use of embodiment 430, wherein the taxane is administered at a dose of 150-200 mg/m² every three weeks.

432. The anti-TIGIT antagonist antibody, PD-1 axis binding antagonist, taxane, and platinum agent for use of any one of embodiments 417-431, wherein the platinum agent is administered at a dose of about 20-200 mg/m² every three weeks and/or wherein the taxane is administered at a dose of about 175 mg/m² every three weeks.

433. The anti-TIGIT antagonist antibody, PD-1 axis binding antagonist, taxane, and platinum agent for use of embodiment 432, wherein the platinum agent is administered at a dose of about 40-120 mg/m² every three weeks.

434. The anti-TIGIT antagonist antibody, PD-1 axis binding antagonist, taxane, and platinum agent for use of embodiment 433, wherein the platinum agent is administered at a dose of about 60-80 mg/m² every three weeks.

435. The anti-TIGIT antagonist antibody, PD-1 axis binding antagonist, taxane, and platinum agent for use of embodiment 434, wherein the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks.

436. The anti-TIGIT antagonist antibody, PD-1 axis binding antagonist, taxane, and platinum agent for use of any one of embodiments 417-435, wherein the length of each of the one or more dosing cycles is 21 days.

437. The anti-TIGIT antagonist antibody, PD-1 axis binding antagonist, taxane, and platinum agent for use of any one of embodiments 417-436, wherein the anti-TIGIT antagonist antibody, PD-1 axis binding antagonist, the taxane, and the platinum agent are administered in each of 4-8 induction phase dosing cycles.

438. The anti-TIGIT antagonist antibody, PD-1 axis binding antagonist, taxane, and platinum agent for use of embodiment 437, wherein the anti-TIGIT antagonist antibody, PD-1 axis binding antagonist, the taxane, and the platinum agent are administered in each of six induction phase dosing cycles.

439. The anti-TIGIT antagonist antibody, PD-1 axis binding antagonist, taxane, and platinum agent for use of embodiment 437 or 438, wherein the anti-TIGIT antagonist antibody and the PD-1 axis binding antagonist are further administered in one or more maintenance phase dosing cycles following the induction phase dosing cycles.

440. The anti-TIGIT antagonist antibody, PD-1 axis binding antagonist, taxane, and platinum agent for use of embodiment 439, wherein the taxane and the platinum agent are omitted from each of the one or more maintenance phase dosing cycles.

441. The anti-TIGIT antagonist antibody, PD-1 axis binding antagonist, taxane, and platinum agent for use of any one of embodiments 437-410, wherein the length of each of the induction phase dosing cycles and/or the one or more maintenance phase dosing cycles is 21 days.

442. The anti-TIGIT antagonist antibody, PD-1 axis binding antagonist, taxane, and platinum agent for use of any one of embodiments 417-423, wherein the anti-TIGIT antagonist antibody is administered at a fixed dose of about 300 mg to about 800 mg every two weeks.

443. The anti-TIGIT antagonist antibody, PD-1 axis binding antagonist, taxane, and platinum agent for use of embodiment 442, wherein the anti-TIGIT antagonist antibody is administered at a fixed dose of about 400 mg to about 500 mg every two weeks.

444. The anti-TIGIT antagonist antibody, PD-1 axis binding antagonist, taxane, and platinum agent for use of embodiment 443, wherein the anti-TIGIT antagonist antibody is administered at a fixed dose of about 420 mg every two weeks.

445. The anti-TIGIT antagonist antibody, PD-1 axis binding antagonist, taxane, and platinum agent for use of any one of embodiments 417-423 and 442-444, wherein the PD-1 axis binding antagonist is administered at a fixed dose of about 200 mg to about 1200 mg every two weeks.

446. The anti-TIGIT antagonist antibody, PD-1 axis binding antagonist, taxane, and platinum agent for use of embodiment 445, wherein the PD-1 axis binding antagonist is administered at a fixed dose of about 800 mg to about 1000 mg every two weeks.

447. The anti-TIGIT antagonist antibody, PD-1 axis binding antagonist, taxane, and platinum agent for use of embodiment 446, wherein the PD-1 axis binding antagonist is administered at a fixed dose of about 840 mg every two weeks.

448. The anti-TIGIT antagonist antibody, PD-1 axis binding antagonist, taxane, and platinum agent for use of embodiment 447, wherein the anti-TIGIT antagonist antibody is administered at a fixed dose of about 420 mg every two weeks and the PD-1 axis binding antagonist is administered at a fixed dose of about 840 mg every two weeks.

449. The anti-TIGIT antagonist antibody, PD-1 axis binding antagonist, taxane, and platinum agent for use of any one of embodiments 442-448, wherein the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

450. The anti-TIGIT antagonist antibody, PD-1 axis binding antagonist, taxane, and platinum agent for use of any one of embodiments 417-423, wherein the anti-TIGIT antagonist antibody is administered at a fixed dose of about 700 mg to about 1000 mg every four weeks.

451. The anti-TIGIT antagonist antibody, PD-1 axis binding antagonist, taxane, and platinum agent for use of embodiment 450, wherein the anti-TIGIT antagonist antibody is administered at a fixed dose of about 800 mg to about 900 mg every four weeks.

452. The anti-TIGIT antagonist antibody, PD-1 axis binding antagonist, taxane, and platinum agent for use of embodiment 451, wherein the anti-TIGIT antagonist antibody is administered at a fixed dose of about 840 mg every four weeks.

453. The anti-TIGIT antagonist antibody, PD-1 axis binding antagonist, taxane, and platinum agent for use of any one of embodiments 417-423 and 450-452, wherein the PD-1 axis binding antagonist is administered at a fixed dose of about 400 mg to about 2000 mg every four weeks.

454. The anti-TIGIT antagonist antibody, PD-1 axis binding antagonist, taxane, and platinum agent for use of embodiment 453, wherein the PD-1 axis binding antagonist is administered at a fixed dose of about 1600 mg to about 1800 mg every four weeks.

455. The anti-TIGIT antagonist antibody, PD-1 axis binding antagonist, taxane, and platinum agent for use of embodiment 454, wherein the PD-1 axis binding antagonist is administered at a fixed dose of about 1680 mg every four weeks.

456. The anti-TIGIT antagonist antibody, PD-1 axis binding antagonist, taxane, and platinum agent for use of embodiment 455, wherein the anti-TIGIT antagonist antibody is administered at a fixed dose of about 840 mg every four weeks and the PD-1 axis binding antagonist is administered at a fixed dose of about 1680 mg every four weeks.

457. The anti-TIGIT antagonist antibody, PD-1 axis binding antagonist, taxane, and platinum agent for use of any one of embodiments 450-456, wherein the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

458. The anti-TIGIT antagonist antibody, PD-1 axis binding antagonist, taxane, and platinum agent for use of any one of embodiments 442-457, wherein the taxane is administered once per week, once every two weeks, once every three weeks, twice every three weeks, once every four weeks, twice every four weeks, or three times every four weeks.

459. The anti-TIGIT antagonist antibody, PD-1 axis binding antagonist, taxane, and platinum agent for use of any one of embodiments 442-458, wherein the platinum agent is administered once per week, once every two weeks, once every three weeks, twice every three weeks, once every four weeks, twice every four weeks, or three times every four weeks.

460. The anti-TIGIT antagonist antibody, PD-1 axis binding antagonist, taxane, and platinum agent for use of any one of embodiments 417-459, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

461. The anti-TIGIT antagonist antibody, PD-1 axis binding antagonist, taxane, and platinum agent for use of embodiment 460, wherein the anti-TIGIT antagonist antibody further comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

462. The anti-TIGIT antagonist antibody, PD-1 axis binding antagonist, taxane, and platinum agent for use of embodiment 460 or 461, wherein the anti-TIGIT antagonist antibody further comprises the following heavy chain variable region FRs:

an FR-H1 comprising the amino acid sequence of XiVQLQQSGPGLVKPSQTLSLTCAISGDSVS (SEQ ID NO: 11), wherein Xi is E or Q;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

463. The anti-TIGIT antagonist antibody, PD-1 axis binding antagonist, taxane, and platinum agent for use of embodiment 462, wherein Xi is E.

464. The anti-TIGIT antagonist antibody, PD-1 axis binding antagonist, taxane, and platinum agent for use of embodiment 462, wherein Xi is Q.

465. The anti-TIGIT antagonist antibody, PD-1 axis binding antagonist, taxane, and platinum agent for use of any one of embodiments 460-464, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

466. The anti-TIGIT antagonist antibody, PD-1 axis binding antagonist, taxane, and platinum agent for use of any one of embodiments 417-465, wherein the anti-TIGIT antagonist antibody comprises:

(a) a VH domain comprising the amino acid sequence of SEQ ID NO: 17 or 18; and

(b) a VL domain comprising the amino acid sequence of SEQ ID NO: 19.

467. The anti-TIGIT antagonist antibody, PD-1 axis binding antagonist, taxane, and platinum agent for use of any one of embodiments 417-466, wherein the anti-TIGIT antagonist antibody is a monoclonal antibody.

468. The anti-TIGIT antagonist antibody, PD-1 axis binding antagonist, taxane, and platinum agent for use of embodiment 467, wherein the anti-TIGIT antagonist antibody is a human antibody.

469. The anti-TIGIT antagonist antibody, PD-1 axis binding antagonist, taxane, and platinum agent for use of any one of embodiments 417-468, wherein the anti-TIGIT antagonist antibody is a full-length antibody.

470. The anti-TIGIT antagonist antibody, PD-1 axis binding antagonist, taxane, and platinum agent for use of any one of embodiments 417-459 and 467-469, wherein the anti-TIGIT antagonist antibody has intact Fc-mediated effector function.

471. The anti-TIGIT antagonist antibody, PD-1 axis binding antagonist, taxane, and platinum agent for use of embodiment 470, wherein the anti-TIGIT antagonist antibody is tiragolumab, vibostolimab, etigilimab, EOS084448, SGN-TGT, or TJ-T6.

472. The anti-TIGIT antagonist antibody, PD-1 axis binding antagonist, taxane, and platinum agent for use of any one of embodiments 417-463 and 465-471, wherein the anti-TIGIT antagonist antibody is tiragolumab.

473. The anti-TIGIT antagonist antibody, PD-1 axis binding antagonist, taxane, and platinum agent for use of any one of embodiments 417-459 and 467-469, wherein the anti-TIGIT antagonist antibody has enhanced Fc-mediated effector function.

474. The anti-TIGIT antagonist antibody, PD-1 axis binding antagonist, taxane, and platinum agent for use of any one of embodiments 417-459, 467-471, and 473, wherein the anti-TIGIT antagonist antibody is SGN-TGT.

475. The anti-TIGIT antagonist antibody, PD-1 axis binding antagonist, taxane, and platinum agent for use of any one of embodiments 417-459 and 467-469, wherein the anti-TIGIT antagonist antibody does not have Fc-mediated effector function.

476. The anti-TIGIT antagonist antibody, PD-1 axis binding antagonist, taxane, and platinum agent for use of any one of embodiments 417-459, 467-469, and 475, wherein the anti-TIGIT antagonist antibody is domvanalimab, BMS-986207, ASP8374, or COM902.

477. The anti-TIGIT antagonist antibody, PD-1 axis binding antagonist, taxane, and platinum agent for use of any one of embodiments 417-468, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

478. The anti-TIGIT antagonist antibody, PD-1 axis binding antagonist, taxane, and platinum agent for use of any one of embodiments 417-459 and 467-476, wherein the anti-TIGIT antagonist antibody is an IgG class antibody.

479. The anti-TIGIT antagonist antibody, PD-1 axis binding antagonist, taxane, and platinum agent for use of embodiment 478, wherein the IgG class antibody is an IgG1 subclass antibody.

480. The anti-TIGIT antagonist antibody, PD-1 axis binding antagonist, taxane, and platinum agent for use of embodiment 479, wherein the anti-TIGIT antagonist antibody is tiragolumab, vibostolimab, etigilimab, EOS084448, SGN-TGT, TJ-T6, BGB-A1217, AB308, domvanalimab, or BMS-986207.

481. The anti-TIGIT antagonist antibody, PD-1 axis binding antagonist, taxane, and platinum agent for use of embodiment 480, wherein the anti-TIGIT antagonist antibody is tiragolumab.

482. The anti-TIGIT antagonist antibody, PD-1 axis binding antagonist, taxane, and platinum agent for use of embodiment 478, wherein the IgG class antibody is an IgG4 subclass antibody.

483. The anti-TIGIT antagonist antibody, PD-1 axis binding antagonist, taxane, and platinum agent for use of embodiment 482, wherein the anti-TIGIT antagonist antibody is ASP8374 or COM902.

484. The anti-TIGIT antagonist antibody, PD-1 axis binding antagonist, taxane, and platinum agent for use of any one of embodiments 417-483, wherein the PD-1 axis binding antagonist is a PD-L1 binding antagonist or a PD-1 binding antagonist.

485. The anti-TIGIT antagonist antibody, PD-1 axis binding antagonist, taxane, and platinum agent for use of embodiment 484, wherein the PD-L1 binding antagonist is an anti-PD-L1 antagonist antibody.

486. The anti-TIGIT antagonist antibody, PD-1 axis binding antagonist, taxane, and platinum agent for use of any one of embodiments 417-485, wherein the anti-PD-L1 antagonist antibody is atezolizumab (MPDL3280A), MSB0010718C, MDX-1105, or MED14736.

487. The anti-TIGIT antagonist antibody, PD-1 axis binding antagonist, taxane, and platinum agent for use of embodiment 486, wherein the anti-PD-L1 antagonist antibody is atezolizumab.

488. The anti-TIGIT antagonist antibody, PD-1 axis binding antagonist, taxane, and platinum agent for use of embodiment 487, wherein the PD-1 binding antagonist is an anti-PD-1 antagonist antibody.

489. The anti-TIGIT antagonist antibody, PD-1 axis binding antagonist, taxane, and platinum agent for use of embodiment 488, wherein the anti-PD-1 antagonist antibody is nivolumab (MDX-1106), pembrolizumab (MK-3475), or AMP-224.

490. The anti-TIGIT antagonist antibody, PD-1 axis binding antagonist, taxane, and platinum agent for use of any one of embodiments 417-485, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

491. The anti-TIGIT antagonist antibody, PD-1 axis binding antagonist, taxane, and platinum agent for use of embodiment 490, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

492. The anti-TIGIT antagonist antibody, PD-1 axis binding antagonist, taxane, and platinum agent for use of any one of embodiments 417-491, wherein the anti-PD-L1 antagonist antibody comprises:

a VH domain comprising the amino acid sequence of SEQ ID NO: 26; and

a VL domain comprising the amino acid sequence of SEQ ID NO: 27.

493. The anti-TIGIT antagonist antibody, PD-1 axis binding antagonist, taxane, and platinum agent for use of any one of embodiments 490-492, wherein the anti-PD-L1 antagonist antibody is a monoclonal antibody.

494. The anti-TIGIT antagonist antibody, PD-1 axis binding antagonist, taxane, and platinum agent for use of embodiment 493, wherein the anti-PD-L1 antagonist antibody is a humanized antibody.

495. The anti-TIGIT antagonist antibody, PD-1 axis binding antagonist, taxane, and platinum agent for use of embodiment 493 or 494, wherein the anti-PD-L1 antagonist antibody is a full-length antibody.

496. The anti-TIGIT antagonist antibody, PD-1 axis binding antagonist, taxane, and platinum agent for use of any one of embodiments 490-494, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

497. The anti-TIGIT antagonist antibody, PD-1 axis binding antagonist, taxane, and platinum agent for use of any one of embodiments 490-494, wherein the anti-PD-L1 antagonist antibody is an IgG class antibody.

498. The anti-TIGIT antagonist antibody, PD-1 axis binding antagonist, taxane, and platinum agent for use of embodiment 497, wherein the IgG class antibody is an IgG1 subclass antibody.

499. The anti-TIGIT antagonist antibody, PD-1 axis binding antagonist, taxane, and platinum agent for use of any one of embodiments 417-498, wherein the taxane is paclitaxel or nab-paclitaxel.

500. The anti-TIGIT antagonist antibody, PD-1 axis binding antagonist, taxane, and platinum agent for use of embodiment 499, wherein the taxane is paclitaxel. 501. The anti-TIGIT antagonist antibody, PD-1 axis binding antagonist, taxane, and platinum agent for use of any one of embodiments 417-500, wherein the platinum agent is cisplatin or carboplatin.

502. The anti-TIGIT antagonist antibody, PD-1 axis binding antagonist, taxane, and platinum agent for use of embodiment 501, wherein the platinum agent is cisplatin.

503. The anti-TIGIT antagonist antibody, PD-1 axis binding antagonist, taxane, and platinum agent for use of any one of embodiments 417-502, wherein the method comprises administering to the subject the PD-1 axis binding antagonist before the anti-TIGIT antagonist antibody.

504. The anti-TIGIT antagonist antibody, PD-1 axis binding antagonist, taxane, and platinum agent for use of embodiment 503, wherein the method comprises a first observation period following administration of the PD-1 axis binding antagonist and a second observation period following administration of the anti-TIGIT antagonist antibody.

505. The anti-TIGIT antagonist antibody, PD-1 axis binding antagonist, taxane, and platinum agent for use of embodiment 504, wherein the first observation period and the second observation period are each between about 30 minutes to about 60 minutes in length.

506. The anti-TIGIT antagonist antibody, PD-1 axis binding antagonist, taxane, and platinum agent for use of any one of embodiments 417-502, wherein the method comprises administering to the subject the anti-TIGIT antagonist antibody before the PD-1 axis binding antagonist.

507. The anti-TIGIT antagonist antibody, PD-1 axis binding antagonist, taxane, and platinum agent for use of embodiment 506, wherein the method comprises a first observation period following administration of the anti-TIGIT antagonist antibody and a second observation period following administration of the PD-1 axis binding antagonist.

508. The anti-TIGIT antagonist antibody, PD-1 axis binding antagonist, taxane, and platinum agent for use of embodiment 507, wherein the first observation period and the second observation period are each between about 30 minutes to about 60 minutes in length.

509. The anti-TIGIT antagonist antibody, PD-1 axis binding antagonist, taxane, and platinum agent for use of any one of embodiments 417-502, wherein the method comprises administering to the subject the anti-TIGIT antagonist antibody and the PD-1 axis binding antagonist simultaneously.

510. The anti-TIGIT antagonist antibody, PD-1 axis binding antagonist, taxane, and platinum agent for use of any one of embodiments 417-510, wherein the anti-TIGIT antagonist antibody and the PD-1 axis binding antagonist are administered before the taxane or the platinum agent.

511. The anti-TIGIT antagonist antibody, PD-1 axis binding antagonist, taxane, and platinum agent for use of embodiment 510, wherein the anti-TIGIT antagonist antibody and the PD-1 axis binding antagonist are administered before the taxane and the platinum agent.

512. The anti-TIGIT antagonist antibody, PD-1 axis binding antagonist, taxane, and platinum agent for use of embodiment 511, wherein the method comprises administering to the subject the taxane before the platinum agent.

513. The anti-TIGIT antagonist antibody, PD-1 axis binding antagonist, taxane, and platinum agent for use of embodiment 512, wherein the method comprises a third observation period following administration of the taxane and a fourth observation period following administration of the platinum agent.

514. The anti-TIGIT antagonist antibody, PD-1 axis binding antagonist, taxane, and platinum agent for use of embodiment 513, wherein the third observation period and the fourth observation period are each between about 30 minutes to about 60 minutes in length.

515. The anti-TIGIT antagonist antibody, PD-1 axis binding antagonist, taxane, and platinum agent for use of any one of embodiments 417-514, wherein the method comprises administering to the subject the anti-TIGIT antagonist antibody, the PD-1 axis binding antagonist, the taxane, and the platinum agent intravenously.

516. The anti-TIGIT antagonist antibody, PD-1 axis binding antagonist, taxane, and platinum agent for use of embodiment 515, wherein the method comprises administering to the subject the anti-TIGIT antagonist antibody by intravenous infusion over 60±10 minutes.

517. The anti-TIGIT antagonist antibody, PD-1 axis binding antagonist, taxane, and platinum agent for use of embodiment 515 or 516, wherein the method comprises administering to the subject the PD-1 axis binding antagonist by intravenous infusion over 60±15 minutes.

518. The anti-TIGIT antagonist antibody, PD-1 axis binding antagonist, taxane, and platinum agent for use of any one of embodiments 515-517, wherein the method comprises administering to the subject the taxane by intravenous infusion over 3 hours±30 minutes.

519. The anti-TIGIT antagonist antibody, PD-1 axis binding antagonist, taxane, and platinum agent for use of any one of embodiments 515-518, wherein the method comprises administering to the subject the platinum agent by intravenous infusion over 1-4 hours.

520. The anti-TIGIT antagonist antibody, PD-1 axis binding antagonist, taxane, and platinum agent for use of any one of embodiments 417-519, wherein an ESCC tumor sample obtained from the subject has been determined to have a detectable expression level of PD-L1.

521. The anti-TIGIT antagonist antibody, PD-1 axis binding antagonist, taxane, and platinum agent for use of embodiment 520, wherein the detectable expression level of PD-L1 is a detectable protein expression level of PD-L1.

522. The anti-TIGIT antagonist antibody, PD-1 axis binding antagonist, taxane, and platinum agent for use of embodiment 521, wherein the detectable protein expression level of PD-L1 has been determined by an immunohistochemical (IHC) assay.

523. The anti-TIGIT antagonist antibody, PD-1 axis binding antagonist, taxane, and platinum agent for use of embodiment 522, wherein the IHC assay uses anti-PD-L1 antibody SP263, 22C3, SP142, or 28-8.

524. The anti-TIGIT antagonist antibody, PD-1 axis binding antagonist, taxane, and platinum agent for use of embodiment 523, wherein the IHC assay uses anti-PD-L1 antibody SP263.

525. The anti-TIGIT antagonist antibody, PD-1 axis binding antagonist, taxane, and platinum agent for use of embodiment 524, wherein the IHC assay is the Ventana SP263 Companion Diagnostic (CDx) assay.

526. The anti-TIGIT antagonist antibody, PD-1 axis binding antagonist, taxane, and platinum agent for use of embodiment 525, wherein the ESCC tumor sample has been determined to have a tumor and tumor-associated immune cell (TIC) score of greater than, or equal to, 1%.

527. The anti-TIGIT antagonist antibody, PD-1 axis binding antagonist, taxane, and platinum agent for use of embodiment 526, wherein the TIC score is greater than, or equal to, 10%.

528. The anti-TIGIT antagonist antibody, PD-1 axis binding antagonist, taxane, and platinum agent for use of embodiment 525 or 526, wherein the ESCC tumor sample has been determined to have a TIC score of less than 10%.

529. The anti-TIGIT antagonist antibody, PD-1 axis binding antagonist, taxane, and platinum agent for use of embodiment 527, wherein the TIC score is greater than, or equal to, 10% and less than 50%.

530. The anti-TIGIT antagonist antibody, PD-1 axis binding antagonist, taxane, and platinum agent for use of embodiment 523, wherein the IHC assay uses the anti-PD-L1 antibody 22C3.

531. The anti-TIGIT antagonist antibody, PD-1 axis binding antagonist, taxane, and platinum agent for use of embodiment 530, wherein the IHC assay is the pharmDx 22C3 IHC assay.

532. The anti-TIGIT antagonist antibody, PD-1 axis binding antagonist, taxane, and platinum agent for use of embodiment 531, wherein the ESCC tumor sample has been determined to have a combined positive score (CPS) of greater than, or equal to, 10 or a TPS of greater than, or equal to, 1%.

533. The anti-TIGIT antagonist antibody, PD-1 axis binding antagonist, taxane, and platinum agent for use of embodiment 523, wherein the IHC assay uses the anti-PD-L1 antibody SP142.

534. The anti-TIGIT antagonist antibody, PD-1 axis binding antagonist, taxane, and platinum agent for use of embodiment 533, wherein the IHC assay is the Ventana SP142 IHC assay.

535. The anti-TIGIT antagonist antibody, PD-1 axis binding antagonist, taxane, and platinum agent for use of embodiment 523, wherein the IHC assay uses the anti-PD-L1 antibody 28-8.

536. The anti-TIGIT antagonist antibody, PD-1 axis binding antagonist, taxane, and platinum agent for use of embodiment 535, wherein the IHC assay is the pharmDx 28-8 IHC assay.

537. The anti-TIGIT antagonist antibody, PD-1 axis binding antagonist, taxane, and platinum agent for use of embodiment 520, wherein the detectable expression level of PD-L1 is a detectable nucleic acid expression level of PD-L1.

538. The anti-TIGIT antagonist antibody, PD-1 axis binding antagonist, taxane, and platinum agent for use of embodiment 537, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

539. The anti-TIGIT antagonist antibody, PD-1 axis binding antagonist, taxane, and platinum agent for use of any one of embodiments 417-538, wherein the advanced ESCC is a locally advanced ESCC.

540. The anti-TIGIT antagonist antibody, PD-1 axis binding antagonist, taxane, and platinum agent for use of any one of embodiments 417-539, wherein the advanced ESCC is a recurrent or metastatic ESCC.

541. The anti-TIGIT antagonist antibody, PD-1 axis binding antagonist, taxane, and platinum agent for use of any one of embodiments 417-540, wherein the advanced ESCC is an unresectable ESCC.

542. The anti-TIGIT antagonist antibody, PD-1 axis binding antagonist, taxane, and platinum agent for use of any one of embodiments 417-541, wherein the treatment results in a progression-free survival (PFS) of about 8 months or more.

543. The anti-TIGIT antagonist antibody, PD-1 axis binding antagonist, taxane, and platinum agent for use of any one of embodiments 417-542, wherein the treatment results in an increase in a PFS of the subject as compared to treatment with the taxane and the platinum agent, without the PD-1 axis binding antagonist and the anti-TIGIT antagonist antibody

544. The anti-TIGIT antagonist antibody, PD-1 axis binding antagonist, taxane, and platinum agent for use of embodiment 543, wherein the treatment extends the PFS of the subject or population of subjects by at least about 2 months or about 4 months.

545. The anti-TIGIT antagonist antibody, PD-1 axis binding antagonist, taxane, and platinum agent for use of embodiment 544, wherein the treatment results in a median PFS of the population of subjects of about 6 months to about 10 months.

546. The anti-TIGIT antagonist antibody, PD-1 axis binding antagonist, taxane, and platinum agent for use of any one of embodiments 417-545, wherein the treatment results in an overall survival (OS) of about 18 months or more.

547. The anti-TIGIT antagonist antibody, PD-1 axis binding antagonist, taxane, and platinum agent for use of any one of embodiments 417-546, wherein the treatment results in an increase in OS of the subject as compared to treatment with the taxane and the platinum agent, without the PD-1 axis binding antagonist and the anti-TIGIT antagonist antibody.

548. The anti-TIGIT antagonist antibody, PD-1 axis binding antagonist, taxane, and platinum agent for use of embodiment 547, wherein the treatment extends the OS of the subject or population of subjects by at least about 4 months or about 6 months.

549. The anti-TIGIT antagonist antibody, PD-1 axis binding antagonist, taxane, and platinum agent for use of embodiment 547, wherein the treatment results in a median OS of the population of subjects of about 14 months to about 20 months.

550. The anti-TIGIT antagonist antibody, PD-1 axis binding antagonist, taxane, and platinum agent for use of any one of embodiments 417-549, wherein the treatment results in an increase in duration of objective response (DOR) in the subject as compared to treatment with the taxane and the platinum agent, without the PD-1 axis binding antagonist and the anti-TIGIT antagonist antibody.

551. The anti-TIGIT antagonist antibody, PD-1 axis binding antagonist, taxane, and platinum agent for use of any one of embodiments 417-550, wherein the treatment results in a complete response or a partial response.

552. Use of an anti-TIGIT antagonist antibody in the manufacture of a medicament for treating a subject having an advanced ESCC in combination with a PD-1 axis binding antagonist, a taxane, and a platinum agent, wherein the treatment is according to the method of any one of embodiments 261-395.

553. Use of a PD-1 axis binding antagonist in the manufacture of a medicament for treating a subject having an advanced ESCC in combination with an anti-TIGIT antagonist antibody, a taxane, and a platinum agent, wherein the treatment is according to the method of any one of embodiments 261-395.

554. The use of embodiment 552 or 553, wherein the anti-TIGIT antagonist antibody and the PD-1 axis binding antagonist are formulated separately.

555. The use of embodiment 552 or 553, wherein the anti-TIGIT antagonist antibody and the PD-1 axis binding antagonist are formulated together.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-283. (canceled)

284. A method for treating a subject or population of subjects having an esophageal squamous cell carcinoma (ESCC), the method comprising administering to the subject or population of subjects one or more dosing cycles of an anti-TIGIT antagonist antibody and a PD-1 axis binding antagonist, wherein the subject or population of subjects previously received definitive chemoradiation treatment for ESCC.

285. The method of claim 284, wherein (a) the definitive chemoradiation treatment was completed no more than 89 days prior to administration with the anti-TIGIT antagonist antibody or the PD-1 axis binding antagonist or (b) the definitive chemoradiation treatment comprises at least two cycles of platinum-based chemotherapy and radiation therapy without evidence of radiographic disease progression.

286. The method of claim 284, wherein no chemotherapy is administered to the subject or population of subjects during the one or more dosing cycles.

287. The method of claim 284, wherein:
(a) the anti-TIGIT antagonist antibody is administered at a fixed dose of about 30 mg to about 1200 mg every three weeks; about 30 mg to about 800 mg every three weeks; or 600 mg every three weeks; or
(b) the PD-1 axis binding antagonist is administered at a fixed dose of about 80 mg to about 1600 mg every three weeks; about 800 mg to about 1400 mg every three weeks; or about 1200 mg every three weeks.

288. The method of claim 287, wherein the anti-TIGIT antagonist antibody is administered at a fixed dose of about 600 mg every three weeks and the PD-1 axis binding antagonist is administered at a fixed dose of about 1200 mg every three weeks.

289. The method of claim 284, wherein:
(a) (i) the anti-TIGIT antagonist antibody is administered at a fixed dose of about 300 mg to about 800 mg every two weeks; about 400 mg to about 500 mg every two weeks; or about 420 mg every two weeks; and (ii) the PD-1 axis binding antagonist is administered at a fixed dose of about 200 mg to about 1200 mg every two weeks; about 800 mg to about 1000 mg every two weeks; or about 840 mg every two weeks; or
(b) (i) the anti-TIGIT antagonist antibody is administered at a fixed dose of about 700 mg to about 1000 mg every four weeks; about 800 mg to about 900 mg every four weeks; or about 840 mg every four weeks; and (ii) the PD-1 axis binding antagonist is administered at a fixed dose of about 400 mg to about 2000 mg every four weeks; about 1600 mg to about 1800 mg every four weeks; or about 1680 mg every four weeks.

290. The method of claim 289, wherein:
(a) the anti-TIGIT antagonist antibody is administered at a fixed dose of about 420 mg every two weeks and the PD-1 axis binding antagonist is administered at a fixed dose of about 840 mg every two weeks; or
(b) the anti-TIGIT antagonist antibody is administered at a fixed dose of about 840 mg every four weeks and the PD-1 axis binding antagonist is administered at a fixed dose of about 1680 mg every four weeks.

291. The method of claim 284, wherein the anti-TIGIT antagonist antibody is a monoclonal antibody and/or a human antibody.

292. The method of claim 284, wherein the anti-TIGIT antagonist antibody is a full-length antibody.

293. The method of claim 284, wherein:
(a) the anti-TIGIT antagonist antibody is tiragolumab, vibostolimab, etigilimab, or EOS084448;
(b) the anti-TIGIT antagonist antibody comprises the following hypervariable regions (HVRs):
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6); and/or
(c) the anti-TIGIT antagonist antibody comprises a VH domain comprising the amino acid sequence of SEQ ID NO: 17 or 18 and a VL domain comprising the amino acid sequence of SEQ ID NO: 19.

294. The method of claim 293, wherein the anti-TIGIT antagonist antibody is tiragolumab.

295. The method of claim 284, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

296. The method of claim 284, wherein the PD-1 axis binding antagonist is a PD-L1 binding antagonist or a PD-1 binding antagonist.

297. The method of claim 296, wherein the PD-L1 binding antagonist is an anti-PD-L1 antagonist antibody.

298. The method of claim 284, wherein:
(a) the anti-PD-L1 antagonist antibody is atezolizumab (MPDL3280A), MSB00107180, MDX-1105, or MED14736;
(b) the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25); and/or
(c) the anti-PD-L1 antagonist antibody comprises a VH domain comprising the amino acid sequence of SEQ ID NO: 26 and a VL domain comprising the amino acid sequence of SEQ ID NO: 27.

299. The method of claim 298, wherein the anti-PD-L1 antagonist antibody is atezolizumab.

300. The method of claim 296, wherein the PD-1 binding antagonist is an anti-PD-1 antagonist antibody.

301. The method of claim 284, wherein the method comprises administering to the subject or population of subjects the anti-TIGIT antagonist antibody and the PD-1 axis binding antagonist on about Day 1 of each of the one or more dosing cycles.

302. The method of claim 284, wherein the method comprises (a) administering to the subject or population of subjects the PD-1 axis binding antagonist before the anti-TIGIT antagonist antibody; (b) administering to the subject or population of subjects the anti-TIGIT antagonist antibody before the PD-1 axis binding antagonist; or (c) administering to the subject or population of subjects the anti-TIGIT antagonist antibody and the PD-1 axis binding antagonist simultaneously.

303. The method of claim 284, wherein an ESCC tumor sample obtained from the subject or population of subjects has been determined to have a detectable expression level of PD-L1.

304. The method of claim 303, wherein the detectable expression level of PD-L1 is a detectable protein expression level of PD-L1 or a detectable nucleic acid expression level of PD-L1.

305. The method of claim 284, wherein the ESCC is a locally advanced ESCC; an unresectable ESCC; a recurrent or metastatic ESCC; or a cervical esophageal tumor.

306. The method of claim 284, wherein the ESCC is a Stage II ESCC, a Stage III ESCC, or a Stage IV ESCC.

307. The method of claim 284, wherein (a) the subject or population of subjects has not been treated previously with cancer immunotherapy; or (b) the subject or population of subjects has completed a previous cancer immunotherapy for ESCC.

308. The method of claim 284, wherein the treatment results in an increase in progression-free survival (PFS), overall survival (OS), and/or duration of objective response (DOR) in the subject or population of subjects as compared to (i) treatment with the PD-1 axis binding antagonist without the anti-TIGIT antagonist antibody; (ii) treatment with the anti-TIGIT antagonist antibody without the PD-1 axis binding antagonist; or (iii) treatment without the anti-TIGIT antagonist antibody and without the PD-1 axis binding antagonist.

309. The method of claim 284, wherein the treatment results in a complete response or a partial response.

310. The method of claim 284, wherein the method comprises administering to the subject or population of subjects at least five dosing cycles.

311. A method for treating a subject or population of subjects having an advanced ESCC for whom surgery is unsuitable, the method comprising administering to the subject or population of subjects one or more dosing cycles of an anti-TIGIT antagonist antibody, a PD-1 axis binding antagonist, a taxane, and a platinum agent.

312. The method of claim 311, wherein the subject or population of subjects (a) has received no prior systemic treatment for advanced ESCC; (b) has received no prior systemic treatment for non-advanced ESCC; or (c) has received prior treatment for non-advanced ESCC, wherein the prior treatment for the non-advanced ESCC was completed at least six months before diagnosis of the advanced ESCC.

313. The method of claim 312, wherein the prior treatment for the non-advanced ESCC comprises a chemoradiotherapy or a chemotherapy.

314. The method of claim 313, wherein the chemoradiotherapy or chemotherapy was administered with curative intent or in an adjuvant or neoadjuvant setting.

315. The method of claim 311, wherein:
(a) the anti-TIGIT antagonist antibody is administered at a fixed dose of about 30 mg to about 1200 mg every three weeks; about 30 mg to about 800 mg every three weeks; or 600 mg every three weeks; or
(b) the PD-1 axis binding antagonist is administered at a fixed dose of about 80 mg to about 1600 mg every three weeks; about 800 mg to about 1400 mg every three weeks; or about 1200 mg every three weeks.

316. The method of claim 311, wherein:
(a) the taxane is administered at a dose of (i) about 100-250 mg/m² every three weeks; (ii) 150-200 mg/m² every three weeks; or (iii) 175 mg/m² every three weeks; or
(b) the platinum agent is administered at a dose of (i) about 20-200 mg/m² every three weeks; (ii) about 40-120 mg/m² every three weeks; or (iii) about 60-80 mg/m² every three weeks.

317. The method of claim 316, wherein the anti-TIGIT antagonist antibody is administered at a fixed dose of about 600 mg every three weeks, the PD-1 axis binding antagonist is administered at a fixed dose of about 1200 mg every three weeks, the taxane is administered at a dose of about 175 mg/m² every three weeks, and the platinum agent is administered at a dose of about 60-80 mg/m² every three weeks.

318. The method of claim 311, wherein the anti-TIGIT antagonist antibody, PD-1 axis binding antagonist, the taxane, and the platinum agent are administered in each of 4-8 induction phase dosing cycles.

319. The method of claim 318, wherein the anti-TIGIT antagonist antibody and the PD-1 axis binding antagonist are further administered in one or more maintenance phase dosing cycles following the induction phase dosing cycles.

320. The method of claim 319, wherein the taxane and the platinum agent are omitted from each of the one or more maintenance phase dosing cycles.

321. The method of claim 311, wherein:
(a) (i) the anti-TIGIT antagonist antibody is administered at a fixed dose of about 300 mg to about 800 mg every two weeks; about 400 mg to about 500 mg every two weeks; or about 420 mg every two weeks; and (ii) the PD-1 axis binding antagonist is administered at a fixed dose of about 200 mg to about 1200 mg every two weeks; about 800 mg to about 1000 mg every two weeks; or about 840 mg every two weeks; or
(b) (i) the anti-TIGIT antagonist antibody is administered at a fixed dose of about 700 mg to about 1000 mg every four weeks; about 800 mg to about 900 mg every four weeks; or about 840 mg every four weeks; and (ii) the PD-1 axis binding antagonist is administered at a fixed dose of about 400 mg to about 2000 mg every four weeks; about 1600 mg to about 1800 mg every four weeks; or about 1680 mg every four weeks.

322. The method of claim 321, wherein (a) the anti-TIGIT antagonist antibody is administered at a fixed dose of about 420 mg every two weeks and the PD-1 axis binding antagonist is administered at a fixed dose of about 840 mg every two weeks; or
(b) the anti-TIGIT antagonist antibody is administered at a fixed dose of about 840 mg every four weeks and the PD-1 axis binding antagonist is administered at a fixed dose of about 1680 mg every four weeks.

323. The method of claim 321, wherein the anti-TIGIT antagonist antibody and the PD-1 axis binding antagonist are further administered in one or more maintenance phase dosing cycles, wherein the taxane and the platinum agent are omitted from each of the one or more maintenance phase dosing cycles.

324. The method of claim 321, wherein (a) the taxane is administered once per week, once every two weeks, once every three weeks, twice every three weeks, once every four weeks, twice every four weeks, or three times every four weeks; or (b) the platinum agent is administered once per week, once every two weeks, once every three weeks, twice every three weeks, once every four weeks, twice every four weeks, or three times every four weeks.

325. The method of claim 311, wherein the anti-TIGIT antagonist antibody is a monoclonal antibody and/or a human antibody.

326. The method of claim 311, wherein the anti-TIGIT antagonist antibody is a full-length antibody.

327. The method of claim 311, wherein:
(a) the anti-TIGIT antagonist antibody is tiragolumab, vibostolimab, etigilimab, or EOS084448;
(b) the anti-TIGIT antagonist antibody comprises the following hypervariable regions (HVRs):
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6); and/or
(c) the anti-TIGIT antagonist antibody comprises a VH domain comprising the amino acid sequence of SEQ ID NO: 17 or 18 and a VL domain comprising the amino acid sequence of SEQ ID NO: 19.

328. The method of claim 327, wherein the anti-TIGIT antagonist antibody is tiragolumab.

329. The method of claim 311, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

330. The method of claim 311, wherein the PD-1 axis binding antagonist is a PD-L1 binding antagonist or a PD-1 binding antagonist.

331. The method of claim 330, wherein the PD-L1 binding antagonist is an anti-PD-L1 antagonist antibody.

332. The method of claim 311, wherein:
(a) the anti-PD-L1 antagonist antibody is atezolizumab (MPDL3280A), MSB00107180, MDX-1105, or MED14736;
(b) the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25); and/or
(c) the anti-PD-L1 antagonist antibody comprises a VH domain comprising the amino acid sequence of SEQ ID NO: 26 and a VL domain comprising the amino acid sequence of SEQ ID NO: 27.

333. The method of claim 332, wherein the anti-PD-L1 antagonist antibody is atezolizumab.

334. The method of claim 330, wherein the PD-1 binding antagonist is an anti-PD-1 antagonist antibody.

335. The method of claim 311, wherein the taxane is paclitaxel or nab-paclitaxel.

336. The method of claim 311, wherein the platinum agent is cisplatin or carboplatin.

337. The method of claim 311, wherein the method comprises (a) administering to the subject or population of subjects the PD-1 axis binding antagonist before the anti-TIGIT antagonist antibody; (b) administering to the subject or population of subjects the anti-TIGIT antagonist antibody before the PD-1 axis binding antagonist; or (c) administering to the subject or population of subjects the anti-TIGIT antagonist antibody and the PD-1 axis binding antagonist simultaneously.

338. The method of claim 311, wherein the anti-TIGIT antagonist antibody and the PD-1 axis binding antagonist are administered before the taxane and/or the platinum agent.

339. The method of claim 338, wherein the method comprises administering to the subject or population of subjects the taxane before the platinum agent.

340. The method of claim 311, wherein an ESCC tumor sample obtained from the subject or population of subjects has been determined to have a detectable expression level of PD-L1.

341. The method of claim 340, wherein the detectable expression level of PD-L1 is a detectable protein expression level of PD-L1 or a detectable nucleic acid expression level of PD-L1.

342. The method of claim 311, wherein the advanced ESCC is a locally advanced ESCC; a recurrent or metastatic ESCC; or an unresectable ESCC.

343. The method of claim 311, wherein the treatment results in:
(a) an increase in a PFS, OS, and/or DOR in the subject or population of subjects as compared to treatment with the taxane and the platinum agent, without the PD-1 axis binding antagonist and the anti-TIGIT antagonist antibody;
(b) an increase in OS of the subject or population of subjects as compared to treatment with the taxane and the platinum agent, without the PD-1 axis binding antagonist and the anti-TIGIT antagonist antibody; or
(c) an increase in duration of objective response (DOR) in the subject or population of subjects as compared to treatment with the taxane and the platinum agent, without the PD-1 axis binding antagonist and the anti-TIGIT antagonist antibody.

344. The method of claim 311, wherein the treatment results in a complete response or a partial response.


SEQUENCE LISTING (34 sequences)

SEQ ID NO: 1 - protein, 7 aa, Artificial Sequence
  Synthetic Construct
SNSAAWN

SEQ ID NO: 2 - protein, 18 aa, Artificial Sequence
  Synthetic Construct
KTYYRFKWYSDYAVSVKG

SEQ ID NO: 3 - protein, 14 aa, Artificial Sequence
  Synthetic Construct
ESTTYDLLAGPFDY

SEQ ID NO: 4 - protein, 17 aa, Artificial Sequence
  Synthetic Construct
KSSQTVLYSSNNKKYLA

SEQ ID NO: 5 - protein, 7 aa, Artificial Sequence
  Synthetic Construct
WASTRES

SEQ ID NO: 6 - protein, 9 aa, Artificial Sequence
  Synthetic Construct
QQYYSTPFT

SEQ ID NO: 7 - protein, 23 aa, Artificial Sequence
  Synthetic Construct
DIVMTQSPDSLAVSLGERATINC

SEQ ID NO: 8 - protein, 15 aa, Artificial Sequence
  Synthetic Construct
WYQQKPGQPPNLLIY

SEQ ID NO: 9 - protein, 32 aa, Artificial Sequence
  Synthetic Construct
GVPDRFSGSGSGTDFTLTISSLQAEDVAVYYC

SEQ ID NO: 10 - protein, 10 aa, Artificial Sequence
  Synthetic Construct
FGPGTKVEIK

SEQ ID NO: 11 - protein, 30 aa, Artificial Sequence
  Synthetic Construct
  MISC_FEATURE (1)..(1): Xaa is Gln or Glu
XVQLQQSGPGLVKPSQTLSLTCAISGDSVS

SEQ ID NO: 12 - protein, 14 aa, Artificial Sequence
  Synthetic Construct
WIRQSPSRGLEWLG

SEQ ID NO: 13 - protein, 32 aa, Artificial Sequence
  Synthetic Construct
RITINPDTSKNQFSLQLNSVTPEDTAVFYCTR

SEQ ID NO: 14 - protein, 11 aa, Artificial Sequence
  Synthetic Construct
WGQGTLVTVSS

SEQ ID NO: 15 - protein, 30 aa, Artificial Sequence
  Synthetic Construct
EVQLQQSGPGLVKPSQTLSLTCAISGDSVS

SEQ ID NO: 16 - protein, 30 aa, Artificial Sequence
  Synthetic Construct
QVQLQQSGPGLVKPSQTLSLTCAISGDSVS

SEQ ID NO: 17 - protein, 126 aa, Artificial Sequence
  Synthetic Construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, Artificial Sequence
  Synthetic Construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, Artificial Sequence
  Synthetic Construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 10 aa, Artificial Sequence
  Synthetic Construct
GFTFSDSWIH

SEQ ID NO: 21 - protein, 18 aa, Artificial Sequence
  Synthetic Construct
AWISPYGGSTYYADSVKG

SEQ ID NO: 22 - protein, 9 aa, Artificial Sequence
  Synthetic Construct
RHWPGGFDY

SEQ ID NO: 23 - protein, 11 aa, Artificial Sequence
  Synthetic Construct
RASQDVSTAVA

SEQ ID NO: 24 - protein, 7 aa, Artificial Sequence
  Synthetic Construct
SASFLYS

SEQ ID NO: 25 - protein, 9 aa, Artificial Sequence
  Synthetic Construct
QQYLYHPAT

SEQ ID NO: 26 - protein, 118 aa, Artificial Sequence [sequence omitted]

SEQ ID NO: 27 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 28 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 456 aa, Homo sapiens
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Homo sapiens
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC